(19) United States
(12) Patent Application Publication
(10) Pub. No.: US 2019/0382849 A1
(43) Pub. Date: Dec. 19, 2019

(54) METHODS, ARRAYS AND USES THEREOF

(71) Applicant: IMMUNOVIA AB, Lund (SE)

(72) Inventors: Carl Borrebaeck, Lund (SE); Linda Dexlin Mellby, Dalby (SE); Andreas Nyberg, Malmo (SE); Christer Wingren, Sodra Sandby (SE)

(21) Appl. No.: 16/479,064

(22) PCT Filed: Jan. 31, 2018
(86) PCT No.: PCT/EP2018/052423

§ 371 Date: Jul. 18, 2019

(30) Foreign Application Priority Data
Jan. 31, 2017 (GB) 1701572.8

(51) Int. Cl. C12Q 1/6886; G16H 50/30; C12Q 1/6813

(52) U.S. Cl. CPC C12Q 1/6886; C12Q 2600/158; C12Q 1/6813; G16H 50/30

(57) ABSTRACT

The present invention provides a method for diagnosing or determining a pancreatic cancer-associated disease state comprising or consisting of the steps of: (a) providing a sample from an individual to be tested; and (b) determining a biomarker signature of the test sample by measuring the presence and/or amount in the test sample of one or more biomarker selected from the group defined in Table A; wherein the presence and/or amount in the test sample of the one or more biomarker selected from the group defined in Table A is indicative of the pancreatic cancer associated disease in the individual; uses and methods of determining a pancreatic cancer-associated disease state, and methods of treating pancreatic cancer, together with arrays and kits for use in the same.

### FIELD OF INVENTION

The present invention provides in vitro methods for determining a pancreatic cancer-associated disease state (such as pancreatic cancer presence, pancreatic cancer risk, pancreatic cancer stage and/or presence of related lesions such as intraductal papillary mucinous neoplasms), as well as arrays and kits for use in such methods.

### BACKGROUND

The incidence of pancreatic ductal adenocarcinoma (PDAC) is increasing and has been the cause of death in 330,400 patients worldwide¹. PDAC is one of the most lethal cancers with a five-year survival of less than 10%²⁻⁴. In 2030 PDAC is thought to become the second leading cause of death of cancer⁵. One factor behind this dismal development is diffuse symptoms resulting in late diagnosis, when only approximately 15% of patients present with a resectable tumor^{2-4, 6, 7}. Consequently, since surgical resection is the only potentially curative treatment for PDAC, earlier detection is required. In line with this, if localized tumors could be resected the five-year survival has been shown to increase from 43% (stage II) to over 50% (stage 1)⁸. Pancreatic tumors have furthermore been reported to be resectable at an asymptomatic stage, six months prior to clinical diagnosis^{9,10.} A recent surveillance study of asymptomatic high-risk patients carrying the CDKN2A mutation resulted in a 75% resection rate and a 24% five-year survival, which is much improved compared to sporadic PDAC patients¹¹. Taken together, it is reasonable to believe that earlier diagnosis would result in increased survival for patients with PDAC^{12, 13} and that asymptomatic high-risk patients would benefit from effective surveillance¹⁴.

The most evaluated biomarker for PDAC thus far, serum CA19-9, suffers from inadequate specificity, with elevated levels in several other indications, as well as a complete absence in patients that are genotypically Lewis a⁻b⁻ (5% of the population). Consequently, the use of CA19-9 by itself is not recommended for screening¹⁵, or as evidence of recurrence¹⁶, but is recommended for disease monitoring after e.g. surgical resection¹⁷. Therefore, the field of cancer diagnostics is increasingly focusing on multiparametric analysis^{18,19} of markers in both diagnostic^{20, 21} and pre-diagnostic samples^{22, 23}, since this approach yields improved sensitivity and specificity, also in combination with CA19-9^{24, 25}. In fact, it has been demonstrated that combinations of immunoregulatory and cancer-associated protein biomarkers can discriminate between late stage III/IV PDAC patients and healthy controls^{26, 27}.

However, there remains a need for improved methods of diagnosing pancreatic cancers such as PDAC, particularly in the early stages of the disease.

### SUMMARY OF THE INVENTION

Accordingly, a first aspect of the invention provides a method for diagnosing or determining a pancreatic cancer-associated disease state comprising or consisting of the steps of:
(a) providing a sample from an individual to be tested; and
(b) determining a biomarker signature of the test sample by measuring the presence and/or amount in the test sample of one or more biomarker(s) selected from the group defined in Table A;

wherein the presence and/or amount in the test sample of the one or more biomarkers selected from the group defined in Table A is indicative of the pancreatic cancer-associated disease state in the individual.

TABLE A
Part (i)
Disks large homolog 1 (DLG1; e.g. UniProt ID Q12959)
Protein kinase C zeta type (PRKCZ; e.g. UniProt ID Q05513)
Part (ii)
Vascular endothelial growth factor (VEGF; e.g. UniProt ID P15692)
Complement C3 (C3; e.g. UniProt ID P01024)
Plasma protease C1 inhibitor (C1INH; e.g. UniProt ID P05155)
Interleukin-4 (IL-4; e.g. UniProt ID P05112)
Interferon gamma (IFNγ; e.g. UniProt ID P01579)
Complement C5 (C5; e.g. UniProt ID P01031)
Protein-tyrosine kinase 6 (PTK6; e.g. UniProt ID Q13882)
Part (iii)
Calcineurin B homologous protein 1 (CHP1; e.g. UniProt ID Q99653)
GTP-binding protein GEM (GEM; e.g. UniProt ID P55040)
Aprataxin and PNK-like factor (APLF; e.g. UniProt ID Q8IW19)
Calcium/calmodulin-dependent protein kinase type IV (CAMK4; e.g.
UniProt ID Q16566)
Membrane-associated guanylate kinase, WW and PDZ domain-containing
protein 1 (MAGI; e.g. UniProt ID Q96QZ7)
Serine/threonine-protein kinase MARK1 (MARK1; e.g. UniProt ID
Q9P0L2)
PR domain zinc finger protein 8 (PRDM8; e.g. UniProt ID Q9NQV8)
Part (iv)
Apolipoprotein A1 (APOA1; e.g. UniProt ID P02647)
Cyclin-dependent kinase 2 (CDK2; e.g. UniProt ID P24941)
HADH2 protein (HADH2; e.g. UniProt ID Q6IBS9)
Interleukin-6 (IL-6; e.g. UniProt ID P05231)
Complement C4 (C4; e.g. UniProt ID P0COL4/5)
Visual system homeobox 2 (VSX2/CHX10; e.g. UniProt ID P58304)
Intercellular adhesion molecule 1 (ICAM-1; e.g. UniProt ID P05362)
Interleukin-13 (IL-13; e.g. UniProt ID P35225)
Lewis x (Lewis x/CD15)
Myomesin-2 (MYOM2; e.g. UniProt ID P54296)
Properdin (Factor P; e.g. UniProt ID P27918)
Sialyl Lewis x (Sialyl Lewis x)
Lymphotoxin-alpha (TNFβ; e.g. UniProt ID P01374)

Thus, in one embodiment, the method comprises determining a biomarker signature of the test sample, which enables a diagnosis to be reached in respect of the individual from which the sample is obtained.

The methods of the invention are suitable for testing a sample from any individual who is suspected of having, or at risk of developing, a pancreatic cancer-associated disease state.

For example, the individual may be from one of the following groups with an elevated risk of having or developing pancreatic cancer:
(i) Individuals with a family history of pancreatic cancer (e.g. within one or two generations on either the maternal or paternal side);
(ii) Individuals diagnosed with new-onset diabetes (e.g. type II), especially those aged 50 years or over; and
(iii) Individuals with symptoms suggestive or consistent with pancreatic cancer, e.g. pain in the upper abdomen or upper back, loss of appetite, weight loss, jaundice (yellow skin and eyes, and dark urine), indigestion, nausea, vomiting and/or extreme tiredness (fatigue)).

By “pancreatic cancer-associated disease state” we include pancreatic cancer presence per se, the risk of having or of developing pancreatic cancer, pancreatic cancer stage and presence of related lesions such as intraductal papillary mucinous neoplasms (see below). In particular, we include the presence and/or stage of pancreatic ductal adenocarcinoma (PDAC).

Thus, in one embodiment, the methods of the invention provide a qualitative result for the detection of pancreatic abnormalities in individuals with increased risk of developing PDAC. In specific embodiment, the methods of the invention permit:
(a) the diagnosis and/or staging of early pancreatic cancer; and
(b) the diagnosis and/or staging of late pancreatic cancer.

Advantageously, the methods of the invention also enable the differentiation between pancreatic cancer and chronic pancreatitis in an individual.

In a further embodiment, the methods of the invention may be used to detect the presence in an individual of intraductal papillary mucinous neoplasms (IPMN). Such lesions, if left untreated, can progress to invasive cancer. Consequently, it is important to detect these lesions, since this may present an opportunity to remove a premalignant lesion. In one embodiment, the IPMN lesions are malignant.

By “biomarker” we include any naturally-occurring biological molecule, or component or fragment thereof, the measurement of which can provide information useful in the diagnosis of pancreatic cancer. Thus, in the context of Table A, the biomarker may be the protein, or a polypeptide fragment or carbohydrate moiety thereof (or, in the case of sialyl Lewis x, a carbohydrate moiety per se). Alternatively, the biomarker may be a nucleic acid molecule, such as a mRNA, cDNA or circulating tumour DNA molecule, which encodes the protein or part thereof.

By “diagnosis” we include determining the presence or absence of a disease state in an individual (e.g., determining whether an individual is or is not suffering from early stage pancreatic cancer or late stage pancreatic cancer).

By “staging” we include determining the stage of a pancreatic cancer, for example, determining whether the pancreatic cancer is stage I, stage II, stage III or stage IV (e.g., stage I, stage II, stage I-II, stage III-IV or stage I-IV).

By “early pancreatic cancer” (or “early stage pancreatic cancer”) we include or mean pancreatic cancer comprising or consisting of stage I and/or stage II pancreatic cancer, for example as determined by the American Joint Committee on Cancer (AJCC) TNM system (e.g., see: http://www.cancer.orq/cancer/pancreaticcancer/detailedquide/pancreatic-cancer-staging and AJCC Cancer Staging Manual (7^{th} ed.), 2011, Edge et al., Springer which are incorporated by reference herein).

The TNM cancer staging system is based on 3 key pieces of information:
T describes the size of the main (primary) tumour and whether it has grown outside the pancreas and into nearby organs.
N describes the spread to nearby (regional) lymph nodes.
M indicates whether the cancer has metastasized (spread) to other organs of the body. (The most common sites of pancreatic cancer spread are the liver, lungs, and the peritoneum—the space around the digestive organs.)

Numbers or letters appear after T, N, and M to provide more details about each of these factors.

T Categories
TX: The main tumour cannot be assessed.
T0: No evidence of a primary tumour.
Tis: Carcinoma in situ (the tumour is confined to the top layers of pancreatic duct cells). (Very few pancreatic tumours are found at this stage.)
T1: The cancer is still within the pancreas and is 2 centimetres (cm) (about % inch) or less across.
T2: The cancer is still within the pancreas but is larger than 2 cm across.
T3: The cancer has grown outside the pancreas into nearby surrounding tissues but not into major blood vessels or nerves.
T4: The cancer has grown beyond the pancreas into nearby large blood vessels or nerves.

N Categories
NX: Nearby (regional) lymph nodes cannot be assessed.
N0: The cancer has not spread to nearby lymph nodes.
N1: The cancer has spread to nearby lymph nodes.

M Categories
M0: The cancer has not spread to distant lymph nodes (other than those near the pancreas) or to distant organs such as the liver, lungs, brain, etc.
M1: The cancer has spread to distant lymph nodes or to distant organs.

Once the T, N, and M categories have been determined, this information is combined to assign an overall stage of 0, I, II, III, or IV (sometimes followed by a letter). This process is called stage grouping.

Stage 0 (Tis, N0, M0):

The tumour is confined to the top layers of pancreatic duct cells and has not invaded deeper tissues. It has not spread outside of the pancreas. These tumours are sometimes referred to as pancreatic carcinoma in situ.

Stage IA (T1, N0, M0): The tumour is confined to the pancreas and is 2 cm across or smaller (T1). It has not spread to nearby lymph nodes (NO) or distant sites (M0).

Stage IB (T2, N0, M0): The tumour is confined to the pancreas and is larger than 2 cm across (T2). It has not spread to nearby lymph nodes (NO) or distant sites (M0).

Stage IIA (T3, N0, M0): The tumour is growing outside the pancreas but not into major blood vessels or nerves (T3). It has not spread to nearby lymph nodes (NO) or distant sites (M0).

Stage IIB (T1-3, N1, M0): The tumour is either confined to the pancreas or growing outside the pancreas but not into major blood vessels or nerves (T1-T3). It has spread to nearby lymph nodes (N1) but not to distant sites (M0).

Stage III (T4, Any N, M0): The tumour is growing outside the pancreas into nearby major blood vessels or nerves (T4). It may or may not have spread to nearby lymph nodes (Any N). It has not spread to distant sites (M0).

Stage IV (Any T, Any N, M1): The cancer has spread to distant sites (M1).

Alternatively or additionally, by “early pancreatic cancer” (or “early stage pancreatic cancer”) we include or mean asymptomatic pancreatic cancer. Common presenting symptoms of pancreatic cancers include jaundice (for tumours of the pancreas head), abdominal pain, weight loss, steatorrhoea, and new-onset diabetes. For example, the pancreatic cancer may be present at least 1 week before symptoms (e.g., common symptoms) are observed or observable, for example, ≥2 weeks, ≥3 weeks, ≥4 weeks, ≥5 weeks, ≥6 weeks, ≥7 weeks, ≥8 weeks, ≥3 months, ≥4 months, ≥5 months, ≥6 months, ≥7 months, 28 months, ≥9 months, ≥10 months, ≥11 months, ≥12 months, ≥18 months, 22 years, ≥3 years, ≥4 years, or ≥5 years, before symptoms are observed or observable.

Thus, by “early pancreatic cancer” (or “early stage pancreatic cancer”) we include pancreatic cancers that are of insufficient size and/or developmental stage to be diagnosed by conventional clinical methods. For example, by “early pancreatic cancer” or “early stage pancreatic cancer” we include or mean pancreatic cancers present at least 1 week before the pancreatic cancer is diagnosed or diagnosable by conventional clinical methods, for example, 22 weeks, ≥3 weeks, ≥4 weeks, ≥5 weeks, ≥6 weeks, ≥7 weeks, ≥8 weeks, 23 months, ≥4 months, ≥5 months, ≥6 months, ≥7 months, ≥8 months, ≥9 months, ≥10 months, ≥11 months, ≥12 months, ≥18 months, 22 years, 23 years, ≥4 years, or ≥5 years, before the pancreatic cancer is diagnosed or diagnosable by convention clinical methods.

The contemporary best practice for clinical pancreatic cancer diagnosis will be well known to the person of skill in the art, however, for a detailed review see Ducreux et al., 2015, ‘Cancer of the pancreas: ESMO Clinical Practice Guidelines for diagnosis, treatment and follow-up’ Annals of Oncology, 26 (Supplement 5): v56-v68 which is incorporated by reference herein.

Conventional clinical diagnoses (e.g., “diagnosed by conventional clinical methods”) include CT scan, ultrasound, endoscopic ultrasound, biopsy (histopathology) and/or physical examination (e.g., of the abdomen and, possibly, local lymph nodes). In one embodiment by “conventional clinical diagnoses” (and the like) we include the pancreatic cancer diagnosis procedures set out in Ducreux et al., 2015, supra.

Conventional clinical diagnoses (and the like) may include or exclude the use of molecular biomarkers present in bodily fluids (such as blood, serum, interstitial fluid, lymph, urine, mucus, saliva, sputum, sweat) and or tissues.

It will be appreciated by persons skilled in the art that the early pancreatic cancer may be a resectable pancreatic cancer.

By “resectable pancreatic cancer” we include or mean that the pancreatic cancer comprises or consists of tumours that are (and/or are considered) capable of being removed by surgery (i.e., are resectable). For example, the pancreatic cancer may be limited to the pancreas (i.e., it does not extend beyond the pancreas and/or have not metastasised).

In one embodiment, the early pancreatic cancer comprises tumours of 30 mm or less in all dimensions (i.e., in this embodiment individuals with early pancreatic cancer do not comprise pancreatic cancer tumours of greater than 30 mm in any dimension), for example, equal to or less than 29 mm, 28 mm, 27 mm, 26 mm, 25 mm, 24 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm or equal to or 0.1 mm in all dimensions. Alternatively or additionally, the pancreatic cancer tumours of 30 mm or less in all dimensions are at least 2 mm in one dimension. Alternatively or additionally, the pancreatic cancer tumours of 30 mm or less in all dimensions are at least 2 mm all dimensions.

It will be appreciated by persons skilled in the art that the methods of the invention will typically be used to provide an initial diagnosis, for example to identify an individual at risk of having or developing pancreatic cancer, after which further clinical investigations (such as biopsy testing, in vivo imaging and the like) may be performed to confirm the diagnosis.

Alternatively, however, the methods of the invention may be used as a stand-alone diagnostic test.

By “sample to be tested”, “test sample” or “control sample” we include a tissue or fluid 5 sample taken or derived from an individual, wherein the sample comprises endogenous proteins and/or nucleic acid molecules and/or carbohydrate moieties. Preferably the sample to be tested is provided from a mammal. The mammal may be any domestic or farm animal. Preferably, the mammal is a rat, mouse, guinea pig, cat, dog, horse or a primate. Most preferably, the mammal is human.

The sample to be tested in the methods of the invention may be a cell, tissue or fluid sample (or derivative thereof) comprising or consisting of blood (fractionated or unfractionated), plasma, plasma cells, serum, tissue cells or equally preferred, protein or nucleic acid derived from a cell or tissue sample. It will be appreciated that the test and control samples should be derived from the same species. Preferably, test and control samples are matched for age, gender and/or lifestyle.

In one embodiment, the sample is a pancreatic tissue sample. In an alternative or additional embodiment, the sample is a sample of pancreatic cells.

Alternatively, the sample may be a blood or serum sample.

In the methods of the invention, step (b) comprises or consists of measuring the presence and/or amount of one or more biomarker(s) listed in Table A, for example at least 2, 3, 4, 5, 6, 7, 8, 9, 10, 11, 12, 13, 14, 15, 16, 17, 18, 19, 20, 21, 22, 23, 24, 25, 26, 27, 28, or all 29 of the biomarkers listed in Table A.

Thus, step (b) may comprise, consist of or exclude measuring the expression of Disks large homolog 1 (DLG1). Alternatively or additionally, step (b) comprises, consists of or excludes measuring the expression of Protein kinase C zeta type (PRKCZ). Alternatively or additionally, step (b) comprises, consists of or excludes measuring the expression of vascular endothelial growth factor (VEGF). Alternatively or additionally, step (b) comprises, consists of or excludes measuring the expression of Complement C3 (C3). Alternatively or additionally, step (b) comprises, consists of or excludes measuring the expression of Plasma protease C1 inhibitor (C1INH). Alternatively or additionally, step (b) comprises, consists of or excludes measuring the expression of Interleukin-4 (IL-4). Alternatively or additionally, step (b) comprises, consists of or excludes measuring the expression of Interferon gamma (IFNγ). Alternatively or additionally, step (b) comprises, consists of or excludes measuring the expression of Complement C5 (C5). Alternatively or additionally, step (b) comprises, consists of or excludes measuring the expression of Protein-tyrosine kinase 6 (PTK6). Alternatively or additionally, step (b) comprises, consists of or excludes measuring the expression of Calcineurin B homologous protein 1 (CHP1). Alternatively or additionally, step (b) comprises, consists of or excludes measuring the expression of GTP-binding protein GEM (GEM). Alternatively or additionally, step (b) comprises, consists of or excludes measuring the expression of Aprataxin and PNK-like factor (APLF). Alternatively or additionally, step (b) comprises, consists of or excludes measuring the expression of Calcium/calmodulin-dependent protein kinase type IV (CAMK4). Alternatively or additionally, step (b) comprises, consists of or excludes measuring the expression of Membrane-associated guanylate kinase, WW and PDZ domain-containing protein 1 (MAGI). Alternatively or additionally, step (b) comprises, consists of or excludes measuring the expression of Serine/threonine-protein kinase MARK1 (MARK1). Alternatively or additionally, step (b) comprises, consists of or excludes measuring the expression of domain zinc finger protein 8 (PRDM8). Alternatively or additionally, step (b) comprises, consists of or excludes measuring the expression of Apolipoprotein A1 (APOA1). Alternatively or additionally, step (b) comprises, consists of or excludes measuring the expression of Cyclin-dependent kinase 2 (CDK2). Alternatively or additionally, step (b) comprises, consists of or excludes measuring the expression of HADH2 protein (HADH2). Alternatively or additionally, step (b) comprises, consists of or excludes measuring the expression of Interleukin-6 (IL-6). Alternatively or additionally, step (b) comprises, consists of or excludes measuring the expression of Complement C4 (C4). Alternatively or additionally, step (b) comprises, consists of or excludes measuring the expression of Visual system homeobox 2 (VSX2/CHX10). Alternatively or additionally, step (b) comprises, consists of or excludes measuring the expression of Intercellular adhesion molecule 1 (ICAM-1). Alternatively or additionally, step (b) comprises, consists of or excludes measuring the expression of Interleukin-13 (IL-13). Alternatively or additionally, step (b) comprises, consists of or excludes measuring the expression of Lewis x (Lewis x/CD15). Alternatively or additionally, step (b) comprises, consists of or excludes measuring the expression of Myomesin-2 (MYOM2). Alternatively or additionally, step (b) comprises, consists of or excludes measuring the expression of Properdin (Factor P). Alternatively or additionally, step (b) comprises, consists of or excludes measuring the expression of Sialyl Lewis x (Sialyl Lewis x). Alternatively or additionally, step (b) comprises, consists of or excludes measuring the expression of Lymphotoxin-alpha (TNFβ).

Thus, step (b) may comprise or consist of measuring the presence and/or amount of one or more biomarker(s) listed in:
(i) Table A, part (i), for example both of the biomarkers listed in Table A(i); and/or
(ii) Table A, part (ii), for example at least 2, 3, 4, 5, 6, 7 or all of the biomarkers listed in Table A(ii); and/or
(iii) Table A, part (iii), for example at least 2, 3, 4, 5, 6 or all of the biomarkers listed in Table A(iii); and/or
(iv) Table A, part (iv), for example at least 2, 3, 4, 5, 6, 7, 8, 9, 10, 11, 12 or all of the biomarkers listed in Table A(iv).

In a further preferred embodiment, the step (b) may comprise or consist of measuring the presence and/or amount of one or more of the following biomarker(s):
(i) the biomarkers listed in Table A, and Complement C1q (C1q; e.g. Uniprot ID P02745, 2746 and/or 2747);
(ii) the biomarkers listed in Table A, excluding Interleukin-6 (IL-6) and/or GTP-binding protein GEM (GEM); and/or
(iii) the biomarkers listed in Table A (excluding IL-6 and GEM) and C1q.

In this sense, Complement C1q may be considered as an additional biomarker within Table A, part (iv) and/or IL-6 and GEM may be considered as biomarkers within Table B (rather than Table A).

Thus, in alternative embodiments of all the aspects of the invention, references herein to the biomarkers in Table A may be regarded as being references to biomarkers listed in Table A (excluding IL-6 and GEM) and C1q. Likewise, references herein to the biomarkers in Table B may be regarded as being references to biomarkers listed in Table B plus IL-6 and GEM, but excluding C1q.

Advantageously, in the methods of the first aspect of the invention, step (b) comprises or consists of determining a biomarker signature of the test sample by measuring the presence and/or amount in the test sample of all of the following biomarkers:
DLG1, PRKCZ, VEGF, C3, C1INH, IL-4, IFNγ, C5, PTK6, CHP1, APLF, CAMK4, MAGI, MARK1, PRDM8, APOA1, CDK2, HADH2, C4, VSX2/CHX10, ICAM-1, IL-13, Lewis x/CD15, MYOM2, Factor P, Sialyl Lewis x, TNF and Complement C1q
(optionally including one or more biomarkers from Table B and/or IL-6 and/or GEM; see below),

wherein the presence and/or amount in the test sample of said biomarkers is indicative of the pancreatic cancer-associated disease state in the individual.

It will be appreciated that step (b) may additionally comprise measuring the presence and/or amount of one or more further biomarkers not listed in Table A, wherein the further biomarkers may provide additional diagnostic information.

For example, step (b) may comprise or consist of measuring the presence and/or amount of one or more biomarker(s) listed in Table B.

TABLE B

| Short name | Full name |
|---|---|
| AKT3 | RAC-gamma serine/threonine-protein kinase |
| Angiomotin | Angiomotin |
| ANM5 | Protein arginine N-methyltransferase 5 |
| APOA4 | Apolipoprotein A4 |
| ApoB-100 | Apolipoprotein B-100 |
| ARHGC | Rho guanine nucleotide exchange factor 12 |
| B-galactosidase | Beta-galactosidase |
| BIRC2 | Baculoviral IAP repeat-containing protein 2 |
| BTK | Tyrosine-protein kinase BTK |
| C1q | Complement C1q |
| CA 19-9 | CA 19-9 |
| CD40 | CD40 Arf-GAP with GTPase, ANK repeat and PH |
| CENTG1 | domain-containing protein 2 |
| CSNK1E | Casein kinase I isoform epsilon |
| Cystatin C | Cystatin C |
| DCNL1 | DCN1-like protein 1 |
| DLG2 | Disks large homolog 2 |
| DLG4 | Disks large homolog 4 |
| DPOLM | DNA-directed DNA/RNA polymerase mu |
| DUSP7 | Dual specificity protein phosphatase 7 |
| Eotaxin | Eotaxin |
| FASN | FASN protein |
| FER | Tyrosine-protein kinase Fer |
| GAK | GAK protein |
| GLP-1R | Glucagon-like peptide 1 receptor |
| GM-CSF | GM-CSF |
| GNAI3 | Guanine nucleotide-binding protein G(k) subunit alpha |
| GORS2 | Golgi reassembly-stacking protein 2 |
| GPRK5 | G protein-coupled receptor kinase 5 |
| Her2/ErbB2 | Receptor tyrosine-protein kinase erbB-2 |
| HLA-DR/DP | HLA-DR/DP |
| IgM | IgM |
| IL-10 | Interleukin-10 |
| IL-11 | Interleukin-11 |
| IL-12 | Interleukin-12 |
| IL-16 | Interleukin-16 |
| IL-18 | Interleukin-18 |
| IL-1a | Interleukin-1a |
| IL-1b | Interleukin-1b |
| IL-1ra | Interleukin-1ra |
| IL-2 | Interleukin-2 |
| IL-3 | Interleukin-3 |
| IL-5 | Interleukin-5 |
| IL-7 | Interleukin-7 |
| IL-8 | Interleukin-8 |
| IL-9 | Interleukin-9 |
| Integrin α-10 | Integrin alpha-10 |
| ITCH | E3 ubiquitin-protein ligase Itchy homolog |
| JAK3 | Tyrosine-protein kinase JAK3 |
| Keratin 19 | Keratin, type I cytoskeletal 19 |
| KIAA0882 | TBC1 domain family member 9 |
| KKCC1 | Calcium/calmodulin-dependent protein kinase 1 |
| KSYK | Tyrosine-protein kinase SYK |
| Leptin | Leptin |
| Lewis y | Lewis y |
| LIN7A | Protein lin-7 homolog A |
| MAP2K2 | Dual specificity mitogen-activated protein kinase 2 |
| MAP2K6 | Dual specificity mitogen-activated protein kinase 6 |
| MAPK1 | Mitogen-activated protein kinase 1 |
| MAPK8 | Mitogen-activated protein kinase 8 |
| MCP-1 | C-C motif chemokine 2 |
| MCP-4 | C-C motif chemokine 13 |
| Mucin-1 | Mucin-1 |
| NOS1 | Nitric oxide synthase, brain |
| OSBPL3 | Oxysterol-binding protein-related protein 3 |
| OTU6B | OTU domain-containing protein 6B |
| OTUB1 | Ubiquitin thioesterase OTUB1 |
| OTUB2 | Ubiquitin thioesterase OTUB2 |
| PAK4 | Serine/threonine-protein kinase PAK 4 |
| PAK5 | Serine/threonine-protein kinase PAK 7 |
| PARP6 | Partitioning defective 6 homolog beta |
| PGAM5 | Serine/threonine-protein phosphatase PGAM5, |
| | mitochondrial |
| PRKG2 | cGMP-dependent protein kinase 2 |
| Procathepsin W | Cathepsin W |
| PSA | Prostate-specific antigen |
| PTN13 | Tyrosine-protein phosphatase non-receptor type 13 |
| PTPN1 | Tyrosine-protein phosphatase non-receptor type 1 |
| PTPRD | Receptor-type tyrosine-protein phosphatase delta |
| PTPRJ | Receptor-type tyrosine-protein phosphatase eta |
| PTPRK | Receptor-type tyrosine-protein phosphatase kappa |
| PTPRN2 | Receptor-type tyrosine-protein phosphatase N2 |
| PTPRT | Receptor-type tyrosine-protein phosphatase T |
| RANTES | C-C motif chemokine 5 |
| RPS6KA2 | Ribosomal protein S6 kinase alpha-2 |
| SHC1 | SHC-transforming protein 1 |
| Sox11a | Transcription factor SOX-11 |
| SPDLY | Protein Spindly |
| TGF-b1 | Transforming growth factor beta-1 |
| TNF-a | Tumor necrosis factor |
| TNFRSF14 | Tumor necrosis factor receptor superfamily member 14 |
| TNFRSF3 | Tumor necrosis factor receptor superfamily member 3 |
| UBP7 | Ubiquitin carboxyl-terminal hydrolase 7 |
| UCHL5 | Ubiquitin carboxyl-terminal hydrolase isozyme L5 |
| UPF3B | Regulator of nonsense transcripts 3B |

For example, step (b) may comprise or consist of measuring the presence and/or amount of at least 2, 3, 4, 5, 6, 7, 8, 9, 10, 15, 20, 25, 30, 35, 40, 45, 50, 55, 60, 65, 70, 75, 80, 85, 90 or all of the biomarkers in Table B.

In one embodiment of the invention, the method is for the diagnosis of early stage pancreatic cancer (e.g., stage I and/or stage II PDAC versus healthy).

For example, step (b) may comprise or consist of measuring the presence and/or amount of one or more biomarker(s) listed in Table A, for example at least 2, 3, 4, 5, 6, 7, 8, 9, 10, 11, 12, 13, 14, 15, 16, 17, 18, 19, 20, 21, 22, 23, 24, 25, 26, 27, 28 or all of the biomarkers in Table A.

Alternatively, or in addition, step (b) may comprise or consist of measuring the presence and/or amount of one or more biomarker(s) listed in Table C, for example at least 2, 3, 4, 5, 6, 7, 8, 9, 10, 11, 12, 13, 14, 15, 16, 17, 18, 19, 20, 21, 22, 23, 24 or all of the biomarkers in Table C.

TABLE C
Selected biomarkers for classification between
non-cancerous and PDAC stages I and II

| Score Rank | Protein Name |
|---|---|
| 1 | Plasma protease C1 inhibitor |
| 2 | Interleukin-4 |
| 3 | Protein-tyrosine kinase 6 |
| 4 | Complement C3 |
| 5 | Serine/threonine-protein kinase MARK1 |
| 6 | HADH2 protein |
| 7 | Properdin |
| 8 | Complement C4 |
| 9 | Cyclin-dependent kinase 2 |
| 10 | Interferon gamma |
| 11 | Calcium/calmodulin-dependent protein kinase kinase 1 |
| 12 | Complement C5 |
| 13 | Vascular endothelial growth factor |
| 14 | Visual system homeobox 2 |
| 15 | PR domain zinc finger protein 8 |
| 16 | Intercellular adhesion molecule 1 |
| 17 | Ubiquitin carboxyl-terminal hydrolase isozyme L5 |
| 18 | Interleukin-6 |
| 19 | Myomesin-2 |
| 20 | Aprataxin and PNK-like factor |
| 21 | Apolipoprotein A1 |
| 22 | Regulator of nonsense transcripts 3B |
| 23 | Lumican |
| 24 | Interleukin-9 |
| 25 | C-C motif chemokine 13 |

In an alternative embodiment of the invention, the method is for the diagnosis of late stage pancreatic cancer (e.g., stage Ill and/or stage IV PDAC versus healthy).

For example, step (b) may comprise or consist of measuring the presence and/or amount of one or more biomarker(s) listed in Table D, for example at least 2, 3, 4, 5, 6, 7, 8, 9, 10, 11, 12, 13, 14, 15, 16, 17, 18, 19, 20, 21, 22, 23 or all of the biomarkers in Table D.

TABLE D
Selected biomarkers for classification between
non-cancerous and PDAC stages III and IV

| Score Rank | Protein Name |
|---|---|
| 1 | Plasma protease C1 inhibitor |
| 2 | Interleukin-4 |
| 3 | Complement C3 |
| 4 | Properdin |
| 5 | Complement C4 |
| 6 | Sialyl Lewis x |
| 7 | Calcineurin B homologous protein 1 |
| 8 | HADH2 protein |
| 9 | Protein-tyrosine kinase 6 |
| 10 | Apolipoprotein A1 |
| 11 | C-C motif chemokine 13 |
| 12 | Membrane-associated guanylate kinase, |
| | WW and PDZ domain-containing protein 1 |
| 13 | Lymphotoxin-alpha |
| 14 | Disks large homolog 1 |
| 15 | Protein kinase C zeta type |
| 16 | Interleukin-13 |
| 17 | Complement C5 |
| 18 | Serine/threonine-protein kinase MARK1 |
| 19 | GTP-binding protein GEM |
| 20 | IgM |
| 21 | Interleukin-8 |
| 22 | Vascular endothelial growth factor |
| 23 | Interleukin-6 |
| 24 | Interleukin-9 |

In a further embodiment of the invention, the method is for differentiating pancreatic cancer from chronic pancreatitis.

For example, step (b) may comprise or consist of measuring the presence and/or amount of one or more biomarker(s) listed in:
(i) Table A, part (i), for example both of the biomarkers listed in Table A(i); and/or
(ii) Table A, part (ii), for example at least 2, 3, 4, 5, 6, 7 or all of the biomarkers listed in Table A(ii); and/or
(iii) Table A, part (iii), for example at least 2, 3, 4, 5, 6 or all of the biomarkers listed in Table A(iii); and/or
(iv) Table A, part (iv), for example at least 2, 3, 4, 5, 6, 7, 8, 9, 10, 11, 12 or all of the biomarkers listed in Table A(iv).

It will be appreciated that step (b) may additionally comprise measuring the presence and/or amount of one or more further biomarkers not listed in Table A, wherein the further biomarkers may provide additional diagnostic information.

For example, step (b) may comprise or consist of measuring the presence and/or amount of one or more biomarker biomarkers selected from the group consisting of IL-4, C4, MAPK9, C1INH, VEGF, PTPRD, KCC4, TNF-α, C1q and BTK.

In a further embodiment of the invention, the method is for detecting intraductal papillary mucinous neoplasms (IPMN) in an individual. In other words, the methods may enable a patient with IPMN to be differentiated from an individual without IPMN, e.g. a healthy individual. In one embodiment, the IPMN lesions are malignant.

For example, step (b) may comprise or consist of measuring the presence and/or amount of one or more biomarker(s) listed in:
(i) Table A, part (i), for example both of the biomarkers listed in Table A(i); and/or
(ii) Table A, part (ii), for example at least 2, 3, 4, 5, 6, 7 or all of the biomarkers listed in Table A(ii); and/or
(iii) Table A, part (iii), for example at least 2, 3, 4, 5, 6 or all of the biomarkers listed in Table A(iii); and/or
(iv) Table A, part (iv), for example at least 2, 3, 4, 5, 6, 7, 8, 9, 10, 11, 12 or all of the biomarkers listed in Table A(iv).

It will be appreciated that step (b) may additionally comprise measuring the presence and/or amount of one or more further biomarkers, such as those listed in Tables B, C and/or D, wherein the further biomarkers may provide additional diagnostic information.

In one preferred embodiment of the first aspect of the invention, step (b) comprises measuring the presence and/or amount of all of the biomarkers listed in Table A, e.g. at the protein level. Use of this ‘full’ consensus biomarker signature allows the diagnosis of pancreatic cancer (e.g., PDAC) at any stage, including early stages of the disease.

It will be appreciated by persons skilled in the art that, in addition to measuring the biomarkers in a sample from an individual to be tested, the methods of the invention may also comprise measuring those same biomarkers in one or more control samples.

Thus, in one embodiment, the method further comprises or consists of the steps of:
(c) providing one or more (negative) control samples from:
   (i) an individual not afflicted with pancreatic cancer; and/or
   (ii) an individual afflicted with pancreatic cancer, wherein the sample was of a different stage to that of that the test sample; and/or
   (iii) an individual afflicted with chronic pancreatitis; and
(d) determining a biomarker signature of the one or more control samples by measuring the presence and/or amount in the control sample of the one or more biomarkers measured in step (b);

wherein the pancreatic cancer-associated disease state is identified in the event that the presence and/or amount in the test sample of the one or more biomarkers measured in step (b) is different from the presence and/or amount in the control sample of the one or more biomarkers measured in step (d).

By “is different to the presence and/or amount in a control sample” we include that the presence and/or amount of the one or more biomarker(s) in the test sample differs from that of the one or more control sample(s) (or to predefined reference values representing the same). Preferably, the presence and/or amount in the test sample differs from the presence or amount in one or more control sample(s) (or mean of the control samples) by at least ±5%, for example, at least ±6%, ±7%, ±8%, +9%, +10%, ±11%, ±12%, ±13%, ±14%, ±15%, ±16%, ±17%, ±18%, ±19%, ±20%, +21%, +22%, +23%, ±24%, ±25%, ±26%, ±27%, ±28%, ±29%, ±30%, ±31%, ±32%, ±33%, +34%, +35%, +36%, ±37%, ±38%, ±39%, ±40%, ±41%, ±42%, ±43%, ±44%, ±45%, ±41%, ±42%, ±43%, ±44%, ±55%, ±60%, ±65%, +66%, +67%, ±68%, ±69%, +70%, +71%, ±72%, +73%, +74%, ±75%, ±76%, ±77%, ±78%, ±79%, +80%, ±81%, ±82%, +83%, +84%, ±85%, +86%, +87%, ±88%, ±89%, ±90%, ±91%, ±92%, ±93%, ±94%, ±95%, ±96%, ±97%, ±98%, +99%, +100%, +125%, ±150%, ±175%, ±200%, ±225%, ±250%, ±275%, ±300%, ±350%, +400%, +500% or at least ±1000% of the one or more control sample(s) (e.g., the negative control sample).

Alternatively or additionally, the presence or amount in the test sample differs from the mean presence or amount in the control samples by at least ≥1 standard deviation from the mean presence or amount in the control samples, for example, ≥1.5, ≥2, ≥3, ≥4, ≥5, ≥6, ≥7, ≥8, ≥9, ≥10, ≥11, ≥12, ≥13, ≥14 or 215 standard deviations from the mean presence or amount in the control samples. Any suitable means may be used for determining standard deviation (e.g., direct, sum of square, Welford's), however, in one embodiment, standard deviation is determined using the direct method (i.e., the square root of [the sum the squares of the samples minus the mean, divided by the number of samples]).

Alternatively or additionally, by “is different to the presence and/or amount in a control sample” we include that the presence or amount in the test sample does not correlate with the amount in the control sample in a statistically significant manner. By “does not correlate with the amount in the control sample in a statistically significant manner” we mean or include that the presence or amount in the test sample correlates with that of the control sample with a p-value of >0.001, for example, >0.002, >0.003, >0.004, >0.005, >0.01, >0.02, >0.03, >0.04 >0.05, >0.06, >0.07, >0.08, >0.09 or >0.1. Any suitable means for determining p-value known to the skilled person can be used, including z-test, t-test, Student's t-test, f-test, Mann-Whitney U test, Wilcoxon signed-rank test and Pearson's chi-squared test.

In one embodiment, the method of the invention may further comprise or consist of the steps of:
(e) providing one or more (positive) control sample from;
   (i) an individual afflicted with pancreatic cancer (i.e., a positive control); and/or
   (ii) an individual afflicted with pancreatic cancer, wherein the sample was of the same stage to that of that the test sample; and
(f) determining a biomarker signature of the control sample by measuring the presence and/or amount in the control sample of the one or more biomarkers measured in step (b);

wherein the pancreatic cancer-associated disease state is identified in the event that the presence and/or amount in the test sample of the one or more biomarkers measured in step (b) corresponds to the presence and/or amount in the control sample of the one or more biomarkers measured in step (f).

Thus, the methods of the invention may comprise steps (c)+(d) and/or steps (e)+(f).

By “corresponds to the presence and/or amount in a control sample” we include that the presence and/or amount is identical to that of a positive control sample; or closer to that of one or more positive control sample than to one or more negative control sample (or to predefined reference values representing the same). Preferably the presence and/or amount is within ±40% of that of the one or more control sample (or mean of the control samples), for example, within ±39%, +38%, ±37%, ±36%, ±35%, ±34%, ±33%, ±32%, ±31%, ±30%, +29%, +28%, ±27%, ±26%, ±25%, +24%, ±23%, ±22%, ±21%, ±20%, ±19%, ±18%, ±17%, ±16%, ±15%, ±14%, ±13%, ±12%, ±11%, ±10%, ±9%, +8%, ±7%, ±6%, ±5%, ±4%, ±3%, ±2%, ±1%, ±0.05% or within 0% of the one or more control sample (e.g., the positive control sample).

Alternatively or additionally, the difference in the presence or amount in the test sample is 55 standard deviation from the mean presence or amount in the control samples, for example, ≤4.5, 54, ≤3.5, ≤3, 52.5, 52, 51.5, ≤1.4, ≤1.3, 51.2, ≤1.1, ≤1, ≤0.9, 0.8, ≤0.7, ≤0.6, ≤0.5, ≤0.4, 50.3, 50.2, 50.1 or 0 standard deviations from the from the mean presence or amount in the control samples, provided that the standard deviation ranges for differing and corresponding biomarker expressions do not overlap (e.g., abut, but no not overlap).

Alternatively or additionally, by “corresponds to the presence and/or amount in a control sample” we include that the presence or amount in the test sample correlates with the amount in the control sample in a statistically significant manner. By “correlates with the amount in the control sample in a statistically significant manner” we mean or include that the presence or amount in the test sample correlates with the that of the control sample with a p-value of ≤0.05, for example, 50.04, 50.03, 50.02, 50.01, 50.005, 50.004, 50.003, ≤0.002, ≤0.001, 50.0005 or ≤0.0001.

Differential expression (up-regulation or down regulation) of biomarkers, or lack thereof, can be determined by any suitable means known to a skilled person. Differential expression is determined to a p value of a least less than 0.05 (p=<0.05), for example, at least <0.04, <0.03, <0.02, <0.01, <0.009, <0.005, <0.001, <0.0001, <0.00001 or at least <0.000001. For example, differential expression may be determined using a support vector machine (SVM).

In one embodiment, the SVM is, or is derived from, the SVM described in Table 6, below.

It will be appreciated by persons skilled in the art that differential expression may relate to a single biomarker or to multiple biomarkers considered in combination (i.e., as a biomarker signature). Thus, a p value may be associated with a single biomarker or with a group of biomarkers. Indeed, proteins having a differential expression p value of greater than 0.05 when considered individually may nevertheless still be useful as biomarkers in accordance with the invention when their expression levels are considered in combination with one or more other biomarkers.

As exemplified in the accompanying Example, the expression of certain proteins in a tissue, blood, serum or plasma test sample may be indicative of pancreatic cancer in an individual. For example, the relative expression of certain serum proteins in a single test sample may be indicative of the presence of pancreatic cancer in an individual.

In an alternative or additional embodiment, the presence and/or amount in the test sample of the one or more biomarkers measured in step (b) may be compared against predetermined reference values representative of the measurements in steps (d) and/or (f), i.e., reference negative and/or positive control values.

As detailed above, the methods of the invention may also comprise measuring, in one or more negative or positive control samples, the presence and/or amount of the one or more biomarkers measured in the test sample in step (b).

For example, one or more negative control samples may be from an individual who was not, at the time the sample was obtained, afflicted with:
(a) a pancreatic cancer, for example adenocarcinoma (e.g., pancreatic ductal adenocarcinoma or tubular papillary pancreatic adenocarcinoma), pancreatic sarcoma, malignant serous cystadenoma, adenosquamous carcinoma, signet ring cell carcinoma, hepatoid carcinoma, colloid carcinoma, undifferentiated carcinoma, and undifferentiated carcinomas with osteoclast-like giant cells; and/or
(b) a non-cancerous pancreatic disease or condition, for example acute pancreatitis, chronic pancreatitis and autoimmune pancreatitis; and/or
(c) any other disease or condition.

Thus, the negative control sample may be obtained from a healthy individual.

Likewise, one or more positive control samples may be from an individual who, at the time the sample was obtained, was afflicted with a pancreatic cancer, for example adenocarcinoma (e.g., pancreatic ductal adenocarcinoma or tubular papillary pancreatic adenocarcinoma), pancreatic sarcoma, malignant serous cystadenoma, adenosquamous carcinoma, signet ring cell carcinoma, hepatoid carcinoma, colloid carcinoma, undifferentiated carcinoma, and undifferentiated carcinomas with osteoclast-like giant cells; and/or a non-cancerous pancreatic disease or condition, for example acute pancreatitis, chronic pancreatitis and autoimmune pancreatitis; and/or any other disease or condition.

In one preferred embodiment of the first aspect of the invention, the method is repeated on 5 the individual. Thus, steps (a) and (b) may be repeated using a sample from the same individual taken at different time to the original sample tested (or the previous method repetition). Such repeated testing may enable disease progression to be assessed, for example to determine the efficacy of the selected treatment regime and (if appropriate) to select an alternative regime to be adopted.

Thus, in one embodiment, the method is repeated using a test sample taken between 1 day to 104 weeks to the previous test sample(s) used, for example, between 1 week to 100 weeks, 1 week to 90 weeks, 1 week to 80 weeks, 1 week to 70 weeks, 1 week to 60 weeks, 1 week to 50 weeks, 1 week to 40 weeks, 1 week to 30 weeks, 1 week to 20 weeks, 1 week 15 to 10 weeks, 1 week to 9 weeks, 1 week to 8 weeks, 1 week to 7 weeks, 1 week to 6 weeks, 1 week to 5 weeks, 1 week to 4 weeks, 1 week to 3 weeks, or 1 week to 2 weeks.

Alternatively or additionally, the method may be repeated using a test sample taken every period from the group consisting of: 1 day, 2 days, 3 day, 4 days, 5 days, 6 days, 7 days, 10 days, 2 weeks, 3 weeks, 4 weeks, 5 weeks, 6 weeks, 7 weeks, 8 weeks, 9 weeks, 10 weeks, 15 weeks, 20 weeks, 25 weeks, 30 weeks, 35 weeks, 40 weeks, 45 weeks, 50 weeks, 55 weeks, 60 weeks, 65 weeks, 70 weeks, 75 weeks, 80 weeks, 85 weeks, 90 weeks, 95 weeks, 100 weeks, 104, weeks, 105 weeks, 110 weeks, 115 weeks, 120 weeks, 125 weeks and 130 weeks.

Alternatively or additionally, the method may be repeated at least once, for example, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23, 24 times or 25 times.

Alternatively or additionally, the method is repeated continuously.

In one embodiment, the method is repeated until pancreatic cancer is diagnosed and/or staged in the individual using the methods of the present invention and/or conventional clinical methods (i.e., until confirmation of the diagnosis is made).

Suitable conventional clinical methods are well known in the art. For example, those methods described in Ducreux et al., 2015, ‘Cancer of the pancreas: ESMO Clinical Practice Guidelines for diagnosis, treatment and follow-up’ Annals of Oncology, 26 (Supplement 5): v56-v68 and/or Freelove & Walling, 2006, ‘Pancreatic Cancer: Diagnosis and Management’ American Family Physician, 73(3):485-492 which are incorporated herein by reference. Thus, the pancreatic cancer diagnosis may be confirmed using one or more method selected from the group consisting of computed tomography (preferably dual-phase helical computed tomography); transabdominal ultrasonography; endoscopic ultrasonography-guided fine-needle aspiration; endoscopic retrograde cholangio-pancreatography; positron emission tomography; magnetic resonance imaging; physical examination; and biopsy.

Alternatively and/or additionally, the pancreatic cancer diagnosis may be confirmed using known biomarker signatures for the diagnosis of pancreatic cancer. For example, the pancreatic cancer may be diagnosed with one or more biomarker or diagnostic method described in the group consisting of: WO 2008/117067 A9; WO 2012/120288 A2; and WO 2015/067969 A2.

In one preferred embodiment of the methods of the invention, step (a) comprises providing a serum sample from an individual to be tested and/or step (b) comprises measuring in the sample the expression of the protein or polypeptide of the one or more biomarker(s). Thus, a biomarker signature for the sample may be determined at the protein level.

In such an embodiment, step (b), (d) and/or step (f) may be performed using one or more first binding agents capable of binding to a biomarker (i.e., protein) listed in Table A. It will be appreciated by persons skilled in the art that the first binding agent may comprise or consist of a single species with specificity for one of the protein biomarkers or a plurality of different species, each with specificity for a different protein biomarker.

Suitable binding agents (also referred to as binding molecules) can be selected from a library, based on their ability to bind a given target molecule, as discussed below.

In one preferred embodiment, at least one type of the binding agents, and more typically all of the types, may comprise or consist of an antibody or antigen-binding fragment of the same, or a variant thereof.

Methods for the production and use of antibodies are well known in the art, for example see Antibodies: A Laboratory Manual, 1988, Harlow & Lane, Cold Spring Harbor Press, ISBN-13: 978-0879693145, Using Antibodies: A Laboratory Manual, 1998, Harlow & Lane, Cold Spring Harbor Press, ISBN-13: 978-0879695446 and Making and Using Antibodies: A Practical Handbook, 2006, Howard & Kaser, CRC Press, ISBN-13: 978-0849335280 (the disclosures of which are incorporated herein by reference).

Thus, a fragment may contain one or more of the variable heavy (V_{H}) or variable light (V_{L}) domains. For example, the term antibody fragment includes Fab-like molecules (Better et al (1988) Science 240, 1041); Fv molecules (Skerra et al (1988) Science 240, 1038); single-chain Fv (scFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544).

For example, the binding agent(s) may be scFv molecules.

The term “antibody variant” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecule capable of binding to an antigen in an immunoassay format that is known to those skilled in the art.

A general review of the techniques involved in the synthesis of antibody fragments which retain their specific binding sites is to be found in Winter & Milstein (1991) Nature 349, 293-299.

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

Conveniently, the binding agent(s) may be immobilised on a surface (e.g., on a multiwell plate or array); see Example below.

In one embodiment of the methods of the invention, step (b), (d) and/or step (f) is performed using an assay comprising a second binding agent capable of binding to the one or more biomarkers, the second binding agent comprising a detectable moiety. For example, an immobilised (first) binding agent may initially be used to ‘trap’ the protein biomarker on to the surface of a microarray, and then a second binding agent may be used to detect the ‘trapped’ protein.

The second binding agent may be as described above in relation to the (first) binding agent, such as an antibody or antigen-binding fragment thereof.

It will be appreciated by skilled person that the one or more biomarkers (e.g., proteins) in the test sample may be labelled with a detectable moiety, prior to performing step (b).

Likewise, the one or more biomarkers in the control sample(s) may be labelled with a detectable moiety.

Alternatively, or in addition, the first and/or second binding agents may be labelled with a detectable moiety.

By a “detectable moiety” we include the meaning that the moiety is one which may be detected and the relative amount and/or location of the moiety (for example, the location on an array) determined.

Suitable detectable moieties are well known in the art. For example, the detectable moiety may be selected from the group consisting of: a fluorescent moiety; a luminescent moiety; a chemiluminescent moiety; a radioactive moiety; an enzymatic moiety.

In one preferred embodiment, the detectable moiety is biotin.

Thus, the detectable moiety may be a fluorescent and/or luminescent and/or chemiluminescent moiety which, when exposed to specific conditions, may be detected.

For example, a fluorescent moiety may need to be exposed to radiation (i.e., light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

In a further alternative, the detectable moiety may be a radioactive atom which is useful in 5 imaging. Suitable radioactive atoms include ^{99m}Tc and ¹²³I for scintigraphic studies. Other readily detectable moieties include, for example, spin labels for magnetic resonance imaging (MRI) such as ¹²³I again, ¹³¹I, ¹¹¹In, ¹⁹F, ¹³C, ¹⁵N, ¹⁷O, gadolinium, manganese or iron. Clearly, the agent to be detected (such as, for example, the one or more biomarkers in the test sample and/or control sample described herein and/or an antibody molecule for use in detecting a selected protein) must have sufficient of the appropriate atomic isotopes in order for the detectable moiety to be readily detectable.

Preferred assays for detecting serum or plasma proteins include enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies. Exemplary sandwich assays are described by David et al in U.S. Pat. Nos. 4,376,110 and 4,486,530, hereby incorporated by reference. Antibody staining of cells on slides may be used in methods well known in cytology laboratory diagnostic tests, as well known to those skilled in the art.

Conveniently, the assay is an ELISA (Enzyme Linked Immunosorbent Assay) which typically involves the use of enzymes giving a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

ELISA methods are well known in the art, for example see The ELISA Guidebook (Methods in Molecular Biology), 2000, Crowther, Humana Press, ISBN-13: 978-0896037281 (the disclosures of which are incorporated by reference).

Alternatively, conjugation with the vitamin biotin is frequently used since this can readily be detected by its reaction with enzyme-linked avidin or streptavidin to which it binds with great specificity and affinity.

In one preferred embodiment, step (b), (d) and/or step (f) may be performed using an array.

Arrays per se are well known in the art. Typically, they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising proteins, polynucleotides and other suitable molecules and/or conducting an immunoassay. The binding processes are well known in the art and generally consist of cross-linking covalently binding or physically adsorbing a protein molecule, polynucleotide or the like to the solid support. By using well-known techniques, such as contact or non-contact printing, masking or photolithography, the location of each spot can be defined. For reviews see Jenkins, R. E., Pennington, S. R. (2001, Proteomics, 2, 13-29) and Lal et al (2002, Drug Discov Today 15; 7(18 Suppl):S143-9).

Typically, the array is a microarray. By “microarray” we include the meaning of an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. The array may also be a macroarray or a nanoarray.

Once suitable binding molecules (discussed above) have been identified and isolated, the skilled person can manufacture an array using methods well known in the art of molecular biology.

Examples of array formats are described below in the Example and references cited therein; e.g., see Steinhauer et al., 2002; Wingren and Borrebaeck, 2008; Wingren et al., 2005, Delfani et al., 2016 (the disclosure of which are incorporated herein by reference).

Thus, in an exemplary embodiment the method comprises:
(i) labelling biomarkers present in the sample (e.g., serum) with biotin;
(ii) contacting the biotin-labelled proteins with an array comprising a plurality of scFv immobilised at discrete locations on its surface, the scFv having specificity for one or more of the proteins in Table A;
(iii) contacting the biotin-labelled proteins (immobilised on the surface-bound scFv) with a streptavidin conjugate comprising a fluorescent dye; and
(iv) detecting the presence of the dye at discrete locations on the array surface

wherein the expression of the dye on the array surface is indicative of the expression of a biomarker from Table A in the sample.

In an alternative embodiment, step (b), (d) and/or (f) comprises measuring the expression of a nucleic acid molecule encoding the one or more biomarkers.

The nucleic acid molecule may be a gene expression intermediate or derivative thereof, such as a mRNA or cDNA.

Thus, measuring the expression of the one or more biomarker(s) in step (b), (d) and/or (f) may be performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation.

For example, measuring the expression of the one or more biomarker(s) in step (b), (d) and/or (f) may be performed using one or more binding moieties, each individually capable of binding selectively to a nucleic acid molecule encoding one of the biomarkers identified in Table A.

Conveniently, the one or more binding moieties each comprise or consist of a nucleic acid molecule, such as DNA, RNA, PNA, LNA, GNA, TNA or PMO.

Advantageously, the one or more binding moieties are 5 to 100 nucleotides in length. For example, 15 to 35 nucleotides in length.

It will be appreciated that the nucleic acid-based binding moieties may comprise a detectable moiety.

Thus, the detectable moiety may be selected from the group consisting of: a fluorescent moiety; a luminescent moiety; a chemiluminescent moiety; a radioactive moiety (for example, a radioactive atom); or an enzymatic moiety.

Alternatively or additionally, the detectable moiety may comprise or consist of a radioactive atom, for example selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

Alternatively or additionally, the detectable moiety of the binding moiety may be a fluorescent moiety.

In a further embodiment, the nucleic acid molecule is a circulating tumour DNA molecule (ctDNA).

Methods suitable for detecting ctDNA are now well-established; for example, see Lewis et al., 2016, World J Gastroenterol. 22(32): 7175-7185, and references cited therein (the disclosures of which are incorporated herein by reference).

As detailed above, the sample provided in step (a) (and/or in step (c) and/or (e)) may be selected from the group consisting of unfractionated blood, plasma, serum, tissue fluid, pancreatic tissue, milk, bile and urine.

Conveniently, the sample provided in step (a), (c) and/or (e) is serum.

By appropriate selection of some or all of the biomarkers in Table A, optionally in conjunction with one or more further biomarkers, the methods of the invention exhibit high predictive accuracy for diagnosis of pancreatic cancer.

Thus, the predictive accuracy of the method, as determined by an ROC AUC value, may be at least 0.50, for example at least 0.55, 0.60, 0.65, 0.70, 0.75, 0.80, 0.85, 0.90, 0.95, 0.96, 0.97, 0.98 or at least 0.99.

Thus, in one embodiment, the predictive accuracy of the method, as determined by an ROC AUC value, is at least 0.90.

In the methods of the invention, the ‘raw’ data obtained in step (b) (and/or in step (d) and/or (e)) undergoes one or more analysis steps before a diagnosis is reached. For example, the raw data may need to be standardised against one or more control values (i.e., normalised).

Typically, diagnosis is performed using a support vector machine (SVM), such as those available from http://cran.r-project.org/web/packages/e1071/index.html (e.g. e1071 1.5-24). However, any other suitable means may also be used.

Support vector machines (SVMs) are a set of related supervised learning methods used for classification and regression. Given a set of training examples, each marked as belonging to one of two categories, an SVM training algorithm builds a model that predicts whether a new example falls into one category or the other. Intuitively, an SVM model is a representation of the examples as points in space, mapped so that the examples of the separate categories are divided by a clear gap that is as wide as possible. New examples are then mapped into that same space and predicted to belong to a category based on which side of the gap they fall on.

More formally, a support vector machine constructs a hyperplane or set of hyperplanes in a high or infinite dimensional space, which can be used for classification, regression or other tasks. Intuitively, a good separation is achieved by the hyperplane that has the largest distance to the nearest training data points of any class (so-called functional margin), since in general the larger the margin the lower the generalization error of the classifier. For more information on SVMs, see for example, Burges, 1998, Data Mining and Knowledge Discovery, 2:121-167.

In one embodiment of the invention, the SVM is ‘trained’ prior to performing the methods of the invention using biomarker profiles from individuals with known disease status (for example, individuals known to have pancreatic cancer, individuals known to have acute inflammatory pancreatitis, individuals known to have chronic pancreatitis or individuals known to be healthy). By running such training samples, the SVM is able to learn what biomarker profiles are associated with pancreatic cancer. Once the training process is complete, the SVM is then able to determine whether or not the biomarker sample tested is from an individual with pancreatic cancer.

However, this training procedure can be by-passed by pre-programming the SVM with the necessary training parameters. For example, diagnoses can be performed according to the known SVM parameters using the SVM algorithm detailed in Table 6, based on the measurement of any or all of the biomarkers listed in Table A.

It will be appreciated by skilled persons that suitable SVM parameters can be determined for any combination of the biomarkers listed in Table A by training an SVM machine with the appropriate selection of data (i.e. biomarker measurements from individuals with known pancreatic cancer status). Alternatively, the data of the Examples and figures may be used to determine a particular pancreatic cancer-associated disease state according to any other suitable statistical method known in the art.

Preferably, the method of the invention has an accuracy of at least 60%, for example 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% accuracy.

Preferably, the method of the invention has a sensitivity of at least 60%, for example 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sensitivity.

Preferably, the method of the invention has a specificity of at least 60%, for example 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% specificity.

By “accuracy” we mean the proportion of correct outcomes of a method, by “sensitivity” we mean the proportion of all pancreatic cancer positive sample that are correctly classified as positives, and by “specificity” we mean the proportion of all pancreatic cancer negative samples that are correctly classified as negatives.

Signal intensities may be quantified using any suitable means known to the skilled person, for example using Array-Pro (Media Cybernetics). Signal intensity data may be normalised (i.e., to adjust technical variation). Normalisation may be performed using any suitable method known to the skilled person. Alternatively or additionally, data are normalised using the empirical Bayes algorithm ComBat (Johnson et al., 2007).

Further statistical analysis of the refined data may be performed using methods well-known in the art, such as PCA, q-value calculation by ANOVA, and/or fold change calculation in Qlucore Omics Explorer.

As described above, a first (‘training’) data set may be used to identify a combination of biomarkers, e.g. from Table A, to serve as a biomarker signature for the diagnosis of pancreatic cancer. Mathematical analysis of the training data set may be performed using known algorithms (such as a backward elimination, or BE, algorithm) to determine the most suitable biomarker signatures. The predictive accuracy of a given biomarker combination (signature) can then be verified against a new (‘verification’) data set. Such methodology is described in detail in the Example.

It will be appreciated by persons skilled in the art that the individual(s) tested may be of any ethnicity or geographic origin. Alternatively, the individual(s) tested may be of a defined sub-population, e.g., based on ethnicity and/or geographic origin. For example, the individual(s) tested may be Caucasian and/or Chinese (e.g., Han ethnicity).

Typically, the sample(s) provided in step (a), (c) and/or (e) are provided before treatment of the pancreatic cancer (e.g., resection, chemotherapy, radiotherapy).

In one embodiment, the individual(s) being tested suffers from one or more condition selected from the group consisting of chronic pancreatitis, hereditary pancreatic ductal adenocarcinoma and Peutz-Jeghers syndrome.

The pancreatic cancer to be diagnosed may be selected from the group consisting of adenocarcinoma, adenosquamous carcinoma, signet ring cell carcinoma, hepatoid carcinoma, colloid carcinoma, undifferentiated carcinoma, and undifferentiated carcinomas with osteoclast-like giant cells. Preferably, the pancreatic cancer is a pancreatic adenocarcinoma. More preferably, the pancreatic cancer is pancreatic ductal adenocarcinoma, also known as exocrine pancreatic cancer.

One preferred embodiment of the first aspect of the invention includes the additional step, following positive diagnosis of the individual with a pancreatic cancer, of providing the individual with pancreatic cancer therapy.

Thus, a related aspect of the invention provides a method of treatment of an individual with a pancreatic cancer comprising the following steps:
(a) diagnosing an individual as having a pancreatic cancer using a method according to the first aspect of the invention; and
(b) treating the individual so diagnosed with a pancreatic cancer therapy (for example, see Thota et al., 2014, Oncology 28(1):70-4, the disclosures of which are incorporated herein by reference).

The pancreatic cancer therapy may be selected from the group consisting of surgery, chemotherapy, immunotherapy, chemoimmunotherapy, thermochemotherapy, radiotherapy and combinations thereof. For example, the pancreatic cancer therapy may be AC chemotherapy; Capecitabine and docetaxel chemotherapy (Taxotere®); CMF chemotherapy; Cyclophosphamide; EC chemotherapy; ECF chemotherapy; E-CMF chemotherapy (Epi-CMF); Eribulin (Halaven®); FEC chemotherapy; FEC-T chemotherapy; Fluorouracil (5FU); GemCarbo chemotherapy; Gemcitabine (Gemzar®); Gemcitabine and cisplatin chemotherapy (GemCis or GemCisplat); GemTaxol chemotherapy; Idarubicin (Zavedos®); Liposomal doxorubicin (DaunoXome®); Mitomycin (Mitomycin C Kyowa®); Mitoxantrone; MM chemotherapy; MMM chemotherapy; Paclitaxel (Taxol®); TAC chemotherapy; Taxotere and cyclophosphamide (TC) chemotherapy; Vinblastine (Velbe®); Vincristine (Oncovin®); Vindesine (Eldisine®); and Vinorelbine (Navelbine®).

Accordingly, a further aspect of the invention provides an antineoplastic agent (or combination thereof) for use in treating pancreatic cancer wherein the dosage regime thereof is determined based on the results of the method of the first aspect of the invention.

A related aspect of the invention provides the use of an antineoplastic agent (or combination thereof) in treating pancreatic cancer wherein the dosage regime thereof is determined based on the results of the method of the first aspect of the invention.

A further related aspect of the invention provides the use of an antineoplastic agent (or combination thereof) in the manufacture of a medicament for treating pancreatic cancer wherein the dosage regime thereof is determined based on the results of the method of the first aspect of the invention.

Thus, the present invention also provides a method of treating pancreatic cancer comprising administering to a patient an effective amount of an antineoplastic agent (or combination thereof) wherein the amount of antineoplastic agent (or combination thereof) effective to treat the pancreatic cancer is determined based on the results of the method of the first aspect of the invention.

In one embodiment, the antineoplastic agent comprises or consists of an alkylating agent (ATC code L01a), an antimetabolite (ATC code L01b), a plant alkaloid or other natural product (ATC code L01c), a cytotoxic antibiotic or a related substance (ATC code L01d), or another antineoplastic agent (ATC code L01x).

Hence, in one embodiment the antineoplastic agent comprises or consists of an alkylating agent selected from the group consisting of a nitrogen mustard analogue (for example cyclophosphamide, chlorambucil, melphalan, chlormethine, ifosfamide, trofosfamide, prednimustine or bendamustine) an alkyl sulfonate (for example busulfan, treosulfan, or mannosulfan) an ethylene imine (for example thiotepa, triaziquone or carboquone) a nitrosourea (for example carmustine, lomustine, semustine, streptozocin, fotemustine, nimustine or ranimustine) an epoxides (for example etoglucid) or another alkylating agent (ATC code L01ax, for example mitobronitol, pipobroman, temozolomide or dacarbazine).

In another embodiment the antineoplastic agent comprises or consists of an antimetabolite selected from the group consisting of a folic acid analogue (for example methotrexate, raltitrexed, pemetrexed or pralatrexate), a purine analogue (for example mercaptopurine, tioguanine, cladribine, fludarabine, clofarabine or nelarabine) or a pyrimidine analogue (for example cytarabine, fluorouracil (5-FU), tegafur, carmofur, gemcitabine, capecitabine, azacitidine or decitabine).

In a still further embodiment the antineoplastic agent comprises or consists of a plant alkaloid or other natural product selected from the group consisting of a vinca alkaloid or a vinca alkaloid analogue (for example vinblastine, vincristine, vindesine, vinorelbine or vinflunine), a podophyllotoxin derivative (for example etoposide or teniposide) a colchicine derivative (for example demecolcine), a taxane (for example paclitaxel, docetaxel or paclitaxel poliglumex) or another plant alkaloids or natural product (ATC code LOlcx, for example trabectedin).

In one embodiment the antineoplastic agent comprises or consists of a cytotoxic antibiotic or related substance selected from the group consisting of an actinomycine (for example dactinomycin), an anthracycline or related substance (for example doxorubicin, daunorubicin, epirubicin, aclarubicin, zorubicin, idarubicin, mitoxantrone, pirarubicin, valrubicin, amrubicin or pixantrone) or another (ATC code L01dc, for example bleomycin, plicamycin, mitomycin or ixabepilone).

In a further embodiment the antineoplastic agent comprises or consists of an antineoplastic agent selected from the group consisting of a platinum compound (for example cisplatin, carboplatin, oxaliplatin, satraplatin or polyplatillen) a methylhydrazine (for example procarbazine) a monoclonal antibody (for example edrecolomab, rituximab, trastuzumab, alemtuzumab, gemtuzumab, cetuximab, bevacizumab, panitumumab, catumaxomab or ofatumumab) a sensitizer used in photodynamic/radiation therapy (for example porfimer sodium, methyl aminolevulinate, aminolevulinic acid, temoporfin or efaproxiral) or a protein kinase inhibitor (for example imatinib, gefitinib, erlotinib, sunitinib, sorafenib, dasatinib, lapatinib, nilotinib, temsirolimus, everolimus, pazopanib, vandetanib, afatinib, masitinib or toceranib).

In a still further embodiment the antineoplastic agent comprises or consists of an antineoplastic agent selected from the group consisting of amsacrine, asparaginase, altretamine, hydroxycarbamide, lonidamine, pentostatin, miltefosine, masoprocol, estramustine, tretinoin, mitoguazone, topotecan, tiazofurine, irinotecan (camptosar), alitretinoin, mitotane, pegaspargase, bexarotene, arsenic trioxide, denileukin diftitox, bortezomib, celecoxib, anagrelide, oblimersen, sitimagene ceradenovec, vorinostat, romidepsin, omacetaxine mepesuccinate, eribulin or folinic acid.

In one embodiment the antineoplastic agent comprises or consists of a combination of one or more antineoplastic agent, for example, one or more antineoplastic agent defined herein. One example of a combination therapy used in the treatment of pancreatic cancer is FOLFIRINOX which is made up of the following four drugs:
FOL—folinic acid (leucovorin);
F—fluorouracil (5-FU);
IRIN—irinotecan (Camptosar); and
OX—oxaliplatin (Eloxatin).

Thus, by combining certain optional embodiments from the above-described methods, the invention may provide a method for diagnosing and treating pancreatic adenocarcinoma (e.g. stage I or II) in an individual, said method comprising:
(a) obtaining or providing a serum or plasma sample for a human patient;
(b) detecting whether one or more (e.g. all) of the protein biomarkers from Table A is/are present in the sample (e.g. by contacting the sample with one or more antibodies, or antigen-binding fragments thereof, each having specificity for one of the biomarkers and detecting binding of said antibodies or fragments to said biomarkers);
(c) diagnosing the patient with pancreatic adenocarcinoma (e.g. stage I or II) based on the amount of the one or more protein biomarkers in the sample; and
(d) administering an effective amount of a chemotherapeutic agent (e.g. gemcitabine) to the diagnosed patient and/or surgically removing the pancreas, in whole or in part, and/or administering radiotherapy.

It will be appreciated that step (b) may, for example, comprise determining the presence and/or amount in the sample of all the biomarkers listed in Table A (excluding IL-6 and GEM) together with C1q. This step may comprise the use of an array, as described herein, e.g. comprising a plurality of scFv having specificity the biomarkers immobilised on the surface of an array plate.

It will be appreciated that step (c) may comprise one or more further clinical investigations (such as testing a biopsy sample and/or in vivo imaging of the patient) in order to confirm or establish the diagnosis.

It will be appreciated that step (d) may comprise administration of combinations of chemotherapeutic agent and/or surgery and/or radiotherapy.

In one preferred embodiment, the patient is diagnosed with resectable pancreatic adenocarcinoma (e.g. stage I or II) and step (d) comprises surgical removal of the pancreas in whole or in part (e.g. using the Whipple procedure to remove the pancreas head or a total pancreatectomy) combined with chemotherapy (e.g. gemcitabine and/or 5-fluorouracil). It will be appreciated that the chemotherapy may be administered before and/or after the surgery.

In one embodiment, such methods permit the diagnosis of early stage pancreatic adenocarcinoma prior to the phenotypic presentation of the disease (i.e. before observable clinical symptoms develop). Thus, the methods may be used to diagnose pancreatic adenocarcinoma in asymptomatic patients, especially those at high risk of developing pancreatic cancer such as those with a family history of the disease, tobacco smokers, obese individuals, diabetics, and individuals with a chronic pancreatitis, chronic hepatitis B infection, cholelithiasis and/or an associated genetic predisposition (e.g. Peutz-Jeghers syndrome, familial atypical multiple mole melanoma syndrome, Lynch syndrome, BRCA1 mutations and/or BRCA2 mutations). Effective monitoring of such high risk individuals can enable early diagnosis of pancreatic adenocarcinoma and so greatly increase the chances of survival.

Another aspect of the invention provides a method for treating a pancreatic cancer-associated disease state in a subject comprising or consisting of administering a pancreatic cancer therapy to a subject, wherein said subject has a biomarker signature of the present invention indicating the presence of the pancreatic cancer-associated disease state in the subject. The pancreatic cancer therapy may be resection, chemotherapy, and/or radiotherapy. In one embodiment, the pancreatic cancer therapy comprises the administration of at least one antineoplastic agent, as described hereinabove.

The method may further comprise (e.g. prior to treating) measuring the presence and/or amount in a test sample of one or more biomarker(s) selected from the group defined in Table A (e.g. all the biomarker in Table A). The method may comprise determining a biomarker signature of a test sample from the subject (e.g. prior to treating), as described hereinabove.

Another aspect of the invention provides a method for detecting a biomarker signature of clinical significance (e.g. of diagnostic and/or prognostic value) in or of a biological sample (e.g. a serum sample), the method comprising steps (a) and (b) as defined above in relation to the first aspect of the invention. Preferably, the biomarker signature comprises or consists of all of the biomarkers in Table A.

A further aspect of the invention provides an array for diagnosing or determining a pancreatic cancer-associated disease state in an individual comprising an agent or agents (such as any of the above-described binding agents) for detecting the presence in a sample of one or more of the biomarkers defined in Table A.

Thus, the array is suitable for performing a method according to the first aspect of the invention.

The array comprises one or more binding agents capable (individually or collectively) of binding to one or more of the biomarkers defined in Table A, either at the protein level or the nucleic acid level.

In one preferred embodiment, the array comprises one or more antibodies, or antigen-binding fragments thereof, capable (individually or collectively) of binding to one or more of the biomarkers defined in Table A at the protein level. For example, the array may comprise scFv molecules capable (collectively) of binding to all of the biomarkers defined in Table A at the protein level.

In an alternative embodiment, the array comprises one or more antibodies, or antigen-binding fragments thereof, capable (individually or collectively) of binding to the following biomarkers:
DLG1, PRKCZ, VEGF, C3, C1INH, IL-4, IFNγ, C5, PTK6, CHP1, APLF, CAMK4, MAGI, MARK1, PRDM8, APOA1, CDK2, HADH2, C4, VSX2/CHX10, ICAM-1, IL-13, Lewis x/CD15, MYOM2, Factor P, Sialyl Lewis x, TNFβ and Complement C1q

(optionally including one or more biomarkers from Table B and/or IL-6 and/or GEM).

It will be appreciated that the array may comprise one or more positive and/or negative control samples. For example, conveniently the array comprises bovine serum albumin as a positive control sample and/or phosphate-buffered saline as a negative control sample.

Conveniently, the array comprises one or more, e.g. all, of the antibodies in Table 7.

Advantageously, the array comprises one or more, e.g. all, of the antibodies in Table 8.

A further aspect of the invention provides use of one or more biomarkers selected from the group defined in Table A as a biomarker for determining a pancreatic cancer associated disease states in an individual.

For example, all of the biomarkers (e.g. proteins) defined in Table A may be used together as a diagnostic signature for determining the presence of pancreatic cancer in an individual.

A further aspect of the invention provides a kit for diagnosing or determining a pancreatic cancer-associated disease state in an individual comprising:
(a) an array according to the invention, or components for making the same; and
(b) instructions for performing the method as defined above (e.g., in the first aspect of the invention).

A further aspect of the invention provides a use of one or more binding moieties to a biomarker as described herein (e.g. in Table A) in the preparation of a kit for diagnosing or determining a pancreatic cancer-associated disease state in an individual. Thus, multiple different binding moieties may be used, each targeted to a different biomarker, in the preparation of such as kit. In one embodiment, the binding moiety is an antibody or antigen-binding fragment thereof (e.g. scFv), as described herein.

A further aspect of the invention provides a method of treating pancreatic cancer in an individual comprising the steps of:
(a) determining a pancreatic cancer associated disease state according to the method defined in any the first aspect of the invention; and
(b) providing the individual with pancreatic cancer therapy.

For example, the pancreatic cancer therapy may be selected from the group consisting of surgery (e.g., resection), chemotherapy, immunotherapy, chemoimmunotherapy and thermochemotherapy (see above).

A further aspect of the invention provides a computer program for operating the methods the invention, for example, for interpreting the expression data of step (c) (and subsequent expression measurement steps) and thereby diagnosing or determining a pancreatic cancer-associated disease state. The computer program may be a programmed SVM. The computer program may be recorded on a suitable computer-readable carrier known to persons skilled in the art. Suitable computer-readable-carriers may include compact discs (including CD-ROMs, DVDs, Blu-ray and the like), floppy discs, flash memory drives, ROM or hard disc drives. The computer program may be installed on a computer suitable for executing the computer program.

Preferred, non-limiting examples which embody certain aspects of the invention will now be described, with reference to the following figures:

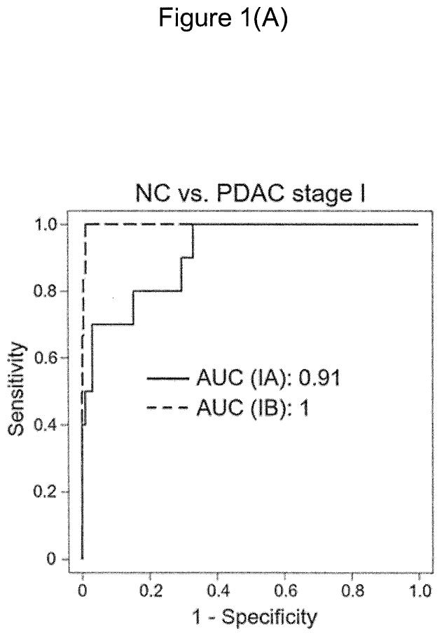

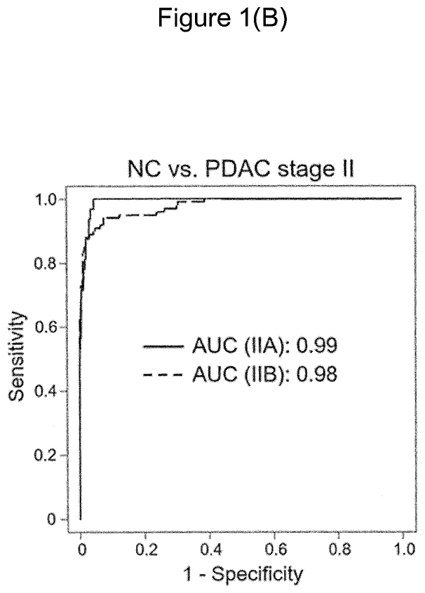

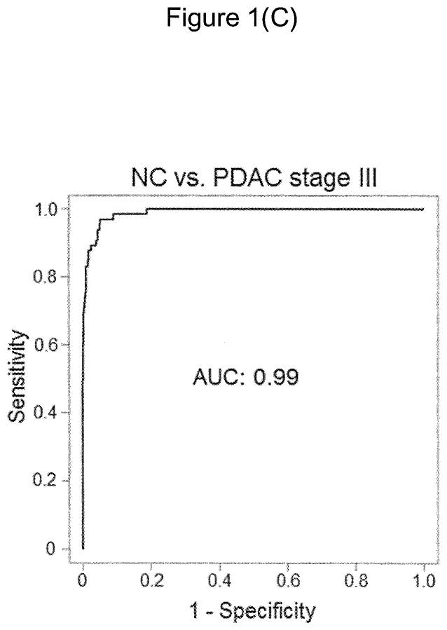

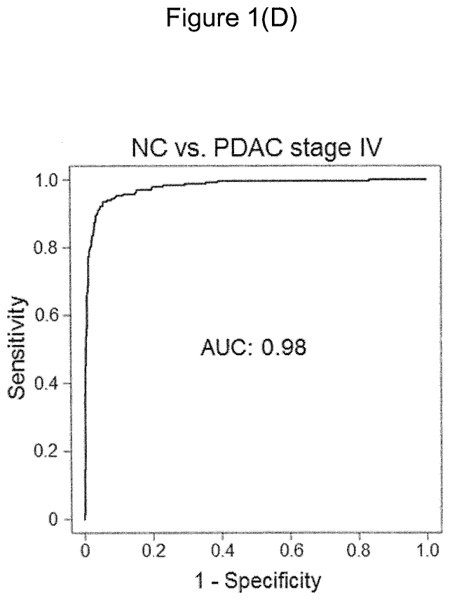

FIG. 1. Classification of individual PDAC stages in the Scandinavian cohort

Data shown are derived when all 349 antibodies were used to classify NC from patient samples of different PDAC stages, using SVM LOO cross validation. The results are presented with ROC-curves and their corresponding AUC-values for (A) stage I, (B) stage II, (C) stage III, and (D) stage IV PDAC.

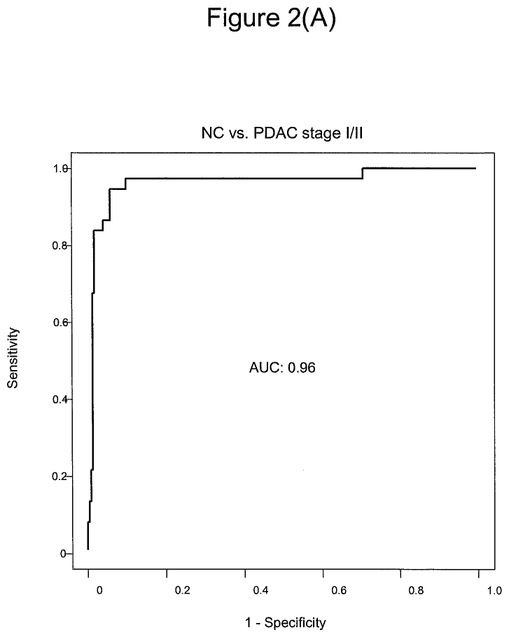

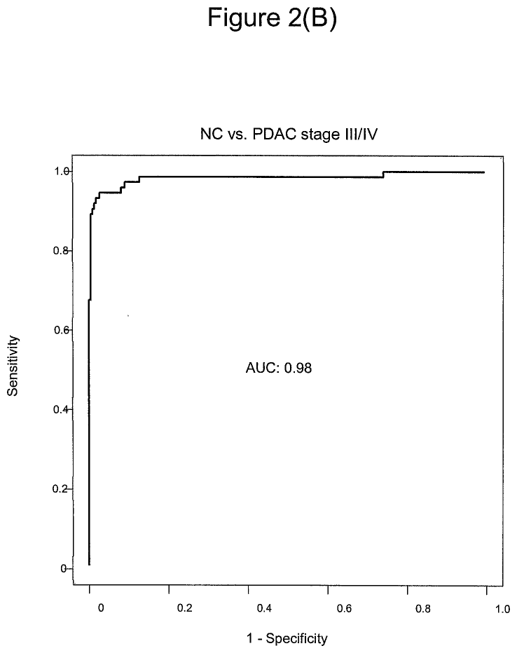

FIG. 2. Classification of PDAC stages in the Scandinavian cohort, using biomarker signatures

Utilizing data from the Scandinavian study, predictive models based on frozen SVM were built. Two biomarker signatures were defined, using the backward elimination algorithm, for classification of (A) NC samples from PDAC stage I/II, and (B) PDAC stage III/IV, respectively. The results are presented as ROC-curves and their corresponding AUC-values.

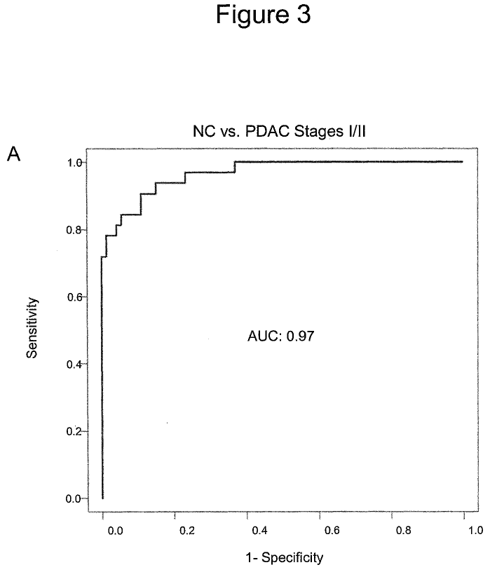

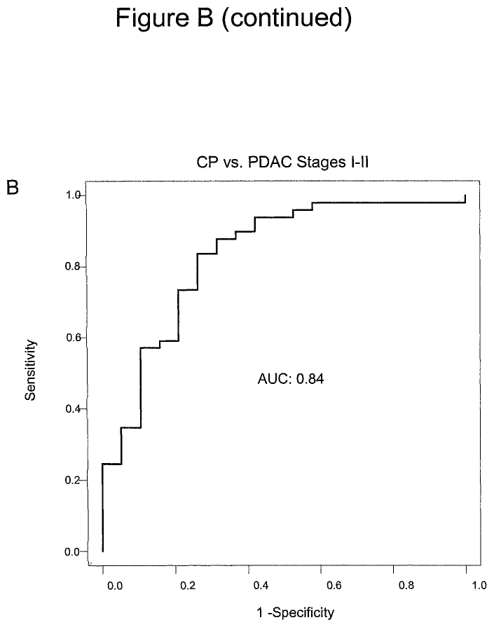

FIG. 3. Validation of the consensus signature in stage I/II PDAC from the US cohort.

The consensus signature generated from the Scandinavian cohort was validated in the independent US cohort, by classifying (A) NC vs. PDAC stage I/II patients, and (B) PDAC stage I/II patients vs. chronic pancreatitis patients. The results are presented as representative ROC-curves and their corresponding AUC-values.

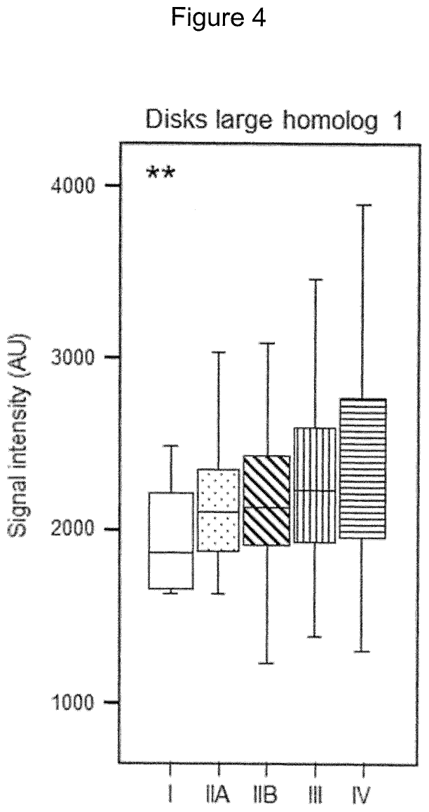

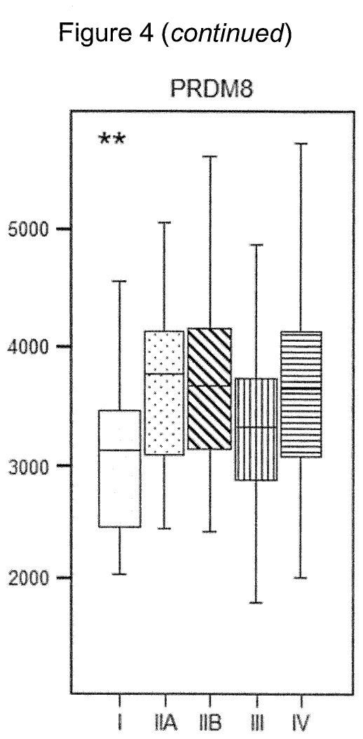

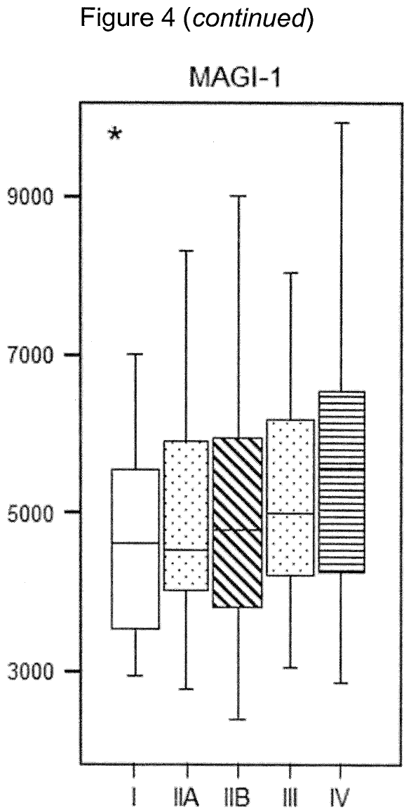

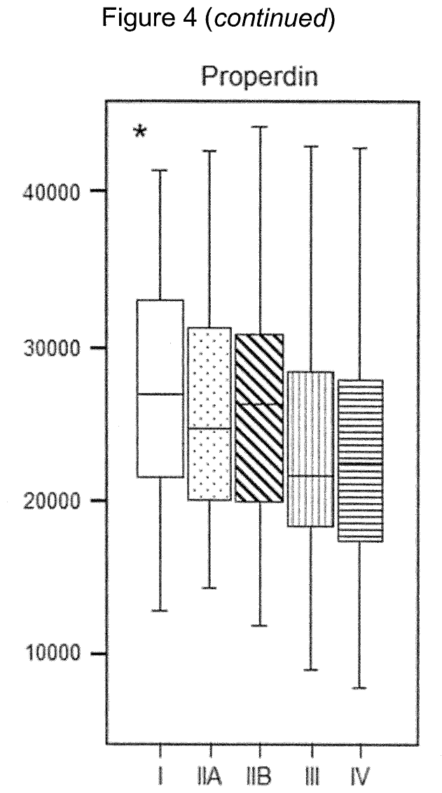

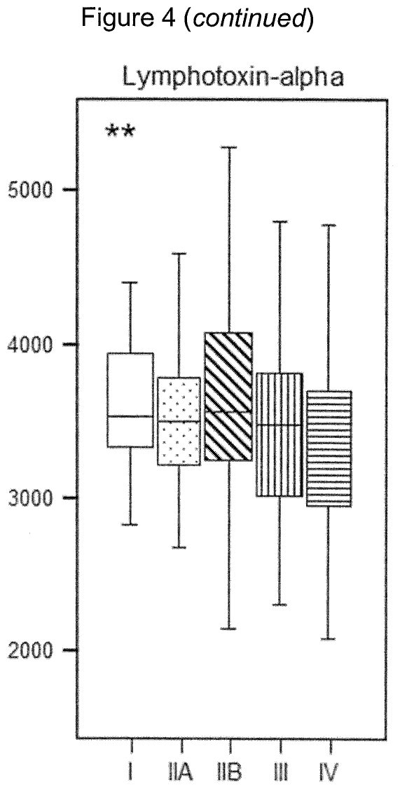

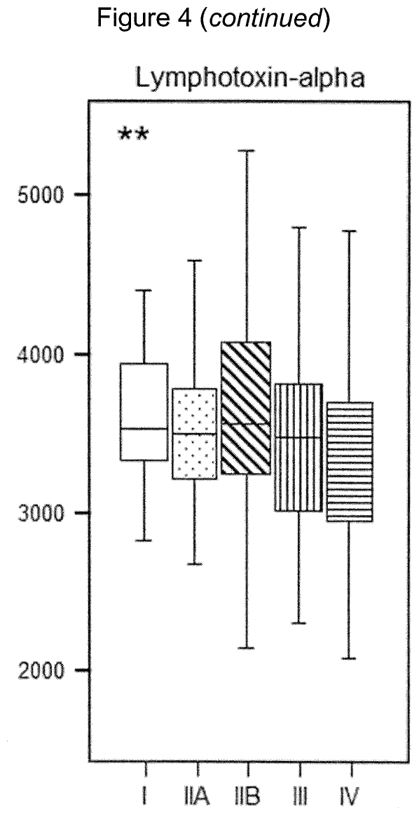

FIG. 4. Serum markers that are differentially expressed between different PDAC stages

Serum markers that were differentially expressed over progression from stage I to IV were identified by multigroup ANOVA. Presented are the most significant markers. Roman numerals indicate PDAC stage. *: p<0.05, q>0.05 and **: p<0.05, q<0.05

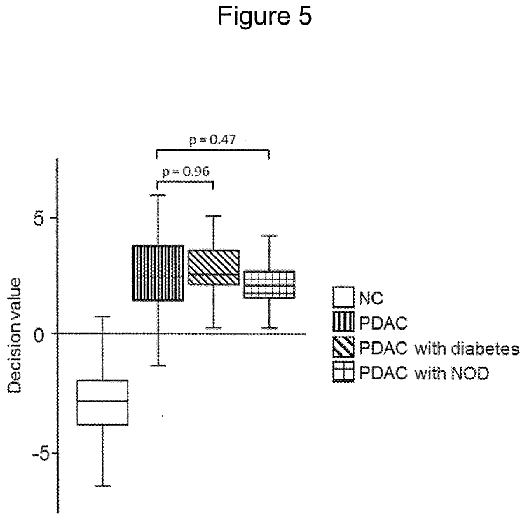

FIG. 5. Influence of diabetes on NC vs. PDAC classification accuracy

Decision values from an SVM model that had been trained on NC vs. PDAC were used to analyse differences between diabetic and non-diabetic PDAC samples in the discovery cohort. Significance values were calculated, using the Wilcoxon signed-rank test.

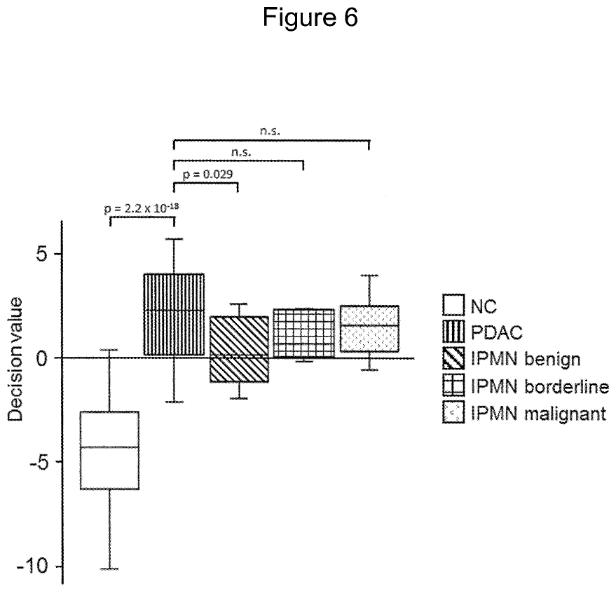

FIG. 6 Classification of IPMN stages from NC samples

The consensus signature was used to classify NC vs. the different IPMN stages. All IPMN samples from the US cohort were fed into an SVM model that had been trained on NC vs. PDAC. Significance values were calculated, using the Wilcoxon signed-rank test. The generated p-values were: NC vs. PDAC: 2.23×10⁻¹⁸; PDAC vs benign IPMN: 0.029; PDAC vs borderline IPMN: 0.284; PDAC vs malignant IPMN: 0.401.

### EXAMPLE

Abstract

Background

Pancreatic ductal adenocarcinoma (PDAC) has a poor prognosis with a 5-year survival of less than 10% due to diffuse symptoms leading to late stage diagnosis. The survival could increase significantly if localized tumours can be detected earlier. Multiparametric analysis of blood samples was used to derive a novel biomarker signature of early stage PDAC. The signature was developed from a large cohort of well-defined early stage (I/11) PDAC patients and subsequently validated in an independent patient cohort.

Methods

A recombinant antibody microarray platform was utilized to decipher a biomarker serum signature associated with PDAC. The discovery study was a case/control study from Scandinavia, consisting of 16 stage I, 132 stage II, 65 stage III, 230 stage IV patients and 888 controls. The identified biomarker signature was subsequently validated in an independent US case/control study cohort with 15 stage I, 75 stage II, 15 stage III, 38 stage IV patients and 219 controls.

Results

Using the Scandinavian case/control study, signatures were created discriminating samples derived from stage I/I and stage III/IV patients vs. controls with ROC-AUC values of 0.96 and 0.98, respectively. Subsequently, a consensus signature consisting of 29 biomarkers was generated based on all PDAC stages and control samples. This signature was then validated in an independent US case/control study and produced a ROC-AUC value of 0.96 using samples collected from PDAC stage I/II patients.

Conclusion

The validated serum signature detected early stage localized PDAC with high sensitivity and specificity, thus paving the way for earlier diagnosis.

### Abbreviations

ANOVA, Analysis of variance; AUC, Area under the curve; BE, Backward elimination; CP, Chronic pancreatitis; CV, Coefficient of variance; GO, gene ontology; IPMN, Intraductal papillary mucinous neoplasms (IPMN); LOO, Leave-one-out; MT-PBS, Phosphate buffered saline with 1% milk and 1% Tween-20; NC, Normal controls; PBS, Phosphate buffered saline; NPV, negative predictive value; PPV, positive predictive value; PBST, Phosphate buffered saline with 1% Tween-20; PCA, principal component analysis; PDAC, Pancreatic ductal adenocarcinoma; ROC, Receiver operating characteristic; RT, Room temperature; scFv, Single-chain fragment variable; SVM, Support vector machine

Introduction

In this study, PDAC stage I-IV patients were analysed in a large retrospective Scandinavian cohort followed by validation in an independent US cohort, aiming at identifying stage I/I1 associated PDAC biomarkers in a simple blood sample.

Methods

Study Designs

The two retrospective studies, performed on PDAC serum samples collected in Scandinavia and the US, were conducted according to the Standards for Reporting Diagnostic Accuracy Studies (STARD)²⁸. PDAC staging was performed according to the American Joint Committee on Cancer (AJCC) guidelines. Blood samples from patients with pancreatic cancer were collected and processed at time of diagnosis, before operation or start of chemotherapy. Blood samples from normal controls (NC) were collected, using the same standard operating procedure (SOP). In both cases, 5 μl of the serum samples was subsequently used for the analysis, utilizing a recombinant antibody microarray platform comprised of 349 human recombinant scFvs directed against 156 antigens (Table 5) (see Supplement Methods, below). The rationale was to target the systemic response to disease as well as the tumor secretome. Consequently, the selected biomarkers were mainly involved in immunoregulation.

Demographics of Study Cohorts

The Scandinavian cohort comprised 443 PDAC cases, 888 NC, and 8 intraductal papillary mucinous neoplasms (IPMN) (Table 1). The cases were diagnostic, and the overall resection rate was around 15%. Sixteen PDAC samples were from stage 1, 132 were from stage II, 65 were from stage III, and 230 were from stage IV patients (Table 1). Of the eight IPMN samples, five were benign and three were malignant.

The US cohort comprised 143 PDAC, 57 chronic pancreatitis (CP), and 20 IPMN cases as well as 219 NC (Table 1). Fifteen of the PDAC samples were from stage I, 75 were from stage II, 15 were from stage III, and 38 were from stage IV patients (Table 1). Of the 20 IPMN cases eight were benign, five were borderline, and seven were malignant. The cases were diagnostic, and the overall resection rate was 18-20%.

Results

Affinity proteomics offer some attractive features, such as delivering a highly sensitive assay using minute volumes of sample. The present approach was based on a recombinant antibody microarray platform comprised of 349 human recombinant scFvs directed against 156 antigens (Table 5). Since the focus was to interrogate the systemic response to PDAC, as well as its secretome, the selected antibodies targeted mainly antigens involved in immunoregulation. Two patient cohorts—one Scandinavian and one North American—including well defined early stage PDAC were utilized to identify and validate a biomarker signature for detection of stage I/I cancer.

First, to interrogate the robustness of the data set in the Scandinavian case/control discovery study, serum samples derived from patients with different PDAC stages were compared to matched healthy controls, using a LOO cross validation strategy. The results demonstrated that the different PDAC stages could be discriminated with high accuracy. The AUC values for NC vs. stages IA, IB, IIA, IIB, III, and IV were 0.91, 1.0, 0.99, 0.98, 0.99, and 0.98, respectively (FIG. 1). Of note, when using information derived from all antibodies on the array the resulting AUC levels, except for stage IA, reached 0.98 or higher.

Classifying PDAC Stage 1/11 with a Defined Biomarker Signature

In order to identify the smallest biomarker signature, discriminating PDAC stage I/II from NC with optimal predictive power, the SVM-based Backward Elimination algorithm was applied on the Scandinavian sample cohort^{26, 29}. Using this approach, biomarkers that do not improve the classification are eliminated resulting in identification of the signature providing the highest possible predictive power separating stage I/I vs. NC. This analysis resulted in a signature comprising only the highest ranked individual biomarkers (Table 4) and the obtained AUC value for stage I/I vs. NC was 0.96 (FIG. 2A), correlating to a specificity/sensitivity combination of 94/95% for NC vs. stage I/I. For comparative reasons, the obtained AUC value for stage III/IV vs. NC was 0.98 (FIG. 2B). These values are based on an investigation of the statistical robustness and classification model stability, where four randomly generated training/test sets were used, resulting in a mean AUC value of 0.963 (range 0.94-0.98) for the classification of NC vs. PDAC stage 1/I. The corresponding value for NC vs. stage III/IV was 0.985 (range 0.98-0.99). Of note, the highest predictive signature did not include e.g. CA19-9, a Sialyl Lewis A antigen commonly involved in analysis of PDAC, since it did not contribute with enough orthogonal information.

Validating the Detection of Early Stage 1/11 PDAC in an Independent Patient Cohort

To obtain the highest predictive accuracy in the validation study, the highest ranked biomarkers (Table 4) were combined to obtain a consensus signature, consisting of 29 biomarkers (Table 2). To validate the consensus signature for detection of early stage I/II PDAC patients, this signature was tested in a consecutive validation study, using samples derived from a completely independent US cohort. This validation analysis demonstrated a highly accurate discrimination of PDAC stage I/II vs. NC, with a ROC-AUC value of 0.963 (range 0.94-0.98), based on the three training sets (FIG. 3A). This correlates to an optimal specificity/sensitivity combination of 95/93% for stage I/II. Corresponding optimal ROC-AUC value for stage III/IV was 0.97 and for stage I-IV was 91/91%.

The capability to discriminate chronic pancreatitis from PDAC was also analysed, since differential diagnosis of pancreatitis vs. PDAC is a potential confounding clinical factor. Classification analysis of chronic pancreatitis from PDAC stage I/II samples resulted in an optimal ROC-AUC value of 0.84 (FIG. 3B).

Influence of Diabetes and Jaundice on Classification of Early Stage PDAC

The influence of diabetes on the classification accuracy was also investigated. In the Scandinavian cohort, 103 (23.3%) of the PDAC patients were diabetic (Table 3), while 38 (26.6%) of the PDAC patients in the US cohort had diabetes, at time of sample collection (Table 3). Newly onset diabetes (NOD), comprised 26.2% of the diabetic patients (n=37), in both cohorts. Decision values from the SVM model were used to analyze any significant differences between diabetic and non-diabetic PDAC samples in the discovery cohort. This analysis indicated that diabetes, including NOD, is not a confounding factor in the classification of NC vs. PDAC (p=0.47 and 0.96, respectively) (FIG. 3). The same approach applied on the validation cohort indicated that jaundice is not to a confounding factor (p=0.21).

Individual Serum Markers Associated with Different PDAC Stages

Individual biomarkers displaying a temporal expression pattern associated with progression from stage I to IV were also analyzed. By interrogating the data with multigroup ANOVA several biomarkers were identified that were differentially expressed in early vs. late stage PDAC patients. These included disks large homolog 1, PRDM8, and MAGI-1, which all displayed increased expression in later stages, while properdin, lymphotoxin-alpha, and IL-2 was more highly expressed in the early stages of PDAC (FIG. 4). Of note, all these biomarkers, except IL-2, were also present in the consensus signature (Table 2).

Classifying Intraductal Papillary Mucinous Neoplasm with the Validated Biomarker Signature

IPMNs frequently progress to invasive cancer if left untreated. Consequently, it is of clinical interest to detect such lesions so that they can be monitored by imaging, since this may present an opportunity for early resection of premalignant lesions. Consequently, the consensus signature was tested for its applicability to discriminate different stages of IPMN vs. NC. Twenty IPMN samples derived from the US patient cohort (Table 1) were classified, using the validated biomarker signature. Of note, the signature classified the borderline and malignant IPMNs as having a cancer profile, while benign IPMNs were classified as non-PDAC (p=0.029) (FIG. 6).

Discussion

The key finding in this study is that a proteomic multiparametric analysis, using minute volumes of serum could discriminate patients with early stage I/I PDAC from controls with high accuracy. The clinical utility and intended use of such a diagnostic approach would potentially be several fold, e.g. surveillance of (i) high-risk patients, such as hereditary PDAC, chronic pancreatitis, and Peutz-Jeghers syndrome patients; (ii) late onset diabetic patients over the age of 50 years, who have up to eight times increased risk for acquiring PDAC within the first three years of diabetes^{30, 31}, and (iii) patients with vague abdominal symptoms, back pain, and weight loss.

WHO has proposed that millions of cancer patients could be saved from premature death if diagnosed and treated earlier. To achieve this, more advanced diagnostic approaches have to be developed and applied to earlier detection of particularly lethal cancers such as PDAC. Despite the fact that the evolutionary trajectory of PDAC disease progression is discussed³²⁻³⁴, the available clinical data today supports the conclusion that earlier diagnosis leads to an overall survival benefit of asymptomatic patients, due to an increased frequency of resectable tumors^{4,8-11, 35}. To demonstrate clinical utility for early diagnosis for PDAC, the test has to display a low frequency of false positives, since this would otherwise inevitably lead to undesired consequences for the patient including anxiety, overtreatment, and increased costs. With this risk in mind, we have performed a large proteomic study on PDAC, including over 1700 case/control samples, and analysed 156 serum proteins derived either from the tumor secretome or from a systemic immune response. To determine clinical utility of a biomarker signature in a population, the prevalence of PDAC affects both the positive predictive value (PPV) (the probability that a positive test indicates disease) and the negative predictive value (NPV) (the probability that a negative test indicates absence of disease). In our US validation cohort, the results suggest that with a specificity as high as 99%, in patients with a higher risk than the general public for PDAC, e.g. first-degree relatives (prevalence 3.75%), and newly onset diabetic patients over 55 years of age (prevalence 1.0%)³⁶, the PPV/NPV would be 0.75/0.99 and 0.46/1.0, respectively. This signature, yielding the highest specificity/sensitivity for discriminating stage I/II from controls, did not include CA19-9, an antigen commonly involved in analysis of PDAC, either alone or in combination with other markers¹⁸. In fact, CA19-9 was analyzed on the antibody microarray but was not selected, since it did not contribute with enough orthogonal information during the backward elimination process.

Since newly onset diabetes in patients over 55 years of age has a significant increased risk of acquiring PDAC³⁷ this can be considered as an early indication of cancer, which could lead to early detection of asymptomatic, early stage PDAC³⁸. Diagnosis of diabetic patients with PDAC would consequently be of importance, since it would contribute to increased resectability and an increased survival in these patients. Consequently, we tested the consensus biomarker signature for its ability to discriminate between diabetic PDAC patients and PDAC without diagnosed diabetes. A support vector machine analysis, based on in total 141 diabetic patients with PDAC from both cohorts, of which 26.2% displayed newly onset diabetes, demonstrated no significant difference between samples derived from diabetic versus non-diabetic PDAC patients (FIG. 5). This implies that the validated biomarker signature potentially could contribute to clinically rule-out PDAC in diabetic patients, although this has to be demonstrated in a clinical study focusing on diabetic patients.

Differential diagnosis of PDAC vs. pancreatitis is sometimes difficult but in a previous study we demonstrated that late stage PDAC could be distinguished from different pancreatic inflammatory indications²⁷. A follow-up study was previously performed on different pancreatitis subtypes, such as acute, chronic, and autoimmune pancreatitis, where biomarkers associated with these subtypes could be identified and distinguished from PDAC³⁹. Even though the number of chronic pancreatitis samples is limited in the current study, we could demonstrate that chronic pancreatitis could be discriminated from early stage I/I PDAC, now with a ROC-AUC of 0.84 (FIG. 3B). Furthermore, correct classification of premalignant lesions of the pancreas (IPMN) represents a considerable clinical value. The present consensus biomarker signature could discriminate samples derived from patients with pathologically staged benign IPMNs from patients with stage I/II PDAC (FIG. 6), while borderline and malignant staged IPMNs were classified as cancer associated and could thus not be discriminated from PDAC. The limitation is that these results are based on a fairly low number of clinical samples but could potentially contribute to the detection of these difficult-to-diagnose lesions, when validated in a larger IPMN case/control study.

Relevant to cancer progression are gradual changes in the tumor microenvironment that can reflect back on the biomarker content in blood. Consequently, the data acquired here was used to identify markers whose expression pattern varied with stage progression, i.e. showed different levels in samples derived from early or late stage PDAC patients. Interestingly, all proteins displayed in FIG. 4, except IL-2, were present in the consensus signature (Table 2). Among the markers that displayed the most significantly increased expression from early to late stage PDAC was DLG1 (disks large homolog 1), a multi-functional scaffolding protein that interacts with e.g. APC, β-catenin, and PTEN to regulate cell proliferation, cytokinesis, migration, and adhesion. Although a candidate tumor suppressor DLG1 has been reported to exhibit oncogenic functions⁴⁰, potentially supported by the present upregulation in late stage PDAC. MAGI-1 (membrane-associated guanylate kinase, WW and PDZ domain-containing protein 1), also exhibited an increased expression in samples derived from late stage PDAC patients and is a scaffolding protein with proposed functions in epithelial cell-to-cell adhesion. Cancer related information in the literature is scarce, but MAGI-1 has been reported to inhibit both apoptosis and stimulate cell proliferation in HPV-induced malignancy⁴¹. PRDM8 (PR domain zinc finger protein 8), also known as BLIMP-1, was increased in samples from late stage patients. This DNA-binding protein regulates e.g. neural and steroid-related transcription, and is a regulator of tumorigenesis in pituitary adenomas, where it most likely contributes to increased tumor invasiveness⁴². This is consistent with our observation of its increased expression in late stage patient samples. Furthermore, lymphotoxin-alpha showed a lower expression in late stage samples. Lymphotoxin-alpha is produced by TH1 type T-cells to induce phagocyte binding to endothelial cells. Some polymorphisms of this protein contribute to increased risk for developing adenocarcinoma⁴³, although mapping previously has shown low protein expression in pancreatic cancer, a finding that could explain its decreased expression during PDAC progression in our study⁴⁴. The positive complement regulator properdin also showed decreased expression in samples from late stage PDAC patients. Properdin supports inflammation and phagocytosis via boosting of the alternative pathway of complement. Although inherently complex, complement activation is generally recognized as protective against cancer. Not only does inhibition of complement activation typically promote cancer cell immune evasion, it has also been shown to hamper the efficacy of cancer immunotherapy^{45, 46}. Decreased expression of properdin is consistent with the immune evasion observed in PDAC. Interleukin-2 (IL-2) exhibited decreased expression in samples from late stage patients. IL-2 stimulates growth and response of activated T-cells and is used in immunotherapy against e.g. renal carcinoma and malignant melanoma. Several studies show that IL-2 treatment in combination with conventional therapy can attenuate pancreatic cancer progression^{47, 48}. Further study of serum proteins that are associated with PDAC progression could potentially reveal mechanistic information on the biology of disease progression.

In summary, this study has succeeded in identifying and validating a biomarker signature based on two large case/control studies of PDAC patients. The findings show that this biomarker signature can detect samples derived from stage I/I PDAC patients with high accuracy, indicating the possibility to diagnose pancreatic cancer at an earlier stage, using a serum biomarker signature.

### REFERENCES

1. Torre L A, Bray F, Siegel R L, Ferlay J, Lortet-Tieulent J, Jemal A. Global cancer statistics, 2012. CA Cancer J Clin. 2015; 65(2):87-108.
2. Kamisawa T, Wood L D, Itoi T, Takaori K. Pancreatic cancer. Lancet. 2016; 388(10039):73-85.
3. Rahib L, Fleshman J M, Matrisian L M, Berlin J D. Evaluation of Pancreatic Cancer Clinical Trials and Benchmarks for Clinically Meaningful Future Trials: A Systematic Review. JAMA Oncol. 2016; 2(9):1209-16.
4. Ryan D P, Hong T S, Bardeesy N. Pancreatic adenocarcinoma. N Engl J Med. 2014; 371(22):2140-1.
5. Rahib L, Smith B D, Aizenberg R, Rosenzweig A B, Fleshman J M, Matrisian L M. Projecting cancer incidence and deaths to 2030: the unexpected burden of thyroid, liver, and pancreas cancers in the United States. Cancer Res. 2014; 74(11):2913-21.
6. Sohn T A, Yeo C J, Cameron J L, Koniaris L, Kaushal S, Abrams R A, et al. Resected adenocarcinoma of the pancreas-616 patients: results, outcomes, and prognostic indicators. J Gastrointest Surg. 2000; 4(6):567-79.
7. Zhang H, Wu X, Zhu F, Shen M, Tian R, Shi C, et al. Systematic review and meta-analysis of minimally invasive versus open approach for pancreaticoduodenectomy. Surg Endosc. 2016; 30(12):5173-84.
8. Matsuno S, Egawa S, Fukuyama S, Motoi F, Sunamura M, Isaji S, et al. Pancreatic Cancer Registry in Japan: 20 years of experience. Pancreas. 2004; 28(3):219-30.
9. Gangi S, Fletcher J G, Nathan M A, Christensen J A, Harmsen W S, Crownhart B S, et al. Time interval between abnormalities seen on C T and the clinical diagnosis of pancreatic cancer: retrospective review of C T scans obtained before diagnosis. AJR Am J Roentgenol. 2004; 182(4):897-903.
10. Pelaez-Luna M, Takahashi N, Fletcher J G, Chari S T. Resectability of presymptomatic pancreatic cancer and its relationship to onset of diabetes: a retrospective review of C T scans and fasting glucose values prior to diagnosis. Am J Gastroenterol. 2007; 102(10):2157-63.
11. Vasen H, Ibrahim I, Ponce C G, Slater E P, Matthai E, Carrato A, et al. Benefit of Surveillance for Pancreatic Cancer in High-Risk Individuals: Outcome of Long-Term Prospective Follow-Up Studies From Three European Expert Centers. J Clin Oncol. 2016; 34(17):2010-9.
12. Hanada K, Okazaki A, Hirano N, Izumi Y, Minami T, Ikemoto J, et al. Effective screening for early diagnosis of pancreatic cancer. Best Pract Res Clin Gastroenterol. 2015; 29(6):929-39.
13. Chari S T, Kelly K, Hollingsworth M A, Thayer S P, Ahlquist D A, Andersen D K, et al. Early detection of sporadic pancreatic cancer: summative review. Pancreas. 2015; 44(5):693-712.
14. Brentnall T A. Progress in the Earlier Detection of Pancreatic Cancer. J Clin Oncol. 2016; 34(17):1973-4.
15. Okano K, Suzuki Y. Strategies for early detection of resectable pancreatic cancer. World J Gastroenterol. 2014; 20(32):11230-40.
16. Locker G Y, Hamilton S, Harris J, Jessup J M, Kemeny N, Macdonald J S, et al. ASCO 2006 update of recommendations for the use of tumor markers in gastrointestinal cancer. J Clin Oncol. 2006; 24(33):5313-27.
17. Galli C, Basso D, Plebani M. CA 19-9: handle with care. Clin Chem Lab Med. 2013; 51(7):1369-83.
18. Borrebaeck C A. Precision diagnostics: moving towards protein biomarker signatures of clinical utility in cancer. Nat Rev Cancer. 2017; 17(3):199-204.
19. Hanash S M, Pitteri S J, Faca V M. Mining the plasma proteome for cancer biomarkers. Nature. 2008; 452(7187):571-9.
20. Radon T P, Massat N J, Jones R, Alrawashdeh W, Dumartin L, Ennis D, et al. Identification of a Three-Biomarker Panel in Urine for Early Detection of Pancreatic Adenocarcinoma. Clin Cancer Res. 2015; 21(15):3512-21.
21. Shaw V E, Lane B, Jenkinson C, Cox T, Greenhalf W, Halloran C M, et al. Serum cytokine biomarker panels for discriminating pancreatic cancer from benign pancreatic disease. Mol Cancer. 2014; 13:114.
22. Mayers J R, Wu C, Clish C B, Kraft P, Torrence M E, Fiske B P, et al. Elevation of circulating branched-chain amino acids is an early event in human pancreatic adenocarcinoma development. Nat Med. 2014; 20(10):1193-8.
23. Jenkinson C, Elliott V L, Evans A, Oldfield L, Jenkins R E, O'Brien D P, et al. Decreased Serum Thrombospondin-1 Levels in Pancreatic Cancer Patients Up to 24 Months Prior to Clinical Diagnosis: Association with Diabetes Mellitus. Clin Cancer Res. 2016; 22(7): 1734-43.
24. Brand R E, Nolen B M, Zeh H J, Allen P J, Eloubeidi M A, Goldberg M, et al. Serum biomarker panels for the detection of pancreatic cancer. Clin Cancer Res. 2011; 17(4):805-16.
25. Kim J, Bamlet W R, Oberg A L, Chaffee K G, Donahue G, Cao X J, et al. Detection of eraly pancreatic ductal adenocarcinoma with thrombospondin-2 and CA19-9 blood markers.
Sci. Transl. Med. 2017; 12; 9(398) doi: 10.1126/scitranslmed.aah5583
26. Gerdtsson A S, Malats N, Sail A, Real F X, Porta M, Skoog P, et al. A Multicenter Trial Defining a Serum Protein Signature Associated with Pancreatic Ductal Adenocarcinoma. Int J Proteomics. 2015; 2015:587250.
27. Wingren C, Sandstrom A, Segersvard R, Carlsson A, Andersson R, Lohr M, et al. Identification of serum biomarker signatures associated with pancreatic cancer. Cancer Res. 2012; 72(10):2481-90.
28. Bossuyt P M, Reitsma J B, Bruns D E, Gatsonis C A, Glasziou P P, Irwig L, et al. STARD 2015: an updated list of essential items for reporting diagnostic accuracy studies. BMJ. 2015; 351:h5527.
29. Carlsson A, Wingren C, Kristensson M, Rose C, Ferno M, Olsson H, et al. Molecular serum portraits in patients with primary breast cancer predict the development of distant metastases. Proc Natl Acad Sci USA. 2011; 108(34):14252-7.
30. Batabyal P, Vander Hoorn S, Christophi C, Nikfarjam M. Association of diabetes mellitus and pancreatic adenocarcinoma: a meta-analysis of 88 studies. Ann Surg Oncol. 2014; 21(7):2453-62.
31. Wang F, Herrington M, Larsson J, Permert J. The relationship between diabetes and pancreatic cancer. Mol Cancer. 2003; 2:4.
32. Lopez-Lazaro M. Pancreatic cancer formation is gradual, ResearchGate 2017, doi10.13140/RG.2.2.16865.92009
33. Notta F, Chan-Seng-Yue M, Lemire M, Li Y, Wilson G W, Connor A A, et al. A renewed model of pancreatic cancer evolution based on genomic rearrangement patterns. Nature. 2016; 538(7625):378-82.
34. Yachida S, Jones S, Bozic I, Antal T, Leary R, Fu B, et al. Distant metastasis occurs late during the genetic evolution of pancreatic cancer. Nature. 2010; 467(7319):1114-7.
35. Shimizu Y, Yasui K, Matsueda K, Yanagisawa A, Yamao K. Small carcinoma of the pancreas is curable: new computed tomography finding, pathological study and postoperative results from a single institute. J Gastroenterol Hepatol. 2005; 20(10):1591-4.
36. Chari S Y, Leibson C L. Rabe K G, Ransom J, De Andrade M, Petersen G M. Gastroenterology 2005, 129(2) 505-511.
37. Aggarwal G, Rabe K G, Petersen G M, Chari S T. New-onset diabetes in pancreatic cancer: A study in the primary care setting. Pancreatology 2012; 12(2) 156-161.
38. Pannala R, Basu A, Petersen G M, Chari S T. New-onset Diabetes: A potential clue to the early diagnosis of pancreatic cancer. Lancet Oncology 2009, 10(1) 88-95.
39. Sandstrom A, Andersson R, Segersvard R, Lohr M, Borrebaeck C A, Wingren C. Serum proteome profiling of pancreatitis using recombinant antibody microarrays reveals disease-associated biomarker signatures. Proteomics Clin Appl. 2012; 6(9-10):486-96.
40. Roberts S, Delury C, Marsh E. The PDZ protein discs-large (DLG): the ‘Jekyll and Hyde’ of the epithelial polarity proteins. FEBS J. 2012; 279(19):3549-58.
41. Kranjec C, Massimi P, Banks L. Restoration of MAGI-1 expression in human papillomavirus-positive tumor cells induces cell growth arrest and apoptosis. J Virol. 2014; 88(13):7155-69.
42. Lan X, Gao H, Wang F, Feng J, Bai J, Zhao P, et al. Whole-exome sequencing identifies variants in invasive pituitary adenomas. Oncol Lett. 2016; 12(4):2319-28.
43. Huang Y, Yu X, Wang L, Zhou S, Sun J, Feng N, et al. Four genetic polymorphisms of lymphotoxin-alpha gene and cancer risk: a systematic review and meta-analysis. PLoS One. 2013; 8(12):e82519.
44. Expression of LTA in cancer—The Human Protein Atlas 2017 [Available from: http://www.proteinatlas.orq/ENSG00000226979-LTA/cancer.]
45. Mamidi S, Hone S, Kirschfink M. The complement system in cancer: Ambivalence between tumour destruction and promotion. Immunobiology. 2017; 222(1):45-54.
46. Pio R, Corrales L, Lambris J D. The role of complement in tumor growth. Adv Exp Med Biol. 2014; 772:229-62.
47. Grande C, Firvida J L, Navas V, Casal J. Interleukin-2 for the treatment of solid tumors other than melanoma and renal cell carcinoma. Anticancer Drugs. 2006; 17(1):1-12.
48. Nobili C, Degrate L, Caprotti R, Franciosi C, Leone B E, Trezzi R, et al. Prolonged survival of a patient affected by pancreatic adenocarcinoma with massive lymphocyte and dendritic cell infiltration after interleukin-2 immunotherapy. Report of a case. Tumori. 2008; 94(3):426-30.

TABLE 1
Demographics of the Scandinavian and North American cohorts
(A) Scandinavian cohort

| Sample | AJCC | No. of | | | | | Tobacco use | Alcohol abuse |
|---|---|---|---|---|---|---|---|---|
| status | Stage | samples | Training set size* | Test set size* | Gender (Men/Women) | Median age (Range) | Y/N (%) | Y/N (%) |
| PDAC | IA | 10 | 8 | 2 | 5/5 | 68.5 (38-80) | 4/6 (40) | 0/10 (0) |
| | IB | 6 | 5 | 1 | 1/5 | 73.5 (51-80) | 3/3 (50) | 1/5 (17) |
| | IIA | 32 | 24 | 8 | 22/10 | 69 (38-88) | 12/20 (38) | 10/22 (31) |
| | IIB | 100 | 75 | 25 | 56/44 | 66 (37-86) | 74/26 (74) | 11/89 (11) |
| | III | 65 | 48 | 17 | 37/28 | 68 (49-86) | 42/23 (65) | 16/49 (25) |
| | IV | 230 | 172 | 58 | 132/98 | 68 (40-89) | 157/73 (68) | 58/172 (25) |
| IPMN | Benign | 5 | — | 5 | 3/2 | 71 (60-77) | 3/4 (43) | 4/1 (80) |
| | Malignant | 3 | — | 3 | 2/1 | 70 (64-70) | 3/0 (100) | 0/3 (0) |
| NC | | 888 | 666 | 222 | 512/376 | 68 (33-96) | 527/357 (60) | 212/676 (24) |

(B) US cohort

| Sample status | AJCC Stage | No. of samples | Training set size* | Test set size* | Gender (Men/Women) | Median age (Range) |
|---|---|---|---|---|---|---|
| PDAC | IA | 5 | 3 | 2 | 3/2 | 67 (56-73) |
| | IB | 10 | 7 | 3 | 7/3 | 69 (38-82) |
| | IIA | 27 | 18 | 9 | 16/11 | 65 (46-87) |
| | IIB | 48 | 32 | 16 | 24/24 | 67 (30-84) |
| | III | 15 | 10 | 5 | 8/7 | 66 (24-83) |
| | IV | 38 | 25 | 13 | 23/15 | 65.5 (35-83) |
| Chronic Pancreatitis | | 57 | 38 | 19 | 26/28 | 55.5 (32-81) |
| IPMN | Benign | 8 | — | 8 | 1/7 | 63 (46-75) |
| | Borderline | 5 | — | 5 | 2/3 | 74 (71-79) |
| | Malignant | 7 | — | 7 | 5/2 | 63 (54-79) |
| NC | | 219 | 146 | 73 | 115/104 | 63 (24-86) |

*Representative set sizes. For further information see section on bioinformatics.

TABLE 2
Consensus validation signature

Protein
Apolipoprotein A1
Aprataxin and PNK-like factor
Calcineurin B homologous protein 1
Calcium/calmodulin-dependent protein kinase type IV
Complement C3
Complement C4
Complement C5
Cyclin-dependent kinase 2
Disks large homolog 1
GTP-binding protein GEM
HADH2 protein
Intercellular adhesion molecule 1
Interferon gamma
Interleukin-13
Interleukin-4
Interleukin-6
Lewis x
Lymphotoxin-alpha
Membrane-associated guanylate kinase,
WW and PDZ domain-containing
protein 1
Myomesin-2
Plasma protease C1 inhibitor
PR domain zinc finger protein 8
Properdin
Protein kinase C zeta type
Protein-tyrosine kinase 6
Serine/threonine-protein kinase MARK1
Sialyl Lewis x
Vascular endothelial growth factor
Visual system homeobox 2

Supplemental Information

Methods

Demographics of Study Cohorts

The controls for the Scandinavian cohort were obtained from the Copenhagen General Population Study and were matched for gender, age, smoking habits, alcohol intake, and date of blood sampling. Two controls were matched per case. None of the controls had developed pancreatic cancer during a 5-year follow-up. Gender balance was 57:43(%) men vs. women in PDAC patients and 58:42(%) men vs. women in NC. The median age of the PDAC and NC subjects were both 68 years. Tobacco use was defined as current or past regular use, while alcohol abuse was defined as current or past abuse. Based on guidelines from the Danish Health Authority, the cut-offs for alcohol abuse were set at 168 g and 252 g alcohol per week for women and men, respectively. The ratio of tobacco users in the PDAC group, control group and all subjects combined were 66%, 60%, and 62%, respectively. The corresponding values for alcohol abuse were 22%, 24%, and 23%, respectively (Table 1). Of all PDAC patients in the Scandinavian cohort, 23.3% suffered from diabetes at the time of sample collection, while 25.0%, 28.7%, 26.2%, and 19.1% of stages I, 11, III, and IV PDAC patients, respectively, had known diabetes at the time of blood sampling (Table 3). Regardless of diabetic status, 70% of the tumors were located in the head, 20% in the body, and 10% in the pancreatic tail (Table 3). These proportions correspond well to the commonly reported data on tumor localization¹. All other parameters, including liver values and blood cell type counts, were comparable between disease stages (Table 3). Staging for the Scandinavian cohort was based on pathologic state of the resected tumor and lymph nodes and CT-scans (abdominal and thorax) in the resected patients and on biopsy and CT-scans for the non-resected patients.

The controls for the US cohort were collected either during a blood drive targeting healthy, non-cancer controls or during an office visit of non-cancer individuals and matched to PDAC patients regarding gender and age at time of sample collection. None of the controls had developed pancreatic cancer during a 5-year follow-up. Gender balance was 56:44(%) men vs. women in PDAC patients, 53:47(%) men vs. women in NC, 48:52(%) men vs. women in chronic pancreatitis (CP) patients, and 40:60(%) men vs. women in IPMN patients. The median age for PDAC, NC, CP, and IPMN subjects were 67, 63, 56, and 69 years, respectively. Staging for the US cohort was based on pathologic state, except in the case where there was no resection, i.e. typically late stage disease. For those patients, staging was based on biopsy or imaging depending on the clinical course. Of all PDAC patients in the US cohort, 26.6% suffered from diabetes at the time of sample collection, while 26.7%, 26.7%, 20.0%, and 28.9% of stages I, II, III, and IV PDAC patients, respectively, had known diabetes at the time of blood sampling (Table 3). IPMN diagnosis in both cohorts were based on surgically obtained pathology. Furthermore, the diagnosis of chronic pancreatitis was made by, 1) symptoms, i.e. pain and/or pancreatic insufficiency as determined by pancreatic elastase, following episodes of acute pancreatitis that were biochemically confirmed with amylase and lipase determinations and had abdominal imaging with CT scan that showed pancreatic and aperi-pancreatic inflammation, and 2) imaging—all patients had ERCP that showed pancreatic ductal changes consistent with chronic pancreatitis and all had CT and/or MRI imaging. All patients went to surgery for drainage procedures.

Sample Collection

The Scandinavian study, denoted the BIOPAC Study “BIOmarkers in patients with PAncreatic Cancer—can they provide new information of the disease and improve diagnosis and prognosis of the patients”, was approved by the Regional Ethics Committees of Copenhagen (VEK ref. KA-2006-0113) and the Danish Data Protection Agency (jr. no. 2006-41-6848, jr. no. 2012-58-004 and HGH-2015-027, I-suite 03960). The serum samples were collected between 2008 and 2014 at Herlev Hospital and Rigshospitalet, Copenhagen, Denmark. At the time of diagnosis, the blood was collected and allowed to clot for at least 30 minutes and then centrifuged at 2330 g for 10 minutes at 4° C. The serum was aliquoted and stored at −80° C. until further analysis. All samples were collected and processed, using the same SOP and analyzed for serum CA19-9, liver enzymes, and blood cell counts. Clinical data was gathered at time of sample collection.

The US study was approved by the Institutional Review Board of Oregon Health and Science University. Blood was collected prior to any treatment, allowed to clot for at least 30 minutes, and centrifuged at 1500 g for 10 minutes at 4° C. All samples were collected and processed, using the same SOP. The serum was aliquoted and stored at −80° C. until further analysis.

Data Acquisition, Quality Control, and Pre-Processing

Signal intensities from the antibody microarray were quantified, using the Array-Pro Analyzer software (Media Cybernetics, Rockville, Md., USA). Local background values were subtracted, and the adjusted intensity values were then used for subsequent data analysis. Data acquisition was performed by trained members of the research team who were blinded to sample classification and clinical data. Each data point represented a background-subtracted signal average of three replicate spots per antibody clone, unless the replicate coefficient of variance (CV) exceeded 15%. In such cases the replicate spot furthest from the mean value was omitted and the average signal of the two remaining replicates was used. The average CVs of replicates were 8.4% and 6.7% in the Scandinavian and US study, respectively.

The raw data from the quality control samples was evaluated on an individual antibody level for inter-slide and inter-day variance by CV-value analysis, box plotting, and 3D principal component analysis (PCA) with analysis of variance (ANOVA) filtering (Qlucore Omics Explorer, Qlucore AB, Lund, Sweden). Once data set homogeneity had been assured the quality control samples were removed from further analysis. Data from PDAC and control samples was transformed by log 2 followed by adjustment and normalization in two steps to reduce technical variation between days and slides. In the first step, day-to-day variation was addressed by applying ComBat (SVA package in the statistical software environment R), a method to adjust batch effects, using empirical Bayes frameworks where the batch covariate is known^{2, 3}. The covariate used was the day of microarray assay. In a second step, array-to-array variation was minimized, by calculating a scaling factor for each array. This factor was based on the 20% of antibodies with the lowest standard deviation of all samples and was calculated by dividing the intensity sum of these antibodies on each array with the average sum across all arrays⁴. The data is available from the corresponding author upon request.

Data Analysis

Two-group classifications were performed, using support vector machine (SVM) analysis in R. PCA, q-value calculation by ANOVA, and fold change calculation were performed, using Qlucore Omics Explorer. Multigroup ANOVA was used to analyze differential expression of individual protein markers in samples from the various PDAC stages included in the Scandinavian cohort. The performance of individual markers was evaluated with Student's t-test, Benjamini-Hochberg procedure for false discovery rate control (q-values), and fold changes. Sensitivities, specificities were calculated from SVM decision values. Positive (PPV), and negative (NPV) predictive values were calculated in relation to prevalence and lifetime risk for risk groups, such as newly onset diabetes (NOD) patients over 55 years of age and first-degree relatives for PDAC patients.

Before defining a biomarker signature that discriminated NC from PDAC Stage I/II, the power to classify individual PDAC stages was evaluated, using a leave-one-out (LOO) cross validation approach in R based on all antibodies⁵. In short, an SVM was designed in 5 which one data point was partitioned into a separate subset (test set) and the remaining data points were used as the training set. The process was repeated one sample at a time, the results were used to create a receiver operating characteristic (ROC) curve, and the corresponding area under the curve (AUC) value was calculated.

Next, to decipher a condensed biomarker signature, the data was divided into a training set including 3/4 of the samples (approximately 1000 samples) and a test set including 1/4 of the samples (approximately 340 samples). The ratio of case vs. control samples within the data sets was retained, but otherwise the sets were randomly generated. Four unique test/training sets were generated, using this approach. An individual sample was only included once in a test set. In order to identify the biomarker signatures, a Backward Elimination (BE) algorithm was applied to each training set in R, excluding one antibody at a time. For each BE iteration, the antibody with the highest Kullback-Leibler (KL) divergence value obtained in the classification analysis was eliminated. Based on KL divergence value analysis, the antibody combinations expressing the lowest values were used to design the predictive biomarker signature. Consequently, BE allows an unbiased selection of markers contributing orthogonal information, compared to other biomarkers⁶. Of note, the BE process sometimes results in that previously defined tumor markers, such as CA19-9 and Sialyl Lewis A in the case of PDAC, are not included in the signature, since they do not contribute with enough orthogonal information. The identified biomarker signature was then used to build a prediction model by frozen SVM in R, using only the training data set⁵. Furthermore, to avoid overfitting, the model was tested on the corresponding test set and its performance was assessed, using ROC curves and AUC values. To further minimize over-interpretation and to ensure robustness this process was performed on all four training and test sets. In this manner, a prediction model classifying NC vs. PDAC stage I/I patients was built and its performance was assessed, using ROC curves and AUC values. As a comparison, this was repeated also for samples derived from NC vs. PDAC stage III/IV patients.

Finally, to obtain a consensus signature with the highest predictive classification accuracy data from all classifications of NC vs. PDAC stage I/I patients as well as NC vs. PDAC stage III/IV were combined. The predictive accuracy of the consensus signature was then validated in an independent US sample cohort.

In the US study used for validation, the data was divided into three training/test sets of approximately 280 samples (training) and approximately 140 samples (test). The ratio of case vs. control samples within the data sets was retained, but otherwise the sets were randomly generated. The consensus signature from the Scandinavian study was used to build prediction models, using only the US training sets. The model was then tested on the corresponding US test set and the performance was assessed, using ROC curves and AUC values. To further minimize over-interpretation and to ensure robustness this process was performed on all three training and test sets. The same approach was used for the classification of chronic pancreatitis vs. PDAC samples, using a frozen SVM and the ROC-AUC value was calculated. Finally, the consensus signature was used to classify NC vs. IPMN patients. All IPMN samples in the validation cohort were fed into an SVM model that had been trained on NC vs. PDAC. To investigate whether bilirubin levels or diabetes were confounding factors in the antibody microarray analysis, patients with jaundice (49.7%) and diabetes (26.6%) were compared to patients without jaundice or without diabetes, respectively.

Sample Labeling

In both studies, the serum samples were labeled with biotin, using a protocol optimized for serum proteomes⁶⁻⁸. Briefly, 5 μl serum samples were diluted 1:45 in PBS to ˜2 mg protein/ml and labeled with 0.6 mM EZ-Link Sulfo-NHS-LC-Biotin (Thermo Fisher Scientific, Waltham, Mass., USA). Unbound biotin was removed by dialysis against PBS for 72 hours using a 3.5 kDa MWCO dialysis membrane (Thermo Fisher Scientific, Waltham, Mass., USA), changing buffer every 24 hours. The labeled serum samples were aliquoted and stored at −20° C. To control for labeling quality, reference serum samples (LGC Standards, Teddington, UK) were labeled alongside patient samples during each biotinylation round. The signals from these quality control (QC) samples were compared with the signals from a batch of identical previously labeled reference serum (see section on microarray assay) to verify that the process had worked as intended.

Antibody Microarray Production

Identical antibody microarrays were utilized in both studies. The arrays comprised 339 human recombinant scFvs directed against 156 known antigens (Table 5). The scFvs, selected and generated from phage display libraries, have previously been shown to display robust on-chip functionality^{7, 9-12}. Alongside the scFvs, two full length monoclonal antibodies against CA19-9 (Meridian Life Science, Memphis, Tenn., USA) were printed on the slides. The majority of the antibodies have previously been tested in array applications¹⁰⁻¹², and their specificity validated, using well-characterized control sera. Furthermore, orthogonal methods such as mass spectrometry, ELISA, MesoScaleDiscovery cytokine assay, cytometric bead assay, and spiking and blocking ELISA have been utilized for assessing antibody specificities¹³⁻¹⁵. The selected scFvs were against serum proteins mostly involved in immune regulation and/or cancer biology.

His-tagged scFvs were produced in E. coli and purified from the periplasm, using a magnetic Ni-particle protein purification system (MagneHis, Promega, Madison, Wis., USA). The elution buffer was exchanged for PBS, using Zeba 96-well spin plates (Pierce, Rockford, Ill., USA). Protein yield was measured using NanoDrop spectrophotometer (Thermo Fisher Scientific, Waltham, Mass., USA). Protein purity was checked by 10% Bis-Tris SDS-PAGE (Invitrogen, Carlsbad, Calif., USA). Antibody microarrays were produced on black MaxiSorp slides (NUNC, Roskilde, Denmark), using a non-contact printer (SciFlexarrayer S11, Scienion, Berlin, Germany). Prior to printing, optimal printing concentration was defined for each scFv clone⁹. To allow for subsequent QC functions, 0.1 mg/ml Cadaverine Alexa Fluor-555 (Life Technologies, Carlsbad, Calif., USA) was added to the printing buffer. Fourteen identical arrays were printed on each slide in two columns of seven arrays. Each array consisted of 34×36 spots with 200 □m spot-to-spot center distance and a spot diameter of 140 Em. Each array consisted of three identical segments separated by rows of BSA-biotin spots. Each antibody was printed in three replicates with one replicate in each segment. Two additional rows of biotin-BSA spots flanked each subarray, one above the subarray and one below it. Nine negative control spots (PBS) were printed in each replicate segment. Ten slides (140 microarrays) were printed, for each round of analysis. In the Scandinavian discovery study a total of 152 slides were printed over 16 printing days. In the validation study a total of 48 slides were printed over five printing days. The slides were stored for eight days in room temperature (RT) before microarray assay.

Microarray Assay

Ten samples were analyzed on each slide. The positioning of the samples was randomized but the ratio of healthy and PDAC samples on each slide was approximately the same for the cohort as a whole. Four positions on each slide were used for QC samples; three for reference sera (two from LGC Standards, Teddington, UK, and one from SeraCare Life Sciences, Milford, Mass., USA) and one for a sample containing a mix of aliquots from healthy and cancer samples included in the study. Each microarray slide was mounted in a hybridization gasket (Schott, Mainz, Germany) and blocked with 1% w/v milk, 1% v/v Tween-20 in sterile D-PBS (MT-PBS) at RT for 1 hour with constant agitation. Meanwhile, aliquots of labeled serum samples were thawed on ice and subsequently diluted 1:10 in MT-PBS. The slides were washed four times with 0.05% Tween-20 in sterile D-PBS (PBST) followed by addition of diluted serum samples to the wells of the gasket. Samples were incubated on the slides at RT for 2 hours with constant agitation. Next, the slides were washed four times with PBST, incubated with 1 □g/ml Streptavidin Alexa-647 (Life Technologies Carlsbad, Calif., USA) in MT-PBS at RT for 1 hour with constant agitation, and again washed four times with PBST. Finally, the slides were dismounted from the hybridization gaskets, immersed in dH₂0 and dried under a stream of N₂. The slides were immediately scanned with a confocal microarray scanner (LS Reloaded, Tecan, Mannedorf, Switzerland) at 10 □m resolution, first at 635 nm, then at 532 nm. The first scan image detected the Alexa-647 (streptavidin) signal and was used for quantification of spot signal intensities. The second scan image measured the Alexa-555 (cadaverine) signal and was used for quality control purposes.

### REFERENCES (FOR SUPPLEMENTAL INFORMATION)

1. Stark A, Eibl G. Pancreatic Ductal Adenocarcinoma 2015 [Available from: https://www.pancreapedia.org/reviews/pancreatic-ductal-adenocarcinoma.
2. Johnson W E, Li C, Rabinovic A. Adjusting batch effects in microarray expression data using empirical Bayes methods. Biostatistics. 2007; 8(1):118-27.
3. Leek J T, Johnson W E, Parker H S, Fertig E J, Jaffe A E, Storey J D. sva: Surrogate Variable Analysis. R package version 3.22.0. 2016.
4. Delfani P, Dexlin Mellby L, Nordstrom M, Holmer A, Ohlsson M, Borrebaeck C A, et al. Technical Advances of the Recombinant Antibody Microarray Technology Platform for Clinical Immunoproteomics. PLoS One. 2016; 11(7):e0159138.
5. Carlsson A, Wingren C, Kristensson M, Rose C, Ferno M, Olsson H, et al. Molecular serum portraits in patients with primary breast cancer predict the development of distant metastases. Proc Natl Acad Sci USA. 2011; 108(34):14252-7.
6. Carlsson A, Persson O, Ingvarsson J, et al. Plasma proteome profiling reveals biomarker patterns associated with prognosis and therapy selection in glioblastoma multiforme patients. Proteomics Clin Appl 2010; 4:591-602.
7. Gerdtsson A S, Malats N, Sail A, et al. A Multicenter Trial Defining a Serum Protein Signature Associated with Pancreatic Ductal Adenocarcinoma. Int J Proteomics 2015; 2015:587250.
8. Wingren C, Ingvarsson J, Dexlin L, Szul D, Borrebaeck C A. Design of recombinant antibody microarrays for complex proteome analysis: choice of sample labeling-tag and solid support. Proteomics 2007; 7:3055-65.
9. Delfani P, Dexlin Mellby L, Nordstrom M, et al. Technical Advances of the Recombinant Antibody Microarray Technology Platform for Clinical Immunoproteomics. PLoS One 2016; 11:e0159138.
10. Steinhauer C, Wingren C, Hager A C, Borrebaeck C A. Single framework recombinant antibody fragments designed for protein chip applications. Biotechniques 2002; Suppl:38-45.
11. Wingren C, Borrebaeck C A. Antibody microarray analysis of directly labelled complex proteomes. Curr Opin Biotechnol 2008; 19:55-61.
12. Wingren C, Steinhauer C, Ingvarsson J, Persson E, Larsson K, Borrebaeck C A. Microarrays based on affinity-tagged single-chain Fv antibodies: sensitive detection of analyte in complex proteomes. Proteomics 2005; 5:1281-91.
13. Borrebaeck C A, Wingren C. Recombinant antibodies for the generation of antibody arrays. Methods Mol Biol 2011; 785:247-62.
14. Olsson N, Wallin S, James P, Borrebaeck C A, Wingren C. Epitope-specificity of recombinant antibodies reveals promiscuous peptide-binding properties. Protein Sci 2012; 21:1897-910.
15. Soderlind E, Strandberg L, Jirholt P, et al. Recombining germline-derived CDR sequences for creating diverse single-framework antibody libraries. Nat Biotechnol 2000; 18:852-6.

TABLE 3
Clinical data
Diabetes and jaundice in the Scandinavian and US cohorts

| | Diabetes | Diabetes | Jaundice |
|---|---|---|---|
| AJCC stage | Scandinavian cohort (%) | US cohort (%) | US cohort (%) |
| IA | 2/10 (20.0) | 1/5 (20.0) | 1/5 (20.0) |
| IB | 2/6 (33.3) | 3/10 (30.0) | 4/10 (40.0) |
| IIA | 7/32 (21.9) | 8/27 (29.6) | 13/27 (48.1) |
| IIB | 31/100 (31.0) | 12/48 (25.0) | 32/48 (66.7) |
| III | 17/65 (26.2) | 3/15 (20.0) | 6/15 (40.0) |
| IV | 44/230 (19.1) | 11/38 (28.9) | 15/38 (39.5) |
| I-IV | 103/443 (23.3) | 38/143 (26.6) | 71/143 (49.7) |

Tumor localization in the Scandinavian cohort

| AJCC stage | Head (%) | Body (%) | Tail (%) | Diffuse (%) | Unknown (%) |
|---|---|---|---|---|---|
| IA | 6 (60) | 3 (30) | — | — | 1 (10) |
| IB | 5 (83) | 1 (17) | — | — | — |
| IIA | 25 (78) | 1 (3) | 4 (13) | 2 (6) | — |
| IIB | 84 (84) | 10 (10) | 3 (3) | 2 (2) | 1 (1) |
| III | 43 (66) | 18 (28) | 1 (2) | 2 (3) | 1 (2) |
| IV | 136 (59) | 46 (20) | 34 (15) | 5 (2) | 9 (4) |

Clinical parameters in the Scandinavian cohorts

| AJCC | CA 19-9 | BASP | Bilirubin | ALAT | ASAT | Platelets | Leukocyte | Neutrophil |
|---|---|---|---|---|---|---|---|---|
| stage | (U/ml) | (U/l) | (μM) | (U/l) | (IU/l) | (PLT/nl) | (WBC/nl) | (ANC/nl) |
| IA | 59 | 77 | 8 | 15 | 29 | 284 | 12.2 | 18.7 |
| IB | 36 | 107 | 8 | 22 | 36.5 | 375 | 6.6 | 9 |
| IIA | 458 | 209 | 28 | 94 | 72 | 300 | 10 | 10 |
| IIB | 217 | 183 | 20 | 59 | 38 | 268 | 10 | 7 |
| III | 601 | 120 | 13 | 35 | 34.5 | 282.5 | 7.7 | 5.4 |
| IV | 1980 | 175 | 13 | 35 | 39 | 314 | 9 | 6.3 |

TABLE 4
Biomarker signatures discriminating
PDAC stages I/II and III/IV from NC
NC vs. PDAC stage I/II

| | |
|---|---|
| 1. | Plasma protease C1 inhibitor |
| 2. | Interleukin-4 |
| 3. | Protein-tyrosine kinase 6 |
| 4. | Complement C3 |
| 5. | Serine/threonine-protein kinase MARK1 |
| 6. | HADH2 protein |
| 7. | Properdin |
| 8. | Complement C4 |
| 9. | Cyclin-dependent kinase 2 |
| 10. | Interferon gamma |
| 11. | Calcium/calmodulin-dependent protein kinase 1 |
| 12. | Complement C5 |
| 13. | Vascular endothelial growth factor |
| 14. | Visual system homeobox 2 |
| 15. | PR domain zinc finger protein 8 |
| 16. | Intercellular adhesion molecule 1 |
| 17. | Ubiquitin carboxyl-terminal hydrolase isozyme L5 |
| 18. | Interleukin-6 |
| 19. | Myomesin-2 |
| 20. | Aprataxin and PNK-like factor |
| 21. | Apolipoprotein A1 |
| 22. | Regulator of nonsense transcripts 3B |
| 23. | Lumican |
| 24. | Interleukin-9 |
| 25. | C-C motif chemokine 13 |

NC vs. PDAC stage III/IV

| | |
|---|---|
| 1. | Plasma protease C1 inhibitor |
| 2. | Interleukin-4 |
| 3. | Complement C3 |
| 4. | Properdin |
| 5. | Complement C4 |
| 6. | Sialyl Lewis X |
| 7. | Calcineurin B homologous protein 1 |
| 8. | HADH2 protein |
| 9. | Protein-tyrosine kinase 6 |
| 10. | Apolipoprotein A1 |
| 11. | C-C motif chemokine 13 |
| 12. | Membrane-associated guanylate kinase, |
| | WW and PDZ domain-containing protein 1 |
| 13. | Lymphotoxin-alpha |
| 14. | Disks large homolog 1 |
| 15. | Protein kinase C zeta type |
| 16. | Interleukin-13 |
| 17. | Complement C5 |
| 18. | Serine/threonine-protein kinase MARK1 |
| 19. | GTP-binding protein GEM |
| 20. | IgM |
| 21. | Interleukin-8 |
| 22. | Vascular endothelial growth factor |
| 23. | Interleukin-6 |
| 24. | Interleukin-9 |

TABLE 5
scFv Specificities

| | | No. of |
|---|---|---|
| Antigen | Full name | scFvs |
| AKT3 | RAC-gamma serine/threonine-protein kinase | 2 |
| Angiomotin | Angiomotin | 2 |
| ANM5 | Protein arginine N-methyltransferase 5 | 2 |
| APLF | Aprataxin and PNK-like factor | 2 |
| APOA4 | Apolipoprotein A4 | 2 |
| APOA1 | Apolipoprotein A1 | 3 |
| ARHGC | Rho guanine nucleotide exchange factor 12 | 1 |
| ATP5B | ATP synthase subunit beta, mitochondrial | 2 |
| β-galactosidase | Beta-galactosidase | 1 |
| BIRC2 | Baculoviral IAP repeat-containing protein 2 | 2 |
| BTK | Tyrosine-protein kinase BTK | 3 |
| C1 esterase | Plasma protease C1 inhibitor | 3 |
| inhibitor | | |
| C1q | Complement C1q | 1 |
| C1s | Complement C1s | 1 |
| C3 | Complement C3 | 4 |
| C4 | Complement C4 | 3 |
| C5 | Complement C5 | 3 |
| CBPP22 | Calcineurin B homologous protein 1 | 2 |
| CD40 | CD40 protein | 4 |
| CD40L | CD40 ligand | 1 |
| CDK2 | Cyclin-dependent kinase 2 | 2 |
| CENTG1 | Arf-GAP with GTPase, ANK repeat and | 2 |
| | PH domain-containing protein 2 | |
| CHEK2 | Serine/threonine-protein kinase Chk2 | 2 |
| CHX10 | Visual system homeobox 2 | 2 |
| CSNK1E | Casein kinase I isoform epsilon | 2 |
| Cystatin C | Cystatin-C | 2 |
| DCNL1 | DCN1-like protein 1 | 2 |
| Digoxin | Digoxin | 1 |
| DLG1 | Disks large homolog 1 | 2 |
| DLG2 | Disks large homolog 2 | 2 |
| DLG4 | Disks large homolog 4 | 2 |
| DPOLM | DNA-directed DNA/RNA polymerase mu | 2 |
| DUSP7 | Dual specificity protein phosphatase 7 | 2 |
| DUSP9 | Dual specificity protein phosphatase 9 | 1 |
| EGFR | Epidermal growth factor receptor | 1 |
| Eotaxin | Eotaxin | 3 |
| Factor B | Complement factor B | 2 |
| FASN | FASN protein | 2 |
| FER | Tyrosine-protein kinase Fer | 2 |
| CA-19-9 | CA-19-9 (Full Ab) | 2 |
| (Full Ab) | | |
| GAK | GAK protein | 2 |
| GEM | GTP-binding protein GEM | 2 |
| GLP-1 | Glucagon-like peptide-1 | 1 |
| GLP-1R | Glucagon-like peptide 1 receptor | 1 |
| GM-CSF | Granulocyte-macrophage colony-stimulating | 4 |
| | factor | |
| GNAI3 | Guanine nucleotide-binding protein G(k) | 2 |
| | subunit alpha | |
| GORS2 | Golgi reassembly-stacking protein 2 | 2 |
| GPRK5 | G protein-coupled receptor kinase 5 | 1 |
| GRIP2 | Glutamate receptor-interacting protein 2 | 3 |
| HADH2 | HADH2 protein | 2 |
| Her2/ErbB2 | Receptor tyrosine-protein kinase erbB-2 | 2 |
| HLA-DR/DP | HLA-DR/DP | 1 |
| ICAM-1 | Intercellular adhesion molecule 1 | 1 |
| IFN-γ | Interferon gamma | 3 |
| IgM | IgM | 4 |
| IL-10 | Interleukin-10 | 3 |
| IL-11 | Interleukin-11 | 3 |
| IL-12 | Interleukin-12 | 4 |
| IL-13 | Interleukin-13 | 3 |
| IL-16 | Interleukin-16 | 3 |
| IL-18 | Interleukin-18 | 3 |
| IL-1-ra | Interleukin-1 receptor antagonist protein | 3 |
| IL-1α | Interleukin-1 alpha | 3 |
| IL-1β | Interleukin-1 beta | 3 |
| IL-2 | Interleukin-2 | 3 |
| IL-3 | Interleukin-3 | 3 |
| IL-4 | Interleukin-4 | 4 |
| IL-5 | Interleukin-5 | 3 |
| IL-6 | Interleukin-6 | 5 |
| IL-7 | Interleukin-7 | 2 |
| IL-8 | Interleukin-8 | 3 |
| IL-9 | Interleukin-9 | 3 |
| INADL | InaD-like protein | 2 |
| Integrin | Integrin alpha-10 | 1 |
| α-10 | | |
| Integrin | Integrin alpha-11 | 1 |
| α-11 | | |
| ITCH | E3 ubiquitin-protein ligase Itchy homolog | 2 |
| JAK3 | Tyrosine-protein kinase JAK3 | 1 |
| KCC2B | Calcium/calmodulin-dependent protein kinase | |
| | type II subunit beta | |
| KCC4 | Calcium/calmodulin-dependent protein kinase | 2 |
| | type IV | |
| Keratin 19 | Keratin, type I cytoskeletal 19 | 2 |
| KIAA0882 | TBC1 domain family member 9 | 3 |
| KKCC1 | Calcium/calmodulin-dependent protein kinase 1 | 2 |
| KRASB | GTPase KRas | 1 |
| KSYK | Tyrosine-protein kinase SYK | 2 |
| LDL | Apolipoprotein B-100 | 2 |
| Leptin | Leptin | 1 |
| Lewis x | Lewis x | 2 |
| Lewis y | Lewis y | 1 |
| LIN7A | Protein lin-7 homolog A | 2 |
| LUM | Lumican | 1 |
| MAGI1 | Membrane-associated guanylate kinase, | 2 |
| | WW and PDZ domain-containing protein 1 | |
| MAP2K2 | Dual specificity mitogen-activated | 2 |
| | protein kinase 2 | |
| MAP2K6 | Dual specificity mitogen-activated | 2 |
| | protein kinase 6 | |
| MAPK9 | Mitogen-activated protein kinase 9 | 3 |
| MARK1 | Serine/threonine-protein kinase MARK1 | 2 |
| MATK | Megakaryocyte-associated tyrosine- | 2 |
| | protein kinase | |
| MCP-1 | C-C motif chemokine 2 | 5 |
| MCP-3 | C-C motif chemokine 7 | 3 |
| MCP-4 | C-C motif chemokine 13 | 3 |
| MD2L1 | Mitotic spindle assembly checkpoint | 2 |
| | protein MAD2A | |
| MK01 | Mitogen-activated protein kinase 1 | 2 |
| MK08 | Mitogen-activated protein kinase 8 | 3 |
| Mucin-1 | Mucin-1 | 4 |
| MYOM2 | Myomesin-2 | 2 |
| NDC80 | Kinetochore protein NDC80 homolog | 2 |
| NOS1 | Nitric oxide synthase, brain | 2 |
| OSBPL3 | Oxysterol-binding protein-related | 2 |
| | protein 3 | |
| OSTP | Osteopontin | 2 |
| OTU6B | OTU domain-containing protein 6B | 2 |
| OTUB1 | Ubiquitin thioesterase OTUB1 | 2 |
| OTUB2 | Ubiquitin thioesterase OTUB2 | 2 |
| P85A | Phosphatidylinositol 3-kinase | 2 |
| | regulatory subunit alpha | |
| PAK4 | Serine/threonine-protein kinase PAK 4 | 2 |
| PAK5 | Serine/threonine-protein kinase PAK 7 | 2 |
| PARP1 | Poly [ADP-ribose] polymerase 1 | 1 |
| PARP6B | Partitioning defective 6 homolog beta | 2 |
| PGAM5 | Serine/threonine-protein phosphatase | 2 |
| | PGAM5, mitochondrial | |
| PRD14 | PR domain zinc finger protein 14 | 3 |
| PRDM8 | PR domain zinc finger protein 8 | 2 |
| PRKCZ | Protein kinase C zeta type | 2 |
| PRKG2 | cGMP-dependent protein kinase 2 | 2 |
| Procathepsin W | Cathepsin W | 1 |
| Properdin | Properdin | 1 |
| PSA | Prostate-specific antigen | 1 |
| PTK6 | Protein-tyrosine kinase 6 | 1 |
| PTN13 | Tyrosine-protein phosphatase non- | 2 |
| | receptor type 13 | |
| PTPN1 | Tyrosine-protein phosphatase non- | 2 |
| | receptor type 1 | |
| PTPRD | Receptor-type tyrosine-protein | 2 |
| | phosphatase delta | |
| PTPRJ | Receptor-type tyrosine-protein | 3 |
| | phosphatase eta | |
| PTPRK | Receptor-type tyrosine-protein | 3 |
| | phosphatase kappa | |
| PTPRN2 | Receptor-type tyrosine-protein | 2 |
| | phosphatase N2 | |
| PTPRO | Receptor-type tyrosine-protein | 2 |
| | phosphatase O | |
| PTPRT | Receptor-type tyrosine-protein | 2 |
| | phosphatase T | |
| RANTES | C-C motif chemokine 5 | 3 |
| RPS6KA2 | Ribosomal protein S6 kinase alpha-2 | 2 |
| SHC1 | SHC-transforming protein 1 | 2 |
| Sialyl | Sialyl Lewis x | 1 |
| Lewis x | | |
| SNTA1 | Alpha-1-syntrophin | 2 |
| Sox11a | Transcription factor SOX-11 | 1 |
| SPDLY | Protein Spindly | 2 |
| STAP1 | Signal-transducing adaptor protein 1 | 2 |
| STAP2 | Signal-transducing adaptor protein 2 | 2 |
| STAT1 | Signal transducer and activator of | 2 |
| | transcription 1-alpha/beta | |
| TENS4 | Tensin-4 | 1 |
| TGF-β1 | Transforming growth factor beta-1 | 3 |
| TNFRSF14 | Tumor necrosis factor receptor | 2 |
| | superfamily member 14 | |
| TNFRSF3 | Tumor necrosis factor receptor | 2 |
| | superfamily member 3 | |
| TNF-α | Tumor necrosis factor | 3 |
| TNF-β | Lymphotoxin-alpha | 4 |
| TOPB1 | DNA topoisomerase 2-binding protein 1 | 2 |
| UBC9 | SUMO-conjugating enzyme UBC9 | 2 |
| UBE2C | Ubiquitin-conjugating enzyme E2 C | 2 |
| UBP7 | Ubiquitin carboxyl-terminal hydrolase 7 | 2 |
| UCHL5 | Ubiquitin carboxyl-terminal hydrolase | 1 |
| | isozyme L5 | |
| UPF3B | Regulator of nonsense transcripts 3B | 2 |
| VEGF | Vascular endothelial growth factor | 4 |

TABLE 6
SVM script

```
(A) LOO (Leave One Out)
rawfile <- read.delim(filnamn)
samplenames <- as.character(rawfile[,1])
groups <- rawfile[,2]
data <- t(rawfile[,-c(1:2)])
ProteinNames <- read.delim(filnamn,header=FALSE)
ProteinNames <- as.character(as.matrix(ProteinNames)[1,])
ProteinNames <- ProteinNames[-(1:2)]
rownames(data) <- ProteinNames
colnames(data) <- samplenames
PairWiseGroups<- as.matrix(read.delim(“Test.txt”,header=FALSE))
wilcoxtest <- function(prot,subset1,subset2){
res <- wilcox.test(prot[subset1],prot[subset2])
res$p.value
}
foldchange <- function(prot,subset1,subset2){
2\hat{ }(mean(prot[subset1]) − mean(prot[subset2]))
}
BenjaminiHochberg <- function(pvalues){
  # This function takes a vector of p-values as input and outputs
  # their q-values. No reordering of the values is performed
  NAindices <- is.na(pvalues)
  Aindices <- !NAindices
  Apvalues <- pvalues[Aindices]
  N <- length(Apvalues)
  orderedindices <- order(Apvalues)
  OrdValues <- Apvalues[orderedindices]
  CorrectedValues <- OrdValues * N /(1:N)
  MinValues <- CorrectedValues
  for (i in 1:N){MinValues[i] <- min(CorrectedValues[i:N])}
  Aqvalues <- numeric(N)
  Aqvalues[orderedindices] <- MinValues
  Qvalues <- pvalues
  Qvalues[Aindices] <- Aqvalues
  return(Qvalues)
}
library(MASS)
library(gplots)
redgreen <- function(n)
  {
  c(
    hsv(h=0/6, v=c( rep( seq(1,0.3,length=5) , c(13,10,8,6,4) ) , 0 ) ) ,
    hsv(h=2/6, v=c( 0, rep( seq(0.3,1,length=5) , c(3,5,7,9,11) ) ) )
  )
  }
pal <- rev(redgreen(100));
library(e1071)
source(“NaiveBayesian”)
svmLOOvalues <- function(data , fac){
n1 <- sum(fac==levels(fac)[1])
n2 <- sum(fac==levels(fac)[2])
nsamples <- n1+n2
ngenes <- nrow(data)
SampleInformation <- paste(levels(fac)[1],“ ”,n1,“, ”,levels(fac)[2],“ ”, n2,sep=“”)
res <- numeric(nsamples)
sign <- numeric(nsamples)
for (i in 1:nsamples){
  svmtrain <- svm(t(data[,−i]) , fac[−i] , kernel=“linear” )
  pred <- predict(svmtrain , t(data[,i]) , decision.values=TRUE)
  res[i] <- as.numeric(attributes(pred)$decision.values)
  facnames <- colnames(attributes(pred)$decision.values)[1]
  if (facnames == paste(levels(fac)[1],“/”,levels(fac)[2],sep=“”)){sign[i] <- 1}
  if (facnames == paste(levels(fac)[2],“/”,levels(fac)[1],sep=“”)){sign[i] <- −1}
}
if (length(unique(sign)) >1){print(“error”)}
res <- sign * res
names <- colnames(data , do.NULL=FALSE)
orden <- order(res , decreasing=TRUE)
Samples <- data.frame(names[orden],res[orden],fac[orden])
ROCdata <- myROC(res,fac)
SenSpe <- SensitivitySpecificity(res,fac)
return(list(SampleInformation=SampleInformation,ROCarea=ROCdata[1],p.value=ROCdata[2],SenSpe <-
SenSpe,samples=Samples))
}
Analysera<- function(group1 ,group2){
  outputfiletxt <- paste(group1,“ versus ”,group2,“.txt” ,sep=“”)
  outputfilepdf <- paste(group1,“ versus ”,group2,“.pdf” ,sep=“”)
  subset1 <- is.element(groups , strsplit(group1,“,”)[[1]])
  subset2 <- is.element(groups , strsplit(group2,“,”)[[1]])
  wilcoxpvalues <- apply(data , 1 , wilcoxtest , subset1 , subset2)
  foldchange <- apply(data , 1 , foldchange , subset1 , subset2)
  QvaluesAll <- BenjaminiHochberg(wilcoxpvalues)
  HugeTable <- cbind(ProteinNames,foldchange,wilcoxpvalues,QvaluesAll)
  write.table(HugeTable, file=outputfiletxt , quote=FALSE, sep=“\t”,row.names=FALSE)
  color <- rep(‘black’, length(subset1))
  color[subset1] <- ‘red’
  color[subset2] <- ‘blue’
  pdf(outputfilepdf)
  Sam <- sammon(dist(t(data[,subset1|subset2])) , k=2)
  plot(Sam$points , type=“n” , xlab = NA , ylab=NA, main=“All proteins” ,asp=1)
  text(Sam$point , labels = colnames(data[,subset1|subset2]), col=color[subset1|subset2])
  heatmap.2(data[,subset1|subset2] , labRow = row.names(data), trace=“none” , labCol =“” , ColSideColors=
color[subset1|subset2],col=pal , na.color= “grey”, key=FALSE , symkey =FALSE , tracecol = “black” , main =“” , dendrogram= ‘both’
, scale =“row” ,cexRow=0.2)
  svmfac <- factor(rep(‘rest’,ncol(data)),levels=c(group1,group2,‘rest’))
  svmfac[subset1] <- group1
  svmfac[subset2] <- group2
  svmResAll <- svmLOOvalues(data[,subset1|subset2] , factor(as.character(svmfac[subset1|subset2]),levels=c(group1,group2)))
  ROCplot(svmResAll , sensspecnumber=4)
  write(“” , file=outputfiletxt , append=TRUE)
  write(“All proteins” , file=outputfiletxt , append=TRUE)
  write(“” , file=outputfiletxt , append=TRUE)
  for (i in 1:5){write.table(svmResAll[i]], file=outputfiletxt , append=TRUE, sep=“\t” , quote=FALSE)
          write( “” , file=outputfiletxt , append=TRUE)
  }
  dev.off( )
}
(B) BE (Backward Elimination)
getWorstAb <- function(errors, abNames, sortDe)
{
  return(abNames[order(errors, decreasing = sortDe)[1]])
}
testModels <- function(models, elimData, averages, svmfac, cvSplitTrn, cvSplitVal, nKfold, nRep, sortDe)
{
  nsamples <- ncol(elimData)
  d0 <- as.numeric(svmfac)−1
  E <- numeric(nsamples)
  analytes <- nrow(elimData)
  errors <- numeric(nrow(elimData))
  nSplits <- length(cvSplitTrn)
  for(k in 1:analytes){
    backup <- elimData[k,]
    elimData[k,] <- averages[k]
    nM <- 1
    aveE <- 0
    aveAuc <- 0
    for (nr in 1:nRep){
      y <- numeric(0)
      d <- numeric(0)
      for (nk in 1:nKfold){
        idx <-cvSplitVal[[nM]]
        pred <- predict(models[[nM]] , t(elimData[,idx]), decision.values=TRUE)
        d <- c(d, d0[idx])
        y <- c(y, as.numeric(attributes(pred)$decision.values))
        nM <- nM + 1
      }
      if (length(d) != nsamples || length(y) != nsamples) {
        stop(“Error: Lengths of prediction and target vector are wrong!”)
      }
      y = 1−(1/(1 + exp(−y)))
      for (i in 1:nsamples){
        E[i] <- −(d[i]*log(y[i])+(1−d[i])*log(1−y[i]))
      }
      aveE <- aveE + sum(E)
      if (sortDe) {
        auroc <- roc(d,y)
        aveAuc <- aveAuc + auroc$auc
      }
    }
    if (sortDe) {
      errors[k] <- aveAuc / nRep
    } else {
      errors[k] <- aveE / nRep
    }
    elimData[k,] <- backup
  }
  return( errors )
}
getNewElimData <- function(errors, elimData, sortDe){
  tasBort <- order(errors,decreasing = sortDe)[1]
  return(elimData[−tasBort,])
}
getSmallestError <- function(errors, sortDe){
  if (sortDe) {
    return(max(errors))
  } else {
    return(min(errors))
  }
}
getNewAverages <- function(errors, averages, sortDe){
  tasBort <- order(errors, decreasing = sortDe)[1]
  return(averages[−tasBort])
}
backElim <- function(filename, resfile, plotfile, group1, group2, nKfold, nRep, nKOut, nRepOut, sortDe){
  rawfile <- read.delim(filename)
  groups <- rawfile[,2]
  samplenames <- as.character(rawfile[,1])
  data <- t(rawfile[,−c(1,2)])
  ProteinNames <- read.delim(filename,header=FALSE)
  ProteinNames <- as.character(as.matrix(ProteinNames)[1,])
  ProteinNames <- ProteinNames[−(1:2)]
  antal <- length(ProteinNames)
  print(ProteinNames)
  rownames(data) <- ProteinNames
  colnames(data) <- samplenames
  subset1 <- is.element(groups , strsplit(group1,“,”)[[1]])
  subset2 <- is.element(groups , strsplit(group2,“,”)[[1]])
  svmfac <- factor(rep(‘rest’,ncol(data)),levels=c(group1,group2,‘rest’))
  svmfac[subset1] <- group1
  svmfac[subset2] <- group2
  svmfac <- svmfac[subset1|subset2]
  smallestErrorPerLength <- rep(NA,antal)
  averages <- apply(data, 1, mean)
  abOrder <- rep(NA,antal)
  elimData <- data[,subset1|subset2]
  nsamples <- ncol(elimData)
  subset1 <- svmfac==group1
  subset2 <- svmfac==group2
  print(paste(nsamples, “samples”),quote=F)
  print(paste(“ ”,sum(subset1), “in”, group1),quote=F)
  print(paste(“ ”,sum(subset2), “in”, group2),quote=F)
  models <- numeric(nsamples)
  borttagna <- 0
  wrst <- 0
  proc <- 0
  m <- 0
  for(i in 1:(antal−1)){
    m <- m+(antal−i)*sqrt(antal−i)
  }
  control <- as.numeric(svmfac)
  checkGr1<- svmfac[subset1]
  if(sum(control[checkGr1])!= sum (control[subset1])){
    stop(“ERROR: Change the order of group1 and group2 in the data file!!!”)
  }
  checkGr2<- svmfac[subset2]
  if(2*(sum(control[checkGr2]))!= sum (control[subset2])) {
    stop(“ERROR: Change the order of group1 and group2 in the data file!!!”)
  }
  cvSplitOuter <- createMultiFolds(svmfac, k=nKOut, times=nRepOut)
  abOrderRank <- vector(‘numeric’, antal)
  names(abOrderRank) <- ProteinNames
  avePerf <- vector(‘numeric’, antal)
  for(no in 1:(nKOut * nRepOut)){
    idxW <- cvSplitOuter[[no]]
    elimDataW <- elimData[,idxW]
    averagesW <- apply(elimDataW, 1, mean)
    svmfacW <- svmfac[idxW]
    nsamplesW <- ncol(elimDataW)
    print(nKfold)
    print(nRep)
    cvSplitTrn <- createMultiFolds(svmfacW, k=nKfold, times=nRep)
    nSplits <- length(cvSplitTrn)
    if (nSplits != nKfold * nRep) {
      stop(“Error: Failure in cvSplits”)
    }
    cvSplitVal <- vector(“list“, length = nSplits)
    idx0 <- c(1:nsamplesW)
    for (i in 1:nSplits) {
      idx <- cvSplitTrn[[i]]
      cvSplitVal[[i]] = idx0[−idx]
    }
    abOrder <- rep(NA,antal)
    smallestErrorPerLength <- rep(NA,antal)
    for(j in 1:(antal−1)){
      start.time <- Sys.time( )
      models <- vector(‘list’, 0)
      for (nM in 1:nSplits){
        idx <- cvSplitTrn[[nM]]
        models[nM] <- list(svm(t(elimDataW[,idx]), svmfacW[idx], kernel=“linear”))
      }
      errors <- testModels(models, elimDataW, averagesW, symfacW,
                cvSplitTrn, cvSplitVal, nKfold, nRep, sortDe)
      wrst<-getWorstAb(errors, row.names(elimDataW), sortDe)
      abOrder[j] <- wrst
      smallestErrorPerLength[j] <- getSmallestError(errors, sortDe)
      averagesW <- getNewAverages(errors, averagesW, sortDe)
      elimDataW <- getNewElimData(errors, elimDataW, sortDe)
      borttagna <- borttagna + 1
      proc <- proc + (antal−j)*sqrt(antal−j)
      end.time <- Sys.time( )
      time.taken <- end.time - start.time
      ans <- sprintf(“(%d): %-30s eliminated, last perf: %.2f, time: %.2f”,
              j, wrst, smallestErrorPerLength[j], time.taken)
      print(ans)
    }
    abOrder[length(abOrder)] <- setdiff(ProteinNames, abOrder)
    for (ii in 1:antal) {
      abOrderRank[abOrder[ii]] <- abOrderRank[abOrder[ii]] + log(ii)
      avePerf[ii] <- avePerf[ii] + smallestErrorPerLength[ii]
    }
  }
  avePerf <- avePerf / (nKOut*nRepOut)
  abOrderRank <- abOrderRank / (nKOut*nRepOut)
  abOrderRank <- abOrderRank[order(abOrderRank)]
  abOrderRank <- exp(abOrderRank)
  write.table(
    cbind(avePerf, abOrderRank, names(abOrderRank)),
    file=resfile, sep=“\t”, quote=F, row.names=F)
  pdf(plotfile)
  plot(avePerf, type =“b”, ylab = “K-L Error”, xlab = “Eliminations”)
}
library(e1071, quietly = TRUE)
library(caret, quietly = TRUE)
library(pROC, quietly = TRUE)
doArgs <- FALSE
useROC <- FALSE
sortDe <- FALSE
if (useROC) {
  sortDe <- TRUE
}
if( doArgs ) {
  args <- commandArgs(trailingOnly = TRUE)
  dataFile <- args[1]
  resultFile <- args[2]
  plotFile <- args[3]
  nKfold <- as.numeric(args[4])
  nRep <- as.numeric(args[5])
  nKOut <- as.numeric(args[6])
  nRepOut <- as.numeric(args[7])
  backElim(dataFile, resultFile, plotFile, “1”, “0”, nKfold, nRep, nKOut, nRepOut, sortDe)
} else {
  dataFile <- ‘Herlev Raw NC & PDAC_RF.txt’
  resultFile <- ‘rnd_rankRes.txt’
  plotFile <- ‘rnd_rankPlot.pdf’
  nKfold <- 10
  nRep <- 5
  nKOut <- 5
  nRepOut <- 1
  backElim(dataFile, resultFile, plotFile, “Normal”, “PDAC”, nKfold, nRep, nKOut, nRepOut, sortDe)
}
```

TABLE 7
Amino acid sequences of the scFv antibodies used in the Examples

| | |
|---|---|
| IL-1a | EVQLLESGGGLVQPGGSLRLSCAASGFTFSSYAMHWVRQAPGKGLEWVSGVSWNGSRTHYADSVKGRFT |
| (1) | ISRDNSKNTLYLQMNSLRAEDTAVYYCARSSGYYSWAFDIWGQGTLVTVSSGGGGSGGGGSGGGGSQSV |
| | LTQPPSASGTPGQRVTISCSGSSSNIGRNTVNWYQQLPGTAPKLLIYGNSNRPSXVPDRFSGSKSGTSA |
| | SLAISGLRSEDEADYYCAAWDDSLNGWAFGGXTKLTVLGEQKLISXXXLSGSAA [SEQ ID NO: 1] |
| IL-1a | EVQLLESGGGLVQPGGSLRLSCAASGFTFSSYSMNWVRQAPGKGLEWVALISYDGSQKYYADSMKGRFT |
| (2) | ISRDNSKNTLYLQMNSLRAEDTAVYYCAKGHTSGTKAYYFDSWGQGTLVTVSSGGGGSGGGGSGGGGSQ |
| | SVLTQPPSASGTPGQRVTISCTGTSSNIGAGYSVHWYQQLPGTAPKLLIYGNSNRPSGVPDRFSGSKSG |
| | TSASLAISGLRSEDXADYYCQSYDSSLSGWVFGGXTKLTVLGEQKLISEEDLSGSAA |
| | [SEQ ID NO: 2] |
| IL-2 | EVQLLESGGGLVQPGGSLRLSCAASGFTFGDYAMSWVRQAPGKGLEWVSSISSRGSYIYFADSVKGRFT |
| (1) | ISRDNSKNTLYLQMNSLRAEDTAVYYCAKKKTGYYGLDAWGQGTLVTVSSGGGGSGGGGSGGGGSQSVL |
| | TQPPSASGTPGQRVTISCTGSSSNIGAGYDVHWYQQLPGTAPKLLIYGNSNRPSGVPDRFSGSKSGTSA |
| | SLAISGLRSEDEADYYCSSYAGSNNLVFGGXTKLTXLGEQKLISXXDLSGSAA [SEQ ID NO: 3] |
| IL-2 | EVXXLESGGGLVQPGGSLRLSCAASGFTFGDYAMSWVRQAPGKGLEWVSSISSRGSYIYFADSVKGRFT |
| (2) | ISRDNSKNTLYLQMNSLRAEDTAVYYCAKKKTGYYGLDAWGQGTLVTVSSGGGGSGGGGSGGGGSQSVL |
| | TQPPSASGTPGQRVTISCTGSSSNIGAGYDVHWYQQLPGTAPKLLIYGNSNRPSGVPDRFSGSKSGTSA |
| | SLAISGLRSEDEADYYCSSYAGSNNLVFGGXXKLTVLGEQKLISEXXLSGSAA [SEQ ID NO: 4] |
| IL-2 | EVQLLESGGGLVQPGGSLRLSCAASGFTFGDYAMSWVRQAPGKGLEWVSSISSRGSYIYFADSVKGRFT |
| (3) | ISRDNSKNTLYLQMNSLRAEDTAVYYCAKKKTGYYGLDAWGQGTLVTVSSGGGGSGGGGSGGGGSQSVL |
| | TQPPSASGTPGQRVTISCTGSSSXIGAGYDVHWYQQLPGTAPKLLIYGNSNRP [SEQ ID NO: 5] |
| IL-3 | EVQLLESGGGLVQPGGSLRLSCAASGFTFGRYTMHWVRQAPGKGLEWVSSISSSSSYIYYADSVKGRFT |
| (1) | ISRDNSKNTLYLQMNSLRAEDTAVYYCARHFFESSGGYFDYWGQGTLVTVSSGGGGSGGGGSGGGGSQS |
| | VLTQPPSASGTPGQRVTISCSGSSSNIGSNTVNWYQQLPGTAPKLLIYRNNQRPSGVPDRFSGSKSGTS |
| | ASLAISGLRSEDXADYYCAAWDDSLNGWVFGGXXKLTVLGEQKLISXXXLSGXAA |
| | [SEQ ID NO: 6] |
| IL-3 | EVQLLESGGGLVQPGGSLRLSCAASGFTFSSYAMSWVRQAPGKGLEWVSAISGSGGSTYYADSVKGRFT |
| (2) | ISRDNSKNTLYLQMNSLRAEDTAVYYCARGGARYDYWGQGTLVTVSSGGGGSGGGGSGGGGSQSVLTQP |
| | PSASGTPGQRVTISCSGSSSNIGSNTVNWYQQLPGTAPKLLIYDNNKRPSGVPDRFSGSKSGTSASLAI |
| | SGLRSEDEADYYCQSYDNILRGVVFGGGTKLTVLXEQKLISEXDLSGSAA [SEQ ID NO: 7] |
| IL-3 | EVXXXESGGGLVQPGGSLRLSCAASGFTFSSYAMSWVRQAPGKGLEWVSAISGRGEYTYYAGSVKGRFT |
| (3) | ISRDNSKNTLYLQMNSLRAEDTAVYYCATGATRFGYWGQGTLVTVSSGGGGSGGGGSGGGGSQSVLTQP |
| | PSASGTPGQRVTISCTGSSSNIGAGYGVQWYQQLPGTAPKLLIYRNNQRPSGVPDRFSGSKSGTSXSLA |
| | ISGLRSEDEADYYCQSYDSSLSYSVFGGXTKLTVLGEQKLISXXDLSGSAA [SEQ ID NO: 8] |
| IL-4 | EVQLLESGGGLVQPGGSLRLSCAASGFTFSNAWMSWVRQAPGKGLEWVSSLHGGGDTFYTDSVKGRFTI |
| (1) | SRDNSKNTLYLQMNSLRAEDTAVYYCASLYGSGSYYYYYYGMDVWGQGTLVTVSSGGGGSGGGGSGGGG |
| | SQSVLTQPPSASGTPGQRVTISCSGNNSNTGNNAVNWYQQLPGTAPKLLIYDNNKRPSGVPDRFSGSKS |
| | GTSASLAISGLRSEDEADYYCCSYAGSYIWVFGGXTKLTVLGEQKLISXEXLSGSAA |
| | [SEQ ID NO: 9] |
| IL-4 | EVQLLESGGGLVQPGGSLRLSCAASGFTFSDYGMHWVRQAPGKGLEWVSGISWNGGKTHYVDSVKGQFT |
| (2) | ISRDNSKNTLYLQMNSLRAEDTAVYYCARDRGYCSNGVCYTILDYWGQGTLVTVSSGGGGSGGGGSGGG |
| | GSQSVLTQPPSASGTPGQRVTISCSGSSSNIGSNTINWYQQLPGTAPKLLIYGNSNRPSGVPDRFSGSK |
| | SGTSASLAISGLRSEDEADYYCQSYDSSLSGWVFGGXTKLXVLXEQKLISXXDLSGSAA |
| | [SEQ ID NO: 10] |
| IL-5 | EVQLLESGGGLVQPGGSLRLSCAASGFTFSSYAMSWVRQAPGKGLEWVSSISSRSNYIYYSDSVKGRFT |
| (1) | ISRDNSKNTLYLQMNSLRAEDTAVYYCARNFRFFDKWGQGTLVTVSSGGGGSGGGGSGGGGSQSVLTQP |
| | PSASGTPGQRVTISCSGGSSXIGANPVSWYQQLPGTAPKLLIYGNSNRP [SEQ ID NO: 11] |
| IL-5 | EVQLLESGGGLVQPGGSLRLSCAASGFTFSSYAMSWVRQAPGKGLEWVSSISSRSNYIYYSDSVKGRFT |
| (2) | ISRDNSKNTLYLQMNSLRAEDTAVYYCARNFRFFDKWGQGTLVTVSSGGGGSGGGGSGGGGSQSVLTQP |
| | PSASGTPGQRVTISCSGGSSNIGANPVSWYQQLPGTAPKLLIYGNSNRPSGVPDRFSGSKSGTSASLAI |
| | SGLRSEDEADYYCQSYDSSLSGSVFGGXTKLTVLXEQKLISEXXLSGSAA [SEQ ID NO: 12] |
| 1L-5 | EVQLLESGGGLVQPGGSLRLSCAASGFTFSSYAMSWVRQAPGKGLEWVSSISSRSNYIYYSDSVKGRFT |
| (3) | ISRDNSKNTLYLQMNSLRAEDTAVYYCARNFRFFDKWGQGTLVTVSSGGGGSGGGGSGGGGSQSVLTQP |
| | PSASGTPGQRVTISCSGGSSNIGANPVSWYQQLPGTAPKLLIYGNSNRPSGVPDRFSGSKSGTSASLAI |
| | SGLRSEDEADYYCQSYDSSLSGSVFGGXTKLTVLGEQKLISXEDLSGSAA [SEQ ID NO: 13] |
| IL-6 | EVQLLESGGGLVQPGGSLRLSCAASGFTFSSYGMHWVRQAPGKGLEWVSGINWNGGSTGYADSVKGRFT |
| (1) | ISRDNSKNTLYLQMNSLRAEDTAVYYCARNRGSSLYYGMDVWGQGTLVTVSSGGGGSGGGGSGGGGSQS |
| | VLTQPPSASGTPGQRVTISCAGSSSNIGSKSVHWYQQLPGTAPKLLIYRNNRRPSGVPDRFSGSXSGTS |
| | XSLAIXGLRSXDXADYYCXXWDDRVNXXXFGGXTXLTVLXXQKLISXXXLSGSXXXPSSSXXLIXXGXX |
| | XXLX-XXLXFTGRXFXTX-LXXX [SEQ ID NO: 14] |
| IL-6 | EVQLLESGGGLVQPGGSLRLSCAASGFTFSNYGMHWVRQAPGKGLEWVSSITSSGDGTYFADSVKGRFT |
| (2) | ISRDNSKNTLYLQMNSLRAEDTAVYYCARAGGIAAAYAFDIWGQGTLVTVSSGGGGSGGGGSGGGGSQS |
| | VLTQPPSASGTPGQRVTISCSGSSSNVGSNYVYWYQQLPGTAPKLLIYDNNKRPSGVPDRFSGSKSGTS |
| | ASLAISGLRSEDEADYYCQSYDSSRWVFGGXTKLTVLGEQXLISEEXLSGSAA [SEQ ID NO: 15] |
| IL-7 | EVQLLESGGGLVQPGGSLRLSCAASGFTFSSYGMHWVRQAPGKGLEWVSGITWNSGSIGYVDSVKGRFT |
| (1) | ISRDNSKNTLYLQMNSLRAEDTAVYYCARGPSVAARRIGRHWYNWFDPWGQGTLVTVSSGGGGSGGGGS |
| | GGGGSQSVLTQPPSASGTPGQRVTISCSGSSSNIGSNSVYWYQQLPGTAPKLLIYDNNKRPSGVPDRFS |
| | GSKSGTSASLAISGLRSEXXADYYCQSYDSSLSGSVFGGXXKLXVLGEQKLISEXXLSGSAA |
| | [SEQ ID NO: 16] |
| IL-7 | EVQLLESGGGLVQPGGSLRLSCAASGFTFSDYNIHWVRQPPGKGLEWVSGVSWNGSRTHYADSVKGQFT |
| (2) | ISRDNSKNTLYLQMNSLRAEDTAVYYCARDPAMVRGVVLPNYYGLDVWGQGTLVTVSSGGGGSGGGGSG |
| | GGGSQSVLTQPPSASGTPGQRVTISCTGSSSNIGAGYDVHWYQQLPGTAPKLLIYGHSNRPSGVPDRFS |
| | GSKSGTSASLAISGLRSEXXADYYCQSYDSSLSYPVFGGXTKLTVLGEQ [SEQ ID NO: 17] |
| IL-8 | EVQLLESGGGLVQPGGSLRLSCAASGFTFDDYGMSWVRQAPGKGLEWVSLISWDGGSTYYADSVKGRFT |
| (1) | ISRDNSKNTLYLQMNSLRAEDTAVYYCARDDLYGMDVWGQGTLVTVSSGGGGSGGGGSGGGGSQSVLTQ |
| | PPSASGTPGQRVTISCTGSSSNIGAGYDVHWYQQLPGTAPKLLIYDNNKRPSXVPDRFSGSKSGTSASL |
| | AISGLRSEDEADYYCAAWDDSLSGWVFGGXTKLTVLXEQKLISEXXLSGSAA [SEQ ID NO: 18] |
| IL-8 | EVQLLESGGGLVQPGGSLRLSCAASGFTFSSYEMNWVRQAPGKGLEWVSSISSSSSYIFYADSMKGRFT |
| (2) | ISRDNSKNTLYLQMNSLRAEDTAVYYCARNESVDPLGGQYFQHWGQGTLVTVSSGGGGSGGGGSGGGGS |
| | QSVLTQPPSASGTPGQRVTISCTGSSSNIGAGYDVHWYQQLPGTAPKLLIYDNNKRPSGVPDRFSGSKS |
| | GTSASLAISGLRSEDEADYYCSAWDDNLDGPVFGGXTKLTVLXEQKLISXXXLSGSAA |
| | [SEQ ID NO: 19] |
| IL-9 | EVQLLESGGGLVQPGGSLRLSCAASGFTFSSYAMSWVRQAPGKGLEWVSSISSSSSYIYYADSVKGRFT |
| (1) | ISRDNSKNTLYLQMNSLRAEDTAVYYCTTFGHWGQGTLVTVSSGGGGSGGGGSGGGGSQSVLTQPPSAS |
| | GTPGQRVTISCSGSGSNIGDNSVNWYQQLPGTAPKLLIYGNNNRPSGVPDRFSGSKSGTSASLAISGLR |
| | SEDEADYYCSSYTSSSVVFGGXTKLTVLXEQKLISEXDLSGSAA [SEQ ID NO: 20] |
| IL-9 | EVQLLESGGGLVQPGGSLRLSCAASGFTFSSYGMHWVRQAPGKGLEWVAVISYDGSNKYYADSVKGRFT |
| (2) | ISRDNSKNTLYLQMNSLRAEDTAVYYCAKSPGGSPYYFDYWGQGTLVTVSSGGGGSGGGGSGGGGSQSV |
| | LTQPPSASGTPGQRVTISCSGSVSNIGSNVVSWYQQLPGTAPKLLIYDNNKRPS |
| | [SEQ ID NO: 21] |
| IL-9 | EVQLLESGGGLVQPGGSLRLSCAASGFTFSSYGMHWVRQAPGKGLEWVAVISYDGSNKYYADSVKGRFT |
| (3) | ISRDNSKNTLYLQMNSLRAEDTAVYYCAKSPGGSPYYFDYWGQGTLVTVSSGGGGSGGGGSGGGGSQSV |
| | LTQPPSASGTPGQRVTISCSGSVSNIGSNVVSWYQQLPGTAPKLLIYDNNKRPSGVPDRFSGSKSGTSA |
| | SLAISGLRSEDEADYYCQSYDSSLGGWVFGGXTKLTVLGEQKLISEXDLSGSAA |
| | [SEQ ID NO: 22] |
| IL-10 | EVQLLESGGGLVQPGGSLRLSCAASGFTFRSYVMSWVRQAPGKGLEWVSAISGSGGSTYYADSVKGRFT |
| (1) | ISRDNSKNTLYLQMNSLRAEDTAVYYCARGKGRWAFDIWGQGTLVTVSSGGGGSGGGGSGGGGSQSVLT |
| | QPPSASGTPGQRVTISCTGSSSNVGAGYDVHWYQQLPGTAPKLLIYRNNQRPSGVPDRFSGSKSGTSAS |
| | LAISGLRSXDXADYYCAAWDDSLSAHVVFGGXTKLTVLGEQKLISXXDLSGSAA |
| | [SEQ ID NO: 23] |
| IL-10 | EVQLLESGGGLVQPGGSLRLSCAASGFTFRSYVMSWVRQAPGKGLEWVSAISGSGGSTYYADSVKGRFT |
| (2) | ISRDNSKNTLYLQMNSLRAEDTAVYYCARGKGRWAFDIWGQGTLVTVSSGGGGSGGGGSGGGGSQSVLT |
| | QPPSASGTPGQRVTISCTGSSSNVGAGYDVHWYQQLPGTAPKLLIYRNNQRPSGVPDRFSGSKSGTSAS |
| | LAISGLRSEDEADYYCAAWDDSLSAHVVFGGXTKLTVLGEQKLISEXDLSGSAA |
| | [SEQ ID NO: 24] |
| IL-10 | EVQLLESGGGLVQPGGSLRLSCAASGFTFSSYAMSWVRQAPGKGLEWVSAISGSGGSTYYADSVKGRFT |
| (3) | ISRDNSKNTLYLQMNSLRAEDTAVYYCARGKGRWAFDIWGQGTLVTVSSGGGGSGGGGSGGGGSQSVLT |
| | QPPSASGTPGQRVTISCTGSSSNIGAGYGVHWYQQLPGTAPKLLIYGNSNRPSGVPDRFSGSKSGTSAS |
| | LAISGLRSEDXADYYCAAWDDSLSGLVFGGXTKLTVLXEQKLISEXXLSGSAA [SEQ ID NO: 25] |
| IL-11 | EVQLLESGGGLVQPGGSLRLSCAASGFTFSNFGMHWVRQAPGKGLEWVAFIRYDGSNKYYADSVKGRFT |
| (1) | ISRDNSKNTLYLQMNSLRAEDTAVYYCARHYYYSETSGHPGGFDPWGQGTLVTVSSGGGGSGGGGSGGG |
| | GSQSVLTQPPSASGTPGQRVTISCSGSSSNIGSYPVNWYQQLPGTAPKLLIYGNSNRPSGVPDRFSGSK |
| | SGTSASLAISGLRSEDEADYYCQXWGTGVFGGXTKLTVLGEQKLISXEXLSGSAA |
| | [SEQ ID NO: 26] |
| IL-11 | EVQLLESGGGLVQPGGSLRLSCAASGFTFSSYGMHWVRQAPGKGLEWVAVISYDGSNKYYADSVKGRFT |
| (2) | ISRDNSKNTLYLQMNSLRAEDTAVYYCARHYYDVSYRGQQDAFDIWGQGTLVTVSSGGGGSGGGGSGGG |
| | GSQSVLTQPPSASGTPGQRVTISCTGSSSNLGSPYDVHWYQQLPGTAPKLLIYRNDQRASGVPDRFSGS |
| | XSGTSASLAISGLRSEDEADYYCAAWDDSLNAWVFGGXTKLTVLGEQKLISEXXLSGSAA |
| | [SEQ ID NO: 27] |
| IL-11 | EVQLLESGGGLVQPGGSLRLSCAASGFTFSDYYMSWIRQAPGKGLEWVAYISGISGYTNYADSVRGRFT |
| (3) | ISRDNSKNTLYLQMNSLRAEDTAVYYCAKSKDWVNGGEMDVWGQGTLVTVSSGGGGSGGGGSGGGGSQS |
| | VLTQPPSASGTPGQRVTISCTGSSSNIGAGYVVHWYQQLPGTAPKLLIYSNNQRPSGVPDRFSGSKSGT |
| | SASLAISGLRSEDEADYYCAAWDDSLRGWVFGGXTKLTVLGEQKLISEEDLSGSAA |
| | [SEQ ID NO: 28] |
| IL-12 | EVQLLESGGGLVQPGGSLRLSCAASGFTFSSYSMNWVRQAPGKGLEWVSAIGTGGGTYYADSVKGRFTI |
| (1) | SRDNSKNTLYLQMNSLRAEDTAVYYCARAFRAFDIWGQGTLVTVSSGGGGSGGGGSGGGGSQSVLTQPP |
| | SASGTPGQRVTISCSGSRSNIGNNFVSWYQQLPGTAPKLLIYGNSNRPSGVPDRFSGSKSGTSASLAIS |
| | GLRSEDEADYYCAAWDDSLSGPVFGGXTKLTVLGEQKLISEXDLSGSAA [SEQ ID NO: 29] |
| IL-12 | EVQLLESGGGLVQPGGSLRLSCAASGFTFSDYYMSWVRQAPGKGLEWVSGVSWNGSRTHYADSVKGQFT |
| (2) | ISRDNSKNTLYLQMNSLRAEDTAVYYCARGSRSSPDAFDIWGQGTLVTVSSGGGGSGGGGSGGGGSQSV |
| | LTQPPSASGTPGQRVTISCTGSSSNIGAGYDVHWYQQLPGTAPKLLIYGNSNRPSGVPDRFSGSKSGTS |
| | ASLAISGLRSEDEADYYCAAWDDRVNGRVFGGGTKLTVLGEQKLISEXXLSGSAA |
| | [SEQ ID NO: 30] |
| IL-13 | EVQLLESGGGLVQPGGSLRLSCAASGFTFSSYSMNWVRQAPGKGLEWVSSISSGSSYIYYADSVKGRFT |
| (1) | ISRDNSKNTLYLQMNSLRAEDTAVYYCARSQGWWTYYYGMDVWGQGTLVTVSSGGGGSGGGGSGGGGSQ |
| | SVLTQPPSASGTPGQRVTISCSGSSSNIGSNTVNWYQQLPGTAPKLLIYSNNQRPSGVPDRFSGSKSGT |
| | SASLAISGLRSEDEADYYCETWGQ [SEQ ID NO: 31] |
| IL-13 | EVQLLESGGGLVQPGGSLRLSCAASGFTFSSYSMNWVRQAPGKGLEWVSSISSGSSYIYYADSVKGRFT |
| (2) | ISRDNSKNTLYLQMNSLRAEDTAVYYCARSQGWWTYYYGMDVWGQGTLVTVSSGGGGSGGGGSGGGGSQ |
| | SVLTQPPSASGTPGQRVTISCSGSSSNIGSNTVNWYQQLPGTAPKLLIYSNNQRPSGVPDRFSGSKSGT |
| | SASLAISGLRSEDXADYYCETXDSNTQIFGGXTKLTVLGEQKLISEEXLSGSAXAHHHHHH-SXRXPIX |
| | XIVSXITIHXXSFXNVVTGKXXALPXXXALQHIPXXXAXXXX[SEQ ID NO: 32] |
| IL-13 | EVQLLESGGGLVQPGGSLRLSCAASGFTFSSYSMNWVRQAPGKGLEWVSSISSGSSYIYYADSVKGRFT |
| (3) | ISRDNSKNTLYLQMNSLRAEDTAVYYCARSQGWWTYYYGMDVWGQGTLVTVSSGGGGSGGGGSGGGGSQ |
| | SVLTQPPSASGTPGQRVTISCSGSSSNIGSNWNWYQQLPGTAPKLLIYSNNQRPSGVPDRFSGSKSGTS |
| | ASLAISGLRSEDEADYYCETWDSNTQIFGGXTKLTVLGEQKLISEXDLSGSAA [SEQ ID NO: 33] |
| VEGF | EVQLLESGGGLVQPGGSLRLSCAASGFTFSSNEMSWIRQAPGKGLEWVSSISGSGGFTYYADSVKGRYT |
| (1) | ISRDNSKNTLYLQMNSLRAEDTAVYYCARETTVRGNAFDIWGQGTLVTVSSGGGGSGGGGSGGGGSQSV |
| | LTQPPSASGTPGQRVTISCTGGSSNIGAGYDVHWYQQLPGTAPKLLIYRNNQRPSGVPDRFSGSXSGTS |
| | ASLAISGLRSEDEADYYCAAWDDSLSVPMFGGXTKLTVLGEQKLISEXDLSGSAA |
| | [SEQ ID NO: 34] |
| VEGF | EVQLLESGGGLVQPGGSLRLSCAASGFTFSSYSMNWVRQAPGKGLEWVSGINWNGGSTGYADSVKGRFT |
| (2) | ISRDNSKNTLYLQMNSLRAEDTAVYYCASSVGGWYEGDNWFDPWGQGTLVTVSSGGGGSGGGGSGGGGS |
| | QSVLTQPPSASGTPGQRVTISCTGSSSNIGAGYDVHWYQQLPGTAPKLLIYGNSNRPSXVPDRFSGSXS |
| | GTSASLAISGLRSEXEADYYCQSYDGSLSGSVFGGXTKLTVLGEXKLISEXXLSGSAA |
| | [SEQ ID NO: 35] |
| TGF- | EVQLLESGGGLVQPGGSLRLSCAASGFTFSTYAMSWVRQAPGKGLEWVAVVSIDGGTTYYGDPVKGRFT |
| β1 | ISRDNSKNTLYLQMNSLRAEDTAVYYCTRGPTLTYYFDYWGQGTLVTVSSGGGGSGGGGSGGGGSQSVL |
| (1) | TQPPSASGTPGQRVTISCSGSSSNIGSNTVNWYQQLPGTAPKLLIYSNNQRPSGVPDRFSGSKSGTSAS |
| | LAISGLRSEDEADYYCQSYDSSLSGWVFGGXTKLXVLGEQKLISEEDLSGSAA [SEQ ID NO: 36] |
| TGF- | EVQLLESGGGLVQPGGSLRLSCAASGFTFGDYAMSWFRQAPGKGLEWVSGVSWNGSRTHYADSVKGRFT |
| β1 | ISRDNSKNTLYLQMNSLRAEDTAVYYCARDGNRPLDYWGQGTLVTVSSGGGGSGGGGSGGGGSQSVLTQ |
| (2) | PPSASGTPGQRVTISCTGSSSNIGAGYDVHWYQQLPGTAPKLLIYGNSNRPSGVPDRFSGSKSGTSASL |
| | AISGLRSEDEADYYCAAWDDRLNGWVFGGGTKLXVLGEQKLISEXDLSGSAA [SEQ ID NO: 37] |
| TGF- | EVQLLESGGGLVQPGGSLRLSCAASGFTFSDYYIGWIRQAPGKGLEWVSGINWNGGSTGYADSVKGRFT |
| β1 | ISRDNSKNTLYLQMNSLRAEDTAVYYCARRSTPSSSWALPDFFDYWGQGTLVTVSSGGGGSGGGGSGGG |
| (3) | VGSQSLTQPPSASGTPGQRVTISCTGSSSNIGANYDVHWYQQLPGTAPKLLIYSNNQRPSGVPDRFSGS |
| | KSGTSASLAISGLRSEDXADYYCQSYDSSLSGWVFGGXTKLTVLGEQKLISXXXLSGSAA |
| | [SEQ ID NO: 38] |
| TNF- | EVQLLESGGGLVQPGGSLRLSCAASGFTFYSSGMYWVRQAPGKGLEWVSAISGSGGSTYYADSVKGRFT |
| α (1) | ISRDNSKNTLYLQMNSLRAEDTAVYYCARHGYSYSFDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGSGTDFTLT |
| | ISSLQPEDFATYYCQQGVFYPHTFXQGTKLEIKRLXDYKDHDGDYKDHDIDYKDDDXKAA |
| | [SEQ ID NO: 39] |
| TNF- | EVQLLESGGGLVQPGGSLRLSCAASGFTFSSYAMSWVRQAPGKGLEWVSAISGSGGSTYYADSVKGRFT |
| α (2) | ISRDNSKNTLYLQMNSLRAEDTAVYYCARYVSHYTAHWYAYFDYWGQGTLVTVSSGGGGSGGGGSGGGG |
| | SDIQMTQSPSSLSASVGDRVTITCRASQSISSYLNWYQQKPGKAPKLLIYAASSLQSGVPSRFSGSGSG |
| | TDFTLTISSLQPEDFATYYCQQAGYHPLTFGQGTKLEIKRLXDYKDHDXDYKDHDIDYXXXXDXAAXHH |
| | HHHH-SPRWXXXFAL--VXLRALXXXXFXXXXXX[SEQ ID NO: 40] |
| TNF- | EVQLLESGGGLVQPGGSLRLSCAASGFTFGSYSMSWVRQAPGKGLEWVSSISSYYGGTSYADSVKGRFT |
| α (3) | ISRDNSKNTLYLQMNSLRAEDTAVYYCARGAYLDYWGQGTLVTVSSGGGGSGGGGSGGGGSDIQMTQSP |
| | SSLSASVGDRVTITCRASQSISSYLNWYQQKPGKAPKLLIYAASSLQSGVPSRFSGSGSGTDFTLTISS |
| | LQPEDFATYYCQQYYFPFTFGQGTKLEIKRLXXYKDHDGDYKDHDIDYKDDDDKAA |
| | [SEQ ID NO: 41] |
| GM- | EVQLLESGGGLVQPGGSLRLSCAASGFTFSSYGMHWVRQAPGKGLEWVAVISYDGSNKYYADSVKGRFT |
| CSF | ISRDNSKNTLYLQMNSLRAEDTAVYYCARVGGMSAPVDYWGQGTLVTVSSGGGGSGGGGSGGGGSQSVL |
| (1) | TQPPSASGTPGQRVTISCSGSSSNIGSNTVNWYQQLPGTAPKLLIYDNNKRPSGVPDRXSGSKSGTSAS |
| | LAISGLRSEDEADYYCAAWDDSLIGLVVFGGXTKLTVLGEQKLISEXXLSGSAA |
| | [SEQ ID NO: 42] |
| GM- | EVQLLESGGGLVQPGGSLRLSCAASGFTFSSYAMSWVRQAPGKGLEWVAVISYDGSNEDSADSVKGRFT |
| CSF | ISRDNSKNTLYLQMNSLRAEDTAVYYCARGPSLRGVSDYWGQGTLVTVSSGGGGSGGGGSGGGGSQSVL |
| (2) | TQPPSASGTPGQRVTISCTGSSSNIGAGYDVHWYQQLPGTAPKLLIYNDNQRPSXVPDRFSGSKSGTSA |
| | SLAISGLRSEDEADYYCQTWGTGINVIFGGXTKLXVLGEQKLISXEDLSGSAA [SEQ ID NO: 43] |
| GM- | EVQLLESGGGLVQPGGSLRLSCAASGFTFSSYAMSWVRQAPGKGLEWVAVISYDGSNEDSADSVKGRFT |
| CSF | ISRDNSKNTLYLQMNSLRAEDTAVYYCARGPSLRGVSDYWGQGTLVTVSSGGGGSGGGGSGGGGSQSVL |
| (3) | TQPPSASGTPGQRVTISCTGSSSNIGAGYDVHWYQQLPGTAPKLLIYNDNQRPSGVPDRFSGSKSGTSA |
| | SLAISGLRSEDXADYYCQTWGTGINVIFGGXTKLTVLGEXKLISEXXLSGSAA [SEQ ID NO: 44] |
| TNF- | EVQLLESGGGLVQPGGSLRLSCAASGFTFGSSYMYWVRQAPGKGLEWVSSIYGSSSSTSYADSVKGRFT |
| β (1) | ISRDNSKNTLYLQMNSLRAEDTAVYYCARGYYWDYMDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGSGTDFTLT |
| | ISSLQPEDFATYYCQQAWDLPTFGQGTKLEIKRLXXYKDHDGDYKDHDIDXXXTMTRRP |
| | [SEQ ID NO: 45] |
| TNF- | EVQLLESGGGLVQPGGSLRLSCAASGFTFDDYGMSWVRQAPGKGLEWVAVISYDGSNKYYADSVKGRFT |
| β (2) | ISRDNSKNTLYLQMNSLRAEDTAVYYCTRHLGSAMGYWGQGTLVTVSSGGGGSGGGGSGGGGSQSVLTQ |
| | PPSASGTPGQRVTISCTGSSSNIGAGYDVHWYQQLPGTAPKLLIYGNSNRPSXVPDRFSGSXSGTSASL |
| | AISGLRSEDEADYYCQSYDSSLSGWVFGGXTKLTVLXEQKLISXXDLSGSAA [SEQ ID NO: 46] |
| IL- | EVQLLESGGGLVQPGGSLRLSCAASGFTFDTHWMSWVRQAPGKGLEWVSAISGSGGSTYYADSVKGRFT |
| 1ra | ISRDNSKNTLYLQMNSLRAEDTAVYYCARHDYGDYRAFDIWGQGTLVTVSSGGGGSGGGGSGGGGSQSV |
| (1) | LTQPPSASGTPGQRVTISCTGSSSNIGAGWVHWYQQLPGTAPKLLIYGNNNRPSGVPDRFSGSKSGTSA |
| | SLAISGLRSEDEADYYCQSYDSSLSGVVFGGXTKLXVLXEQKLISXEDLSGSAA |
| | [SEQ ID NO: 47] |
| IL- | EVQLLESGGGLVQPGGSLRLSCAASGFTFSKYAMTWVRQAPGKGLEWVSAISGSGGNTYYADSVKGRFT |
| 1ra | ISRDNSKNTLYLQMNSLRAEDTAVYYCARLVRGLYYGMDVWGQGTLVTVSSGGGGSGGGGSGGGGSQSV |
| (2) | LTQPPSASGTPGQRVTISCSGSSSNIGNNAVNWYQQLPGTAPKLLIYGNSNRPSGVPDRFSGSKSGTSA |
| | SLAISGLRSXDEADYYCQTXGTGPVVFGGXTKLTVLGEQKLISXXXXSGSAA [SEQ ID NO: 48] |
| IL- | EVQLLESGGGLVQPGGSLRLSCAVSGFTFSSYSMNWVRQAPGKGLEWVAGIGGRGATTYYVDSVKGRFT |
| 1ra | ISRDNSKNTLYLQMNSLRAEDTAVYYCARLRVVPAARFDYWGQGTLVTVSSGGGGSGGGGSGGGGSQSV |
| (3) | LTQPPSASGTPGQRVTISCSGSSSNIGSNTVNWYQQLPGTAPKLLIYGNSNRPSGVPDRFSGSKSGTSA |
| | SLAISGLRSEDEADYYCQSYDSSLSGPPWVFGGXXKLXVLXEQKLISEEDLSGSAA |
| | [SEQ ID NO: 49] |
| IL-16 | EVQLLESGGGLVQPGGSLRLSCAASGFTFSNHAMSWVRQAPGKGLEWVSGVSWNGSRTHYADSVKGRFT |
| (1) | ISRDNSKNTLYLQMNSLRAEDTAVYYCARAALVQGVKHAFEIWGQGTLVTVSSGGGGSGGGGSGGGGSQ |
| | SVLTQPPSASGTPGQRVTISCSGSSSNIGSNTVNWYQQLPGTALKLLIYRNNQRPSGVPDRFSGSKSGT |
| | SASLAISGLRSEDEADYYCASWDDRLSGLVFGGXTKLTVLGEQKLISEXDLSGSAA |
| | [SEQ ID NO: 50] |
| IL-16 | EVQLLESGGGLVQPGGSLRLSCAASGFTFSNHAMSWVRQAPGKGLEWVSGVSWNGSRTHYADSVKGRFT |
| (2) | ISRDNSKNTLYLQMNSLRAEDTAVYYCARAALVQGVKHAFEIWGQGTLVTVSSGGGGSGGGGSGGGGSQ |
| | SVLTQPPSASGTPGQRVTISCSGSSSNIGSNTVNWYQQLPGTALKLLIYRNNQRPSGVPDRFSGSXSGT |
| | SASLAISGLRSEXEADYYCASWDDRLSGLVFGGXTKLTVLXEQKLISEEDLSGSAA |
| | [SEQ ID NO: 51] |
| IL-18 | EVQLLESGGGLVQPGGSLRLSCAASGFTFSSYGMHWVRQAPGKGLEWVSGINWNGGSTGYADSVKGRFT |
| (1) | ISRDNSKNTLYLQMNSLRAEDTAVYYCARDLRGGRFDPWGQGTLVTVSSGGGGSGGGGSGGGGSQSVLT |
| | QPPSASGTPGQRVTISCTGSSSNIGAGYVVHWYQQLPGTAPKLLIYRNNQRPSGVPDRFSGSXSGTSAS |
| | LAISGLRSEDEADYYCSSXAGSKNLIFGGXTKLTVLGEQKLISXXXLSGSAA [SEQ ID NO: 52] |
| IL-18 | EVQLLESGRGLVQPGGSLRLSCAASGFTFSSYGMHWVRQAPGKGLEWVSAIGTGGDTYYADSVMGRFTI |
| (2) | SRDNSKNTLYLQMNSLRAEDTAVYYCARSPRRGATAGTFDYWGQGTLVTVSSGGGGSGGGGSGGGGSQS |
| | VLTQPPSASGTPGQRVTISCSGSSSNIGSNIVNWYQQLPGTAPKLLIYRNNQRPSGVPDRFSGSKSGTS |
| | ASLAISGLRSEDEADYYCXSYDNSLSGWVFGGXXKLXVLGEXKLISEXDLSGSAA |
| | [SEQ ID NO: 53] |
| MCP- | EVQLLESGGGLVQPGGSLRLSCAASGFTFSSYGMHWVRQAPGKGLEWVSGISWNGGKTHYVDSVKGQFT |
| 4 (1) | ISRDNSKNTLYLQMNSLRAEDTAVYYCARGGYSSGWAFDYWGQGTLVTVSSGGGGSGGGGSGGGGSQSV |
| | LTQPPSASGTPGQRVTISCSGRSSNIESNTVNWYQQLPGTAPKLLIYGNSNRPSGVPDRFSGSKSGTSA |
| | SLAISGLRSEDEADYYCAAWDDRLNAVVFGGXTKLTVLGEQKLISEXDLSGSAA |
| | [SEQ ID NO: 54] |
| IFN-γ | EVQLLESGGGLVQPGGSLRLSCAASGFTFSSYGMHWVRQAPGKGLEWVSGVSWNGSRTHYADSVKGRFT |
| (1) | ISRDNSKNTLYLQMNSLRAEDTAVYYCARGRTGHGWKYYFDLWGQGTLVTVSSGGGGSGGGGSGGGGSQ |
| | SVLTQPPSASGTPGQRVTISCSGSSSNIGNNAVNWYQQLPGTAPKLLIYGNSNRPSXVPDRFSGSXSGT |
| | SASLAISGLRSEDEADYYCQXWGTGLGVFGGXTKLTVLGEXKLISEEXLSGSAAv |
| | [SEQ ID NO: 55] |
| IFN-γ | EVQLLESGGGLVQPGGSLRLSCAASGFTFSRHGFHWVRQGPGKGLEWVSGVSWNGSRTHYADSVKGRFT |
| (2) | ISRDNSKNTLYLQMNSLRAEDTAVYYCARGNWYRAFDIWGQGTLVTVSSGGGGSGGGGSGGGGSQSVLT |
| | QPPSASGTPGQRVTISCSGGSSHIGRNFISWYQQLPGTAPKLLIYAGNSRP [SEQ ID NO: 56] |
| IL-1b | EVQLLESGGGLVQPGGSLRLSCAASGFTFSSYAMSWVRQAPGKGLEWVSYISSSGSTIYYADSVKGRST |
| (1) | ISRDNSKNTLYLQMNSLRAEDTAVYYCARVRQNSGSYAYWGQGTLVTVSSGGGGSGGGGSGGGGSQSVL |
| | TQPPSASGTPGQRVTISCTGTSSNIGAPYDVHWYQQLPGTAPKLLIYGNSNRPSGVPDRFSGSKSGTSA |
| | SLAISGLRSEDXADYYCQSYDSSLSAVVFGGXTKLTVLGEQKLISEXXLSGSAA |
| | [SEQ ID NO: 57] |
| IL-1b | EVQLLESGGGLVQPGGSLRLSCAASGFTFSRYVMTWVRQAPGKGLEWVSLISGGGSATYYADSMKGRFT |
| (2) | ISRDNSKNTLYLQMNSLRAEDTAVYYCAKRVPYDSSGYYPDAFDIWGQGTLVTVSSGGGGSGGGGSGGG |
| | GSQSVLTQPPSASGTPGQRVTISCTGSSSNIGAGYDVHWYQQLPGTAPKLLIYGNSNRPSGVPDQFSGS |
| | KSGTSASLAISGLRSEDEADYYCAAWDDSLNGPVFGGXTXLTXLXEQKLISEEXLSGSAA |
| | [SEQ ID NO: 58] |
| IL-1b | EVQLLESGGGLVQPGGSLRLSCAASGFTFSSYWMSWVRQAPGKGLEWVAVVSYDGNNKYYADSRKGRFT |
| (3) | ISRDNSKNTLYLQMNSLRAEDTAMYYCASYWYTSGWYPYGMDVWGQGTLGTVSSGGGGSGGGGSGGGGS |
| | QSVLTQPPSASGTPGQRVTISCTGSSSNIGAGYDLHWYQQLPGTAPKLLIYRNNQRPSGVPDRFSGSKS |
| | GTSASLAISGLRSEDEADYYCSSYVDNNNLVFGGXTKLTVLXEQKLISEXXLSGSAA |
| | [SEQ ID NO: 59] |
| Eotaxin | EVQLLESGGGLVQPGGSLRLSCAASGFTFSSYWMSWVRQAPGKGLEWVSGVSWNGSRTHYADSVKGRFT |
| (1) | ISRDNSKNTLYLQMNSLRAEDTAVYYCVKGKGTIAMPGRARVGWWGQGTLVTVSSGGGGSGGGGSGGGG |
| | SQSVLTQPPSASGTPGQRVTISCSGSSSNIGNNAVNWYQQLPGTAPKLLIYANSNRPSGVPDRFSGSXS |
| | GTSASLAISGLRSEDEADYYCAAWDDSLSGPVFGGXTKLTVLGEQKLISXXDLSXSAA |
| | [SEQ ID NO: 60] |
| Eotaxin | EVQLLESGGGLVQPGGSLRLSCAASGFTFSAYWMTWVRQAPGKGLEWVSVIYSGGSTYYADSVKGRFTI |
| (2) | SRDNSKNTLYLQMNSLRAEDTAVYYCARQTQQEYFDYWGQGTLVTVSSGGGGSGGGGSGGGGSQSVLTQ |
| | PPSASGTPGQRVTISCFGSNSNIGSSTVNWYQQLPGTAPKLLIYDNDKRPSGVPDRFSGSXSGTSASLA |
| | ISGLRSEDEADYYCAAWDDSLNGPVFGGXTKLTVLGEQKLISXXXLSGSXAAHHHHHH-SPRXPIRPIV |
| | SXXTIHWPSFYNVXTGKXXXLPNXIXXXHIPLSPAXXIXXXPXXXXX[SEQ ID NO: 61] |
| Eotaxin | EVQLLESGGGLVQPGGSLRLSCAASGFTFRGYAMSWVRQAPGKGLEWVSGVSWNGSRTHYADSVKGRFT |
| (3) | ISRDNSKNTLYLQMNSLRAEDTAVYYCARAPAVAGWFDPWGQGTLVTVSSGGGGSGGGGSGGGGSQSVL |
| | TQPPSASGTPGQRVTISCSGSSSNIGSHTVNWYQQLPGTAPKLLIYRNNQRPSGVPDRFSGSKSGTSAS |
| | LAISGLRSEDXADYYCAAWDDSLSGRVXGGGXKLTVLGEQKLISEEDLSGSAA [SEQ ID NO: 62] |
| RANTES | EVQLLESGGGLVQPGGSLRLSCAASGFTFSSYGMHWVRQAPGKGLEWVAVISNDGTKKDYADSVKGRFT |
| (1) | ISRDNSKNTLYLQMNSLRAEDTAVYYCARDASGYDDYYFDYWGQGTLVTVSSGGGGSGGGGSGGGGSQS |
| | VLTQPPSASGTPGQRVTISCTGSSSNIGAGSDVHWYQQLPGTAPKLLIYRDDQRSSGVPDRFSGSKSGT |
| | SAFLAISGLRSEDEADYYCQSYDNSLSGWVIGGXTKLTVLGEQKLISEXXLSGSAA |
| | [SEQ ID NO: 63] |
| RANTES | EVQLLESGGGLVQPGGSLRLSCAASGFTFSSYAMSWVRQAPGKGLEWVSAISGSGGSTYYADSVKGRFT |
| (2) | ISRDNSKNTLYLQMNSLRAEDTAVYYCARDNDYSSDTFDYWGQGTLVTVSSGGGGSGGGGSGGGGSQSV |
| | LTQPPSAFGTPGQRVTISCSGSSSNIGSDYVYWYQQLPGTAPKLLIYSDNQRP [SEQ ID NO: 64] |
| RANTES | EVQLLESGGGLVQPGGSLRLSCAASGFTFSNYGMNWVRQAPGKGLEWVSGVSWNGSRTHYVDSVKRRFT |
| (3) | ISRDNSKNTLYLQMNSLRAEDTAVYYCARPRLRSHNYYGMDVWGQGTLVTVSSGGGGSGGGGSGGGGSQ |
| | SVLTQPPSASGTPGQRVTISCSGSSFKSGKNYVSWYQQLPGTAPKLLIYRNNQRPSGVPDRFSGSKSGT |
| | SASLAISGLRSEDEADYYCAAWDVRVKGVIFGGXTKLTVLGEQKLISEXDLSGSAA |
| | [SEQ ID NO: 65] |
| MCP- | EVQLLESGGGLVQPGGSLRLSCAASGFTFSSYAMSWVRQAPGKGLEWVSGVSWNGSRTHYADSVKGRFT |
| 1 (1) | ISRDNSKNTLYLQMNSLRAEDTAVYYCARGGHQQLGQWGQGTLVTVSSGGGGSGGGGSGGGGSQSVLTQ |
| | PPSASGTPGQRVTISCSGSSSNIGNNYVSWYQQLPGTAPKLLIYRDSRRPSGVPDRFSGSKSGTSASLA |
| | ISGLRSEXEADYYCAAWDDSLKGWLFGGXTKLTVLXEQKLISEXXLSGSAA [SEQ ID NO: 66] |
| MCP- | EVQLLESGGGLVQPGGSLRLSCAASGFTFSSYAMSWVRQAPGKGLEWVSYISSSSSYTNYADSVKGRFT |
| 1 (2) | ISRDNSKNTLYLQMNSLRAEDTAVYYCARFRYNSGKMFDYWGQGTLVTVSSGGGGSGGGGSGGGGSQSV |
| | LTQPPSASGTPGQRVTISCSGSSSNIGRNTVNWYQQLPGTAPKLLIYGNSNRRSGVPDRFSGSKSGTSA |
| | SLAISGLRSEDEADYYCAAWDDSLSGVVFGGXTKLTVLXEQKLISEXDLSGSAA |
| | [SEQ ID NO: 67] |
| MCP- | EVQLLESGGGLVQPGGSLRLSCAASGFTFSNYGMHWVRQAPGKGLEWVAVISYDGSNKYYADSVKGRFT |
| 1 (3) | ISRDNSKNTLYLQMNSLRAEDTAVYYCAKSHYYDTTSFDYWGQGTLVTVSSGGGGSGGGGSGGGGSQSV |
| | LTQPPSASGTPGQRVTISCSGSSSNIGTNPVNWYQQLPGTAPKLLIYDNNKRPSGVPDRFSGSKSGTSA |
| | SLAISGLRSEDXADYYCAAWDDSLSGVVFGGXTKLTVLGEQKLISXEDLSGSAA |
| | [SEQ ID NO: 68] |
| MCP- | EVQLLESGGGLVQPGGSLRLSCAASGFTFSTYGMHWVRQAPGKGLEWVSGVSWNGSRTHYVNSVKRRFT |
| 3 (1) | ISRDNSKNTLYLQMNSLRAEDTAVYYCARVAPGSGKRLRAFDIWGQGTLVTVSSGGGGSGGGGSGGGGS |
| | QSVLTQPPSASGTPGQRVTISCSGSSSNIGNNAVNWYQQLPGTAPKLLIYEVSKRPPGVPDRFSGSKSG |
| | TSASLAISGLRSEDXADYYCSSYAGSSKWVFGGXTKLTVLGEQKLISEEDLSGSAA |
| | [SEQ ID NO: 69] |
| MCP- | EVQLLESGGGLVQPGGSLRLSCAASGFTLSSNYMSWVRQAPGKGLEWVSGISASGHSTHYADSGKARFT |
| 3 (2) | ISRDNSKNTLYLQMNSLRAEDTAVYYCARGKSLAYWGQGTLVTVSSGGGGSGGGGSGGGGSQSVLTQPP |
| | SASGTPGQRVTISCSGSSSNIGNNAVNWYQQLPGTAPKLLIYRNNQRPSGVPDRFSGSKSGTSASLAIS |
| | GLRSEDEADYYCAAWDDSLSVVVFGGXTKLTVLGEQKLISXXXLSGSAA [SEQ ID NO: 70] |
| MCP- | EVQLLESGGGLVQPGGSLRLSCAASGFTFSIYWMSWVRQAPGKGLEWVAYIGGISNIVSYSDSVKGRFT |
| 3 (3) | ISRDNSKNTLYLQMNSLRAEDTAVYYCAKAPGYSSGWGWFDPWGQGTLVTVSSGGGGSGGGGSGGGGSQ |
| | SVLTQPPSASGTPGQRVTISCSGSSSNIGTNSVFWYQQLPGTAPKLLIYGNNNRPSGVPDRFSGSKSGT |
| | SASLAISGLRSEDXADYYCMIWHSSASVFGXXTKLTVLGEQKLISEXXLSGSAA |
| | [SEQ ID NO: 71] |
| B- | EVQLLESGGGLVQPGGSLRLSCAASGFTFSSYAMHWVRQAPGKGLEWVAVIAYDGINEYYGDSVKGRFT |
| galacto- | ISRDNSKNTLYLQMNSLRAEDTAVYYCARGGIYHGFDIWGQGTLVTVSSGGGGSGGGGSGGGGSQSVLT |
| sidase | QPPSASGTPGQRVTISCSGSSSNIGSNYVYWYQQLPGTAPKLLIYDNHKRPSGVPDRFSGSXSGTSASL |
| | AISGLRSEDEADYYCAAWDDNSWVFGGXTKLTVLGXYKDDDDKAA [SEQ ID NO: 72] |
| Angio- | EVQLLESGGGLVQPGGSLRLSCAASGFTFSDHYMDWVRQAPGKGLEWVSGVSWNGSRTHYADSVKGRFT |
| motin | ISRDNSKNTLYLQMNSLRAEDTAVYYCARDTWAYGAFDIWGQGTLVTVSSGGGGSGGGGSGGGGSQSVL |
| (1) | TQPPSASGTPGQRVTISCSGSNSNIGRNTVNWYQQLPGTAPKLLIYRDNQRPSGVPDRFSGSXSGTPAS |
| | LAISGLRSEDXADYYCAAWDVSLNGWVFGGXTKLTVLGDYXDHDGDYKDHDIDXXDDDDXXAAHHHHH |
| | H-SPRWXIRPIVSRITIXWXXFYXVXXXKXX [SEQ ID NO: 73] |
| Angio- | EVQLLESGGGLVQPGGSLRLSCAASGFTFNDYYMTWIRQAPGKGLEWVSYISSSGSTIYYADSVKGRFT |
| motin | ISRDNSKNTLYLQMNSLRAEDTAVYYCARERLPDVFDVWGQGTLVTVSSGGGGSGGGGSGGGGSQSVLT |
| (2) | QPPSASGTPGQRVTISCSGSGSNIGTNSVSWYQQLPGTAPKLLIYFDDLLPSGVPDRFSGSKSGTSASL |
| | AISGLRSEDEADYYCAAWDDSLSGVVFGGXTKLTVLGXYKDHDGDYKDHDIDYKDDDXKAXAHHHHHH- |
| | SPRXXXRXIVSXIXIHXXXFYNXXTGKTXXXXXXIXXAAXXXFXX[SEQ ID NO: 74] |
| Leptin | EVQLLESGGGLVQPGGSLRLSCAASGFTFGDFAMSWVRQAPGKGLEWVANIKQDGSVKYYVDSVKGRFT |
| | ISRDNSKNTLYLQMNSLRAEDTAVYYCARFLAGFYYGMDVWGQGTLVTVSSGGGGSGGGGSGGGGSQSV |
| | LTQPPSASGTPGQRVTISCSGSDSNIGGNTVNWYQQLPGMAPKLLIYYDDLLPSGVPDRFSGSKSGTSA |
| | SLAISGLRSEDEADYYCAAYDDTMNGWGFGGXTKLTVLGXYKDXDDKAA [SEQ ID NO: 75] |
| Integrin | EVQLLESGGGLVQPGGSLRLSCAASGFTFSTYNMNWVRQAPGKGLEWVSTISGSGGRTYYADSVKGRFT |
| a10 | ISRDNSKNTLYLQMNSLRAEDTAVYYCARDRVATLDAFDIWGQGTLVTVSSGGGGSGGGGSGGGGSQSV |
| | LTQPPSASGTPGQRVTISCSGSSSNIGSNSVSWYQQLPGTAPKLLIYSNNQRPSGVPDRFSGSKSGTSA |
| | SLAISGLRSEDEADYYCAAWDDSLSGVVFGGXTKLTVLGEQKLISEXDLSGSAA |
| | [SEQ ID NO: 76] |
| Integrin | EVQLLESGGGLVQPGGSLRLSCAASGFTFRRDWMSWVRQVPGKGLEWVSVISGSDGSTYYADSVKGRFT |
| a11 | ISRDNSKNTLYLQMNSLRAEDTAVYYCASYSPLGNWFDSWGQGTLVTVSSGGGGSGGGGSGGGGSQSVL |
| | TQPPSASGTPGQRVTISCTGSSSNIGAGYDVHWYQQLPGTAPKLLIYSDTYRPSGVPDRFSGSXSGTSA |
| | SLAISGLRSEDEADYYCQSYDSSLXGFVVFGGXTKLTVLXEQKLISEXXLSGSAA |
| | [SEQ ID NO: 77] |
| IgM | EVQLLESGGGLVQPGGSLRLSCAASGFTFSDYYMSWIRQAPGKGLEWVSAIGSGPYYAHSVRDRFTISR |
| (1) | DNSKNTLYLQMNSLRAEDTAVYYCARGGVEASFDYWGQGTLVTVSSGGGGSGGGGSGGGGSQSVLTQPP |
| | SASGTPGQRVTISCTGSSSNIGAGYDVHWYQQLPGTAPKLLIYGNTNRPSGVPNRFSGSKSGTSASLAI |
| | SGLRSEDEADYYCQSYDNDLSGWVFGGXTKLXVLGEQKLISXXXLSGSAA [SEQ ID NO: 78] |
| LDL | EVQLLESGGGLVQPGGSLRLSCAASGFTFSDYYMSWVRQAPGKGLEWVSGVSWNGSRTHYADSVKGRFT |
| (1) | ISRDNSKNTLYLQMNSLRAEDTAVYYCARAARYSYYYYGMDVWGQGTLVTVSSGGGGSGGGGSGGGGSQ |
| | SVLTQPPSASGTPGQRVTISCSGSSSNIGNNAVNWYQQLPGTAPKLLIYGNDRRPSGVPDRFSGSKSGT |
| | SASLAISGLRSEDEADYYCQTWGTGRGVFGGGTKLTVLGEQKLISEXXLSGSAA |
| | [SEQ ID NO: 79] |
| LDL | EVQLLESGGGLVQPGGSLRLSCAASGFTFSNAWMSWVRQAPGKGLEWVSSISTSSNYIYYADSVKGRFT |
| (2) | ISRDNSKNTLYLQMNSLRAEDTAVYYCARVKKYSSGWYSNYAFDIWGQGTLVTVSSGGGGSGGGGSGGG |
| | GSQSVLTQPPSASGTPGQRVTISCSGSSSSIGNNFVSWYQQLPGTAPKLLIYDNNKRPSXVPDRFSGSX |
| | SGTSASLAISGLRSEDXADYYCAAWDDSLNGWVFGGXTKLTVLXXYKDHDGDYXDHDIDYKDXXDKAA |
| | [SEQ ID NO: 80] |
| PSA | EVQLLESGGGLVQPGGSLRLSCAASGFTFRSYEMNWVRQAPGKGLEWVAVIGGNGVDTDYADSVKGRFT |
| | ISRDNSKNTLYLQMNSLRAEDTAVYYCVREEVDFWSGYYSYGMDVWGQGTLVTVSSGGGGSGGGGSGGG |
| | GSQSVLTQPPSASGTPGQRVTISCSGSSSNIGDNFVSWYQQLPGTAPKLLIYRTNGRPSGVPDRFSGSX |
| | SGTSASLAISGLRSEDEADYYCATWDDNLNGRVVFGGXTKLWLGDYKDXXDKAA |
| | [SEQ ID NO: 81] |
| Lewis | EVQLLESGGGLVQPGGSLRLSCAASGFTFSNYWMHWVRQAPGKGLEWVANIKEDGSEKYYVDSVKGRFT |
| x (1) | ISRDNSKNTLYLQMNSLRAEDTAVYYCAREGETSFGLDVWGQGTLVTVSSGGGGSGGGGSGGGGSQSVL |
| | TQPPSASGTPGQRVTISCSGSSSNIGSNTVNWYQQLPGTAPKLLIYSNNQRPSGVPDRFSGSKSGTSAS |
| | LAISGLRSEDEADYYCASWDDSLSGWVFGGXTKLTVLGDYKDDDDKAA [SEQ ID NO: 82] |
| Lewis | EVQLLESGGGLVQPGGSLRLSCAASGFTFSRYWMHWVRQAPGKGLEWVANIKPDGSEQYYADSVKGRFT |
| x (2) | ISRDNSKNTLYLQMNSLRAEDTAVYYCAREGLSSGWSYGMDVWGQGTLVTVSSGGGGSGGGGSGGGGSQ |
| | SVLTQPPSASGTPGQRVTISCSGSNSNIGSNTVNWYQQLPGTAPKLLIYTNINRPSGVPDRFSGSKSGT |
| | SASLAISGLRSXDEADYYCATWDDSLSGWVFGGXTKLTVLGXYKDXXDKAA [SEQ ID NO: 83] |
| Lewis | EVQLLESGGGLVQSGGSLRLSCAASGFTFSSYTLHWVRQAPGKGLEYVSAISSNGGSTYYADSVKGRFT |
| y | ISRDNSKNTLYLQMNSLRAEDTAVYYCASDVYGDYPRGLDYWGQGTLVTVSSGGGGSGGGGSGGGGSQS |
| | VLTQPPSASGTPGQRVTISCSGTTSNIGSNYVHWYQQLPGTAPKLLIYGNNNRPSGVPDRFSGSKSGTS |
| | ASLAISGLRSEDXADYYCQSYDRSLGGLRVFGGXTKLTVLXDYKXDDDKAA [SEQ ID NO: 84] |
| Sialyl | EVQLLESGGGLVQPGGSLRLSCAASGFTLSSYAMSWVRQAPGKGLEWVSSISSGNSYIYYADSVKGRFT |
| x | ISRDNSKNTLYLQMNSLRAEDTAVYYCARGRGRGGGFELWGQGTLVTVSSGGGGSGGGGSGGGGSQSVL |
| | TQPPSASGTPGQRVTISCSGSSSNIGTYTVNWYQQLPGTAPKLLIYSNNQRPSGVPDRFSGSKSGTSAS |
| | LAISGLRSEXEADYYCSSNAGIDNILFGGXTKLTVLGEQKLISEXDLSGSXAAHHHHXXXXXXXXIXXX |
| | XXXXXXXXXXXXXXXLXX [SEQ ID NO: 85] |
| Pro- | EVQLLESGGGLVQPGGSLRLSCAASGFTFSSYAMSWVRQAPGKGLEWVSSMSASGGSTYYADSVKGRFT |
| cathepsin | ISRDNSKNTLYLQMNSLRAEDTAVYYCARDRGSYGMDVWGQGTLVTVSSGGGGSGGGGSGGGGSQSVLT |
| W | QPPSASGTPGQRVTISCSGSTSNIGSYAVNWYQQLPGTAPKLLIYGNNNRPSGVPDRFSGSXSGTSASL |
| | AISGPRSEDEADYYCAAWDDSLNGGVFGGXTKLTVLGXYKXDDDKAA [SEQ ID NO: 86] |
| BTK | EVQLLESGGGLVQPGGSLRLSCAASGFTFSNYAMSWVRQAPGKGLEWVSGINWNGGSTGYADSVKGRFT |
| (1) | ISRDNSKNTLYLQMNSLRAEDTAVYYCAKHLKRYSGSSYLFDYWGQGTLVTVSSGGGGSGGGGSGGGGS |
| | QSVLTQPPSASGTPGQRVTISCSGSSSXIGSNYVYWYQQLPGTAPKLLIY [SEQ ID NO: 87] |
| Digoxin | EVQLLESGGGLVQPGGSLRLSCAASGFTFSSYAMSWVRQAPGKGLEWVAVIWHDGSSKYYADSVKGRFT |
| | ISRDNSKNTLYLQMNSLRAEDTAVYYCARATGDGFDYWGQGTLVTVSSGGGGSGGGGSGGGGSQSVLTQ |
| | PPSASGTPGQRVTISCSGSSSNIGSNYVYWYQQLPGTAPKLLIYRNNQRPSGVPDRFSGSKSGTSASLA |
| | ISGLRSEDEADYYCAAWDDSLNGVVFGGXTKLTVLGEQKLISXXXLSXSAA [SEQ ID NO: 88] |
| GLP- | EVQLLESGGGLVQPGGSLRLSCAASGFTFRSYGMHWVRQAPGKGLEWVSGLSWNSAGTGYADSVKGRFT |
| 1 R | ISRDNSKNTLYLQMNSLRAEDTAVYYCAKEMGNNWDHIDYWGQGTLVTVSSGGGGSGGGGSGGGGSQSV |
| | LTQPPSASGTPGQRVTISCTGSSSNIGAGYDVHWYQQLPGTAPKLLIYGNSNRPSGVPDRFSGSKSGTS |
| | ASLAISGLRSEDEADYYCAAWDDGLSGPVFGGGTKLTXLGEQKLISEEDLSGSAA |
| | [SEQ ID NO: 89] |
| GLP- | EVQLLESGGGLVQPGGSLRLSCAASGFTFNSYGMHWVRQAPGKGLEWVSAISGSGGSTYYAESVKGRST |
| 1 | ISRDNSKNTLYLQMNSLRAEDTAVYYCVTRNAVFGFDVWGQGTLVTVSSGGGGSGGGGSGGGGSQSVLT |
| | QPPSASGTPGQRVTISCTGSSSNIGAGFDVHWYQQLPGTAPKLLIYDNNKRPSGVPDRFSGSKSGTSAS |
| | LAISGLRSEDEADYYCQSFDSSLSGVVIGGXTKLTVLXEQKLISXEXLSGSAA [SEQ ID NO: 90] |
| C1q | EVQLLESGGGLVQPGGSLRLSCAASGFTFDDYGMSWVRQVPGKGLEWVSAISGSGATTFYAHSVQGRFT |
| | ISRDNSKNTLYLQMNSLRAEDTAVYYCARGGRGYDWPSGAFDIWGQGTLVTVSSGGGGSGGGGSGGGGS |
| | QSVLTQPPSASGTPGQRVTISCTGSSSNIGAGYDVHWYQQLPGTAPKLLIYENNKRPSXVPDRFSGSKS |
| | GTSASLAISGLRSEDXADYYCAAWDDSVNGYVVFGGXTKLTVLGEQKLISEXXLSGSAAXXHHHHH-SP |
| | RWPIRPIXSRXTIXXPSFYXXXXXXTXXLPXXIXXXHXPXXXXXX [SEQ ID NO: 91] |
| C1s | EVQLLESGGGLVQPGGSLRLSCAASGFTFSSYAMSWVRQAPGKGLEWVSGVSWNGSRTHYADSVKGRFT |
| | ISRDNSKNTLYLQMNSLRAEDTAVYYCARHMKAAAYVFEIWGQGTLVTVSSGGGGSGGGGSGGGGSQSV |
| | LTQPPSASGTPGQRVTISCSGSSSNIGSTAVNWYQQLPGTAPKLLIYSNNKRPSGVPDRFSGSXSGTSA |
| | SLAISGLRSEDEADYYCAAWDDRLNGNVLFGGXXKLTVLXEQXLISXXXLSGSAA |
| | [SEQ ID NO: 92] |
| C3 | EVQLLESGGGLVQPGGSLRLSCAASGFTFSSYAMSWVRQAPGKGLEWVSSVTGSGGGTYYADSVFGRFT |
| (1) | ISRDNSKNTLYLQMNSLRAEDTAVYYCARYRWFGNDAFDIWGQGTLVTVSSGGGGSGGGGSGGGGSQSV |
| | LTQPPSASGTPGQRVTISCSGSASNLGMHFVSWYQQLPGTAPKLLIYGNSNRPSGVPDRFSGSKSGTSA |
| | SLAISGLRSEDEADYYCAAWDDTLNIWVFGGXTKLTVLGEQKLISXXXLSGSAA |
| | [SEQ ID NO: 93] |
| C3 | EVQLLESGGGLVQPGGSLRLSCAASGFTFSTYRMIWVRQAPGKGLEWVSSISGSNTYIHYADSVRGRFT |
| (2) | ISRDNSKNTLYLQMNSLRAEDTAVYYCARDRHPLLPSGMDVWGQGTLVTVSSGGGGSGGGGSGGGGSQS |
| | VLTQPPSASGTPGQRVTISCSGSSSNIGKHPVNWYQQLPGTAPKLLIYRNDQRPSGVPDRFSGSKSGTS |
| | ASLAISGLRSEDXADYYCQSYDSSLSGSWVFGGXTKLTVLGXQKLISEEDLSGSAA |
| | [SEQ ID NO: 94] |
| C4 | EVQLLESGGGLVQPGGSLRLSCAASGFTFSSYPMSWVRQAPGKGLEWVSTLYAGGWTSYADSVWGRFTI |
| (1) | SRDNSKNTLYLQMNSLRAEDTAVYYCARPKVESLSRYGMDVWGQGTLVTVSSGGGGSGGGGSGGGGSQS |
| | VLTQPPSASGTPGQRVTISCTGSSSNIGAGYVVHWYQQLPGTAPKLLIYDNSKRPSGVPDRFSGSKSGT |
| | SASLAISGLRSEDEADYYCQSYDSSLSGVVFGGXTKLTVLXEQKLISEXXLSGSAA |
| | [SEQ ID NO: 95] |
| C5 | EVQLLESGGGLVQPGGSLRLSCAASGFTFSSYRMNWVRQAPGKGLEWVSAISGSGGSTYYADSVKGRFT |
| (1) | ISRDNSKNTLYLQMNSLRAEDTAVYYCARGGGWFSGHYYFDYWGQGTLVTVSSGGGGSGGGGSGGGGSQ |
| | SVLTQPPSASGTPGQRVTISCTGATSNIGAGYDVHWYQQLPGTAPKLLIYRNNQRPSXVPDRFSGSXSG |
| | TSASLAIXGLRSEDXADYYCQSYDSSLRHWVFXGXXKLTVLXEQKLISEXXLSGSXA |
| | [SEQ ID NO: 96] |
| C5 | EVQLLESGGGLVQPGGSLRLSCAASGFTFSAYSMNWVRQAPGKGLEWVSGVSWNGSRTHYADSVKGRFT |
| (2) | ISRDNSKNTLYLQMNSLRAEDTAVYYCARENSGFFDYWGQGTLVTVSSGGGGSGGGGSGGGGSQSVLTQ |
| | PPSASGTPGQRVTISCTGSSSNIGSNTVNWYQQLPGTAPKLLIYGNSNRPSGVPDRFSGSKSGTSASLT |
| | ISGLRSEDXADYYCAAWDDSLSGWVFGGXTKLTVLXEQKLISEEXLSGSAA [SEQ ID NO: 97] |
| C1 | EVQLLESGGGLVQPGGSSLSCAASGFTFSDYYMSWIRQAPGKGLEWVSGISRGGEYTFYVDSVKGRFTI |
| inh. | SRDNSKNTLYLQMNSLRAEDTAVYYCARDPGGLDAFDIWGQGTLVTVSSGGGGSGGGGSGGGGSQSVLT |
| (1) | QPPSASGTPGQRVTISCTGSSSNIGARYDVQWYQQLPGTAPKLLIYGNSNRPSGVPDRFSGSKSGTSAS |
| | LAISGLRSEXXADYYCASWDDSLSGPVFGGXTKLTVLXEQKLISEXXLSXSAA [SEQ ID NO: 98] |
| Factor | EVQLLESGGGLVQPGGSLRLSCAASGFTFSSYSMNWVRQAPGKGLEWVAVISYDGRFIYYSDSVKGRFT |
| B | ISRDNSKNTLYLQMNSLRAEDTAVYYCARSYGGNLAMDVWGQGTLVTVSSGGGGSGGGGSGGGGSQSVL |
| (1) | TQPPSASGTPGQRVTISCTGSSSNIGAGYDVHWYQQLPGTAPKLLIYDNNKRPSGVPDRFSGSNSGTSA |
| | SLAISGLRSEDXADYYCAAWDDRLNGRVVFGGXTKLTVLGEQKLISEXDLSGSAA |
| | [SEQ ID NO: 99] |
| IL-12 | EVQLLESGGGLVQPGGSLRLSCAASGFTFSRYGMHWVRQAPGKGLEWVASIRGNARGSFYADSVKGRFT |
| (3) | ISRDNSKNTLYLQMNSLRAEDTAVYYCAKGDSSGWYFFDYWGQGTLVTVSSGGGGSGGGGSGGGGSQSV |
| | LTQPPSASGTPGQRVTISCTGSDSXIGAGFDVHWYQQLPGTAPKLLIYGNNNRPSGVPDRFSGSKSGTS |
| | ASLAISGLRSEDXADYYCQSYDTSLSGVLFGGXXKLTVLGEQKLISEXDLSGSAA |
| | [SEQ ID NO: 100] |
| IL-12 | EVQLLESGGGLVQPGGSLRLSCAASGFTFSDYGMHWVRQAPGKGLEWVSTVSGSGDNTYYADSVKGRFT |
| (4) | ISRDNSKNTLYLQMNSLRAEDTAVYYCTTTWRYWGQGTLVTVSSGGGGSGGGGSGGGGSQSVLTQPPSA |
| | SGTPGQRVTISCSGSSSNIGSNTVNWYQQLPGTAPKLLIYGNSNRPSGVPDRFSGSKSGTSASLAISGL |
| | RSEDEADYYCQSYDSSLSGWVFGGXTKLTVLXEQKLISXEDLSGSAA [SEQ ID NO: 101] |
| IL-16 | EVXLLESGGGLVQPGGSLRLSCAASGFTFSSYGMHWVRQAPGKGLEWVSGINWNGGSTGYADSVKGRFT |
| (3) | ISRDNSKNTLYLQMNSLRAEDTAVYYCARERGDAFDIWGQGTLVTVSSGGGGSGGGGSGGGGSQSVLTQ |
| | PPSASGTPGQRVTISCSGSSSNIGSNWNWYQQLPGTAPKLLIYSDNQRPSGVPDRFSGSKSGTSASLAI |
| | SGLRSXXEADYYCAAWXDSLNGPWVFGGXTKLXVLGEQKLISEEDLSGSAA [SEQ ID NO: 102] |
| IL-18 | EVQLLESGGGLVQPGGSLRLSCAASGFTFSRYGMHWVRQAPGKGLEWVAVISYDGSNKYYADSVKGRFT |
| (3) | ISRDNSKNTLYLQMNSLRAEDTAVYYCARHGYGDSRSAFDIWGQGTLVTVSSGGGGSGGGGSGGGGSQS |
| | VLTQPPSASGTPGQRVTISCTGSSSNIGAGYDVHWYQQLPGTAPKLLIYRNNQRPSGVPDRFSGSXSGT |
| | SASLAISGLRSEXXADYYCQSYDSSLSRWVFGGXTKLXVLGEQKLISXXXLSXSAA |
| | [SEQ ID NO: 103] |
| IL-1a | EVQLLESGGGLVQPGGSLRLSCAASGFTFSSYSMNWVRQAPGKGLEWVSYISSSSSYTNYADSVKGRFT |
| (3) | ISRDNSKNTLYLQMNSLRAEDTAVYYCARSVTRRAGYYYYYSGMDVWGQGTLVTVSSGGGGSGGGGSGG |
| | GGSQSVLTQPPSASGTPGQRVTISCSGSSSXIGSNTVNWYQQLPGTAPKLLIYRNNQRPSXVPDRFSGS |
| | XSGTSASLAISGLRSEDEAXYYCSSXAGSNSXVFGGXTKLTVLGEQKLISXXXLSGSAA |
| | [SEQ ID NO: 104] |
| IL-6 | EVQLLESGGGLVQPGGSLRLSCAASGFTFSNYGMHWVRQAPGKGLEWVSSITSSGDGTYFADSVKGRFT |
| (3) | ISRDNSKNTLYLQMNSLRAEDTAVYYCARAGGIAAAYAFDIWGQGTLVTVSSGGGGSGGGGSGGGGSQS |
| | VLTQPPSASGTPGQRVTISCSGSSSNVGSNYVYWYQQLPGTAPKLLIYDNNKRPSGVPDRFSGSKSGTS |
| | ASLAISGLRSEXEADYYCQSYDSSRWVFGGXTKLTVLGEQKLISEXXLSGSAA |
| | [SEQ ID NO: 105] |
| IL-6 | EVQLLESGGGLVQPGGSLRLSCAASGFTFSSNYMSWVRQAPGKGLEWVSSISSSSTIYYADSVKGRFTI |
| (4) | SRDNSKNTLYLQMNSLRAEDTAVYYCARQPASGTYDAFDIWGQGTLVTVSSGGGGSGGGGSGGGGSQSV |
| | LTQPPSXSGTPGQRVTISCTGSSSNIGAGYDVHWYQQLPGTAPKLLIYYDDLLPSGVPDRFSGSKSGTS |
| | ASLAISXLRSEDEADYYCAVWDDSLSGWVFGGXTKLTVLXEQKLISXXDLSGSAXAHHHHHHXSPRXXI |
| | RPIVSXITIHXXVVLXRRDWEXPXXTQLNXXXAHXPFXXXXNX [SEQ ID NO: 106] |
| IL-8 | EVQXLESGGGLVQPGGSLRLSCAASGFTFDDYGMSWVRQAPGKGLEWVSLISWDGGSTYYADSVKGRFT |
| (3) | ISRDNSKNTLYLQMNSLRAEDTAVYYCARDDLYGMDVWGQGTLVTVSSGGGGSGGGGSGGGGSQSVLTQ |
| | PPSASGTPGQRVTISCTGSSSNIGAGYDVHWYQQLPGTAPKLLIYDNNKRPSGVPDRFSGSKSGTSASL |
| | AISGLRSXDEADYYCAAWDDSLSGWVFGGXTKLTVLGEQKLISEXXLSGSAA [SEQ ID NO: 107] |
| MCP- | EVQLLESGGGLVQPGGSLRLSCAASGFTFSSYGMHWVRQAPGKGLEWVSGISWNGGKTHYVDSVKGQFT |
| 4 (3) | ISRDNSKNTLYLQMNSLRAEDTAVYYCARGGYSSGWAFDYWGQGTLVTVSSGGGGSGGGGSGGGGSQSV |
| | LTQPPSASGTPGQRVTISCSGRSSNIESNTVNWYQQLPGTAPKLLIYGNSNRPSGVPDRFSGSKSGTSA |
| | SLAISGLRSEXEADYYCAAWDDRLNAVVFGGXTKLXVLXEQKLISEXXLSGSAA |
| | [SEQ ID NO: 108] |
| Properdin | EVQLLESGGGLVQPGGSLRLSCAASGFTFSSNYMSWVRQAPGKGLEWVSAISGSGGSTYYADSVKGRFT |
| | ISRDNSKNTLYLQMNSLRAEDTAVYYCAKGGSGWYDYFDYWGQGTLVTVSSGGGGSGGGGSGGGGSQSV |
| | LTQPPSASGTPGQRVTISCTGSSSNIGAGYDVHWYQQLPGTAPKLLIYRNNQRPSGVPDRFSGSKSGTS |
| | ASLAISGLRSEDEAXYYCAAXDDGLNSPVFGGGTKLXVLXEQKLISEEDLSGSAXAHHHHHH-SPRXXI |
| | RPIVSRITIHWXXFXXXXXGKTXXXPXLXXXXXXPPFX [SEQ ID NO: 109] |
| TNF- | EVQLLESGGGLVQPGGSLRLSCAASGFTFSSYGMHWVRQAPGKGLEWVSAISGSGGSTYYADSVKGRFT |
| β (3) | ISRDNSKNTLYLQMNSLRAEDTAVYYCARGGWGPRSAFDIWGQGTLVTVSSGGGGSGGGGSGGGGSQSV |
| | LTQPPSASGTPGQRVTISCSGSSSNIGSNTVTWYQQLPGTAPKLLIYGNTNRLSGVPDRFSGSKSGTSA |
| | SLAISGLRSEDEADYYCEAWDDKLFGPVFGGXTXLTVLXEQKLISEXXLSGSAA |
| | [SEQ ID NO: 110] |
| TNF- | EVQLLESGGGLVQPGGSLRLSCAASGFTFSSYWMSWVRQAPGKGLEWVSGVNWNGSRTHYADSVKGRFT |
| β (4) | ISRDNSKNTLYLQMNSLRAEDTAVYYCASIRANYYYGMDVWGQGTLVTVSSGGGGSGGGGSGGGGSQSV |
| | LTQPPSASGTPGQRVTISCSGGSSNIGSHPVNWYQQLPGTAPKLLIYGNSNRPSGVPDRFSGSKSGTSA |
| | SLAISGLRSEDEADYYCAAWDASLSGWVFGGGXKLTVLXEXKLISXXXLSGSAA |
| | [SEQ ID NO: 111] |
| VFGF | EVQLLESGGGLVQPGGSLRLSCAASGFTFSSYEMNWVRQAPGKGLEWVSGISGSGGFTYYADSVKGRFT |
| (3) | ISRDNSKNTLYLQMNSLRAEDTAMYYCAREGYQDAFDIWGQGTLVTVSSGGGGSGGGGSGGGGSQSVLT |
| | QPPSASGTPGQRVTISCTGSSSNIGAGYDVHWYQQLPGTAPKLLIYSNNQRPSXVPDRFSGSXSGTSAS |
| | LAISGLRSEDXADYYCAAWDDSLSGPPWVFGGGXKLXVLXEQKLISXXXLSGSXAAHHHHHH-SPRXPI |
| | RPIVSXIXIHWPXFYNVXXXXTXXXPXLX [SEQ ID NO: 112] |
| VFGF | EVQLLESGGGLVQPGGSLRLSCAASGFTFXXXYXSWVRQAPGKGLEWVSXISWXXGSIGYADSVKGRFT |
| (4) | ISRDNSKNTLYLQMNSLRAEDTAVYYCXXXXXXXXNYFDYWGQGTLVTVSSGGGGSGGGGSGGGGSQSV |
| | LTQPPSASGTPGQRVTISCSGSNSNIGGNFVYWYQQLPGTAPKLLIYENSKRPSXVPDRFSGSXSGTSA |
| | SLAISGLRSEDXADYYCAAWDDSLXXVVFGGXTKLTVLGEQKLISXXXLSGSAA |
| | [SEQ ID NO: 113] |
| IL-4 | EVQLLESGGGLVQPGGSLRLSCAASGFTFSNAWMSWVRQAPGKGLEWVSAISGSGGSTYYADSVKGRFT |
| (3) | ISRDNSKNTLYLQMNSLRAEDTAVYYCARAIAARPFDYWGQGTLVTVSSGGGGSGGGGSGGGGSQSVLT |
| | QPPSASGTPGQRVTISCTGATSNIGAGYDIHWYQQLPGTAPKLLIYSTNNRPSGVPDRFSGSKSGTSAS |
| | LAISGLRSEDXADYYCAAWDDSLNGPVFGGXXKLTVLGEQKLISEXDLSGSAA |
| | [SEQ ID NO: 114] |
| CD40 | EVQLLESGGGLVQPGGSLRLSCAASGFTFSAYWMHWVRQAPGKGLEWVSGISGGGGSTYYADSVKGRFT |
| (2) | ISRDNSKNTLYLQMNSLRAEDTAVYYCARMTPWYYGMDVWGQGTLVTVSSGGGGSGGGGSGGGGSQSML |
| | TQPPSASGTPGQRVTISCSGSTS [SEQ ID NO: 115] |
| CD40 | EVQLLESGGGLVQPGGSLRLSCAASGFTFSTYGMHWVRQAPGKGLEWLSYISGGSSYIFYADSVRGRFT |
| (3) | ISRDNSENALYLQMNSLRAEDTAVYYCARILRGGSGMDLWGQGTLVTVSSGGGGSGGGGSGGGGSQSVL |
| | TQPPXXSGTPGQRVTISC [SEQ ID NO: 116] |
| CD40 | EVQLLESGGGLVQPGGSLRLSCAASGFTFSTYGMHWVRQAPGKGLEWLSYISGGSSYIFYADSVRGRFT |
| (4) | ISRDNSENALYLQMNSLRAEDTAVYYCARILRGGSGMDLWGQGTLVTVSSGGGGSGGGGSGGGGSQSVL |
| | TQPPSASGTPGQRVTISCTGSSSNIGAGYDVYWYQQLPGTAPKLLIYGNINRPSGVPDRFSGSKSGTSA |
| | SLAISGLRSEDXADYYCAAWDDSLXGLVFGGXXKLTVLXXYKDDDDKAA [SEQ ID NO: 117] |
| IgM | EVQLLESGGGLVQPGGSLRLSCAASGFTFSSYAMSWVRQAPGKGLEWVSGISWNSGSIGYADSVKGRFT |
| (3) | ISRDNSKNTLYLQMNSLRAEDTAVYYCARGDYSSSPGGYYYYMDVWGQGTLVTVSSGGGGSGGGGSGGG |
| | GSQSVLTQPPSASGTPGQRVTISCSGSSSXIGSNTVNWYQQLPGTAPKLLIYGNSNRPSXVPDRFSGXX |
| | SGTSASLAIXGLRSXDXADYYCSSXXSTNTVIFGGXTKLTVLGEQKLISXXDLSGSAA |
| | [SEQ ID NO: 118] |
| IgM | EVQLLESGGGLVQPGGSLRLSCAASGFTFGSYEMNWVRQAPGKGLEWVSVIYSGGSTYYADSVFGRFTI |
| (5) | SRDNSKNTLYLQMNSLRAEDTAVYYCARDTNPYYYYGMDVWGQGTLVTVSSGGGGSGGGGSGGGGSQSV |
| | LTQPPSASGTPGQRVTISCSGSSSNIGNNAVNWYQQLPGTAPKLLIYRNNQRPSXVPDRFSGSXSGTSA |
| | SLAISGLRSEDEADYYCQSYDSSLNGQVFGGXTKLTVLXEQKLISXEXLSGSAA |
| | [SEQ ID NO: 119] |
| HLA- | EVQLLESGGGLVQPGGSLRLSCAASGFTFSSYAMSWVRQAPGKGLEWVSAISGSGGSTYYADSVKGRFT |
| DR/ | ISRDNSKNTLYLQMNSLRAEDTAVYYCARDGLLPLDYWGQGTLVTVSSGGGGSGGGGSGGGGSQSVLTQ |
| DP | PPSASGTPGQRVTISCSGGSSNIGGNAVNWYQQLPGTAPKLLIYENNKRPSXVPDRFSGSXSGTSASLA |
| | ISGLRSEDXADYYCSSYAVSNNFEVLFGGXTKLTVLXEQKLISXXDLSGSAA [SEQ ID NO: 120] |
| ICAM- | EVCILLESGGGLVQPGGSLRLSCAASGFTFSNAWMSWVRQAPGKGLEWVAFIWYDGSNKYYADSVKGRF |
| 1 | TISRDNSKNTLYLQMNSLRAEDTAVYYCARYSGWYFDYWGQGTLVTVSSGGGGSGGGGSGGGGSQSVLT |
| | QPPSASGTPGQRVTISCTGSSSXIGAGYDVHWYQQLPGTAPKLLIYDNNNRPSXVPDRFSGSXSGTSAS |
| | LAISGLRSEDEADYYCQSYDSSLSAWLFGGXTKLTVLGEQKLISXXDLSGSXAAHHHHHH-SPRWPIRX |
| | IVSXXTIXXPXFYXVXXXKPXXTXLXRXXAHPXX [SEQ ID NO: 121] |
| IgM | EVQLLESGGGLVQPGGSLRLSCAASGFTFSDYYMSWIRQAPGKGLEWVSAIGSGPYYAHSVRDRFTISR |
| (2) | DNSKNTLYLQMNSLRAEDTAVYYCARGGVEASFDYWGQGTLVTVSSGGGGSGGGGSGGGGSQSVLTQPP |
| | SASGTPGQRVTISCTGSSSNIGAGYDVHWYQQLPGTAPKLLIYGNTNRPSXVPNRFSGSXSGTSASLAI |
| | SGLRSEDEADYYCQSYDNDLSGWVFGGXTKLITVLGEQKLISEEXLSGSAA [SEQ ID NO: 122] |
| MUC- | EVQLLESGGGLVQPGGSLRLSCAASGFTFKNYWMSWVRQAPGKGLEWVSDISGGGGTTYIADSVKGRFT |
| 1 (2) | ISRDNSKNTLYLQMNSLRAEDTAVYYCARIHSGSYYFDYWGQGTLVTVSSGGGGSGGGGSGGGGSQSVL |
| | TQPPSASGTPGQRVTISCTGSSSNIGAGYDVHWYQQLPGTAPKLLIYKNNQRPSGVPDRFSGSKSGTSA |
| | SLAIXGLRSEDEADYYCA [SEQ ID NO: 123] |
| MUC- | EVQLLESGGGLVQPGGSLRLSCAASGFTFKNYWMSWVRQAPGKGLEWVSDISGGGGTTYIADSVKGRFT |
| 1 (4) | ISRDNSKNTLYLQMNSLRAEDTAVYYCARIHSGSYYFDYWGQGTLVTVSSGGGGSVGGGSGGGGSQSVL |
| | TQPPSASGTSGQRVTISCTGSSSNIGAGYDVHWYQQLPGTAPKLLIYKNNQRPSGVPDRFSGSXSGTSA |
| | SLAISGLRSEDEADYYCAAXDDSLNGPVFGGXTKLTVLGDYKDHDGDYXDHDIDXXDXDXKAA |
| | [SEQ ID NO: 124] |
| MUC- | EVQLLESGGGLVQPGGSLRLSCAASGFTFKNYWMSWVRQAPGKGLEWVSDISGGGGTTYIADSVKGRFT |
| 1 (5) | ISRDNSKNTLYLQMNSLRAEDTAVYYCARIHSGSYYFDYWGQGTLVTVSSGGGGSGGGGSGGGGSQSVL |
| | TQPPSASGTPGQRVTISCTGSSSNIGADYDVHWYQQLPGTAPKLLIYKNNQRPSGVPDRFSGSKSGTSA |
| | SLAISGLRSEDXADYYCAVWDDSLNGPXFGGXTKLTVLXDYKXHDGDYKDHDIDXKDDDDKAA |
| | [SEQ ID NO: 125] |
| MUC- | EVQLLESGGGLVQPGGSLRLSCAASGFTFKNYWMSWVRQAPGKGLEWVSDISGGGGITYIADSVKGRFT |
| 1 (6) | ISRDNSRNTLYLQMNSLRAEDTAVYYCARIHSGSYYFDYWGQGTLVTVSSGGGGSGGGGSGGGGSQSVL |
| | TQPPSASGTPGQKVTISCTGSSSNIGAGYDVHWYQQLPGTAPKLLIYKNNQRPSGVPDRFSGSRSGTSA |
| | SLAISGLRSEDEADYYCAAWDDSLNGPVFGGXXKLTVLXDYXDHDGDYKDHDIDYKXXDDKAA |
| | [SEQ ID NO: 126] |
| MCP- | QSVLTQPASASGTPGQRVTISCTGNSSNIGAGYDVHWYQQLPGTAPKLLIYRNNQRPSGVPDRFSGSKS |
| 1 (5) | GTSASLAISGLLSEDEADYYCAAWDYSLNGWVFGGGTKLTVLG [SEQ ID NO: 127] |
| MCP- | QSVLTQPSSASGTPGQRVTISCTGNSSNIGAGYDVHWYQQLPGTAPNLLIYRNNQRPSGVPDRFSGSKS |
| 1 (6) | GTSASLAISGLRSEDEADYYCAAWDDSLNGWVFGGGTKLTVLGQ [SEQ ID NO: 128] |
| Cystatin | EVQLLESGGGLVQPGGSLRLSCAASGFTFSSYAMNWVRQAPGKGLEWVGLISYDGRTTYYADSVKGRST |
| C (1) | ISRDNSKNTLYLQMNSLRAEDTAVYYCATTTGTTLDYWGQGTLVTVSSGGGGSGGGGSGGGGSQSVLTQ |
| | PPSASGTPGQRVTISCTGSSSNIGAGYDVHWYQQLPGTAPKLLIYGNTNRPSXVPDRFSGSXSGTSASL |
| | AISGLRSEDEADYYCAAWDDSLYGWVFGGXTKLTVLGDYXDHDGDYXDHDIDXXDDDDKAA |
| | [SEQ ID NO: 129] |
| Cystatin | EVQLLESGGGLVQPGGSLRLSCAASGFTFSSYAMNWVRQAPGKGLEWVGLISYDGRTTYYADSVKGRST |
| C (4) | ISRDNSKNTLYLQMNSLRAEDTAVYYCATTTGTTLDYWGQGTLVTVSSGGGGSGGGGSGGGGSQSVLTQ |
| | PPSASGTPGQRVTISCTGSSSNIGAGYDVHWYQQLPGTAPKLLIYGNTNRPSXVPDRFSGSXSGTSXSL |
| | AISGLRSXDEADYYCAAWDDSLYGWVFGG [SEQ ID NO: 130] |
| Apo- | EVQLLESGGGLVQPGGSLRLSCAASGFTFSNNGMHWVRQAPGKGLEWVSAISASGGSTYYADSVKGRFT |
| A1 | ISRDNSKNTLYLQMNSLRAEDTAVYYCATHGGSSYDAFDIWGQGTLVTVSSGGGGSGGGGSGGGGSQSV |
| (1) | LTQPPSASGTPGQRVTISCTGSSSNIGAGYVVHWYQQLPGTAPKLLIYG [SEQ ID NO: 131] |
| Apo- | EVQLLESGGGLVQPGGSLRLSCAASGFTFRDYYMSWIRQAPGKGLEWVAVTSYDGSKKYYADSVKGRFT |
| A1 | ISRDNSKNTLYLQMNSLRAEDTAVYYCAKDYADDSIAAPAFDIWGQGTLVTVSSGGGGSGGGGSGGGGS |
| (2) | QSVLTQPPSASGTPGQRVTISCTGSSSXIGAGYDVHWYQQLPGTAPKLLIYGNSNRPS |
| | [SEQ ID NO: 132] |
| Apo- | EVXXLESGGGLVQPGGSLRLSCAASGFTFRDYYMSWIRQAPGKGLEWVAVTSYDGSKKYYADSVKGRFT |
| A1 | ISRDNSKNTLYLQMNSLRAEDTAVYYCAKDYADDSIAAPAFDIWGQGTLVTVSSGGGGSGGGGSGGGGS |
| (3) | QSVLTQPPSASGTPGQRVTISCTGSSSNIGAGYDVHWYQQLPGTAPKLLIYGNSNRPSGVPDRFSGSKS |
| | GTSASLAISGLRSXDEAXYYCQSYDSSLSVVFGGGTKLTVLXXYXDHDGDYKDHDIDYXDDXXXAXAHH |
| | HHHH-SPXXXIRXXXSXXTIHXXXXXXXXDWXXXXXXXXX[SEQ ID NO: 133] |
| Factor | EVQLLESGGGLVQPGGSLRLSCAASGFTFSSYSMNWVRQAPGKGLEWVAVISYDGRFIYYSDSVKGRFT |
| B (2) | ISRDNSKNTLYLQMNSLRAEDTAVYYCARSYGGNLAMDVWGQGTLVTVSSGGGGSGGGGSGGGGSQSVL |
| | TQPPSASGTPGQRVTISCTGSSSNIGAGYDVHWYQQLPGTAPKLLIYDNNKRPSGVPDRFSGSNSGTSA |
| | SLAISGLRSEDEADYYCAAWDDRLNGRVVFGGXTKLTVLGDYXDHDGDYKDHDIDXKDDDXKAA |
| | [SEQ ID NO: 134] |
| C1 | EVQLLESGGGLVQPGGSLRLSCAASGFTFSNAWMSWVRQAPGKGLEWVSAISGSGGSTYYADSVKGRFT |
| inh. | ISRDNSKNTLYLQMNSLRAEDTAVYYCARNRGNWGTYYFDYWGQGTLVTVSSGGGGSGGGGSGGGGSQS |
| (2) | VLTQPPSASGTPGQRVTISCSGSSSNIGSNYVSWYQQLPGTAPKLLIYGSSNRPSGVPDRFSGSXSGTS |
| | ASLAISGLRSEDEADYYCQSYDSSLSDHVVFGGXTKLTVLXDYXDHDGDYKDHDIDXXDDDDXAA |
| | [SEQ ID NO: 135] |
| C1 | EVQLLESGGGLVQPGGSLRLSCAASGFTFSNAWMSWVRQAPGKGLEWVSAISGSGGSTYYADSVKGRFT |
| inh. | ISRDNSKNTLYLQMNSLRAEDTAVYYCARNRGNWGTYYFDYWGQGTLVTVSSGGGGSGGGGSGGGGSQS |
| (3) | VLTQPPSASGTPGQRVTISCSGSSSXIGSNYVSWYQQLPGTAPKLLIYGSSNRPSXVPDRFSGSXSGTS |
| | ASLAISGLRSEDXADYYCQSYDSSLSDHVVFGGXTKLTVLGDYXDHDGDYKDHDXDXXDDXXXAA |
| | [SEQ ID NO: 136] |
| C5 | EVQLLESGGGLVQPGGSLRLSCAASGFTFSSYGMHWVRQAPGKGLEWVSYISSSGSTIYYADSVKGRFT |
| (3) | ISRDNSKNTLYLQMNSLRAEDTAVYYCLTLGGYWGQGTLVTVSSGGGGSGGGGSGGGGSQSVLTQPPSA |
| | SGTPGQRVTISCSGSSSNIGSNTVNWYQQLPGTAPKLLIYSNNQRPSGVPDRFSGSKSGTSASLAISGL |
| | RSXDEADYYCQSYDSSLSGWVFGGXTKLTVLXDYKXHDGDYKDHDIDXKDDDXXAA |
| | [SEQ ID NO: 137] |
| C4 | EVQLLESGGGLVQPGGSLRLSCAASGFTFSSYAMSWVRQAPGKGLEWVSAISGSGGSTYYADSVKGRFT |
| (3) | ISRDNSKNTLYLQMNSLRAEDTAVYYCARGWSTSSFDYWGQGTLVTVSSGGGGSGGGGSGGGGSQSVLT |
| | QPPSASGTPGQRVTISCSGSSSXIGNHYVSWYQQLPGTATKLLIYXDDLLPSXVPDRFSGSXSGTSASL |
| | AIXGLRSEDEADYYCAAWDDRSGQVLFGGXTKLTVLGDYXDHDGDYXDHDIDXXDDDXKAXAHHHHHH- |
| | XXRWPIRPXVSXXTIHXXXFXXXXXXKT [SEQ ID NO: 138] |
| C4 | EVQLLESGGGLVQPGGSLRLSCAASGFTFSSYSMNWVRQAPGKGLEWVSGISGSGGSTYYADSVKGRFT |
| (4) | ISRDNSKNTLYLQMNSLRAEDTAVYYCAKHSGYGFDIWGQGTLVTVSSGGGGSGGGGSGGGGSQSVLTQ |
| | PPSASGTPGQRVTISCSGGASXLGMHFVSWYQQLPGTAPKLLIYYDDLLPSGVPDRFSGXXSGTSASLA |
| | ISGLRSEDEADYYCAAWDDSLNGWVFGGXTKLXVLGDYXDXXGDYKDHDIDXKDXXXXAXAHXHHHH-S |
| | PXWXXRPIVXXITXXXXVXLQRXDWXXPXVXXXXXXXXXXPX [SEQ ID NO: 139] |
| C3 | EVQLLESGGGLVQPGGSLRLSCAASGFTFSSYAMNWVRQAPGKGLEWVANINQDGSTKFYVDSVKGRFT |
| (3) | ISRDNSKNTLYLQMNSLRAEDTAVYYCARDTGGNYLGGYYYYGMDVWGQGTLVTVSSGGGGSGGGGSGG |
| | GGSQSVLTQPPSASGTPGQRVTISCSGSSSNIGSNYVYWYQQLPGTAPKLLIYRNDQRPSXVPDRFSGS |
| | XSGTSASLAISGLRSXDXADYYCSSYAGNNNLVFGGXTKLTVLGDYXDHDGDYKDHDIDYXDXDXXAA |
| | [SEQ ID NO: 140] |
| C3 | EVQLLESGGGLVQPGGSLRLSCAASGFTFSDHYMDWVRQAPGKGLEWVSGISGNGATIDYADSVKGRFT |
| (4) | ISRDNSKNTLYLQMNSLRAEDTAVYYCARPSITAAGSEDAFDLWGQGTLVTVSSGGGGSGGGGSGGGGS |
| | QSVLTQPPSASGTPGQRVTISCSGSSSNIGSNYVYWYQQLPGTAPKLLIYGNSNRPSGVPDRFSGSKSG |
| | TSASLAISGLRSXDGADYYCQSYDSSLSGWVIGGXTKLTVLGXYXDHDGDYKDXDIDYKDDXXKAA |
| | [SEQ ID NO: 141] |
| MYO | EVQLLESGGGLVQPGGSLRLSCAASGFTFSSYAMSWVRQAPGKGLEWVSGISGSGGSTYYADSVKGRFT |
| M2 | ISRDNSKNTLYLQMNSLRAEDTAVYYCARGVVAGSWGQGTLVTVSSGGGGSGGGGSGGGGSQSVLTQPP |
| (1) | SASGTPGQRVTISCSGSSSXIGNNAVNWYQQLPGTAPKLLIYDNNKRPSXVPDRFSGXXSGTSXSLAIX |
| | GLRSEDEADYYCA [SEQ ID NO: 142] |
| MYO | EVQLLESGGGLVQPGGSLRLSCAASGFTFSNEWMAWVRQAPGKGLEWVSSISSSSSYIYYADSVKGRFT |
| M2 | ISRDNSKNTLYLQMNSLRAEDTAVYYCAGTYHDFWSATYWGQGTLVTVSSGGGGSGGGGSGGGGSQSVL |
| (2) | TQPPSASGTPGQRVTISCSGSSSNIGSNTVNWYQQLPGTAPKLLIYGNSNRPSXVPDRFSGSXSGTSAS |
| | LAISGLRSEDXADYYCAAWDDSLNGWVFGGXTKLTVLGD [SEQ ID NO: 143] |
| LUM | EVQLLESGGGLVQPGGSLRLSCAASGFTFSSNYMSWVRQAPGKGLEWVSAISASGTYTYYTDSVNGRFT |
| | ISRDNSKNTLYLQMNSLRAEDTAVYYCARVNTVGLGTPFDNWGQGTLVTVSSGGGGSGGGGSGGGGSQS |
| | VLTQPPSASGTPGQRVTISCSGSSSNIGSNTVNWYQQLPGTAPKLLIYGNRNRPSGVPDRFSGSXSGTS |
| | ASLAISGLRSEDEADYYCAAWDDSLSGWVFGGXTKLTVLXDYXDHDGDYKDHDIDXXXDDXXAA |
| | [SEQ ID NO: 144] |
| DUSP | EVQLLESGGGLVQPGGSLRLSCAASGFTFSSYGFHWVRQAPGKGLEWVAVISYDGSNKYYADSVKGRFT |
| 9 | ISRDNSKNTLYLQMNSLRAEDTAVYYCARGEFGVYWGQGTLVTVSSGGGGSGGGGSGGGGSQSVLTQPP |
| | SASGTPGQRVTISCSGSSSNIGSNYVYWYQQLPGTAPKLLIYGNRNRPSGVPDRFSGSXSGTSASLAIS |
| | GLRSEDEADYYCSSYAGSNNFEVVFGGXTKLTVLGDYXDHDGDYKDHDIDYKDDDXKAA |
| | [SEQ ID NO: 145] |
| CHX1 | EVQLLESGGGLVQPGGSLRLSCAASGFTFSSYAMSWVRQAPGKGLEWVAVISYDGSNKYYADSVKGRFT |
| 0 (3) | ISRDNSKNTLYLQMNSLRAEDTAVYYCARNYGDSINWFDPWGQGTLVTVSSGGGGSGGGGSGGGGSQSV |
| | LTQPPSASGTPGQRVTISCSGSSSXIRSNIVNWYQQLPGTAPKLLIYGNSNRPSXVPDRFSGXXSGTSX |
| | SLAISGLRSEDXADYYCAXWDDSLN [SEQ ID NO: 146] |
| ATP- | EVQLLESGGGLVQPGGSLRLSCAASGFTFSSYAMHWVRQAPGKGLEWVAVISYDGSKTYHADSVFGRFT |
| 5B | ISRDNSKNTLYLQMNSLRAEDTAVYYCARHLRPYYFDYWGQGTLVTVSSGGGGSGGGGSGGGGSQSVLT |
| (1) | QPPSASGTPGQRVTISCSGSSSXIGSNTVNWYQQLPGTAPKLLIYGNSNRPSXVPDRFSGXXSGTSASL |
| | AISGLRSEDXADYYCSAWDDRLRGRVFGG [SEQ ID NO: 147] |
| ATP- | EVQLLESGGGLVQPGGSLRLSCAASGFTFSSYGMHWVRQAPGKGLEWVSLISSASSYIYHADSVKGRFT |
| 5B | ISRDNSKNTLYLQMNSLRAEDTAVYYCARAGRVCTNGVCHTTFDYWGQGTLVTVSSGGGGSGGGGSGGG |
| (2) | GSQSVLTQPPSASGTPGQRVTISCSGDRSNIGSNTVNWYQQLPGTAPKLLIYGNSNRPSGVPXRFSGSX |
| | SGTSXSLAISGLRSEDEADYYCQSYDSSLSAVVFGGXTKLTVLGDYXXHDXXYKDHDIDYXXDXDXAXA |
| | HXHHHH-SPRXXXXPIVSXXXXXXXXXXXXXXLXKXXXXPTXXXXXXXX [SEQ ID NO: 148] |
| Sox1 | EVQLLESGGGLVQPGGSLRLSCAASGFTFSDFWMSWVRQAPGKGLEWVSSISGGGGTAFYVDSVKGRFT |
| 1a | ISRDNSKNTLYLQMNSLRAEDTALYYCARMTDLESGDAFDIWGQGTLVTVSSGGGGSGGGGSGGGGSQS |
| | VLTQPPSASGTPGQRVTISCSGSSSNIGSNYVNWYQQLPGTAPKLLIYNDNVRPSGVPDRFSGSXSGTS |
| | ASLAISGLRSEDXADYYCQXWGTGVFGGXTKLTVLXDYXDHDGDXXDHDIDXKDXDXKAA |
| | [SEQ ID NO: 149] |
| TBC1 | EVQLLESGGGLVQPGGSLRLSCAASGFTFSSYSMSWVRQAPGKGLEWVAVISYDGSNKYYADSVKGRFT |
| D9 | ISRDNSKNTLYLQMNSLRAEDTAVYYCARDRTRGSTALDIWGQGTLVTVSSGGGGSGGGGSGGGGSQSV |
| (1) | LTQPPSASGTPGQRVTISCSGSSSYIGSNYVYWYQQLPGTAPKLLIYRNNQRPXXVPDRFSGXXSGTSA |
| | SLAISGLRSEDEADYYCAAWDDSLSGWVFGGXTKLTVLGD [SEQ ID NO: 150] |
| UPF3 | EVQLLESGGGLVQPGGSLRLSCAASGFTFSDYYMTINIRQAPGKGLEWVSDISWNGSRTHYADSVKGRF |
| B (1) | TISRDNSKNTLYLQMNSLRAEDTAVYYCSSHLVYWGQGTLVTVSSGGGGSGGGGSGGGGSQSVLTQPPS |
| | ASGTPGQRVTISCTGSSSNIGAGYDVHWYQQLPGTAPKLLIYDNNKRPSXVPDRFSGSXSGTSASLAIX |
| | GLRSEXXADYYCQTYDSSLSGSVVFGGXTKLITVLGDYXDHDXDY [SEQ ID NO: 151] |
| UPF3 | EVQLLESGGGLVQPGGSLRLSCAASGFTFSSYAMSWVRQAPGKGLEWVSYISSSSSYANYADSVKGRFT |
| B (2) | ISRDNSKNTLYLQMNSLRAEDTAVYYCARLGVYSGTYLFAFDIWGQGTLVTVSSGGGGSGGGGSGGGGS |
| | QSVLTQPPSASGTPGQRVTISCTGSSSXIGAGYDVHWYQQLPGTAPKLLIYGNSNRPSXVPDRFSGSXS |
| | GTSASLAISGLRSXDEADYYCQSRDSSLSGWVFGGXTKLTVLGD [SEQ ID NO: 152] |
| Apo- | EVQLLESGGGLVQPGGSLRLSCAASGFTFSDYYMSWVRQAPGKGLEWVSGVSWNGSRTHYADSVKGRFT |
| A4 | ISRDNSKNTLYLQMNSLRAEDTAVYYCARVAYDIDAFDMWGQGTLVTVSSGGGGSGGGGSGGGGSQSVL |
| (2) | TQPPSASGTPGQRVTISCSGSFSNIGSNWYWYQQLPGTAPKLLIYENNKRPSGVPDRFSGSXSGTSASL |
| | AISGLRSEDEADYYCAAWDDSLNGPMFGGXTKLTVLXDYKDHDGDYKDHDIDYKDDXXXXAAHHHHHH- |
| | SPRWXIRPXXSXXTIHXXXXLXXXD [SEQ ID NO: 153] |
| Apo- | EVQLLESGGGLVQPGGSLRLSCAASGFTFSSYSMNWVRQAPGKGLEWVSAITGSGNATFYADSVKGRFT |
| A4 | ISRDNSKNTLYLQMNSLRAEDTAVYYCTTGATTRWGQGTLVTVSSGGGGSGGGGSGGGGSQSVLTQPPS |
| (3) | ASGTPGQRVTISCSGSRSNIGSNHVFWYQQLPGTAPKLLIYENNKRPSGVPDRFSGSXSGTSASLAISG |
| | LRSEDXADYYCAAWDDSLSGWVFGG [SEQ ID NO: 154] |
| TBC1 | EVQLLESGGGLVQPGGSLRLSCAASGFTFSNAWMSWVRQAPGKGLEWVSFISSSSSYIYYADSVKGRFT |
| D9 | ISRDNSKNTLYLQMNSLRAEDTAVYYCARVNLVGCTNGVCNGHDYWGQGTLVTVSSGGGGSGGGGSGGG |
| (2) | GSQSVLTQPPSASGTPGQRVTISCSGSSSXIGSNTVNWYQQLPGTAPKLLIYDNNKRP |
| | [SEQ ID NO: 155] |
| TBC1 | EVQLLESGGGLVQPGGSLRLSCAASGFTFGDYAMSWVRQAPGKGLEWVSAISGSGGSTYYADSVKGRFT |
| D9 | ISRDNSKNTLYLQMNSLRAEDTAVYYCAKGRTMASHWGQGTLVTVSSGGGGSGGGGSGGGGSQSVLTQP |
| (3) | PSASGTPGQRVTISCSGSSSXIGNNHVSWYQQLPGTAPKLLIYGNSNRPSXVPDRFSGSXSGTSASLAI |
| | SGLRSEDXADYYCAAWDNSLKVWMFGG [SEQ ID NO: 156] |
| ORP- | EVQLLESGGGLVQPGGSLRLSCAASGFTFSSNYMSWVRQAPGKGLEWVSYISGNSGYTNYADSVKGRFT |
| 3 (1) | ISRDNSKNTLYLQMNSLRAEDTAVYYCARHAGSYDMYGMDVWGQGTLVTVSSGGGGSGGGGSGGGGSQS |
| | VLTQPPSASGTPGQRVTISCSGSTSXIGSHYVYWYQQLPGTAPKLLIYGNSNRPXXVPDRFSGXXSGTS |
| | XSLAISGLRSEDXADYYCQSYDSRLSGWVFGG [SEQ ID NO: 157] |
| ORP- | EVQLLESGGGLVQPGGSLRLSCAASGFTFSSYAMHWVRQAPGKGLEWVAVISYDGSNKYYADSVKGRFT |
| 3 (2) | ISRDNSKNTLYLQMNSLRAEDTAVYYCARKSSLDVWGQGTLVTVSSGGGGSGGGGSGGGGSQSVLTQPP |
| | SASGTPGQRVTISCSGSSSXIGNNYVSWYQQLPGTAPKLLIYDDNKRPSGVPDRFSGSXSDTSASLAIS |
| | GLRSEDEADYYCAAWDDSLXGRVFGGXTKLTVLG [SEQ ID NO: 158] |
| PKB | EVQLLESGGGLVQPGGSLRLSCAASGFTFGSSYMSWVRQAPGKGLEWVSSISSGGSYTYYADSVKGRFT |
| gamma | ISRDNSKNTLYLQMNSLRAEDTAVYYCARYHASWGRYLDYWGQGTLVTVSSGGGGSGGGGSGGGGSDIQ |
| (1) | MTQSPSSLSASVGDRVTITCRASQSISSYLNWYQQKPGKAPKLLIYAASSLQSGVPSRFSGSGSGTDFT |
| | LTISSLQPEDFATYYCQQVSSWLSTFGQGTKLEIKRLGDYKDHDGDYKDHDIDYKDDDDKAAAHHHHH |
| | H* [SEQ ID NO: 159] |
| PKB | EVQLLESGGGLVQPGGSLRLSCAASGFTFSYSGMGWVRQAPGKGLEWVSYISSGSYYTGYADSVKGRFT |
| gamma | ISRDNSKNTLYLQMNSLRAEDTAVYYCARYSGWRHGFDYWGQGTLVTVSSGGGGSGGGGSGGGGSDIQM |
| (2) | TQSPSSLSASVGDRVTITCRASQSISSYLNWYQQKPGKAPKWYAASSLQSGVPSRFSGSGSGTDFTLTI |
| | SSLQPEDFATYYCQQATVSPSTFGQGTKLEIKRLGDYKDHDGDYKDHDIDYKDDDDKAAAHHHHHH* |
| | [SEQ ID NO: 160] |
| BTK | EVQLLESGGGLVQPGGSLRLSCAASGFTFSSYAMSWVRQAPGKGLEWVSAISGSGGSTYYADSVKGRFT |
| (3) | ISRDNSKNTLYLQMNSLRAEDTAVYYCARGYYGMDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQTWYLPTFGQGTKLEIKRLGDYKDHDGDYKDHDIDYKDDDDKAAAHHHHHH* |
| | [SEQ ID NO: 161] |
| BTK | EVQLLESGGGLVQPGGSLRLSCAASGFTFSSYAMSWVRQAPGKGLEWVSAISGSGGSTYYADSVKGRFT |
| (4) | ISRDNSKNTLYLQMNSLRAEDTAVYYCARGYYGLDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQSGAVPPTFGQGTKLEIKRLGDYKDHDGDYKDHDIDYKDDDDKAAAHHHHHH* |
| | [SEQ ID NO: 162] |
| CDK- | EVQLLESGGGLVQPGGSLRLSCAASGFTFSSYAMSWVRQAPGKGLEWVSGISSYYGYTYYADSVKGRFT |
| 2 (1) | ISRDNSKNTLYLQMNSLRAEDTAVYYCARVYGYYMDYWGQGTLVTVSSGGGGSGGGGSGGGGSDIQMTQ |
| | SPSSLSASVGDRVTITCRASQSISSYLNWYQQKPGKAPKLLIYAASSLQSGVPSRFSGSGSGTDFTLTI |
| | SSLQPEDFATYYCQQSSSLYTFGQGTKLEIKRLGDYKDHDGDYKDHDIDYKDDDDKAAAHHHHHH* |
| | [SEQ ID NO: 163] |
| CDK- | EVQLLESGGGLVQPGGSLRLSCAASGFTFGSSYMGWVRQAPGKGLEWVSSIYGSSSYTSYADSVKGRFT |
| 2 (2) | ISRDNSKNTLYLQMNSLRAEDTAVYYCARWYWSWSSFDYWGQGTLVTVSSGGGGSGGGGSGGGGSDIQM |
| | TQSPSSLSASVGDRVTITCRASQSISSYLNWYQQKPGKAPKLLIYAASSLQSGVPSRFSGSGSGTDFTL |
| | TISSLQPEDFATYYCQQGGWPYTFGQGTKLEIKRLGDYKDHDGDYKDHDIDYKDDDDKAAAHHHHHH* |
| | [SEQ ID NO: 164] |
| GM- | EVQLLESGGGLVQPGGSLRLSCAASGFTFSSYGMSWVRQAPGKGLEWVSYIGSGYYYTSYADSVKGRFT |
| CSF | ISRDNSKNTLYLQMNSLRAEDTAVYYCARGGYFDYWGQGTLVTVSSGGGGSGGGGSGGGGSDIQMTQSP |
| (4) | SSLSASVGDRVTITCRASQSISSYLNWYQQKPGKAPKLLIYAASSLQSGVPSRFSGSGSGTDFTLTISS |
| | LQPEDFATYYCQQGYHLPTFGQGTKLEIKRLGDYKDHDGDYKDHDIDYKDDDDKAAAHHHHHH* |
| | [SEQ ID NO: 165] |
| FASN | EVQLLESGGGLVQPGGSLRLSCAASGFTFGYSGMSWVRQAPGKGLEWVSYIGGGYGSTSYADSVKGRFT |
| (1) | ISRDNSKNTLYLQMNSLRAEDTAVYYCARWSWHHGSYTMDYWGQGTLVTVSSGGGGSGGGGSGGGGSDI |
| | QMTQSPSSLSASVGDRVTITCRASQSISSYLNWYQQKPGKAPKLLIYAASSLQSGVPSRFSGSGSGTDF |
| | TLTISSLQPEDFATYYCQQGYSWFLPTFGQGTKLEIKRLGDYKDHDGDYKDHDIDYKDDDDKAAAHHHH |
| | HH* [SEQ ID NO: 166] |
| FASN | EVQLLESGGGLVQPGGSLRLSCAASGFTFSGSSMSWVRQAPGKGLEWVSSIYYGSGYTYYADSVKGRFT |
| (2) | ISRDNSKNTLYLQMNSLRAEDTAVYYCARGTYLDYWGQGTLVTVSSGGGGSGGGGSGGGGSDIQMTQSP |
| | SSLSASVGDRVTITCRASQSISSYLNWYQQKPGKAPKLLIYAASSLQSGVPSRFSGSGSGTDFTLTISS |
| | LQPEDFATYYCQQHTWWSSYLHTFGQGTKLEIKRLGDYKDHDGDYKDHDIDYKDDDDKAAAHHHHHH* |
| | [SEQ ID NO: 167] |
| GAK | EVQLLESGGGLVQPGGSLRLSCAASGFTFGSSYMSWVRQAPGKGLEWVSSISSSSYGTYYADSVKGRFT |
| (1) | ISRDNSKNTLYLQMNSLRAEDTAVYYCARNPGFDYWGQGTLVTVSSGGGGSGGGGSGGGGSDIQMTQSP |
| | SSLSASVGDRVTITCRASQSISSYLNWYQQKPGKAPKLLIYAASSLQSGVPSRFSGSGSGTDFTLTISS |
| | LQPEDFATYYCQQHYWLPTFGQGTKLEIKRLGDYKDHDGDYKDHDIDYKDDDDKAAAHHHHHH* |
| | [SEQ ID NO: 168] |
| GAK | EVQLLESGGGLVQPGGSLRLSCAASGFTFGSSYMSWVRQAPGKGLEWVSGISSSGYGTYYADSVKGRFT |
| (3) | ISRDNSKNTLYLQMNSLRAEDTAVYYCARGYYHYGYAGFYFDYWGQGTLVTVSSGGGGSGGGGSGGGGS |
| | DIQMTQSPSSLSASVGDRVTITCRASQSISSYLNWYQQKPGKAPKLLIYAASSLQSGVPSRFSGSGSGT |
| | DFTLTISSLQPEDFATYYCQQSAYLFTFGQGTKLEIKRLGDYKDHDGDYKDHDIDYKDDDDKAAAHHHH |
| | HH* [SEQ ID NO: 169] |
| HAD | EVQLLESGGGLVQPGGSLRLSCAASGFTFSSYYMSWVRQAPGKGLEWVSSISSGGYGTGYADSVKGRFT |
| H2 | ISRDNSKNTLYLQMNSLRAEDTAVYYCARSYGWGPLDYWGQGTLVTVSSGGGGSGGGGSGGGGSDIQMT |
| (1) | QSPSSLSASVGDRVTITCRASQSISSYLNWYQQKPGKAPKLLIYAASSLQSGVPSRFSGSGSGTDFTLT |
| | ISSLQPEDFATYYCQQGYNWPYTFGQGTKLEIKRLGDYKDHDGDYKDHDIDYKDDDDKAAAHHHHHH* |
| | SEQ ID NO: 170] |
| HAD | EVQLLESGGGLVQPGGSLRLSCAASGFTFGSSYMSWVRQAPGKGLEWVSSISSYGYYTGYADSVKGRFT |
| H2 | ISRDNSKNTLYLQMNSLRAEDTAVYYCARSYGSWYFDYWGQGTLVTVSSGGGGSGGGGSGGGGSDIQMT |
| (3) | QSPSSLSASVGDRVTITCRASQSISSYLNWYQQKPGKAPKLLIYAASSLQSGVPSRFSGSGSGTDFTLT |
| | ISSLCIPEDFATYYCQQGFVGPSTFGQGTKLEIKRLGDYKDHDGDYKDHDIDYKDDDDKAAAHHHHHH* |
| | [SEQ ID NO: 171] |
| IL-6 | EVQLLESGGGLVQPGGSLRLSCAASGFTFGYSSMGWVRQAPGKGLEWVSGISSYGYGTYYADSVKGRFT |
| (7) | ISRDNSKNTLYLQMNSLRAEDTAVYYCARYHSGWGMDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGSGTDFTLT |
| | ISSLQPEDFATYYCQQSFAYLHTFGQGTKLEIKRLGDYKDHDGDYKDHDIDYKDDDDKAAAHHHHHH* |
| | [SEQ ID NO: 172] |
| Keratin | EVQLLESGGGLVQPGGSLRLSCAASGFTFGYYYMYWVRQAPGKGLEWVSSIGSSGSSTSYADSVKGRFT |
| 19 | ISRDNSKNTLYLQMNSLRAEDTAVYYCARGHAFFDYWGQGTLVWSSGGGGSGGGGSGGGGSDIQMTQSP |
| (1) | SSLSASVGDRVTITCRASQSISSYLNWYQQKPGKAPKLLIYAASSLQSGVPSRFSGSGSGTDFTLTISS |
| | LQPEDFATYYCQQVWYWPPTFGQGTKLEXKRLXDYKDHDGDYKXHDIDYKDDDDKAA |
| | [SEQ ID NO: 173] |
| Keratin | EVQLLESGGGLVQPGGSLRLSCAASGFTFYGSSMSWVRQAPGKGLEWVSYIGSDSSYTSYADSVKGRFT |
| 19 | ISRDNSKNTLYLQMNSLRAEDTAVYYCARSYWSVFDYWGQGTLVTVSSGGGGSGGGGSGGGGSDIQMTQ |
| (2) | SPSSLSASVGDRVTITCRASQSISSYLNWYQQKPGKAPKLLIYAASSLQSGVPSRFSGSGSGTDFTLTI |
| | SSLQPEDFATYYCQQSYWSWLPTFGQGXKLEIKRLXDYKDHDGDYKDHDIDYKDDDDKAA |
| | [SEQ ID NO: 174] |
| KSYK | EVQLLESGGGLVQPGGSLRLSCAASGFTFSSYAMSWVRQAPGKGLEWVSGISSGYYYTYYADSVKGRFT |
| (1) | ISRDNSKNTLYLQMNSLRAEDTAVYYCARVGWYWNGLDYWGQGTLVTVSSGGGGSGGGGSGGGGSDIQM |
| | TQSPSSLSASVGDRVTITCRASQSISSYLNWYQQKPGKAPKLLIYAASSLQSGVPSRFSGSGSGTDFTL |
| | TISSLCIPEDFATYYCQQSSVLPTFGQGTKLEIKRLGDYKDHDGDYKDHDIDYKDDDDKAAAHHHHHH* |
| | [SEQ ID NO: 175] |
| KSYK | EVQLLESGGGLVQPGGSLRLSCAASGFTFSSYAMSWVRQAPGKGLEWVSAISGSGGSTYYADSVKGRFT |
| (2) | ISRDNSKNTLYLQMNSLRAEDTAVYYCARGYYGLDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLCIPEDFATYYCQQSGAVPPTFGQGTKLEIKRLGDYKDHDGDYKDHDIDYKDDDDKAAAHHHHHH* |
| | [SEQ ID NO: 176] |
| MAT | EVQLLESGGGLVQPGGSLRLSCAASGFTFYGYSMYWVRQAPGKGLEWVSYIGSYSGSTSYADSVKGRFT |
| K (2) | ISRDNSKNTLYLQMNSLRAEDTAVYYCARYYHYYHTWLDYWGQGTLVTVSSGGGGSGGGGSGGGGSDIQ |
| | MTQSPSSLSASVGDRVTITCRASQSISSYLNWYQQKPGKAPKLLIYAASSLQSGVPSRFSGSGSGTDFI |
| | LTISSLQPEDFATYYCQQGFYPFTFGQGTKLEIKRLGDYKDHDGDYKDHDIDYKDDDDKAAAHHHHHH* |
| | [SEQ ID NO: 177] |
| MAT | EVQLLESGGGLVQPGGSLRLSCAASGFTFSSYAMSWVRQAPGKGLEWVSAISGSGGSTYYADSVKGRFT |
| K (3) | ISRDNSKNTLYLQMNSLRAEDTAVYYCARYPFYDYSVVGGRWPSYGIDYWGQGTLVTVSSGGGGSGGGG |
| | SGGGGSDIQMTQSPSSLSASVGDRVTITCRASQSISSYLNWYQQKPGKAPKLLIYAASSLQSGVPSRFS |
| | GSGSGTDFTLTISSLQPEDFATYYCQQWNSGVWLHTFGQGTKLEIKRLGDYKDHDGDYKDHDIDYKDDD |
| | DKAAAHHHHHH* [SEQ ID NO: 178] |
| MAP | EVQLLESGGGLVQPGGSLRLSCAASGFTFSSSYMYWVRQAPGKGLEWVSSISGGGYGTYYADSVKGRFT |
| K1 | ISRDNSKNTLYLQMNSLRAEDTAVYYCARAYPYVGSGIDYWGQGTLVTVSSGGGGSGGGGSGGGGSDIQ |
| (1) | MTQSPSSLSASVGDRVTITCRASQSISSYLNWYQQKPGKAPKLLIYAASSLQSGVPSRFSGSGSGTDFI |
| | LTISSLQPEDFATYYCQQGYSLPTFGQGTKLEIKRLGDYKDHDGDYKDHDIDYKDDDDKAAAHHHHHH* |
| | [SEQ ID NO: 179] |
| MAP | EVQLLESGGGLVQPGGSLRLSCAASGFTFYYSYMGWVRQAPGKGLEWVSSISGSSYGTSYADSVKGRFT |
| K1 | ISRDNSKNTLYLQMNSLRAEDTAVYYCARGTGSVIDYWGQGTLVTVSSGGGGSGGGGSGGGGSDIQMTQ |
| (2) | SPSSLSASVGDRVTITCRASQSISSYLNWYQQKPGKAPKLLIYAASSLQSGVPSRFSGSGSGTDFTLTI |
| | SSLQPEDFATYYCQQSYSTPYTFGQGTKLEIKRLGDYKDHDGDYKDHDIDYKDDDDKAAAHHHHHH* |
| | [SEQ ID NO: 180] |
| MAP | EVQLLESGGGLVQPGGSLRLSCAASGFTFYGSSMSWVRQAPGKGLEWVSGISGYGYYTSYADSVKGRFT |
| K8 | ISRDNSKNTLYLQMNSLRAEDTAVYYCARYSASGFYFDYWGQGTLVTVSSGGGGSGGGGSGGGGSDIQM |
| (1) | TQSPSSLSASVGDRVTITCRASQSISSYLNWYQQKPGKAPKLLIYAASSLQSGVPSRFSGSGSGTDFTL |
| | TISSLQPEDFATYYCQQSYVYPLTFGQGTKLEIKRLGDYKDHDGDYKDHDIDYKDDDDKAAAHHHHHH* |
| | [SEQ ID NO: 181] |
| MAP | EVQLLESGGGLVQPGGSLRLSCAASGFTFSSYSMGWVRQAPGKGLEWVSGISSYGYYTYYADSVKGRFT |
| K8 | ISRDNSKNTLYLQMNSLRAEDTAVYYCARHYTTGYYIDYWGQGTLVTVSSGGGGSGGGGSGGGGSDIQM |
| (2) | TQSPSSLSASVGDRVTITCRASQSISSYLNWYQQKPGKAPKLLIYAASSLQSGVPSRFSGSGSGTDFTL |
| | TISSLQPEDFATYYCQQGFNVPYTFGQGTKLEIKRLGDYKDHDGDYKDHDIDYKDDDDKAAAHHHHHH* |
| | [SEQ ID NO: 182] |
| MAP | EVQLLESGGGLVQPGGSLRLSCAASGFTFSGSGMYWVRQAPGKGLEWVSYISGSGSYTDYADSVKGRFT |
| K8 | ISRDNSKNTLYLQMNSLRAEDTAVYYCARSGSFDYWGQGTLVTVSSGGGGSGGGGSGGGGSDIQMTQSP |
| (3) | SSLSASVGDRVTITCRASQSISSYLNWYQQKPGKAPKLLIYAASSLQSGVPSRFSGSGSGTDFTLTISS |
| | LQPEDFATYYCQQVSSSLYTFGQGTKLEIKRLGDYKDHDGDYKDHDIDYKDDDDKAAAHHHHHH* |
| | [SEQ ID NO: 183] |
| Osteo- | EVQLLESGGGLVQPGGSLRLSCAASGFTFSSYAMSWVRQAPGKGLEWVSAISGSGGSTYYADSVKGRFT |
| pontin | ISRDNSKNTLYLQMNSLRAEDTAVYYCARAYSWFDYWGQGTLVTVSSGGGGSGGGGSGGGGSDIQMTQS |
| (1) | PSSLSASVGDRVTITCRASQSISSYLNWYQQKPGKAPKLLIYAASSLQSGVPSRFSGSGSGTDFTLTIS |
| | SLQPEDFATYYCQQVAGYYHYPFTFGQGTKLEIKRLGDYKDHDGDYKDHDIDYKDDDDKAAAHHHHHH* |
| | [SEQ ID NO: 184] |
| Osteo- | EVQLLESGGGLVQPGGSLRLSCAASGFTFSSYAMSWVRQAPGKGLEWVSAISGSGGSTYYADSVKGRFT |
| pontin | ISRDNSKNTLYLQMNSLRAEDTAVYYCARYHYNYYMDYWGQGTLVTVSSGGGGSGGGGSGGGGSDIQMT |
| (2) | QSPSSLSASVGDRVTITCRASQSISSYLNWYQQKPGKAPKLLIYAASSLQSGVPSRFSGSGSGTDFTLT |
| | ISSLQPEDFATYYCQQYSYLLTFGQGTKLEIKRLGDYKDHDGDYKDHDIDYKDDDDKAAAHHHHHH* |
| | [SEQ ID NO: 185] |
| P85A | EVQLLESGGGLVQPGGSLRLSCAASGFTFYSSSMSWVRQAPGKGLEWVSGISSSYSYTYYADSVKGRFT |
| (1) | ISRDNSKNTLYLQMNSLRAEDTAVYYCARYSSYGSFDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGSGTDFTLT |
| | ISSLQPEDFATYYCQQSSAFPSTFGQGTKLEIKRLGDYKDHDGDYKDHDIDYKDDDDKAAAHHHHHH* |
| | [SEQ ID NO: 186] |
| P85A | EVQLLESGGGLVQPGGSLRLSCAASGFTFSSYSMGWVRQAPGKGLEWVSYIGYSSGSTYYADSVKGRFT |
| (2) | ISRDNSKNTLYLQMNSLRAEDTAVYYCARDRYSYFDYWGQGTLVTVSSGGGGSGGGGSGGGGSDIQMTQ |
| | SPSSLSASVGDRVTITCRASQSISSYLNWYQQKPGKAPKWYAASSLQSGVPSRFSGSGSGTDFTLTISS |
| | LQPEDFATYYCQQWSYGPLTFGQGTKLEIKRLGDYKDHDGDYKDHDIDYKDDDDKAAAHHHHHH* |
| | [SEQ ID NO: 187] |
| PTK6 | EVQLLESGGGLVQPGGSLRLSCAASGFTFSSYAMSWVRQAPGKGLEWVSAISGSGGSTYYADSVKGRFT |
| | ISRDNSKNTLYLQMNSLRAEDTAVYYCARGGHGLDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQGSDVPFTFGQGTKLEIKRLGDYKDHDGDYKDHDIDYKDDDDKAAAHHHHHH* |
| | [SEQ ID NO: 188] |
| PTPN | EVQLLESGGGLVQPGGSLRLSCAASGFTFGYSSMSWVRQAPGKGLEWVSSISYSGSGTGYADSVKGRFT |
| 1 (1) | ISRDNSKNTLYLQMNSLRAEDTAVYYCARGWYPHPGHWYIDYWGQGTLVTVSSGGGGSGGGGSGGGGSD |
| | IQMTQSPSSLSASVGDRVTITCRASQSISSYLNWYQQKPGKAPKLLIYAASSLQSGVPSRFSGSGSGTD |
| | FTLTISSLQPEDFATYYCQQHFSLPTFGQGTKLEIKRLGDYKDHDGDYKDHDIDYKDDDDKAAAHHHHH |
| | H* [SEQ ID NO: 189] |
| PTPN | EVQLLESGGGLVQPGGSLRLSCAASGFTFSYYSMSWVRQAPGKGLEWVSSISSSGGGTSYADSVKGRFT |
| 1 (3) | ISRDNSKNTLYLQMNSLRAEDTAVYYCARWFSSAFDYWGQGTLVTVSSGGGGSGGGGSGGGGSDIQMTQ |
| | SPSSLSASVGDRVTITCRASQSISSYLNWYQQKPGKAPKLLIYAASSLQSGVPSRFSGSGSGTDFTLTI |
| | SSLQPEDFATYYCQQGVPPYTFGQGTKLEIKRLGDYKDHDGDYKDHDIDYKDDDDKAAAHHHHHH* |
| | [SEQ ID NO: 190] |
| RPS6 | EVQLLESGGGLVQPGGSLRLSCAASGFTFSSYAMSWVRQAPGKGLEWVSAISGSGGSTYYADSVKGRFT |
| KA2 | ISRDNSKNTLYLQMNSLRAEDTAVYYCARRYSGYFDYWGQGTLVTVSSGGGGSGGGGSGGGGSDIQMTQ |
| (1) | SPSSLSASVGDRVTITCRASQSISSYLNWYQQKPGKAPKLLIYAASSLQSGVPSRFSGSGSGTDFTLTI |
| | SSLQPEDFATYYCQQNWWGLPTFGQGTKLEIKRLGDYKDHDGDYKDHDIDYKDDDDKAAAHHHHHH* |
| | [SEQ ID NO: 191] |
| RPS6 | EVQLLESGGGLVQPGGSLRLSCAASGFTFYGSYMYWVRQAPGKGLEWVSGISPYSSSTYYADSVKGRFT |
| KA2 | ISRDNSKNTLYLQMNSLRAEDTAVYYCARGFPFIDYWGQGTLVTVSSGGGGSGGGGSGGGGSDIQMTQS |
| (2) | PSSLSASVGDRVTITCRASQSISSYLNWYQQKPGKAPKLLIYAASSLQSGVPSRFSGSGSGTDFTLTIS |
| | SLQPEDFATYYCQQNGVGLLTFGQGTKLEIKRLGDYKDHDGDYKDHDIDYKDDDDKAAAHHHHHH* |
| | [SEQ ID NO: 192] |
| STAP | EVQLLESGGGLVQPGGSLRLSCAASGFTFYGGSMYWVRQAPGKGLEWVSSISSGGSYTYYADSVKGRFT |
| 2 (1) | ISRDNSKNTLYLQMNSLRAEDTAVYYCARVGYDTMDYWGQGTLVTVSSGGGGSGGGGSGGGGSDIQMTQ |
| | SPSSLSASVGDRVTITCRASQSISSYLNWYQQKPGKAPKLLIYAASSLQSGVPSRFSGSGSGTDFTLTI |
| | SSLQPEDFATYYCQQRWYWWYLFTFGQGTKLEIKRLGDYKDHDGDYKDHDIDYKDDDDKAAAHHHHHH* |
| | [SEQ ID NO: 193] |
| STAP | EVQLLESGGGLVQPGGSLRLSCAASGFTFYYGGMSWVRQAPGKGLEWVSSIYYSSGSTSYADSVKGRFT |
| 2 (4) | ISRDNSKNTLYLQMNSLRAEDTAVYYCARHGWDDNGFDYWGQGTLVTVSSGGGGSGGGGSGGGGSDIQM |
| | TQSPSSLSASVGDRVTITCRASQSISSYLNWYQQKPGKAPKLLIYAASSLQSGVPSRFSGSGSGTDFTL |
| | TISSLQPEDFATYYCQQVYSYLFTFGQGTKLEIKRLGDYKDHDGDYKDHDIDYKDDDDKAAAHHHHHH* |
| | [SEQ ID NO: 194] |
| STAT | EVQLLESGGGLVQPGGSLRLSCAASGFTFGYYGMSWVRQAPGKGLEWVSGIYGSYYSTYYADSVKGRFT |
| 1 (1) | ISRDNSKNTLYLQMNSLRAEDTAVYYCARHSWDYYFDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGSGTDFTLT |
| | ISSLQPEDFATYYCQQYHYGYGSPYTFGQGTKLEIKRLGDYKDHDGDYKDHDIDYKDDDDKAAAHHHHH |
| | H* [SEQ ID NO: 195] |
| STAT | EVQLLESGGGLVQPGGSLRLSCAASGFTFSYSSMYWVRQAPGKGLEWVSSISSYGHSTYYADSVKGRFT |
| 1 (2) | ISRDNSKNTLYLQMNSLRAEDTAVYYCARSWGYYHYLDYWGQGTLVTVSSGGGGSGGGGSGGGGSDIQM |
| | TQSPSSLSASVGDRVTITCRASQSISSYLNWYQQKPGKAPKLLIYAASSLQSGVPSRFSGSGSGTDFTL |
| | TISSLQPEDFATYYCQQTWHPYPSTFGQGTKLEIKRLGDYKDHDGDYKDHDIDYKDDDDKAAAHHHHH |
| | H* [SEQ ID NO: 196] |
| TENS | EVQLLESGGGLVQPGGSLRLSCAASGFTFSGYSMSWVRQAPGKGLEWVSSISSGYYSTYYADSVKGRFT |
| 4 | ISRDNSKNTLYLQMNSLRAEDTAVYYCARVYWGSPWFNPAMDYWGQGTLVTVSSGGGGSGGGGSGGGGS |
| | DIQMTQSPSSLSASVGDRVTITCRASQSISSYLNWYQQKPGKAPKLLIYAASSLQSGVPSRFSGSGSGT |
| | DFTLTISSLQPEDFATYYCQQWYYWVPPTFGQGTKLEIKRLGDYKDHDGDYKDHDIDYKDDDDKAAAHH |
| | HHHH* [SEQ ID NO: 197] |
| TNFR | EVQLLESGGGLVQPGGSLRLSCAASGFTFSSFAMHWVRQAPGKGLEWVSAISGSGGSTYYADSVKGRFT |
| SF14 | ISRDNSKNTLYLQMNSLRAEDTAVYYCASRSTLYYYYGMDVWGQGTLVTVSSGGGGSGGGGSGGGGSQS |
| (1) | VLTQPPSASGTPGQRVTISCSGSTSNIGNSHVYWYQQLPGTAPKLLIYGNSNRPSXVPDRFSGSXSGTS |
| | ASLAISGLRSEDEADYYCSSXAGSNNLVFGGXTKLTVLGEQKLISXXDLSGSAA |
| | [SEQ ID NO: 198] |
| TNFR | EVQLLESGGGLVQPGGSLRLSCAASGFTFSSYAMSWVRQAPGKGLEWVSAISGSGGSTYYADSVKGRFT |
| SF14 | ISRDNSKNTLYLQMNSLRAEDTAVYYCARSPYYGMDVWGQGTLVTVSSGGGGSGGGGSGGGGSQSVLTQ |
| (2) | PPSASGTPGQRVTISCTGSSSNIGAGYDVHWYQQLPGTAPKLLIYRNDQRPSGVPDRFSGSKSGTSASL |
| | AISGLRSEDEADYYCSSYGGRDNVVFGGXTKLTVLXEQKLISXXXLSGSAA |
| | [SEQ ID NO: 199] |
| TNFR | EVQLLESGGGLVQPGGSLRLSCAASGFTFSDYYMSWIRQAPGKGLEWVSGLSGSAGRTHYADSVRGRFT |
| SF3 | ISRDNSKNTLYLQMNSLRAEDTAMYYCASSLFDYWGQGTLVTVSSGGGGSGGGGSGGGGSQSVLTQPPS |
| (1) | ASGTPGQRVTISCSGSSSNIGSNYVYWYQQLPGTAPKLLIYSNNQRPSGVPDRFSGSKSGTSASLAISG |
| | LRSEDXADYYCAAWDDSLNAVVFGGXTKLTVLGEQKLISEXDLSGSAA [SEQ ID NO: 200] |
| TNFR | EVQLLESGGGLVQPGGSLRLSCAASGFTFSDYYMNWVRQAPGKGLEWVSGINWNSDDIDYVDSVKGRFT |
| SF3 | ISRDNSKNTLYLQMNSLRAEDTAMYYCAIDSRYSSGWSFEYWGQGTLVTVSSGGGGSGGGGSGGGGSQS |
| (2) | VLTQPPSASGTPGQRVTISCSGSTSNIGNSHVYWYQQLPGTAPKLLIYSNNQRPSGVPDRFSGSKSGTS |
| | ASLAISGLRSEDEADYYCQSYDSSLSGVVFGGXTKLTVLGEQKLISXXXLSGSAA |
| | [SEQ ID NO: 201] |
| UBC9 | EVQLLESGGGLVQPGGSLRLSCAASGFTFGYGSMSWVRQAPGKGLEWVSSISYYGSSTGYADSVKGRFT |
| (1) | ISRDNSKNTLYLQMNSLRAEDTAVYYCARGVVWLDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQGPYPPTFGQGTKLEIKRLGDYKDHDGDYKDHDIDYKDDDDKAAAHHHHHH* |
| | [SEQ ID NO: 202] |
| UBC9 | EVQLLESGGGLVQPGGSLRLSCAASGFTFYGSSMSWVRQAPGKGLEWVSGISYSSSYTSYADSVKGRFT |
| (2) | ISRDNSKNTLYLQMNSLRAEDTAVYYCARATSYWFTYFGVIDYWGQGTLVTVSSGGGGSGGGGSGGGGS |
| | DIQMTQSPSSLSASVGDRVTITCRASQSISSYLNWYQQKPGKAPKLLIYAASSLQSGVPSRFSGSGSGT |
| | DFTLTISSLQPEDFATYYCQQSWSPHTFGQGTKLEIKRLGDYKDHDGDYKDHDIDYKDDDDKAAAHHHH |
| | HH* [SEQ ID NO: 203] |
| UBE2 | EVQLLESGGGLVQPGGSLRLSCAASGFTFGYYSMSWVRQAPGKGLEWVSAISGSGGSTYYADSVKGRFT |
| C (1) | ISRDNSKNTLYLQMNSLRAEDTAVYYCARGGFWYGGYMDYWGQGTLVTVSSGGGGSGGGGSGGGGSDIQ |
| | MTQSPSSLSASVGDRVTITCRASQSISSYLNWYQQKPGKAPKLLIYAASSLQSGVPSRFSGSGSGTDFT |
| | LTISSLQPEDFATYYCQQSSGPHPFTFGQGTKLEIKRLGDYKDHDGDYKDHDIDYKDDDDKAAAHHHHH |
| | H* [SEQ ID NO: 204] |
| UBE2 | EVQLLESGGGLVQPGGSLRLSCAASGFTFSSYSMSWVRQAPGKGLEWVSSISGYSYGTYYADSVKGRFT |
| C (2) | ISRDNSKNTLYLQMNSLRAEDTAVYYCARWHHPYYFDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGSGTDFTLT |
| | ISSLQPEDFATYYCQQGSVLSTFGQGTKLEIKRLGDYKDHDGDYKDHDIDYKDDDDKAAAHHHHHH* |
| | [SEQ ID NO: 205] |
| UCHL | EVQLLESGGGLVQPGGSLRLSCAASGFTFSSYAMSWVRQAPGKGLEWVSAISGSGGSTYYADSVKGRFT |
| 5 | ISRDNSKNTLYLQMNSLRAEDTAVYYCARGWSLDYWGQGTLVTVSSGGGGSGGGGSGGGGSDIQMTQSP |
| | SSLSASVGDRVTITCRASQSISSYLNWYQQKPGKAPKLLIYAASSLQSGVPSRFSGSGSGTDFTLTISS |
| | LQPEDFATYYCQQVHFLPTFGQGTKLEIKRLGDYKDHDGDYKDHDIDYKDDDDKAAAHHHHHH* |
| | [SEQ ID NO: 206] |
| Her2/ | EVQLVESGGGLVQPGGSLRLSCAASGFNIKDTYIHWVRQAPGKGLEWVARIYPTNGYTRYADSVKGRFT |
| Erb | ISADTSKNTAYLQMNSLRAEDTAVYYCSRWGGDGFYAMDYWGQGTLVTVSSGGGGSGGGGSGGGGSDIQ |
| B2 (4) | MTQSPSSLSASVGDRVTITCRASQDVNTAVAWYQQKPGKAPKLLIYSASFLYSGVPSRFSGSRSGTDFT |
| | LTISSLQPEDFATYYCQQHYTTPPTFGQGTKVEIKRLGDYKDHDGDYKDHDIDYKDDDDKAAAHHHHH |
| | H* [SEQ ID NO: 207] |
| EGFR | QVQLKQSGPGLVQPSQSLSITCTVSGFSLTNYGVHWVRQSPGKGLEWLGVIWSGGNTDYNTPFTSRLSI |
| | NKDNSKSQVFFKMNSLQSNDTAIYYCARALTYYDYEFAYWGQGTLVTVSSGGGGSGGGGSGGGGSDILL |
| | TQSPVILSVSPGERVSFSCRASQSIGTNIHWYQQRTNGSPRLLIKYASESISGIPSRFSGSGSGTDFTL |
| | SINSVESEDIADYYCQQNNNWPTTFGAGTKLELKLGDYKDHDGDYKDHDIDYKDDDDKAAAHHHHHH* |
| | [SEQ ID NO: 208] |
| CHP1 | EVQLLESGGGLVQPGGSLRLSCAASGFTFYYYGMSWVRQAPGKGLEWVSGIGYGYGTYADSVKGRFTIS |
| (1) | RDNSKNTLYLQMNSLRAEDTAVYYCARDSYSSPYYSLDYWGQGTLVTVSSGGGGSGGGGSGGGGSDIQM |
| | TQSPSSLSASVGDRVTITCRASQSISSYLNWYQQKPGKAPKLLIYAASSLQSGVPSRFSGSGSGTDFTL |
| | TISSLQPEDFATYYCQQSYSTPYTFGQGTKLEIKRLGDYKDHDGDYKDHDIDYKDDDDKAAAHHHHHH* |
| | [SEQ ID NO: 209] |
| CHP1 | EVQLLESGGGLVQPGGSLRLSCAASGFTFYSYSMGWVRQAPGKGLEWVSSIGGSGYYTYYADSVKGRFT |
| (2) | ISRDNSKNTLYLQMNSLRAEDTAVYYCARNYNYYYGSYIDYWGQGTLVTVSSGGGGSGGGGSGGGGSDI |
| | QMTQSPSSLSASVGDRVTITCRASQSISSYLNWYQQKPGKAPKLLIYAASSLQSGVPSRFSGSGSGTDF |
| | TLTISSLQPEDFATYYCQQSFYPHTFGQGTKLEIKRLGDYKDHDGDYKDHDIDYKDDDDKAAAHHHHH |
| | H* [SEQ ID NO: 210] |
| AGA | EVQLLESGGGLVQPGGSLRLSCAASGFTFGSYSMHWVRQAPGKGLEWVSSISSYSYSTYYADSVKGRFT |
| P-2 | ISRDNSKNTLYLQMNSLRAEDTAVYYCARGGAYYTNPFDYWGQGTLVTVSSGGGGSGGGGSGGGGSDIQ |
| (1) | MTQSPSSLSASVGDRVTITCRASQSISSYLNWYQQKPGKAPKLLIYAASSLQSGVPSRFSGSGSGTDFT |
| | LTISSLQPEDFATYYCQQPGFYSLPTFGQGTKLEIKRLGDYKDHDGDYKDHDIDYKDDDDKAAAHHHHH |
| | H* [SEQ ID NO: 211] |
| AGA | EVQLLESGGGLVQPGGSLRLSCAASGFTFGGYSMYWVRQAPGKGLEWVSYISSGSSSTYYADSVKGRFT |
| P-2 | ISRDNSKNTLYLQMNSLRAEDTAVYYCARGGGWYDYDFFDYWGQGTLVTVSSGGGGSGGGGSGGGGSDI |
| (3) | QMTQSPSSLSASVGDRVTITCRASQSISSYLNWYQQKPGKAPKLLIYAASSLQSGVPSRFSGSGSGTDF |
| | TLTISSLQPEDFATYYCQQSYSTPYTFGQGTKLEIKRLGDYKDHDGDYKDHDIDYKDDDDKAAAHHHHH |
| | H* [SEQ ID NO: 212] |
| MAP | EVQLLESGGGLVQPGGSLRLSCAASGFTFSYGDMSWVRQAPGKGLEWVSGISSGGSSTYYADSVKGRFT |
| K9 | ISRDNSKNTLYLQMNSLRAEDTAVYYCARGYGYAWYFDYWGQGTLVTVSSGGGGSGGGGSGGGGSDIQM |
| (1) | TQSPSSLSASVGDRVTITCRASQSISSYLNWYQQKPGKAPKLLIYAASSLQSGVPSRFSGSGSGTDFTL |
| | TISSLQPEDFATYYCQQWWHPYTFGQGTKLEIKRLGDYKDHDGDYKDHDIDYKDDDDKAAAHHHHHH* |
| | [SEQ ID NO: 213] |
| MAP | EVQLLESGGGLVQPGGSLRLSCAASGFTFGYGGMSWVRQAPGKGLEWVSSIYGSSSSTYYADSVKGRFT |
| K9 | ISRDNSKNTLYLQMNSLRAEDTAVYYCARHWRSVYFDYWGQGTLVTVSSGGGGSGGGGSGGGGSDIQMT |
| (2) | QSPSSLSASVGDRVTITCRASQSISSYLNWYQQKPGKAPKLLIYAASSLQSGVPSRFSGSGSGTDFTLT |
| | ISSLQPEDFATYYCQQGWGSPLTFGQGTKLEIKRLGDYKDHDGDYKDHDIDYKDDDDKAAAHHHHHH* |
| | [SEQ ID NO: 214] |
| MAP | EVQLLESGGGLVQPGGSLRLSCAASGFTFSGSYMSWVRQAPGKGLEWVSSIYGYSSYTYYADSVKGRFT |
| K9 | ISRDNSKNTLYLQMNSLRAEDTAVYYCARGSYLDYWGQGTLVTVSSGGGGSGGGGSGGGGSDIQMTQSP |
| (5) | SSLSASVGDRVTITCRASQSISSYLNWYQQKPGKAPKLLIYAASSLQSGVPSRFSGSGSGTDFTLTISS |
| | LQPEDFATYYCQQYWYPYTFGQGTKLEIKRLGDYKDHDGDYKDHDIDYKDDDDKAAAHHHHHH* |
| | [SEQ ID NO: 215] |
| PAK- | EVQLLESGGGLVQPGGSLRLSCAASGFTFSGYSMSWVRQAPGKGLEWVSSISSSYSSTYYADSVKGRFT |
| 7 (1) | ISRDNSKNTLYLQMNSLRAEDTAVYYCARGSFGFDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQYYYGVLXTFGQGTKLEIKRLGDYKDHDGDYKDHDIDYKDDDDKAAAHHHHHH* |
| | [SEQ ID NO: 216] |
| PAK- | EVQLLESGGGLVQPGGSLRLSCAASGFTFYYSSMSWVRQAPGKGLEWVSGISGGYSSTYYADSVKGRFT |
| 7 (2) | ISRDNSKNTLYLQMNSLRAEDTAVYYCARGFGVMDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQSYSTPYTFGQGTKLEIKRLGDYKDHDGDYKDHDIDYKDDDDKAAAHHHHHH* |
| | [SEQ ID NO: 217] |
| GEM | EVQLLESGGGLVQPGGSLRLSCAASGFTFYYSYMYWVRQAPGKGLEWVSAISGSGGSTYYADSVKGRFT |
| (1) | ISRDNSKNTLYLQMNSLRAEDTAVYYCARFYYYGFNGSFDYWGQGTLVTVSSGGGGSGGGGSGGGGSDI |
| | QMTQSPSSLSASVGDRVTITCRASQSISSYLNWYQQKPGKAPKLLIYAASSLQSGVPSRFSGSGSGTDF |
| | TLTISSLQPEDFATYYCQQSYSTPYTFGQGTKLEIKRLGDYKDHDGDYKDHDIDYKDDDDKAAAHHHHH |
| | H* [SEQ ID NO: 218] |
| GEM | EVQLLESGGGLVQPGGSLRLSCAASGFTFYGSYMGWVRQAPGKGLEWVSGISSYSYSTYYADSVKGRFT |
| (2) | ISRDNSKNTLYLQMNSLRAEDTAVYYCARYSPFHWYFDYWGQGTLVTVSSGGGGSGGGGSGGGGSDIQM |
| | TQSPSSLSASVGDRVTITCRASQSISSYLNWYQQKPGKAPKLLIYAASSLQSGVPSRFSGSGSGTDFTL |
| | TISSLQPEDFATYYCQQSFRDPPHTFGQGTKLEIKRLGDYKDHDGDYKDHDIDYKDDDDKAAAHHHHH |
| | H* [SEQ ID NO: 219] |
| GNAI | EVQLLESGGGLVQPGGSLRLSCAASGFTFSSYAMSWVRQAPGKGLEWVSYISGGYGYTSYADSVKGRFT |
| 3 (1) | ISRDNSKNTLYLQMNSLRAEDTAVYYCARVVYDSSYFDYWGQGTLVTVSSGGGGSGGGGSGGGGSDIQM |
| | TQSPSSLSASVGDRVTITCRASQSISSYLNWYQQKPGKAPKLLIYAASSLQSGVPSRFSGSGSGTDFTL |
| | TISSLQPEDFATYYCQQAYYGFPSTFGQGTKLEIKRLGDYKDHDGDYKDHDIDYKDDDDKAAAHHHHH |
| | H* [SEQ ID NO: 220] |
| GNAI | EVQLLESGGGLVQPGGSLRLSCAASGFTFSSSSMSWVRQAPGKGLEWVSGISGYGGGTGYADSVKGRFT |
| 3 (2) | ISRDNSKNTLYLQMNSLRAEDTAVYYCARSSYFVYFDYWGQGTLVTVSSGGGGSGGGGSGGGGSDIQMT |
| | QSPSSLSASVGDRVTITCRASQSISSYLNWYQQKPGKAPKWYAASSLQSGVPSRFSGSGSGTDFTLTIS |
| | SLQPEDFATYYCQQPYGYPYTFGQGTKLEIKRLGDYKDHDGDYKDHDIDYKDDDDKAAAHHHHHH* |
| | [SEQ ID NO: 221] |
| MAP | EVQLLESGGGLVQPGGSLRLSCAASGFTFSSYAMSWVRQAPGKGLEWVSAISGSGGSTYYADSVKGRFT |
| 2K6 | ISRDNSKNTLYLQMNSLRAEDTAVYYCARSHTVYFDYWGQGTLVTVSSGGGGSGGGGSGGGGSDIQMTQ |
| (3) | SPSSLSASVGDRVTITCRASQSISSYLNWYQQKPGKAPKWYAASSLQSGVPSRFSGSGSGTDFTLTISS |
| | LQPEDFATYYCQQSYSTPYTFGQGTKLEIKRLGDYKDHDGDYKDHDIDYKDDDDKAAAHHHHHH* |
| | [SEQ ID NO: 222] |
| MAP | EVQLLESGGGLVQPGGSLRLSCAASGFTFSSYAMSWVRQAPGKGLEWVSGISSYGGSTSYADSVKGRFT |
| 2K6 | ISRDNSKNTLYLQMNSLRAEDTAVYYCARFGHAFPAFDYWGQGTLVTVSSGGGGSGGGGSGGGGSDIQM |
| (4) | TQSPSSLSASVGDRVTITCRASQSISSYLNWYQQKPGKAPKLLIYAASSLQSGVPSRFSGSGSGTDFTL |
| | TISSLQPEDFATYYCQQSYSTPYTFGQGTKLEIKRLGDYKDHDGDYKDHDIDYKDDDDKAAAHHHHHH* |
| | [SEQ ID NO: 223] |
| MAP | EVQLLESGGGLVQPGGSLRLSCAASGFTFGYYSMSWVRQAPGKGLEWVSSISSSSSYTYYADSVKGRFT |
| 2K2 | ISRDNSKNTLYLQMNSLRAEDTAVYYCARHYSSFDYWGQGTLVTVSSGGGGSGGGGSGGGGSDIQMTQS |
| (1) | PSSLSASVGDRVTITCRASQSISSYLNWYQQKPGKAPKLLIYAASSLQSGVPSRFSGSGSGTDFTLTIS |
| | SLQPEDFATYYCQQSYWYPFTFGQGTKLEIKRLGDYKDHDGDYKDHDIDYKDDDDKAAAHHHHHH* |
| | [SEQ ID NO: 224] |
| MAP | EVQLLESGGGLVQPGGSLRLSCAASGFTFYYSSMSWVRQAPGKGLEWVSSIYGGGGSTSYADSVKGRFT |
| 2K2 | ISRDNSKNTLYLQMNSLRAEDTAVYYCARGPGHVIDYWGQGTLVTVSSGGGGSGGGGSGGGGSDIQMTQ |
| (2) | SPSSLSASVGDRVTITCRASQSISSYLNWYQQKPGKAPKLLIYAASSLQSGVPSRFSGSGSGTDFTLTI |
| | SSLQPEDFATYYCQQSYYVPFTFGQGTKLEIKRLGDYKDHDGDYKDHDIDYKDDDDKAAAHHHHHH* |
| | [SEQ ID NO: 225] |
| KRAS | EVQLLESGGGLVQPGGSLRLSCAASGFTFSGSYMYWVRQAPGKGLEWVSSIGSSYGYTYYADSVKGRFT |
| | ISRDNSKNTLYLQMNSLRAEDTAVYYCARYGYFSFDYWGQGTLVTVSSGGGGSGGGGSGGGGSDIQMTQ |
| | SPSSLSASVGDRVTITCRASQSISSYLNWYQQKPGKAPKLLIYAASSLQSGVPSRFSGSGSGTDFTLTI |
| | SSLQPEDFATYYCQQDHYLSTFGQGTKLEIKRLGDYKDHDGDYKDHDIDYKDDDDKAAAHHHHHH* |
| | [SEQ ID NO: 226] |
| PTPRO | EVQLLESGGGLVQPGGSLRLSCAASGFTFSYSGMGWVRQAPGKGLEWVSYISSYGYSTYYADSVKGRFT |
| (1) | ISRDNSKNTLYLQMNSLRAEDTAVYYCARSVSGGVIDYWGQGTLVTVSSGGGGSGGGGSGGGGSDIQMT |
| | QSPSSLSASVGDRVTITCRASQSIXXYLNWYQQKPGKAPKLLIYAAXSLQSGVPSRFSGSGSGTDFTLT |
| | ISSLQPEDFATYYCQQWVHYPYTFGQGTKLEIKRLGDYKDHDGDYKDHDIDYKDDDDKAAAHHHHHH* |
| | [SEQ ID NO: 227] |
| PTPRO | EVQLLESGGGLVQPGGSLRLSCAASGFTFSGYYXSWVRQAPGKGLEWVSSISGGSYSKSYADSVKGRFT |
| (4) | ISRDNSKNTLYLQMNSLRAEDTAVYYCARYSYYFDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQYAAYGLLTFGQGTKLEIKRLGDYKDHDGDYKDHDIDYKDDDDKAAAHHHHHH* |
| | [SEQ ID NO: 228] |
| PAR- | EVQLLESGGGLVQPGGSLRLSCAASGFTFSSYAMSWVRQAPGKGLEWVSAISGSGGSTYYADSVKGRFT |
| 6B | ISRDNSKNTLYLQMNSLRAEDTAVYYCARWNVWGHWGGPYSGVGLDYWGQGTLVTVSSGGGGSGGGGSG |
| (1) | GGGSDIQMTQSPSSLSASVGDRVTITCRASQSISSYLNWYQQKPGKAPKLLIYAASSLQSGVPSRFSGS |
| | GSGTDFTLTISSLQPEDFATYYCQQPYYPFTEGQGTKLEIKRLGDYKDHDGDYKDHDIDYKDDDDKAAA |
| | HHHHHH*[SEQ ID NO: 229] |
| PAR- | EVQLLES GGGLVQPGGSLRLSCAASGFTFSYYYMYWVRQAPGKGLEWVSYIYSYYYGTYYADSVKGRF |
| 6B | TISRDNSKNTLYLQMNSLRAEDTAVYYCARYAYYLDYWGQGTLVTVSSGGGGSGGGGSGGGGSDIQMTQ |
| (2) | SPSSLSASVGDRVTITCRASQSISSYLNWYQQKPGKAPKLLIYAASSLQSGVPSRFSGSGSGTDFTLTI |
| | SSLQPEDFATYYCQQHWSYGLYTFGQGTKLEIKRLGDYKDHDGDYKDHDIDYKDDDDKAAAHHHHHH* |
| | [SEQ ID NO: 230] |
| PRD- | EVQLLESGGGLVQPGGSLRLSCAASGFTFGSYGMSWVRQAPGKGLEWVSSIYSGYSYTYYADSVKGRFT |
| 14 | ISRDNSKNTLYLQMNSLRAEDTAVYYCARHGPYRGPGSMDYWGQGTLVTVSSGGGGSGGGGSGGGGSDI |
| (1) | QMTQSPSSLSASVGDRVTITCRASQSISSYLNWYQQKPGKAPKLLIYAASSLQSGVPSRFSGSGSGTDF |
| | TLTISSLQPEDFATYYCQQSWWLLTFGQGTKLEIKRLGDYKDHDGDYKDHDIDYKDDDDKAAAHHHHH |
| | H* [SEQ ID NO: 231] |
| PRD- | EVQLLESGGGLVQPGGSLRLSCAASGFTFYSGGMSWVRQAPGKGLEWVSSISGGYYYTYYADSVKGRFT |
| 14 | ISRDNSKNTLYLQMNSLRAEDTAVYYCARYVYGVGIDYWGQGTLVTVSSGGGGSGGGGSGGGGSDIQMT |
| (2) | QSPSSLSASVGDRVTITCRASQSISSYLNWYQQKPGKAPKLLIYAASSLQSGVPSRFSGSGSGTDFTLT |
| | ISSLQPEDFATYYCQQGYWLFTFGQGTKLEIKRLGDYKDHDGDYKDHDIDYKDDDDKAAAHHHHHH* |
| | [SEQ ID NO: 232] |
| PRD- | EVQLLESGGGLVQPGGSLRLSCAASGFTFSGYSMYWVRQAPGKGLEWVSSIYGYYGGTSYADSVKGRFT |
| 14 | ISRDNSKNTLYLQMNSLRAEDTAVYYCARGHPWFYMDYWGQGTLVTVSSGGGGSGGGGSGGGGSDIQMT |
| (3) | QSPSSLSASVGDRVTITCRASQSISSYLNWYQQKPGKAPKLLIYAASSLQSGVPSRFSGSGSGTDFTLT |
| | ISSLQPEDFATYYCQQSYWLYTFGQGTKLEIKRLGDYKDHDGDYKDHDIDYKDDDDKAAAHHHHHH* |
| | [SEQ ID NO: 233] |
| TOPB | EVQLLESGGGLVQPGGSLRLSCAASGFTFSGSSMSWVRQAPGKGLEWVSSISSGGSSTGYADSVKGRFT |
| P-1 | ISRDNSKNTLYLQMNSLRAEDTAVYYCARFSWGHWSSFFDYWGQGTLVTVSSGGGGSGGGGSGGGGSDI |
| (1) | QMTQSPSSLSASVGDRVTITCRASQSISSYLNWYQQKPGKAPKLLIYAASSLQSGVPSRFSGSGSGTDF |
| | TLTISSLQPEDFATYYCQQVYDLLTFGQGTKLEIKRLGDYKDHDGDYKDHDIDYKDDDDKAAAHHHHH |
| | H* [SEQ ID NO: 234] |
| TOPB | EVQLLESGGGLVQPGGSLRLSCAASGFTFGSSSMSWVRQAPGKGLEWVSSISYGSSSTSYADSVKGRFT |
| P-1 | ISRDNSKNTLYLQMNSLRAEDTAVYYCARGGYGFDYWGQGTLVTVSSGGGGSGGGGSGGGGSDIQMTQS |
| (2) | PSSLSASVGDRVTITCRASXXLNWYQQKPGKAPKLLIYXXXXSLQSGVPSRFSGSGSGTDFTLTISSLQ |
| | PEDFATYYCQQGXXXGXXTFGQGTKLEIKRLGDYKDHDGDYKDHDIDYKDDDDKAAAHHHHHH* |
| | [SEQ ID NO: 235] |
| USP- | EVQLLESGGGLVQPGGSLRLSCAASGFTFGSSSMYWVRQAPGKGLEWVSSISYYGYSTYYADSVKGRFT |
| 7 (1) | ISRDNSKNTLYLQMNSLRAEDTAVYYCARGSGIDYWGQGTLVTVSSGGGGSGGGGSGGGGSDIQMTQSP |
| | SSLSASVGDRVTITCRASQSISSYLNWYQQKPGKAPKLLIYAASSLQSGVPSRFSGSGSGTDFTLTISS |
| | LQPEDFATYYCQQWSVYGLYTFGQGTKLEIKRLGDYKDHDGDYKDHDIDYKDDDDKAAAHHHHHH* |
| | [SEQ ID NO: 236] |
| USP- | EVQLLESGGGLVQPGGSLRLSCAASGFTFSSYAMSWVRQAPGKGLEWVSAISGSGGSTYYADSVKGRFT |
| 7 (4) | ISRDNSKNTLYLQMNSLRAEDTAVYYCARVYSYPGPSSWGYFSSIDYWGQGTLVTVSSGGGGSGGGGSG |
| | GGGSDIQMTQSPSSLSASVGDRVTITCRASQSISSYLNWYQQKPGKAPKLLIYAASSLQSGVPSRFSGS |
| | GSGTDFTLTISSLQPEDFATYYCQQSYGYPHTFGQGTKLEIKRLGDYKDHDGDYKDHDIDYKDDDDKAA |
| | AHHHHHH* [SEQ ID NO: 237] |
| PARP- | EVQLLESGGGLVQPGGSLRLSCAASGFTFSGSGMGWVRQAPGKGLEWVSYIGYYSSGTYYADSVKGRFT |
| 1 | ISRDNSKNTLYLQMNSLRAEDTAVYYCARHHSFGYLDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGSGTDFTLT |
| | ISSLQPEDFATYYCQQGVHWSLHTFGQGTKLEIKRLGDYKDHDGDYKDHDIDYKDDDDKAAAHHHHHH* |
| | [SEQ ID NO: 238] |
| GRIP- | EVQLLESGGGLVQPGGSLRLSCAASGFTFSSYAMSWVRQAPGKGLEWVSAISGSGGSTYYADSVKGRFT |
| 2 (1) | ISRDNSKNTLYLQMNSLRAEDTAVYYCARIHFYGLDYWGQGTLVTVSSGGGGSGGGGSGGGGSDIQMTQ |
| | SPSSLSASVGDRVTITCRASQSISSYLNWYQQKPGKAPKLLIYAASSLQSGVPSRFSGSGSGTDFTLTI |
| | SSLQPEDFATYYCQQSYSTPYTFGQGTKLEIKRLGDYKDHDGDYKDHDIDYKDDDDKAAAHHHHHH* |
| | [SEQ ID NO: 239] |
| GRIP- | EVQLLESGGGLVQPGGSLRLSCAASGFTFSSYAMSWVRQAPGKGLEWVSAISGSGGSTYYADSVKGRFT |
| 2 (7) | ISRDNSKNTLYLQMNSLRAEDTAVYYCARGSYMDYWGQGTLVTVSSGGGGSGGGGSGGGGSDIQMTQSP |
| | SSLSASVGDRVTITCRASQSISSYLNWYQQKPGKAPKLLIYAASSLQSGVPSRFSGSGSGTDFTLTISS |
| | LQPEDFATYYCQQSYGPPTFGQGTKLEIKRLGDYKDHDGDYKDHDIDYKDDDDKAAAHHHHHH* |
| | [SEQ ID NO: 240] |
| GRIP- | EVQLLESGGGLVQPGGSLRLSCAASGFTFSSYAMSWVRQAPGKGLEWVSAISGSGGSTYYADSVKGRFT |
| 2 (8) | ISRDNSKNTLYLQMNSLRAEDTAVYYCARHSGPFFDYWGQGTLVTVSSGGGGSGGGGSGGGGSDIQMTQ |
| | SPSSLSASVGDRVTITCRASQSISSYLNWYQQKPGKAPKLLIYAASSLQSGVPSRFSGSGSGTDFTLTI |
| | SSLQPEDFATYYCQQGYSLHTFGQGTKLEIKRLGDYKDHDGDYKDHDIDYKDDDDKAAAHHHHHH* |
| | [SEQ ID NO: 241] |
| MAD | EVQLLESGGGLVQPGGSLRLSCAASGFTFGSSYMGWVRQAPGKGLEWVSGISGGGYGTYYADSVKGRFT |
| 2L1 | ISRDNSKNTLYLQMNSLRAEDTAVYYCARAPGGHYYGYFYFDYWGQGTLVTVSSGGGGSGGGGSGGGGS |
| (1) | DIQMTQSPSSLSASVGDRVTITCRASQSISSYLNWYQQKPGKAPKLLIYAASSLQSGVPSRFSGSGSGT |
| | DFTLTISSLQPEDFATYYCQQXXXXAHTFGQGTKLEIKRLGDYKDHDGDYKDHDIDYKDDDDKAAAHHH |
| | HHH* [SEQ ID NO: 242] |
| MAD | EVQLLESGGGLVQPGGSLRLSCAASGFTFYYSSMYWVRQAPGKGLEWVSGISSGGSGTYYADSVKGRFT |
| 2L1 | ISRDNSKNTLYLQMNSLRAEDTAVYYCARSFSYSSYLDYWGQGTLVTVSSGGGGSGGGGSGGGGSDIQM |
| (2) | TQSPSSLSASVGDRVTITCRASQSISSYLNWYQQKPGKAPKWYAASSLQSGVPSRFSGSGSGTDFILTI |
| | SSLQPEDFATYYCQQGGXXPTFGQGTKLEIKRLGDYKDHDGDYKDHDIDYKDDDDKAAAHHHHHH* |
| | [SEQ ID NO: 243] |
| HsHe | EVQLLESGGGLVQPGGSLRLSCAASGFTFSGSSMYWVRQAPGKGLEWVSGIGSYGGYTYYADSVKGRFT |
| c1 | ISRDNSKNTLYLQMNSLRAEDTAVYYCARDGTAVGSYFYFDYWGQGTLVTVSSGGGGSGGGGSGGGGSD |
| (2) | IQMTQSPSSLSASVGDRVTITCRASQSISSYLNWYQQKPGKAPKLLIYAASSLQSGVPSRFSGSGSGTD |
| | FTLTISSLQPEDFATYYCQQYYYYPHTFGQGTKLEIKRLGDYKDHDGDYKDHDIDYKDDDDKAAAHHHH |
| | HH* [SEQ ID NO: 244] |
| HsHe | EVQLLESGGGLVQPGGSLRLSCAASGFTFSSYAMSWVRQAPGKGLEWVSGIGSGGYYTSYADSVKGRFT |
| c1 | ISRDNSKNTLYLQMNSLRAEDTAVYYCARFYFVASPGGNLDYWGQGTLVTVSSGGGGSGGGGSGGGGSD |
| (3) | IQMTQSPSSLSASVGDRVTITCRASQSISSYLNWYQQKPGKAPKLLIYAASSLQSGVPSRFSGSGSGTD |
| | FTLTISSLQPEDFATYYCQQYSSPPTFGQGTKLEIKRLGDYKDHDGDYKDHDIDYKDDDDKAAAHHHHH |
| | H* [SEQ ID NO: 245] |
| Spindly | EVQLLESGGGLVQPGGSLRLSCAASGFTFSSYYMSWVRQAPGKGLEWVSSIDYSSYYTGYADSVKGRFT |
| (1) | ISRDNSKNTLYLQMNSLRAEDTAVYYCARGSYFDYWGQGTLVTVSSGGGGSGGGGSGGGGSDIQMTQSP |
| | SSLSASVGDRVTITCRASQSISSYLNWYQQKPGKAPKLLIYAASSLQSGVPSRFSGSGSGTDFTLTISS |
| | LQPEDFATYYCQQGSPLYTFGQGTKLEIKRLGDYKDHDGDYKDHDIDYKDDDDKAAAHHHHHH* |
| | [SEQ ID NO: 246] |
| Spindly | EVQLLESGGGLVQPGGSLRLSCAASGFTFYSYYMGWVRQAPGKGLEWVSSISYSGSGTYYADSVKGRFT |
| (2) | ISRDNSKNTLYLQMNSLRAEDTAVYYCARASYGTSYYYGYTIDYWGQGTLVTVSSGGGGSGGGGSGGGG |
| | SDIQMTQSPSSLSASVGDRVTITCRASQSISSYLNWYQQKPGKAPKLLIYAASSLQSGVPSRFSGSGSG |
| | TDFTLTISSLQPEDFATYYCQQSYAGPSTFGQGTKLEIKRLGDYKDHDGDYKDHDIDYKDDDDKAAAHH |
| | HHHH* [SEQ ID NO: 247] |
| PTPR | EVQLLESGGGLVQPGGSLRLSCAASGFTFSYSGMSWVRQAPGKGLEWVSSIGGSSSYTYYADSVKGRFT |
| K (1) | ISRDNSKNTLYLQMNSLRAEDTAVYYCARYSWGYYDAIDYWGQGTLVTVSSGGGGSGGGGSGGGGSDIQ |
| | MTQSPSSLSASVGDRVTITCRASQSISSYLNWYQQKPGKAPKLLIYAASSLQSGVPSRFSGSGSGTDFT |
| | LTISSLQPEDFATYYCQQSWWGHALYTFGQGTKLEIKRLGDYKDHDGDYKDHDIDYKDDDDKAAAHHHH |
| | HH* [SEQ ID NO: 248] |
| PTPR | EVQLLESGGGLVQPGGSLRLSCAASGFTFYYSSMSWVRQAPGKGLEWVSSIGYGSGYTYYADSVKGRFT |
| K (7) | ISRDNSKNTLYLQMNSLRAEDTAVYYCARVGASGSFDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGSGTDFTLT |
| | ISSLQPEDFATYYCQQYHYYSLYTFGQGTKLEIKRLGDYKDHDGDYKDHDIDYKDDDDKAAAHHHHHH* |
| | [SEQ ID NO: 249] |
| PTPR | EVQLLESGGGLVQPGGSLRLSCAASGFTFSSSSMGWVRQAPGKGLEWVSYISGYSYYTYYADSVKGRFT |
| K (8) | ISRDNSKNTLYLQMNSLRAEDTAVYYCARSSGTYGYYIDYWGQGTLVTVSSGGGGSGGGGSGGGGSDIQ |
| | MTQSPSSLSASVGDRVTITCRASQSISSYLNWYQQKPGKAPKLLIYAASSLQSGVPSRFSGSGSGTDFT |
| | LTISSLQPEDFATYYCQQGSYFPSTFGQGTKLEIKRLGDYKDHDGDYKDHDIDYKDDDDKAAAHHHHH |
| | H* [SEQ ID NO: 250] |
| PTPR | EVQLLESGGGLVQPGGSLRLSCAASGFTFGGYSMSWVRQAPGKGLEWVSGISYGYGSTYYADSVKGRFT |
| T (1) | ISRDNSKNTLYLQMNSLRAEDTAVYYCARGGPSYSMDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGSGTDFTLT |
| | ISSLQPEDFATYYCQQSAYLSTFGQGTKLEIKRLGDYKDHDGDYKDHDIDYKDDDDKAAAHHHHHH* |
| | [SEQ ID NO: 251] |
| PTPR | EVQLLESGGGLVQPGGSLRLSCAASGFTFSSSGMYWVRQAPGKGLEWVSGIYSYGSYTSYADSVKGRFT |
| 1(2) | ISRDNSKNTLYLQMNSLRAEDTAVYYCARVNYFGIDYWGQGTLVTVSSGGGGSGGGGSGGGGSDIQMTQ |
| | SPSSLSASVGDRVTITCRASQSISSYLNWYQQKPGKAPKLLIYAASSLQSGVPSRFSGSGSGTDFTLTI |
| | SSLQPEDFATYYCQQSWHSPPTFGQGTKLEIKRLGDYKDHDGDYKDHDIDYKDDDDKAAAHHHHHH* |
| | [SEQ ID NO: 252] |
| PGA | EVQLLESGGGLVQPGGSLRLSCAASGFTFYSSYMYWVRQAPGKGLEWVSGISSYGGSTSYADSVKGRFT |
| M-5 | ISRDNSKNTLYLQMNSLRAEDTAVYYCARYGPGSVFDYWGQGTLVTVSSGGGGSGGGGSGGGGSDIQMT |
| (1) | QSPSSLSASVGDRVTITCRASQSISSYLNWYQQKPGKAPKLLIYAASSLQSGVPSRFSGSGSGTDFILT |
| | ISSLQPEDFATYYCQQVYVAYSYPYTFGQGTKLEIKRLGDYKDHDGDYKDHDIDYKDDDDKAAAHHHHH |
| | H* [SEQ ID NO: 253] |
| PGA | EVQLLESGGGLVQPGGSLRLSCAASGFTFSGSYMYWVRQAPGKGLEWVSGISSSGDYTSYADSVKGRFT |
| M-5 | ISRDNSKNTLYLQMNSLRAEDTAVYYCARVGYHSFFDYWGQGTLVTVSSGGGGSGGGGSGGGGSDIQMT |
| (2) | QSPSSLSASVGDRVTITCRASQSISSYLNWYQQKPGKAPKLLIYAASSLQSGVPSRFSGSGSGTDFTLT |
| | ISSLQPEDFATYYCQQYYWYLHTFGQGTKLEIKRLGDYKDHDGDYKDHDIDYKDDDDKAAAHHHHHH* |
| | [SEQ ID NO: 254] |
| PTPR | EVQLLESGGGLVQPGGSLRLSCAASGFTFSSYAMSWVRQAPGKGLEWVSYISSSGGYTYYADSVKGRFT |
| J (1) | ISRDNSKNTLYLQMNSLRAEDTAVYYCARWAPWPYGYSMDYWGQGTLVTVSSGGGGSGGGGSGGGGSDI |
| | QMTQSPSSLSASVGDRVTITCRASQSISSYLNWYQQKPGKAPKLLIYAASSLQSGVPSRFSGSGSGTDF |
| | TLTISSLQPEDFATYYCQQSSYSLSTFGQGTKLEIKRLGDYKDHDGDYKDHDIDYKDDDDKAAAHHHHH |
| | H* [SEQ ID NO: 255] |
| PTPR | EVQLLESGGGLVQPGGSLRLSCAASGFTFSSGGMSWVRQAPGKGLEWVSSIYYSSSSTSYADSVKGRFT |
| J (7) | ISRDNSKNTLYLQMNSLRAEDTAVYYCARGYGYYMDYWGQGTLVTVSSGGGGSGGGGSGGGGSDIQMTQ |
| | SPSSLSASVGDRVTITCRASQSISSYLNWYQQKPGKAPKLLIYAASSLQSGVPSRFSGSGSGTDFTLTI |
| | SSLQEDFATYYCQQGSYAYLSTFGQGTKLEIKRLGDYKDHDGDYKDHDIDYKDDDDKAAAHHHHHH* |
| | [SEQ ID NO: 256] |
| PTPR | EVQLLESGGGLVQPGGSLRLSCAASGFTFSSGGMYWVRQAPGKGLEWVSSIYGSSSSTYYADSVKGRFT |
| J (8) | ISRDNSKNTLYLQMNSLRAEDTAVYYCARGPYASGYYYLDYWGQGTLVTVSSGGGGSGGGGSGGGGSDI |
| | QMTQSPSSLSASVGDRVTITCRASQSISSYLNWYQQKPGKAPKLLIYAASSLQSGVPSRFSGSGSGTDF |
| | TLTISSLQPEDFATYYCQQVNYSSSYPFTFGQGTKLEIKRLGDYKDHDGDYKDHDIDYKDDDDKAAAHH |
| | HHHH* [SEQ ID NO: 257] |
| ANM | EVQLLESGGGLVQPGGSLRLSCAASGFTFSSYAMSWVRQAPGKGLEWVSGISGGSYYTGYADSVKGRFT |
| 5 (1) | ISRDNSKNTLYLQMNSLRAEDTAVYYCARYTSAYYHAYFDYWGQGTLVTVSSGGGGSGGGGSGGGGSDI |
| | QMTQSPSSLSASVGDRVTITCRASQSISSYLNWYQQKPGKAPKLLIYAASSLQSGVPSRFSGSGSGTDF |
| | TLTISSLQPEDFATYYCQQSYSTPYTFGQGTKLEIKRLGDYXDHDGDYKDHDIDXKDDDDKAA |
| | [SEQ ID NO: 258] |
| ANM | EVQLLESGGGLVQPGGSLRLSCAASGFTFSYSSMGWVRQAPGKGLEWVSSIGGYSGYTYYADSVKGRFT |
| 5 (2) | ISRDNSKNTLYLQMNSLRAEDTAVYYCARGSWYLDYWGQGTLVTVSSGGGGSGGGGSGGGGSDIQMTQS |
| | PSSLSASVGDRVTITCRASQSISSYLNWYQQKPGKAPKWYAASSLQSGVPSRFSGSGSGTDFTLTISSL |
| | CIPEDFATYYCQQYGGYPPHTFGQGTKLEIKRLGDYKDHDGDYKDHDIDYKDDDDKAA |
| | [SEQ ID NO: 259] |
| APLF | EVQLLESGGGLVQPGGSLRLSCAASGFTFSSYAMSWVRQAPGKGLEWVSAISGSGGSTYYADSVKGRFT |
| (1) | ISRDNSKNTLYLQMNSLRAEDTAVYYCARGYYDLDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQSYSTPYTFGQGXKLEIKRLGDYKDHDGDYXDHDIDXXDDDXXAA |
| | [SEQ ID NO: 260] |
| APLF | EVQLLESGGGLVQPGGSLRLSCAASGFTFSSYAMSWVRQAPGKGLEWVSAISGSGGSTYYADSVKGRFT |
| (2) | ISRDNSKNTLYLQMNSLRAEDTAVYYCARGYYDMDYWGQGTLVTVSSGGGGSGGGGSGGGGSDIQMTQS |
| | PSSLSASVGDRVTITCRASQSISSYLNWYQQKPGKAPKWYAASSLQSGVPSRFSGSGSGTDFTLTISSL |
| | QPEDFATYYCQQSYSTPYTFGQGXKLEIKRLGXYKDHDGDYKDHDIDYXDDDDKAA |
| | [SEQ ID NO: 261] |
| ARH | EVQLLESGGGLVQPGGSLRLSCAASGFTFGSYSMSWVRQAPGKGLEWVSSISYGGSYTSYADSVKGRFT |
| GC-1 | ISRDNSKNTLYLQMNSLRAEDTAVYYCARVYGGYYYGYIDYWGQGTLVTVSSGGGGSGGGGSGGGGSDI |
| (1) | QMTQSPSSLSASVGDRVTITCRASQSISSYLNWYQQKPGKAPKLLIYAASSLQSGVPSRFSGSGSGTDF |
| | TLTISSLQPEDFATYYCQQWYADFPYTFGQGTKLEIKRLXDYKDHDGDYKDHDIDYXDXDDKAA |
| | [SEQ ID NO: 262] |
| BIRC | EVQLLESGGGLVQPGGSLRLSCAASGFTFYSSSMSWVRQAPGKGLEWVSSISSYSGYTSYADSVKGRFT |
| 2 (1) | ISRDNSKNTLYLQMNSLRAEDTAVYYCARDYWGSLDYWGQGTLVTVSSGGGGSGGGGSGGGGSDIQMTQ |
| | SPSSLSASVGDRVTITCRASQSISSYLNWYQQKPGKAPKLLIYAASSLQSGVPSRFSGSGSGTDFTLTI |
| | SSLQPEDFATYYCQQHGYSYPLTFGQGTKLEIKRLGDYKDHDGDYXDHDIDXKDDXXKAA |
| | [SEQ ID NO: 263] |
| BIRC | EVQLLESGGGLVQPGGSLRLSCAASGFTFGGYGMGWVRQAPGKGLEWVSSISSYSSYTYYADSVKGRFT |
| 2 (2) | ISRDNSKNTLYLQMNSLRAEDTAVYYCARTYYDYFDYWGQGTLVTVSSGGGGSGGGGSGGGGSDIQMTQ |
| | SPSSLSASVGDRVTITCRASQSISSYLNWYQQKPGKAPKLLIYAASSLQSGVPSRFSGSGSGTDFTLTI |
| | SSLQPEDFATYYCQQGYYYPYTFGQGTKLEIKRLGDYKDHDGDYKDHDIDYXDDDDKAA |
| | [SEQ ID NO: 264] |
| DCNL | EVQLLESGGGLVQPGGSLRLSCAASGFTFPYYYMSWVRQAPGKGLEWVSSIGSYSGGTSYADSVKGRFT |
| 1 (1) | ISRDNSKNTLYLQMNSLRAEDTAVYYCARVYRVYFYSGFDYWGQGTLVTVSSGGGGSGGGGSGGGGSDI |
| | QMTQSPSSLSASVGDRVTITCRASQSISSYLNWYQQKPGKAPKLLIYAASSLQSGVPSRFSGSGSGTDF |
| | TLTISSLQPEDFATYYCQQSYSTPYTFGQGTKLEIKRLXDYKDHDGDYKDHDIDYXDXDDXAXAHHHHH |
| | H-SPRXXSXSPXXESYYARXXXVVXQXXXXENXXXXXXXXXXXHPPFXXXXXXXX |
| | [SEQ ID NO: 265] |
| DCNL | EVQLLESGGGLVQPGGSLRLSCAASGFTFYYSYMSWVRQAPGKGLEWVSYISPGSGYTYYADSVKGRFT |
| 1 (2) | ISRDNSKNTLYLQMNSLRAEDTAVYYCARFYWGYSSYLDYWGQGTLVTVSSGGGGSGGGGSGGGGSDIQ |
| | MTQSPSSLSASVGDRVTITCRASQSISSYLNWYQQKPGKAPKLLIYAASSLQSGVPSRFSGSGSGTDFT |
| | LTISSLQPEDFATYYCQQVYYRGLPTFGQGXKLEIKRXGXYKDHDGDXKDHDXDXXMXMXXRP |
| | [SEQ ID NO: 266] |
| DLG1- | EVQLLESGGGLVQPGGSLRLSCAASGFTFSSYYMYWVRQAPGKGLEWVSGISSSGGSTYYADSVKGRFT |
| 1 (1) | ISRDNSKNTLYLQMNSLRAEDTAVYYCARNRDYSSYDGGYMDYWGQGTLVTVSSGGGGSGGGGSGGGGS |
| | DIQMTQSPSSLSASVGDRVTITCRASQSISSYLNWYQQKPGKAPKLLIYAASSLQSGVPSRFSGSGSGT |
| | DFTLTISSLQPEXFATYYCQQGYSYYPLTFGQGTKLEIKRLXDYKDHDGDYXDHDXDXXMTMXRRP |
| | [SEQ ID NO: 267] |
| DLG1- | EVQLLESGGGLVQPGGSLRLSCAASGFTFSSYSMYWVRQAPGKGLEWVSSISYGGYYTYYADSVKGRFT |
| 1 (2) | ISRDNSKNTLYLQMNSLRAEDTAVYYCARNGAFGYPYLDYWGQGTLVTVSSGGGGSGGGGSGGGGSDIQ |
| | MTQSPSSLSASVGDRVTITCRASQSISSYLNWYQQKPGKAPKLLIYAASSLQSGVPSRFSGSGSGTDFT |
| | LTISSLQPEDFATYYCQQSYSTPYTFGQGTKLEIKRLXXYKDHDGDYKDHDIDXXXXXDXAA |
| | [SEQ ID NO: 268] |
| DLG2- | EVQLLESGGGLVQPGGSLRLSCAASGFTFSSYSMYWVRQAPGKGLEWVSGISSYSYYTSYADSVKGRFT |
| 1 (1) | ISRDNSKNTLYLQMNSLRAEDTAVYYCARAFHGTPSIDYWGQGTLVTVSSGGGGSGGGGSGGGGSDIQM |
| | TQSPSSLSASVGDRVTITCRASQSISSYLNWYQQKPGKAPKLLIYAASSLQSGVPSRFSGSGSGTDFTL |
| | TIXSLQPEDFATYYCQXSNFXGLPTFXQGXKLEIXRLX-X-RX-R-LXXS-HRLQX-XXXXG |
| | [SEQ ID NO: 269] |
| DLG2- | EVQLLESGGGLVQPGGSLRLSCAASGFTFSGYSMSWVRQAPGKGLEWVSYISYSGYDTYYADSVKGRFT |
| 1 (2) | ISRDNSKNTLYLQMNSLRAEDTAVYYCARSYYSYGYWHIDYWGQGTLVTVSSGGGGSGGGGSGGGGSDI |
| | QMTQSPSSLSASVGDRVTITCRASQSISSYLNWYQQKPGKAPKLLIYAASSLQSGVPSRFSGSGSGTDF |
| | TLTISSLQPEDFATYYCQQNSYSGPFTFGQGTXLEXKRLGXYKDHDGDYKDHDIDYXDDXXXXAAXXXH |
| | HX-SPRXXXXXXXXEXYYARSLAVXXXRXDWXX [SEQ ID NO: 270] |
| DPOL | EVQLLESGGGLVQPGGSLRLSCAASGFTFGSYSMYWVRQAPGKGLEWVSSISYGGGYTSYADSVKGRFT |
| M (1) | ISRDNSKNTLYLQMNSLRAEDTAVYYCARGYYYMDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQSWSPFTFGQGXKLEIKRLXXYKDHDGDYXDHDIXXXDDDDXAA |
| | [SEQ ID NO: 271] |
| DPOL | EVQLLESGGGLVQPGGSLRLSCAASGFTFGYYSMGWVRQAPGKGLEWVSGISYYSGYTSYADSVKGRFT |
| M (2) | ISRDNSKNTLYLQMNSLRAEDTAVYYCARVGTWFTAFDYWGQGTLVTVSSGGGGSGGGGSGGGGSDIQM |
| | TQSPSSLSASVGDRVTITCRASQSISSYLNWYQQKPGKAPKLLIYAASSLQSGVPSRFSGSGSGTDFTL |
| | TISSLQPEDFATYYCQQYYVPFTFGQGTKLEIKRLGDYKDHDGDYKDHDIDXXXDDDKAA |
| | [SEQ ID NO: 272] |
| DLG4- | EVQLLESGGGLVQPGGSLRLSCAASGFTFGSYYMSWVRQAPGKGLEWVSGISGSYGYTYYADSVKGRFT |
| 2 (1) | ISRDNSKNTLYLQMNSLRAEDTAVYYCARVSSGSAAFDYWGQGTLVTVSSGGGGSGGGGSGGGGSDIQM |
| | TQSPSSLSASVGDRVTITCRASQSISSYLNWYQQKPGKAPKLLIYAASSLQSGVPSRFXGSGSXTDFTL |
| | TIXXLQPEXFATYYCQQYFSLLTFGQGTXXXDQTPXXX-RP-RXLXXS-HXXXX-XXXGXXXXXXXXXX |
| | XXXXXXX [SEQ ID NO: 273] |
| DLG4- | EVQLLESGGGLVQPGGSLRLSCAASGFTFYSSYMYWVRQAPGKGLEWVSGISSSSGSTYYADSVKGRFT |
| 2 (2) | ISRDNSKNTLYLQMNSLRAEDTAVYYCARYGYYSWGFDYWGQGTLVTVSSGGGGSGGGGSGGGGSDIQM |
| | TQSPSSLSASVGDRVTITCRASQSISSYLNWYQQKPGKAPKLLIYAASSLQSGVPSRFSGSGSGTDFTL |
| | TISSLQPEDFATYYCQQGAGFPPTFGQGTKLEIKRLGDYKDHDGDYKDHDIXXRMXXXRRP |
| | [SEQ ID NO: 274] |
| GOR | EVQLLESGGGLVQPGGSLRLSCAASGFTFSSYYMGWVRQAPGKGLEWVSSIYGYGGGTYYADSVKGRFT |
| S2-1 | ISRDNSKNTLYLQMNSLRAEDTAVYYCARGGSYFDYWGQGTLVTVSSGGGGSGGGGSGGGGSDIQMTQS |
| (1) | PSSLSASVGDRVTITCRASQSISSYLNWYQQKPGKAPKLLIYAASSLQSGVPSRFSGSGSGTDFTLTIS |
| | SLQPEDFATYYCQQVYGPLTFGQGTKLEIKRLX-L-RP-RXX-XIMTSXXRXXXTRRP |
| | [SEQ ID NO: 275] |
| GOR | EVQLLESGGGLVQPGGSLRLSCAASGFTFSYYSMGWVRQAPGKGLEWVSSISYSGYYTYYADSVKGRFT |
| S2-1 | ISRDNSKNTLYLQMNSLRAEDTAVYYCARSGHHVSFDYWGQGTLVTVSSGGGGSGGGGSGGGGSDIQMT |
| (2) | QSPSSLSASVGDRVTITCRASQSISSYLNWYQQKPGKAPKLLIYAASSLQSGVPSRFSGSGSGTDFTLT |
| | ISSLQPEDFATYYCQQGFYPFTFGQGTKLEIKRLGDYKDHDGDYKDHDIDYXDDDDKAA |
| | [SEQ ID NO: 276] |
| INAD | EVQLLESGGGLVQPGGSLRLSCAASGFTFSSYAMSWVRQAPGKGLEWVSAISGSGGSTYYADSVKGRFT |
| L-1 | ISRDNSKNTLYLQMNSLRAEDTAVYYCARDYVSAYGGYFDYWGQGTLVTVSSGGGGSGGGGSGGGGSDI |
| (1) | QMTQSPSSLSASVGDRVTITCRASQSISSYLNWYQQKPGKAPKLLIYAASSLQSGVPSRFSGSGSGTDF |
| | TLTISSLQPEXFATYYCQQGYGSLHTFXQGXKLEIKRLXXYKDHXGDYXDHDIDXKDXDDKAA |
| | [SEQ ID NO: 277] |
| INAD | EVQLLESGGGLVQPGGSLRLSCAASGFTFSSYAMSWVRQAPGKGLEWVSAISGSGGSTYYADSVKGRFT |
| L-1 | ISRDNSKNTLYLQMNSLRAEDTAVYYCARWTSGGYLDYWGQGTLVTVSSGGGGSGGGGSGGGGSDIQMT |
| (2) | QSPSSLSASVGDRVTITCRASQSISSYLNWYQQKPGKAPKLLIYAASXLQSGVPSRFSGSGSGTDFTLT |
| | IXSLQPEDFXXYYCQQGWSLLXFGQGXXXXRSNA-VXIXXMIVXIKIXXXXXRMXXXXXXPX |
| | [SEQ ID NO: 278] |
| KCC2 | EVQLLESGGGLVQPGGSLRLSCAASGFTFSSYAMSWVRQAPGKGLEWVSAISGSGGSTYYADSVKGRFT |
| B-3 | ISRDNSKNTLYLQMNSLRAEDTAVYYCARDFSYGFDYWGQGTLVTVSSGGGGSGGGGSGGGGSDIQMTQ |
| (2) | SPSSLSASVGDRVTITCRASQSISSYLNWYQQKPGKAPKLLIYAASSLQSGVPSRFSGSGSGTDFTLTI |
| | SSLQPEXFATYYCQQGSDDPYTFGQGXKLEIKRLGXYKDHDGDYXDHDIDXXDXXXXAA |
| | [SEQ ID NO: 279] |
| KCC2 | EVQLLESGGGLVQPGGSLRLSCAASGFTFYSSYMYWVRQAPGKGLEWVSSIGGSSYSTYYADSVKGRFT |
| B-3 | ISRDNSKNTLYLQMNSLRAEDTAVYYCARFHDYWALDYWGQGTLVTVSSGGGGSGGGGSGGGGSDIQMT |
| (1) | QSPSSLSASVGDRVTITCRASQSISSYLNWYQQKPGKAPKLLIYAASSLQSGVPSRFSGSGSGTDFTLT |
| | ISSLQPEXFATYYCXQSTVWYLPTFGQGXXLEIXXXXDYKDHDX-L-XS-HXLXXMXXXXRP |
| | [SEQ ID NO: 280] |
| ITCH- | EVQLLESGGGLVQPGGSLRLSCAASGFTFYYSYMYWVRQAPGKGLEWVSGIGGYGGSTSYADSVKGRFT |
| 2 (1) | ISRDNSKNTLYLQMNSLRAEDTAVYYCARVVGAGDFDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGXGTDFTLT |
| | ISSLQPEDFATYYCQQYFFYYLHTFXXGXXXWXSNA-XXXKXMXXXYXDHXIXXXXDDXXXXRPXXXXX |
| | LIXXXXXPXXXX [SEQ ID NO: 281] |
| ITCH- | EVQLLESGGGLVQPGGSLRLSCAASGFTFYSYGMYWVRQAPGKGLEWVSYIPGYGYSTSYADSVKGRFT |
| 2 (2) | ISRDNSKNTLYLQMNSLRAEDTAVYYCARSFGYGDFDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGSGTXFTLT |
| | IXSLQPEDFATYYCQQYDYSPYTFGXGXKLEIXRLGXYKDHDGDYXXXDIDYXG-XXXXXXPXSSSXXI |
| | XXXXXSXXP [SEQ ID NO: 282] |
| KCC4 | EVQLLESGGGLVQPGGSLRLSCAASGFTFGGGYMGWVRQAPGKGLEWVSYISGSGSYTYYADSVKGRFT |
| (1) | ISRDNSKNTLYLQMNSLRAEDTAVYYCARYSSVSSYSYFDYWGQGTLVTVSSGGGGSGGGGSGGGGSDI |
| | QMTQSPSSLSASVGDRVTITCRASQSISSYLNWYQQKPGKAPKLLIYAASSLQSGVPSRFSGSGSGTDF |
| | TLTISSLQPEDFATYYCQQSVHLPTFGQGTKLEIKRLGDYXDHDGDYXDHDIXXXDXXXKAA |
| | [SEQ ID NO: 283] |
| KCC4 | EVQLLESGGGLVQPGGSLRLSCAASGFTFPYSYMYWVRQAPGKGLEWVSAISGSGGSTYYADSVKGRFT |
| (2) | ISRDNSKNTLYLQMNSLRAEDTAVYYCARYASYSAIDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGSGTDFTLT |
| | ISSLQPEXFATYYCQQTGGLYTFGQGXKLXIKRLGXYKDHDXDYXXXDXDYXDDDXXAXXXXHHXXXSP |
| | RWXSXXPXSXSXXXRXX [SEQ ID NO: 284] |
| KKCC | EVQLLESGGGLVQPGGSLRLSCAASGFTFYYSYMYWVRQAPGKGLEWVSAISGSGGSTYYADSVKGRFT |
| 1-1 | ISRDNSKNTLYLQMNSLRAEDTAVYYCARSYTPASYRFDYWGQGTLVTVSSGGGGSGGGGSGGGGSDIQ |
| (1) | MTQSPSSLSASVGDRVTITCRASQSISSYLNWYQQKPGKAPKLLIYAASSLQSGVPSRFSGSGSGTDFT |
| | LTISSLQPEDFATYYCQQSYSTPYTFGQGXKLEIKRLGDYXDHDGDYKDHDXDXKDDDXXXXRPSXSSX |
| | LITXXXLXXALX-VVLXALXXPXFYXVXTXKTXXXXXXXXXXXHPXXXXX [SEQ ID NO: 285] |
| KKCC | EVQLLESGGGLVQPGGSLRLSCAASGFTFGGSGMGWVRQAPGKGLEWVSSIGYGYSSTYYADSVKGRFT |
| 1-1 | ISRDNSKNTLYLQMNSLRAEDTAVYYCARYSYHYYPDFDYWGQGTLVTVSSGGGGSGGGGSGGGGSDIQ |
| (2) | MTQSPSSLSASVGDRVTITCRASQSISSYLNWYQQKPGKAPKLLIYAASSLQSGVPSRFSGSGSGTDFT |
| | LTISSLQPEDFATYYCQQVFRYPLTFGQGXKLEIKRLXXYXDHDGXYXDHDIDYXDXXXXAA |
| | [SEQ ID NO: 826] |
| LIN7 | EVQLLESGGGLVQPGGSLRLSCAASGFTFSGYSMSWVRQAPGKGLEWVSSISSGSYSTSYADSVKGRFT |
| A (1) | ISRDNSKNTLYLQMNSLRAEDTAVYYCARGYSYFDRSFDYWGQGTLVTVSSGGGGSGGGGSGGGGSDIQ |
| | MTQSPSSLSASVGDRVTITCRASQSISSYLNWYQQKPGKAPKLLIYAASSLQSGVPSRFSGSGSGTDFT |
| | LTISSLQPEDFATYYCQQNPGPFTFGQGTKLEIKRLGXYKDHDGDYKDHDIDYXDDDDXAA |
| | [SEQ ID NO: 287] |
| LIN7 | EVQLLESGGGLVQPGGSLRLSCAASGFTFYSYGMSWVRQAPGKGLEWVSSISSSYSYTYYADSVKGRFT |
| A (2) | ISRDNSKNTLYLQMNSLRAEDTAVYYCARGYYGGFDYWGQGTLVTVSSGGGGSGGGGSGGGGSDIQQSX |
| | MTQSPSSLSASVGDRVTITCRASQSISSYLNWYQQKPGKAPKLLIYAASSLQSGVPSRFSGSGSGTDFT |
| | LTISSLQPEXFATYYCQYTPFTFGQGXKLEIKRLGDYKDHDGDYKDHDIDXKDDDDXAA |
| | [SEQ ID NO: 288] |
| SNTA | EVQLLESGGGLVQPGGSLRLSCAASGFTFSSYSMSWVRQAPGKGLEWVSGIYGGGSSTYYADSVKGRFT |
| 1 (1) | ISRDNSKNTLYLQMNSLRAEDTAVYYCARPSSSGSYVMDYWGQGTLVTVSSGGGGSGGGGSGGGGSDIQ |
| | MTQSPSSLSASVGDRVTITCRASQSISSYLNWYQQKPGKAPKLLIYAASSLQSGVPSRFSGSGSGTDFT |
| | LTISSLQPEDFATYYCQQSYSPHTFGQGXKLEIKRLGXYKDHDGXYKDHDXXXXDXDDXAA |
| | [SEQ ID NO: 289] |
| SNTA | EVQLLESGGGLVQPGGSLRLSCAASGFTFSSYAMSWVRQAPGKGLEWVSAISGSGGSTYYADSVKGRFT |
| 1 (2) | ISRDNSKNTLYLQMNSLRAEDTAVYYCARYPVYYYFDYWGQGTLVTVSSGGGGSGGGGSGGGGSDIQMT |
| | QSPSSLSASVGDRVTITCRASQSISSYLNWYQQKPGKAPKLLIYAASSLQSGVPSRFSGSGSGTDFTLT |
| | ISSLQPEDFATYYCQQSYVPLSTFGQGTKLEIKRLGXYKDHDGDYKDHDIDXXDDDDXAA |
| | [SEQ ID NO: 290] |
| MAG | EVQLLESGGGLVQPGGSLRLSCAASGFTFSYSSMSWVRQAPGKGLEWVSGISGSGYSTYYADSVKGRFT |
| I1-1 | ISRDNSKNTLYLQMNSLRAEDTAVYYCARGYSGFDYWGQGTLVTVSSGGGGSGGGGSGGGGSDIQMTQS |
| (1) | PSSLSASVGDRVTITCRASQSISSYLNWYQQKPGKAPKLLIYAASSLQSGVPSRFSGSGSGTDFTLTIS |
| | SLQPEDFATYYCQQYGYATLPTFGQGXKLEIKRLGXYKDHDGDYKDHDIDYXXDDDXAA |
| | [SEQ ID NO: 291] |
| MAG | EVQLLESGGGLVQPGGSLRLSCAASGFTFSSYAMSWVRQAPGKGLEWVSGISGSYGYTSYADSVKGRFT |
| I1-1 | ISRDNSKNTLYLQMNSLRAEDTAVYYCARGYYSYYDGPIDYWGQGTLVTVSSGGGGSGGGGSGGGGSDI |
| (2) | QMTQSPSSLSASVGDRVTITCRASQSISSYLNWYQQKPGKAPKLLIYAASSLQSGVPSRFSGSGSGTDF |
| | TLTISSLQPEDFATYYCQQSYSTPYTFXQGTKLEIKRLXDYKDHXGXYKDHDIDXXDDDXXAXAHXHXH |
| | XXSPRXXSXXPXSESXXXRXXAVVXXXXDWENPXXXNXIAXXXXPXXXXXXXX |
| | [SEQ ID NO: 292] |
| MAR | EVQLLESGGGLVQPGGSLRLSCAASGFTFSGYGMGWVRQAPGKGLEWVSSISYYGGGTGYADSVKGRFT |
| K1-1 | ISRDNSKNTLYLQMNSLRAEDTAVYYCARFDDFYASHYGIYIDYWGQGTLVTVSSGGGGSGGGGSGGGG |
| (1) | SDIQMTQSPSSLSASVGDRVTITCRASQSISSYLNWYQQKPGKAPKLLIYAASSLQSGVPSRFSGSGSG |
| | TDFTLTISSLQPEDFATYYCQQASSLFTFGXXXKLEIKRLXDYKDHDXDYKDHDIDYKXDXDKAXXHHH |
| | HHX-SXXXXSXSPYSEXXYXRXLXXRFXTXXXGKPXVTXXXRXXXXPPFXXWXXXXXX |
| | [SEQ ID NO: 293] |
| MAR | EVQLLESGGGLVQPGGSLRLSCAASGFTFSSYAMSWVRQAPGKGLEWVSAISGSGGSTYYADSVKGRFT |
| K1-1 | ISRDNSKNTLYLQMNSLRAEDTAVYYCARYSYGSYFDYWGQGTLVTVSSGGGGSGGGGSGGGGSDIQMT |
| (2) | QSPSSLSASVGDRVTITCRASQSISSYLNWYQQKPGKAPKLLIYAASSLQSGVPSRFSGSGSGTDFTLT |
| | ISSLQPEDFATYYCQQYHGWPPTFXQGTKLEIKRLGDYKDHDGDYKDHDIDXXDDDDKAAXHHHHHH- |
| | SPRXXSXXPYSESXYARXLAVVLXVXTGKTXXYXXXSPXXXSPFXXLAXXRXXXXXXXX |
| | [SEQ ID NO: 294] |
| OTU | EVQLLESGGGLVQPGGSLRLSCAASGFTFYSSSMYWVRQAPGKGLEWVSGISSYGSYTSYADSVKGRFT |
| 6B | ISRDNSKNTLYLQMNSLRAEDTAVYYCARGPFFDYWGQGTLVTVSSGGGGSGGGGSGGGGSDIQMTQSP |
| (1) | SSLSASVGDRVTITCRASQSISSYLNWYQQKPGKAPKLLIYAASSLQSGVPSRFSGSGSGTDFTLTISS |
| | LQPEDFATYYCQQFPNPHTFGQGXRLEIKRLGDYKDHDGDYKDHDIXXXDXDDXAA |
| | [SEQ ID NO: 295] |
| OTU | EVQLLESGGGLVQPGGSLRLSCAASGFTFSSSGMSWVRQAPGKGLEWVSSISGSYGYTSYADSVKGRFT |
| 6B | ISRDNSKNTLYLQMNSLRAEDTAVYYCARSYSYVYGFDYWGQGTLVTVSSGGGGSGGGGSGGGGSDIQM |
| (2) | TQSPSSLSASVGDRVTITCRASQSISSYLNWYQQKPGKAPKLLIYAASSLQSGVPSRFSGSGSGTDFTL |
| | TISSLQPEDFATYYCQQNVHLYTFGQGXKLEIKRLGDYKDHDGDYKDHDIDXXDDXDXAA |
| | [SEQ ID NO: 296] |
| NOS | EVQLLESGGGLVQPGGSLRLSCAASGFTFGSYYMYWVRQAPGKGLEWVSGISSYSGYTYYADSVKGRFT |
| 1-1 | ISRDNSKNTLYLQMNSLRAEDTAVYYCARSADSGGFDYWGQGTLVTVSSGGGGSGGGGSGGGGSDIQMT |
| (1) | QSPSSLSASVGDRVTITCRASQSISSYLNWYQQKPGKAPKLLIYAASSLQSGVPSRFSGSGSGXXFTLT |
| | ISSLQPEXFATYYCQQSYFYGLPTFXQGTKLEIXRLXXYXDHDXDXXXHDIXYXD |
| | [SEQ ID NO: 297] |
| NOS | EVQLLESGGGLVQPGGSLRLSCAASGFTFSSYAMSWVRQAPGKGLEWVSAISGSGGSTYYADSVKGRFT |
| 1-1 | ISRDNSKNTLYLQMNSLRAEDTAVYYCARPGYYGAAYYRSFIDYWGQGTLVTVSSGGGGSGGGGSGGGG |
| (2) | SDIQMTQSPSSLSASVGDRVTITCRASQSISSYLNWYQQKPGKAPKLLIYAASSLQSGVPSRFSGSGSG |
| | TDFTLTISSLQPEXFATYYCQQSYSTPYTFGQGXKLEIKRLGXYKDHDXXYKDHDIDXXXXDDKAA |
| | [SEQ ID NO: 298] |
| OTU | EVQLLESGGGLVQPGGSLRLSCAASGFTFYSSSMSWVRQAPGKGLEWVSGISSSGYSTGYADSVKGRFT |
| B1-1 | ISRDNSKNTLYLQMNSLRAEDTAVYYCARYGGYHTYLDYWGQGTLVTVSSGGGGSGGGGSGGGGSDIQM |
| (1) | TQSPSSLSASVGDRVTITCRASQSISSYLNWYQQKPGKAPKLLIYAASSLQSGVPSRFSGSGSGTDFTL |
| | TISSLQPEDFATYYCQQYSYTSLFTFXXGXXLXIKRXXXYKXHXXDYKDHDXXXXXDXXXAXXXHHXH |
| | X-SXX [SEQ ID NO: 299] |
| OTU | EVQLLESGGGLVQPGGSLRLSCAASGFTFSSYAMSWVRQAPGKGLEWVSAISGSGGSTYYADSVKGRFT |
| B1-1 | ISRDNSKNTLYLQMNSLRAEDTAVYYCARYHYYAGFDYWGQGTLVTVSSGGGGSGGGGSGGGGSDIQMT |
| (2) | QSPSSLSASVGDRVTITCRASQSISSYLNWYQQKPGKAPKLLIYAASSLQSGVPSRFSGSGSGTDFTLT |
| | ISSLQPEXFATYYCXQSGSFLPTFGQGXXXEIKRLGXYXXHXXXYKXHXIDXXMXMXRXXPIXXIXXXX |
| | XX [SEQ ID NO: 300] |
| OTU | EVQLLESGGGLVQPGGSLRLSCAASGFTFGGYSMSWVRQAPGKGLEWVSGIYSYSYSTSYADSVKGRFT |
| B2-1 | ISRDNSKNTLYLQMNSLRAEDTAVYYCARHPNWSYFDYWGQGTLVTVSSGGGGSGGGGSGGGGSDIQMT |
| (1) | QSPSSLSASVGDRVTITCRASQSISSYLNWYQQKPGKAPKLLIYAASSLQSGVPSRFSGSGSGTDFTLT |
| | ISSLQPEDFATYYCQQXYSTPYTFGQGXKLXXXRLXDYKDHDXDYKDHDXDXXDDXXXXAAHXHHHX-X |
| | PRWXSXXPYXEXXYXRSLXXXFXXXXLGKPXXYXXXXXXXXSPFXXXX [SEQ ID NO: 301] |
| OTU | EVQLLESGGGLVQPGGSLRLSCAASGFTFYGSYMYWVRQAPGKGLEWVSYISGSSGSTSYADSVKGRFT |
| B2-1 | ISRDNSKNTLYLQMNSLRAEDTAVYYCARYGHASFDYWGQGTLVTVSSGGGGSGGGGSGGGGSDIQMTQ |
| (2) | SPSSLXASVGDRVTITCRASXXISSYLNWYXXKPGKAPKLLIYAASXLQSGVPSRFXXSGSGTXFTLTI |
| | XXLXPXXFATYYCQXGSXLPXFGXXXXXXXXXXXXYKXXXXX [SEQ ID NO: 302] |
| PAK4- | EVQLLESGGGLVQPGGSLRLSCAASGFTFSSYSMYWVRQAPGKGLEWVSSISGSGSYTYYADSVKGRFT |
| 1 (1) | ISRDNSKNTLYLQMNSLRAEDTAVYYCARYYHRSHRFPLDYWGQGTLVTVSSGGGGSGGGGSGGGGSDI |
| | QMTQSPSSLSASVGDRVTITCRASQSISSYLNWYQQKPGKAPKLLIYAASSLQSGVPSRFSGSGSGTDF |
| | TLTISSLQPEDFATYYCQQSSYPFTFGXXPSWRXNA-VIIKTMTVIIKIMTSIXXMTMXRRP |
| | [SEQ ID NO: 303] |
| PAK4- | EVQLLESGGGLVQPGGSLRLSCAASGFTFGYYSMSWVRQAPGKGLEWVSSIYSYSSGTYADSVKGRFTI |
| 1 (2) | SRDNSKNTLYLQMNSLRAEDTAVYYCARGSSVIDYWGQGTLVTVSSGGGGSGGGGSGGGGSDIQMTQSP |
| | SSLSASVGDRVTITCRASQSISSYLNWYQQKPGKAPKLLIYAASSLQSGVPSRFSGSGSGTDFTLTISS |
| | LQPEDFATYYCQQSYSTPYTFGQGXKLEIKRLGXYKDHDGDYKDHDXDXXDDDXXAA |
| | [SEQ ID NO: 304] |
| PRD | EVQLLESGGGLVQPGGSLRLSCAASGFTFSGYGMSWVRQAPGKGLEWVSSIGGSYYSTGYADSVKGRFT |
| M8-1 | ISRDNSKNTLYLQMNSLRAEDTAVYYCARSYSVHYPYYHDHFDYWGQGTLVTVSSGGGGSGGGGSGGGG |
| (1) | SDIQMTQSPSSLSASVGDRVTITCRASQSISSYLNWYQQKPGKAPKLLIYAASSLQSGVPSRFSGSGSG |
| | TDFTLTISSLQPEXFATYYCQQYFYPYTFGQGXKLEIKRLGDYXDHDGDYKDHDIXXXDDXXXAA |
| | [SEQ ID NO: 305] |
| PRD | EVQLLESGGGLVQPGGSLRLSCAASGFTFSYSSMYWVRQAPGKGLEWVSYISGYGGYTSYADSVKGRFT |
| M8-1 | ISRDNSKNTLYLQMNSLRAEDTAVYYCARDVGYYFWSGHSMDYWGQGTLVTVSSGGGGSGGGGSGGGGS |
| (2) | DIQMTQSPSSLSASVGDRVTITCRASQSIXSYLNWYQXKPXKAPKLLIYAASXLQSGVPSRFXXXGXXX |
| | XFTLTIXXLXPEXFXXXYCXXXSSXXTFXQXXXLXXXRX [SEQ ID NO: 306] |
| PTN1 | EVQLLESGGGLVQPGGSLRLSCAASGFTFSSYAMSWVRQAPGKGLEWVSGISGYSSMYADSVKGRFTIS |
| 3-1 | RDNSKNTLYLQMNSLRAEDTAVYYCARVYYPGHSMDYWGQGTLVTVSSGGGGSGGGGSGGGGSDIQMTQ |
| (1) | SPSSLSASVGDRVTITCRASQSISSYLNWYQQKPGKAPKWYAASSLQSGVPSRFSGSGSGTDFTLTISS |
| | LQPEDFATYYCQQSVHYPHTFGQGTKLEIKRLGXYKXHXGDYKDHDXDXXXXXXXXAAXXHHHH-SPRX |
| | XXXXPYSXXXXXRXXXXXFXTXXXXKPXXXXX [SEQ ID NO: 307] |
| PTN1 | EVQLLESGGGLVQPGGSLRLSCAASGFTFGSYYMSWVRQAPGKGLEWVSGIGSYSSYTGYADSVKGRFT |
| 3-1 | ISRDNSKNTLYLQMNSLRAEDTAVYYCARGHSYYYSPPFDYWGQGTLVTVSSGGGGSGGGGSGGGGSDI |
| (2) | QMTQSPSSLSASVGDRVTITCRASQSISSYLNWYQQKPGKAPKLLIYAASSLQSGVPSRFSGSGSGTDF |
| | TLTISSLQPEXFATYYCQQDGYSPFTFXQGXKLXIKRLXXYKDXDGXYXXHDIDYKXDXXXXA |
| | [SEQ ID NO: 308] |
| CHEK | EVQLLESGGGLVQPGGSLRLSCAASGFTFGSYSMSWVRQAPGKGLEWVSGISGSYSSTYYADSVKGFTI |
| 2 (1) | SRDNSKNTLYLQMNSLRAEDTAVYYCARGDSWVFDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQSYSYPFTFGQGTKLEIKRLGXYKDHDGDYKDHDIDYXDDDXKAA |
| | [SEQ ID NO: 309] |
| CHEK | EVQLLESGGGLVQPGGSLRLSCAASGFTFYYSGMSWVRQAPGKGLEWVSGIGYSGYYTSYADSVKGRFT |
| 2 (2) | ISRDNSKNTLYLQMNSLRAEDTAVYYCARSSYYGTSGYVFDYWGQGTLVTVSSGGGGSGGGGSGGGGSD |
| | IQMTQSPSSLSASVGDRVTITCRASQSISSYLNWYQQKPGKAPKLLIYAASSLQSGVPSRFXGSGSGTD |
| | FTLTISSLQPEDFATYYCQQADVYPLTFGQGXKLEXKRLGXYXDHDGXXKDHDIXXXDDXXXRP |
| | [SEQ ID NO: 310] |
| CSNK | EVQLLESGGGLVQPGGSLRLSCAASGFTFGSYSMSWVRQAPGKGLEWVSSISGGYSYTSYADSVKGRFT |
| 1E | ISRDNSKNTLYLQMNSLRAEDTAVYYCARSFSYYYGVFLDYWGQGTLVTVSSGGGGSGGGGSGGGGSDI |
| (1) | QMTQSPSSLSASVGDRVTITCRASQSISSYLNWYQQKPGKAPKLLIYAASSLQSGVPSRFSGSGSGTDF |
| | TLTISSLQPEDFATYYCQQGYSSYPLTFGQGXKLEIKRLGDYKDHDGXYKDHDIDYXDDXDXAA |
| | [SEQ ID NO: 311] |
| CSNK | EVQLLESGGGLVQPGGSLRLSCAASGFTFSSYAMSWVRQAPGKGLEWVSGIYSSYGGTYYADSVKGRFT |
| 1E | ISRDNSKNTLYLQMNSLRAEDTAVYYCARGVYMDYWGQGTLVTVSSGGGGSGGGGSGGGGSDIQMTQSP |
| (2) | SSLSASVGDRVTITCRASQSISSYLNWYQQKPGKAPKWYAASSLQSGVPSRFSGSGSGTDFTLTISSLQ |
| | PEXFATYYCQQVIFPFTFGQGXKLEIKRLGXYKDHDGDYXDHDIXYXDDXXKAAXHHHHHH*SPRWXXX |
| | SPYSXSXYXRSLAXVLXRXXWXNPXXXXXIXXXHIXXXXXXX [SEQ ID NO: 312] |
| DUSP | EVQLLESGGGLVQPGGSLRLSCAASGFTFSSYAMSWVRQAPGKGLEWVSAISGSGGSTYYADSVKGRFT |
| 7 (1) | ISRDNSKNTLYLQMNSLRAEDTAVYYCARGGRGIDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQSYSTPYTFGXXXKLEIKRLXDYXDHDGDYXXHDXDXXMTXXXG |
| | [SEQ ID NO: 313] |
| DUSP | EVQLLESGGGLVQPGGSLRLSCAASGFTFSSYAMSWVRQAPGKGLEWVSAISGSGGSTYYADSVKGRFT |
| 7 (2) | ISRDNSKNTLYLQMNSLRAEDTAVYYCARXXXXXXYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQSYSTPYTFXQGXKLXXXRXGXYXXXXX [SEQ ID NO: 314] |
| FER | EVQLLESGGGLVQPGGSLRLSCAASGFTFGYSYMHWVRQAPGKGLEWVSYISSYGGYTGYADSVKGRFT |
| (1) | ISRDNSKNTLYLQMNSLRAEDTAVYYCARSSVDSVVWYGGYIDYWGQGTLVTVSSGGGGSGGGGSGGGG |
| | SDIQMTQSPSSLSASVGDRVTITCRASQSISSYLNWYQQKPGKAPKLLIYAASSLQSGVPSRFSGSGSG |
| | TDFTLTISSLQPEDFATYYCQQSGFNSPHTFGQGXKLEIKRLGXYKDHDGXYKDHDIDXXDDDDXAXAX |
| | HHHHH*SPRWXSXSPYSESXYXRXLXXXXXXXDWEXXXXXXLXRXXXXPXFXXXXX |
| | [SEQ ID NO: 315] |
| FER | EVQLLESGGG LVQPGGSLRLSCAASGFTFGGYYMYWVRQAPGKGLEWVSGISYSGSYTYYADSVKGRF |
| (2) | TISRDNSKNTLYLQMNSLRAEDTAVYYCARSYGPVYYSSSLDYWGQGTLVTVSSGGGGSGGGGSGGGGS |
| | DIQMTQSPSSLSASVGDRVTITCRASQSISSYLNWYQQKPGKAPKLLIYAASSLQSGVPSRFSGSGSGT |
| | DFTLTISSLQPEDFATYYCQQSYSTPYTFXQGXKLEIKRLGXYKDHDXDYXDHDXXXXDXDXXRP |
| | [SEQ ID NO: 316] |
| GRK5 | EVQLLESGGGLVQPGGSLRLSCAASGFTFYGYGMSWVRQAPGKGLEWVSSISGGGYGTYYADSVKGRFT |
| (1) | ISRDNSKNTLYLQMNSLRAEDTAVYYCARGYSYYSGYFDYWGQGTLVTVSSGGGGSGGGGSGGGGSDIQ |
| | MTQSPSSLSASVGDRVTITCRASQSISSYLNWYQQKPGKAPKLLIYAASSLQSGVPSRFSGSGSGTDFT |
| | LTISSLQPEDFATYYCQQSVGYLSTFGQGXKLEIKRLGXYKDHDGDYKDHDIDYXXDXXKAA |
| | [SEQ ID NO: 317] |
| PRKC | EVQLLESGGGLVQPGGSLRLSCAASGFTFSSYAMSWVRQAPGKGLEWVSAISGSGGSTYYADSVKGRFT |
| Z (1) | ISRDNSKNTLYLQMNSLRAEDTAVYYCARGSHGLDYWGQGTLVTVSSGGGGSGGGGSGGGGSDIQMTQS |
| | PSSLSASVGDRVTITCRASQSISSYLNWYQQKPGKAPKWYAASSLQSGVPSRFSGSGSGTDFTLTIXXL |
| | QPEXFATYYCQQSYSTPYTFGQGXKLXIKRLXXYKDHDGDYKXHDXDXXMTXXRXPPXXXIXIDXXXWX |
| | XXXXYXEXXX [SEQ ID NO: 318] |
| PRKC | EVQLLESGGGLVQPGGSLRLSCAASGFTFSSYAMSWVRQAPGKGLEWVSAISGSGGSTYYADSVKGRFT |
| Z (2) | ISRDNSKNTLYLQMNSLRAEDTAVYYCARGGHGIDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEXFATYYCQQSYSTPYTFGXGTKLEIXRLGXYKXHDGDXKDHDIXYXXDXXXXPPIIIIIXDHXG |
| | XXX [SEQ ID NO: 319] |
| PRKG | EVQLLESGGGLVQPGGSLRLSCAASGFTFGYSGMSWVRQAPGKGLEWVSYIYGSSGYTYYADSVKGRFT |
| 2 (1) | ISRDNSKNTLYLQMNSLRAEDTAVYYCARSGPSYYYPPVYLDYWGQGTLVTVSSGGGGSGGGGSGGGGS |
| | DIQMTQSPSSLSASVGDRVTITCRASQSISSYLNWYQQKPGKAPKLLIYAASSLQSGVPSRFSGSGSGT |
| | DFTLTISSLQPEXFATYYCQQSYSTPYTFGQGTKLEIKRXGXYXDHDGDYKXHDIXXXXDXXXAA |
| | [SEQ ID NO: 320] |
| PRKG | EVQLLESGGGLVQPGGSLRLSCAASGFTFSSSSMGWVRQAPGKGLEWVSYISGYSSSTYYADSVKGRFT |
| 2 (2) | ISRDNSKNTLYLQMNSLRAEDTAVYYCARPTYGPGSARVYIDYWGQGTLVTVSSGGGGSGGGGSGGGGS |
| | DIQMTQSPSSLSASVGDRVTITCRASQSISSYLNWYQQKPGKAPKLLIYAASSLQSGVPSRFSGSGSGT |
| | DFTLTISSLQPEXFATYYCQQSGYPFTFGQGXKLEIKRLGXYKDHDGDYXDHDIDYXDXDXKAA |
| | [SEQ ID NO: 321] |
| PTPR | EVQLLESGGGLVQPGGSLRLSCAASGFTFSSYAMSWVRQAPGKGLEWVSAISGSGGSTYYADSVKGRFT |
| D (1) | ISRDNSKNTLYLQMNSLRAEDTAVYYCARYGFVTYPYGGGYLDYWGQGTLVTVSSGGGGSGGGGSGGGG |
| | SDIQMTQSPSSLSASVGDRVTITCRASQSISSYLNWYQQKPGKAPKLLIYAASSLQSGVPSRFSGSGSG |
| | TDFTLTISSLQPEXFATYYCQQSYSTPYTFXQGXKLEIXRLGDYKXHDGDYKDHDXDXXXXXXXAXAHH |
| | HHHHXSPRXXSXSPXSESXYXRSLAVVXXXXDWENXXVXXXXXXXXXPXXXXXXXXXXX |
| | [SEQ ID NO: 322] |
| PTPR | EVQLLESGGGLVQPGGSLRLSCAASGFTFGYSDMYWVRQAPGKGLEWVSGIGYSGYYTSYADSVKGRFT |
| D (2) | ISRDNSKNTLYLQMNSLRAEDTAVYYCARNASGVYSYAYFDYWGQGTLVTVSSGGGGSGGGGSGGGGSD |
| | IQMTQSPSSLSASVGDRVTITCRASQSISSYLNWYQQKPGKAPKLLIYAASSLQSGVPSRFSGSGSGTD |
| | FTLTISSLQPEXFATYYCQQYSYPFTFGQGTKLEXKRXXDYKDHDXDYXXHDXXXXXXXXXGXXSSSSS |
| | LXTAVXLXFXXXXVXXXXLXXXXFXXX*XXXPXLPXLIXXXXXPFXXXXXXXXXAXXXX |
| | [SEQ ID NO: 323] |
| PTPP | EVQLLESGGGLVQPGGSLRLSCAASGFTFSSYAMSWVRQAPGKGLEWVSAISGSGGSTYYADSVKGRFT |
| RN2 | ISRDNSKNTLYLQMNSLRAEDTAVYYCARGPASSAYFDYWGQGTLVTVSSGGGGSGGGGSGGGGSDIQM |
| (1) | TQSPSSLSASVGDRVTITCRASQSISSYLNWYQQKPGKAPKWYAASSLQSGVPSRFSGSGSGTDFTLTI |
| | SSLCIPEDFATYYCQQSYSTPYTFXQGTKLEIKRLXDYKDHDGDYXDHDXDYKDDXXXAA |
| | [SEQ ID NO: 324] |
| PTPP | EVQLLESGGGLVQPGGSLRLSCAASGFTFSGYSMSWVRQAPGKGLEWVSSISGSYGSTYYADSVKGRFT |
| RN2 | ISRDNSKNTLYLQMNSLRAEDTAVYYCARYGYPYFGMDYWGQGTLVTVSSGGGGSGGGGSGGGGSDIQM |
| (2) | TQSPSSLSASVGDRVTITCRASQSISSYLNWYQQKPGKAPKLLIYAASSLQSGVPSRFSGSGSGTDFTL |
| | TISSLQPEDFATYYCQQSYSTPYTFGQGXKLEIKRLGDYKDHDGXYKDHDIDYKDXXDXAA |
| | [SEQ ID NO: 325] |
| SHC1 | EVQLLESGGGLVQPGGSLRLSCAASGFTFSSYAMSWVRQAPGKGLEWVSYISYYSGYTYYADSVKGRFT |
| (1) | ISRDNSKNTLYLQMNSLRAEDTAVYYCARHYYGGFDYWGQGTLVTVSSGGGGSGGGGSGGGGSDIQMTQ |
| | SPSSLSASVGDRVTITCRASQSISSYLNWYQQKPGKAPKLLIYAASSLQSGVPSRFSGSGSGTDFTLTI |
| | SSLQPEXFATYYCQQGYTLYTFGQGXKLEIKRLGXYKDHDXDYKDHDXXXX*XXXAA |
| | [SEQ ID NO: 326] |
| SHC1 | EVQLLESGGGLVQPGGSLRLSCAASGFTFSSSYMSWVRQAPGKGLEWVSSIGGSGYSTYYADSVKGRFT |
| (2) | ISRDNSKNTLYLQMNSLRAEDTAVYYCARSYHYYIDYWGQGTLVTVSSGGGGSGGGGSGGGGSDIQMTQ |
| | SPSSLSASVGDRVTITCRASQSISSYLNWYQQKPGKAPKWYAASSLQSGVPSRFSGSGSGTDFILTISS |
| | LQPEDFATYYCQQPYFPPTFGXGTXLEXKRLGDYKXHDXDYKXHDIXXXMTMXRRP |
| | [SEQ ID NO: 327] |
| STAP | EVQLLESGGGLVQPGGSLRLSCAASGFTFSGYSMGWVRQAPGKGLEWVSGISSYYYGTSYADSVKGRFT |
| 1 (1) | ISRDNSKNTLYLQMNSLRAEDTAVYYCARSWIVGSSWDGDAFDYWGQGTLVTVSSGGGGSGGGGSGGGG |
| | SDIQMTQSPSSLSASVGDRVTITCRASQSISSYLNWYQQKPGKAPKLLIYAASSLQSGVPSRFSGSGSG |
| | TDFTLTISSLQPEDFATYYCQQSYWYPLTFGQGTKLEIKRLGDYKDHDGDYKDHDIDYKDDDDKAAAHH |
| | HHHH* [SEQ ID NO: 328] |
| STAP | EVQLLESGGGLVQPGGSLRLSCAASGFTFSSYAMSWVRQAPGKGLEWVSAISGSGGSTYYADSVKGRFT |
| 2 (2) | ISRDNSKNTLYLQMNSLRAEDTAVYYCARGGGYFDYWGQGTLVTVSSGGGGSGGGGSGGGGSDIQMTQS |
| | PSSLSASVGDRVTITCRASQSISSYLNWYQQKPGKAPKLLIYAASSLQSGVPSRFSGSGSGTDFTLTIS |
| | SLQPEDFATYYCQQSYSTPYTFGQGTKLEIKRLGDYKDHDGDYXDHDXDXXMXXXRRP |
| | [SEQ ID NO: 329] |

*The structure of the scFv antibodies is described in Söderlind et al., 2000, ‘Recombining germline-derived CDR sequences for creating diverse single-framework antibody libraries’ Nature Biotechnol., 18(8):852-6, which is incorporated herein by reference in its entirety.

TABLE 8

| | | Exemplary |
|---|---|---|
| Antigen | Publication name | SEQ ID NO. |
| Interleukin-4 | IL-4 (2) | 10 |
| Interleukin-13 | IL-13 (2) | 32 |
| Vascular endothelial | VEGF (2) | 35 |
| growth factor | | |
| Lymphotoxin-alpha | TNF-b (2) | 46 |
| Interferon gamma | IFN-γ (3) | 55 or 56 |
| Lewis X | Lewis x (2) | 83 |
| Sialyl Lewis X | Sialyl x | 85 |
| Complement C1q | C1q | 91 |
| Complement C5 | C5 (2) | 97 |
| Plasma protease C1 | C1 inh. (1) | 98 |
| inhibitor | | |
| Properdin | Properdin | 109 |
| Vascular endothelial | VEGF (3) | 112 |
| growth factor | | |
| Interleukin-4 | IL-4 (3) | 114 |
| Intercellular adhesion | ICAM-1 | 121 |
| molecule 1 | | |
| Apolipoprotein A1 | Apo-A1 (2) | 132 |
| Apolipoprotein A1 | Apo-A1 (3) | 133 |
| Plasma protease C1 | C1 inh. (2) | 135 |
| inhibitor | | |
| Plasma protease C1 | C1 inh. (3) | 136 |
| inhibitor | | |
| Complement C4 | C4 (3) | 138 |
| Complement C3 | C3 (3) | 140 |
| Myomesin-2 | MYOM2 (2) | 143 |
| Visual system homeobox 2 | CHX10 (3) | 146 |
| Cyclin-dependent kinase 2 | CDK-2 (2) | 164 |
| HADH2 protein | HADH2 (3) | 171 |
| Protein-tyrosine kinase 6 | PTK6 | 188 |
| Calcineurin B homologous | CHP1 (2) | 210 |
| protein 1 | | |
| Aprataxin and PNK-like | APLF (2) | 261 |
| factor | | |
| Disks large homolog 1 | DLG1-1 (2) | 268 |
| Calcium/calmodulin- | KCC4 (1) | 283 |
| dependent protein kinase | | |
| type IV | | |
| Membrane-associated | MAGI1-1 (1) | 291 |
| guanylate kinase, WW and | | |
| PDZ domain-containing | | |
| protein 1 | | |
| Serine/threonine-protein | MARK1-1 (2) | 292 |
| kinase MARK1 | | |
| PR domain zinc finger | PRDM8-1 (1) | 305 |
| protein 8 | | |
| Protein kinase C zeta type | PRKCZ (2) | 319 |

1. A method for diagnosing or determining a pancreatic cancer-associated disease state comprising or consisting of the steps of:
(a) providing a sample from an individual to be tested; and
(b) measuring the presence and/or amount in the test sample of one or more biomarkers selected from the group defined in Table A;
wherein the presence and/or amount in the test sample of the one or more biomarkers selected from the group defined in Table A is indicative of the pancreatic cancer-associated disease state in the individual.

2. The method according to claim 1 wherein the sample in step (a) is blood or serum.

3. The method according to claim 1 or 2 wherein the sample in step (a) is from a patient in one of the following risk groups:
(a) Individuals with a family history of pancreatic cancer;
(b) Individuals diagnosed with new-onset diabetes type II; or
(c) Individuals with symptoms suggestive or consistent with pancreatic cancer.

4. The method according to any one of the preceding claims wherein step (b) comprises or consists of measuring the presence and/or amount of one or more biomarker(s) listed in Table A, part (i) and/or part (iii).

5. The method according to any one of the preceding claims wherein the method is for:
(i) diagnosis and/or staging of early pancreatic cancer;
(ii) identifying individuals at risk of having or developing pancreatic cancer;
(iii) diagnosis and/or staging of pancreatic cancer;
(iv) differentiating between pancreatic cancer and chronic pancreatitis; and/or
(v) detecting the presence of intraductal papillary mucinous neoplasms.

6. The method according to any one of the preceding claims wherein the pancreatic cancer is pancreatic adenocarcinoma.

7. The method according to any one of the preceding claims wherein step (b) comprises or consists of measuring the presence and/or amount of one or more biomarker(s) listed in Table A, for example at least 2, 3, 4, 5, 6, 7, 8, 9, 10, 11, 12, 13, 14, 15, 16, 17, 18, 19, 20, 21, 22, 23, 24, 25, 26, 27, 28, or all 29 of the biomarkers listed in Table A.

8. The method according to any one of the preceding claims wherein step (b) comprises or consists of measuring the presence and/or amount of.
(i) the biomarkers listed in Table A and Complement C1q (C1q; e.g. Uniprot ID P02745, 2746 and/or 2747);
(ii) the biomarkers listed in Table A, excluding Interleukin-6 (IL-6) and/or GTP-binding protein GEM (GEM); or
(iii) the biomarkers listed in Table A (excluding IL-6 and GEM) and C1q.

9. The method according to any one of the preceding claims wherein step (b) comprises or consists of measuring the presence and/or amount of the following biomarkers:
DLG1, PRKCZ, VEGF, C3, C1INH, IL-4, IFNγ, C5, PTK6, CHP1, APLF, CAMK4, MAGI, MARK1, PRDM8, APOA1, CDK2, HADH2, C4, VSX2/CHX10, ICAM-1, IL-13, Lewis x/CD15, MYOM2, Factor P, Sialyl Lewis x, TNFβ and Complement C1q
(optionally including one or more biomarkers from Table B and/or IL-6 and/or GEM).

10. The method according to any one of the preceding claims wherein step (b) comprises or consists of measuring the presence and/or amount of one or more additional biomarker(s) listed in Table B, for example at least 2, 3, 4, 5, 6, 7, 8, 9, 10, 15, 20, 25, 30, 35, 40, 45, 50, 55, 60, 65, 70,75, 80, 85, 90 or all of the biomarkers in Table B.

11. The method according to any one of the preceding claims wherein the pancreatic cancer-associated disease state is early stage pancreatic cancer.

12. The method according to claim 11 wherein the method is for the diagnosis of stage I and/or stage II pancreatic cancer.

13. The method according to claim 12 wherein step (b) comprises or consists of measuring the presence and/or amount of one or more biomarker listed in:
(i) Table A, part (i), for example both of the biomarkers listed in Table A(i); and/or
(ii) Table A, part (ii), for example at least 2, 3, 4, 5, 6, 7 or all of the biomarkers listed in Table A(ii); and/or
(iii) Table A, part (iii), for example at least 2, 3, 4, 5, 6 or all of the biomarkers listed in Table A(iii); and/or
(iv) Table A, part (iv), for example at least 2, 3, 4, 5, 6, 7, 8, 9, 10, 11, 12 or all of the biomarkers listed in Table A(iv).

14. The method according to claim 12 or 13 wherein step (b) comprises or consists of measuring the presence and/or amount of one or more biomarker listed in Table C, for example at least 2, 3, 4, 5, 6, 7, 8, 9, 10, 11, 12, 13, 14, 15, 16, 17, 18, 19, 20, 21, 22, 23, 24 or all of the biomarkers in Table C.

15. The method according to any one of the preceding claims wherein the pancreatic cancer-associated disease state is late stage pancreatic cancer.

16. The method according to claim 15 wherein the method is for the diagnosis of stage III and/or stage IV pancreatic cancer.

17. The method according to claim 16 wherein step (b) comprises or consists of measuring the presence and/or amount of one or more biomarker listed in:
(i) Table A, part (i), for example both of the biomarkers listed in Table A(i); and/or
(ii) Table A, part (ii), for example at least 2, 3, 4, 5, 6, 7 or all of the biomarkers listed in Table A(ii); and/or
(iii) Table A, part (iii), for example at least 2, 3, 4, 5, 6 or all of the biomarkers listed in Table A(iii); and/or
(iv) Table A, part (iv), for example at least 2, 3, 4, 5, 6, 7, 8, 9, 10, 11, 12 or all of the biomarkers listed in Table A(iv).

18. The method according to claim 16 or 17 wherein step (b) comprises or consists of measuring the presence and/or amount of one or more biomarker listed in Table D, for example at least 2, 3, 4, 5, 6, 7, 8, 9, 10, 11, 12, 13, 14, 15, 16, 17, 18, 19, 20, 21, 22, 23 or all of the biomarkers in Table D.

19. The method according to any one of the preceding claims wherein the method is for differentiating pancreatic cancer from chronic pancreatitis.

20. The method according to claim 19 wherein step (b) comprises or consists of measuring the presence and/or amount of one or more biomarkers listed in:
(i) Table A, part (i), for example both of the biomarkers listed in Table A(i); and/or
(ii) Table A, part (ii), for example at least 2, 3, 4, 5, 6, 7 or all of the biomarkers listed in Table A(ii); and/or
(iii) Table A, part (iii), for example at least 2, 3, 4, 5, 6 or all of the biomarkers listed in Table A(iii); and/or
(iv) Table A, part (iv), for example at least 2, 3, 4, 5, 6, 7, 8, 9, 10, 11, 12 or all of the biomarkers listed in Table A(iv).

21. The method according to claim 19 or 20 wherein step (b) comprises or consists of measuring the presence and/or amount of one or more biomarkers selected from the group consisting of IL-4, C4, MAPK9, C1INH, VEGF, PTPRD, KCC4, TNF-α, C1q and BTK.

22. The method according to any one of the preceding claims wherein the method is for detecting the presence of intraductal papillary mucinous neoplasms, for example malignant IPMNs.

23. The method according to claim 22 wherein step (b) comprises or consists of measuring the presence and/or amount of one or more biomarkers listed in:
(i) Table A, part (i), for example both of the biomarkers listed in Table A(i); and/or
(ii) Table A, part (ii), for example at least 2, 3, 4, 5, 6, 7 or all of the biomarkers listed in Table A(ii); and/or
(iii) Table A, part (iii), for example at least 2, 3, 4, 5, 6 or all of the biomarkers listed in Table A(iii); and/or
(iv) Table A, part (iv), for example at least 2, 3, 4, 5, 6, 7, 8, 9, 10, 11, 12 or all of the biomarkers listed in Table A(iv).

24. The method according to any one of the preceding claims wherein step (b) comprises measuring the presence and/or amount of all of the biomarkers listed in Table A (e.g. at the protein, mRNA and/or ctDNA level).

25. The method according to any one of the preceding claims wherein step (b) comprises measuring the presence and/or amount of DLG1, PRKCZ, VEGF, C3, C1INH, IL-4, IFNγ, C5, PTK6, CHP1, APLF, CAMK4, MAGI, MARK1, PRDM8, APOA1, CDK2, HADH2, C4, VSX2/CHX10, ICAM-1, IL-13, Lewis x/CD15, MYOM2, Factor P, Sialyl Lewis x, TNFβ and Complement C1q.

26. The method according to any one of the preceding claims further comprising or consisting of the steps of:
(c) providing one or more control samples from:
i. an individual not afflicted with pancreatic cancer; and/or
ii. an individual afflicted with pancreatic cancer, wherein the sample was of a different stage to that of the test sample; and/or
iii. an individual afflicted with chronic pancreatitis; and
(d) determining a biomarker signature of the one or more control samples by measuring the presence and/or amount in the control sample of the one or more biomarkers measured in step (b);
wherein the pancreatic cancer-associated disease state is identified in the event that the presence and/or amount in the test sample of the one or more biomarkers measured in step (b) is different from the presence and/or amount in the control sample of the one or more biomarkers measured in step (d).

27. The method according to any one of the preceding claims further comprising or consisting of the steps of:
(e) providing one or more control samples from;
i. an individual afflicted with pancreatic cancer; and/or
ii. an individual afflicted with pancreatic cancer, wherein the sample was of the same stage to that of that the test sample;
(f) determining a biomarker signature of the control sample by measuring the presence and/or amount in the control sample of the one or more biomarkers measured in step (b);
wherein the pancreatic cancer-associated disease state is identified in the event that the presence and/or amount in the test sample of the one or more biomarkers measured in step (b) corresponds to the presence and/or amount in the control sample of the one or more biomarkers measured in step (f).

28. The method according to claim 26 wherein the individual not afflicted with pancreatic cancer is a healthy individual.

29. The method according to claim 26 or 27 wherein the one or more individual afflicted with pancreatic cancer is afflicted with a pancreatic cancer selected from the group consisting of adenocarcinoma (e.g., pancreatic ductal adenocarcinoma or tubular papillary pancreatic adenocarcinoma), pancreatic sarcoma, malignant serous cystadenoma, adenosquamous carcinoma, signet ring cell carcinoma, hepatoid carcinoma, colloid carcinoma, undifferentiated carcinoma, and undifferentiated carcinomas with osteoclast-like giant cells.

30. The method according to any one of the preceding claims wherein the pancreatic cancer is pancreatic ductal adenocarcinoma.

31. The method according to any one of the preceding claims wherein the method is repeated.

32. The method according to claim 31 wherein the method is repeated using a test sample taken from the same individual at a different time period to the previous test sample(s) used.

33. The method according to claim 32 wherein the method is repeated using a test sample taken between 1 day to 104 weeks to the previous test sample(s) used, for example, between 1 week to 100 weeks, 1 week to 90 weeks, 1 week to 80 weeks, 1 week to 70 weeks, 1 week to 60 weeks, 1 week to 50 weeks, 1 week to 40 weeks, 1 week to 30 weeks, 1 week to 20 weeks, 1 week to 10 weeks, 1 week to 9 weeks, 1 week to 8 weeks, 1 week to 7 weeks, 1 week to 6 weeks, 1 week to 5 weeks, 1 week to 4 weeks, 1 week to 3 weeks, or 1 week to 2 weeks.

34. The method according to claim 32 or 33 wherein the method is repeated using a test sample taken every period from the group consisting of: 1 day, 2 days, 3 day, 4 days, 5 days, 6 days, 7 days, 10 days, 2 weeks, 3 weeks, 4 weeks, 5 weeks, 6 weeks, 7 weeks, 8 weeks, 9 weeks, 10 weeks, 15 weeks, 20 weeks, 25 weeks, 30 weeks, 35 weeks, 40 weeks, 45 weeks, 50 weeks, 55 weeks, 60 weeks, 65 weeks, 70 weeks, 75 weeks, 80 weeks, 85 weeks, 90 weeks, 95 weeks, 100 weeks, 104, weeks, 105 weeks, 110 weeks, 115 weeks, 120 weeks, 125 weeks and 130 weeks.

35. The method according to any one of claims 32 to 34 wherein the method is repeated at least once, for example, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23, 24 times or 25 times.

36. The method according to any one of claims 32 to 35 wherein the method is repeated until pancreatic cancer is diagnosed in the individual using conventional clinical methods.

37. The method according to any one of the preceding claims wherein step (b) comprises measuring the expression of the protein or polypeptide of the one or more biomarker(s).

38. The method according to claim 37 wherein step (b), (d) and/or step (f) is performed using one or more first binding agent capable of binding to a biomarker protein or polypeptide listed in Table A.

39. The method according to claim 38 wherein the first binding agent comprises or consists of an antibody or an antigen-binding fragment thereof.

40. The method according to claim 39 wherein the antibody or antigen-binding fragment thereof is a recombinant antibody or antigen-binding fragment thereof.

41. The method according to claim 39 or 40 wherein the antibody or antigen-binding fragment thereof is selected from the group consisting of: scFv; Fab; a binding domain of an immunoglobulin molecule.

42. The method according to any one of claims 38 to 41 wherein the first binding agent is immobilised on a surface.

43. The method according to any one of claims 27 to 42 wherein the one or more biomarkers in the test and/or control sample(s) are labelled with a detectable moiety.

44. The method according to claim 43 wherein the detectable moiety is selected from the group consisting of: a fluorescent moiety; a luminescent moiety; a chemiluminescent moiety; a radioactive moiety; an enzymatic moiety.

45. The method according to claim 43 or 44 wherein the detectable moiety is biotin.

46. The method according to any one of claims 41 to 45 wherein step (b), (d) and/or step (f) is performed using an assay comprising a second binding agent capable of binding to the one or more biomarkers, the second binding agent comprising a detectable moiety.

47. The method according to claim 46 wherein the second binding agent comprises or consists of an antibody or an antigen-binding fragment thereof.

48. The method according to claim 47 wherein the antibody or antigen-binding fragment thereof is a recombinant antibody or antigen-binding fragment thereof.

49. The method according to claim 47 or 48 wherein the antibody or antigen-binding fragment thereof is selected from the group consisting of: scFv; Fab; a binding domain of an immunoglobulin molecule.

50. The method according to any one of claims 46 to 49 wherein the detectable moiety is selected from the group consisting of: a fluorescent moiety; a luminescent moiety; a chemiluminescent moiety; a radioactive moiety; an enzymatic moiety.

51. The method according to claim 50 wherein the detectable moiety is fluorescent moiety (for example an Alexa Fluor dye, e.g. Alexa647).

52. The method according to any one of the preceding claims wherein the method comprises or consists of an ELISA (Enzyme Linked Immunosorbent Assay).

53. The method according to any one of the preceding claims wherein step (b), (d) and/or step (f) is performed using an array.

54. The method according to claim 53 wherein the array is selected from the group consisting of: macroarray; microarray; nanoarray.

55. The method according to any one of claims 37 to 54 wherein the method comprises:
(i) labelling biomarkers present in the sample with biotin;
(ii) contacting the biotin-labelled proteins with an array comprising a plurality of scFv immobilised at discrete locations on its surface, the scFv having specificity for one or more of the proteins in Table A;
(iii) contacting the biotin-labelled proteins (immobilised on the scFv) with a streptavidin conjugate comprising a fluorescent dye; and
(iv) detecting the presence of the dye at discrete locations on the array surface wherein the expression of the dye on the array surface is indicative of the expression of a biomarker from Table A in the sample.

56. The method according to any one of claims 1 to 36 wherein step (b), (d) and/or (f) comprises measuring the expression of a nucleic acid molecule encoding the one or more biomarkers.

57. The method according to claim 56, wherein the nucleic acid molecule an mRNA molecule.

58. The method according to claim 56, wherein the nucleic acid molecule a DNA molecule.

59. The method according to claim 58, wherein the nucleic acid molecule a cDNA or ctDNA molecule.

60. The method according to any one of claims 56 to 59, wherein measuring the expression of the one or more biomarker(s) in step (b), (d) and/or (f) is performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation.

61. The method according to any one of claims 56 to 60, wherein measuring the expression of the one or more biomarker(s) in step (b) is determined using a DNA microarray.

62. The method according to any one of claims 56 to 61, wherein measuring the expression of the one or more biomarker(s) in step (b), (d) and/or (f) is performed using one or more binding moieties, each individually capable of binding selectively to a nucleic acid molecule encoding one of the biomarkers identified in Table A.

63. The method according to claim 62, wherein the one or more binding moieties each comprise or consist of a nucleic acid molecule.

64. The method according to claim 63 wherein, the one or more binding moieties each comprise or consist of DNA, RNA, PNA, LNA, GNA, TNA or PMO.

65. The method according to claim 63 or 64, wherein the one or more binding moieties each comprise or consist of DNA.

66. The method according to any one of claims 63 to 65 wherein the one or more binding moieties are 5 to 100 nucleotides in length, for example 15 to 35 nucleotides in length.

67. The method according to any one of claims 63 to 66 wherein the binding moiety comprises a detectable moiety.

68. The method according to claim 67 wherein the detectable moiety is selected from the group consisting of: a fluorescent moiety; a luminescent moiety; a chemiluminescent moiety; a radioactive moiety (for example, a radioactive atom); or an enzymatic moiety.

69. The method according to claim 68 wherein the detectable moiety comprises or consists of a radioactive atom.

70. The method according to claim 69 wherein the radioactive atom is selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

71. The method according to claim 68 wherein the detectable moiety of the binding moiety is a fluorescent moiety.

72. The method according to any one of the preceding claims wherein the sample provided in step (a), (c) and/or (e) is selected from the group consisting of unfractionated blood, plasma, serum, tissue fluid, pancreatic tissue, milk, bile and urine.

73. The method according to claim 72, wherein the sample provided in step (a), (c) and/or (e) is selected from the group consisting of unfractionated blood, plasma and serum.

74. The method according to claim 72 or 73, wherein the sample provided in step (a), (c) and/or (e) is serum.

75. The method according to any one of the preceding claims wherein the predictive accuracy of the method, as determined by an ROC AUC value, is at least 0.50, for example at least 0.55, 0.60, 0.65, 0.70, 0.75, 0.80, 0.85, 0.90, 0.95, 0.96, 0.97, 0.98 or at least 0.99.

76. The method according to claim 75 wherein the predictive accuracy of the method, as determined by an ROC AUC value, is at least 0.70.

77. The method according to any one of the preceding claims further comprising one or more further clinical investigations (such as testing a biopsy sample and/or in vivo imaging of the patient) in order to confirm or establish the diagnosis.

78. The method according to any one of the preceding claims wherein, in the event that the individual is diagnosed with pancreatic cancer, the method comprises step (g) of providing the individual with a pancreatic cancer therapy.

79. The method according to claim 78 wherein the pancreatic cancer therapy is selected from the group consisting of surgery, chemotherapy, radiotherapy, immunotherapy, chemoimmunotherapy, thermochemotherapy and combinations thereof.

80. The method according to claim 78 or 79 wherein the pancreatic cancer therapy comprises or consists of surgical removal of the pancreas in whole or in part (flor example, using the Whipple procedure to remove the pancreas head or a total pancreatectomy) combined with chemotherapy (for example, gemcitabine and/or 5-fluorouracil).

81. An array for determining the presence of, or risk of having, pancreatic cancer in an individual comprising an agent or agents for detecting the presence in a protein and/or nucleic acid sample from the individual of one or more of the biomarkers defined in Table A.

82. The array according to claim 81 wherein the agent or agents for detecting the presence in a sample of one or more of the biomarkers defined in Table A is/are one or more binding agents as defined in any one of claims 39 to 42 or 63 to 71.

83. The array according to claim 81 or 82 wherein the array comprises agents capable of binding to all of the biomarkers defined in Table A.

84. The array according to claim 81 or 82 wherein the array comprises agents capable of binding to the following biomarkers;
DLG1, PRKCZ, VEGF, C3, C1INH, IL-4, IFNγ, C5, PTK6, CHP1, APLF, CAMK4, MAGI, MARK1, PRDM8, APOA1, CDK2, HADH2, C4, VSX2/CHX10, ICAM-1, IL-13, Lewis x/CD15, MYOM2, Factor P, Sialyl Lewis x, TNFβ and Complement C1q
(optionally including one or more biomarkers from Table B and/or IL-6 and/or GEM).

85. The array according to any one of claims 81 to 84 wherein the array comprises antibodies, or antigen-binding fragments thereof, capable of binding to all of the biomarkers at the protein level.

86. The array according to claim 85 wherein the array comprises one or more of the antibodies identified in Table 7.

87. The array according to claim 85 wherein the array comprises or consists of all of the antibodies in Table 8.

88. The array according to any one of claims 81 to 84 wherein the array comprises agents capable of binding to all of the biomarkers at the mRNA and/or DNA level.

89. The array according to any one of claims 81 to 88 further comprising a positive control sample (such as bovine serum albumin).

90. The array according to any one of claims 81 to 89 further comprising a negative control sample (such as phosphate-buffered saline).

91. Use of one or more biomarkers selected from the group defined in Table A as biomarkers for determining the presence of, or risk of having, pancreatic cancer in an individual.

92. The use according to claim 91 wherein the one or more biomarkers comprise(s) the following biomarkers:
DLG1, PRKCZ, VEGF, C3, C1INH, IL-4, IFNγ, C5, PTK6, CHP1, APLF, CAMK4, MAGI, MARK1, PRDM8, APOA1, CDK2, HADH2, C4, VSX2/CHX10, ICAM-1, IL-13, Lewis x/CD15, MYOM2, Factor P, Sialyl Lewis x, TNFβ and Complement C1q
(optionally including one or more biomarkers from Table B plus IL-6 and GEM).

93. The use according to claim 91 or 92 wherein all of the biomarkers defined in Table A are used together as a diagnostic signature for determining the presence of pancreatic cancer in an individual.

94. A kit for determining the presence of, or risk of having, pancreatic cancer comprising:
(a) an array according to any one of claims 81 to 90, or components for making the same; and
(b) instructions for performing the method as defined in any one of claims 1 to 80.

95. A method of treating pancreatic cancer in an individual comprising the steps of:
(a) diagnosing pancreatic cancer according to the method defined in any one of claims 1 to 80; and
(b) providing the individual with pancreatic cancer therapy.

96. The method according to claim 95 wherein step (a) further comprises comprise one or more further clinical investigations (such as testing a biopsy sample and/or in vivo imaging of the patient) in order to confirm or establish the diagnosis.

97. The method according to claim 95 or 96 wherein the pancreatic cancer therapy is selected from the group consisting of surgery (e.g., resection), chemotherapy, immunotherapy, chemoimmunotherapy and thermochemotherapy.

98. The method of any one of claims 95 to 97 wherein the pancreatic cancer therapy comprises surgical removal of the pancreas in whole or in part (e.g. using the Whipple procedure to remove the pancreas head or a total pancreatectomy) combined with chemotherapy (e.g. gemcitabine and/or 5-fluorouracil).

99. A method or use for determining the presence of pancreatic cancer in an individual substantially as described herein.

100. An array or kit for determining the presence of pancreatic cancer in an individual substantially as described herein.
